(19) United States
(12) Patent Application Publication
(10) Pub. No.: US 2026/0174751 A1
(43) Pub. Date: Jun. 25, 2026

(54) METHOD OF IMPROVING SKELETAL MUSCLE FUNCTION

(71) Applicant: Ascendis Pharma Growth Disorders A/S, Hellerup (DK)

(72) Inventors: Bent Winding, Hellerup (DK); Kennett Sprogøe, Hellerup (DK); Mads Jens Kjelgaard-Hansen, Hellerup (DK)

(21) Appl. No.: 19/125,690

(22) PCT Filed: Nov. 10, 2023
(86) PCT No.: PCT/EP2023/081497

§ 371 Date: Apr. 29, 2025

Related U.S. Application Data
(63) Continuation-in-part of application No. PCT/EP2022/085414, filed on Dec. 12, 2022.

(30) Foreign Application Priority Data
Dec. 13, 2021 (EP) 21214054.5
Nov. 14, 2022 (EP) 22207210.0
Dec. 12, 2022 (WO) PCT/EP2022/085414
Mar. 20, 2023 (EP) 23162994.0
Jun. 21, 2023 (EP) 23180740.5

(51) Int. Cl. A61K 31/506; A61K 9/00; A61K 47/60

(52) U.S. Cl. CPC A61K 31/506; A61K 9/0019; A61K 47/60; A61K 2300/00

(57) ABSTRACT

The present invention relates to methods of improving muscle function in a subject suffering from a disease or condition in which muscle function is impaired, the method comprising administering an effective amount of, an inhibitor of FGFR3 signaling, an NPR-B agonist or an NPR-C agonist to said subject.

### FIELD OF INVENTION

The present invention relates to methods for improving muscle function in a subject in need thereof, particularly for therapeutic use in conditions associated with a loss of muscle strength, muscle condition, and/or muscle pain.

### BACKGROUND OF THE INVENTION

Achondroplasia is the most common form of non-lethal skeletal dysplasia, affecting more than 250,000 people worldwide. Achondroplasia is caused by a pathogenic variant in the gene that codes for fibroblast growth factor receptor 3 (FGFR3) and is transmitted as an autosomal dominant trait. FGFR3 is a negative regulator of long bone development and is highly expressed in the growth plate chondrocytes.

Infants with achondroplasia typically have weak muscle tone (hypotonia) which results in delayed motor skill development, and weak skeletal muscles and contributes to spinal deformities such as kyphosis (Takken et al., Journal of Pediatrics 2017, Vol 150, pp 26-30; Patient Guide to Achondroplasia, John Hopkins Dept. Orthapaedic Surgery, 2003). Muscle weakness is also a characteristic of achondroplasia in adults (Sims et al., J Appl Physiol (1985) 2018 Mar. 1; 124(3):696-703; de Vries Am J Med Genet. 2021; 185A:1023-1032).

Spinal deformities in people with achondroplasia require treatment during infancy, childhood, and adulthood: Infants may require a brace or body cast, and corrective surgery may be required during childhood. In adulthood, kyphosis can lead to spinal stenosis, and surgery is often required to straighten the spine and allow bones of the spine to grow together.

Inhibition of FGFR3 signaling has been a focus for the development of therapeutic interventions for treatment of achondroplasia. FGFR3 inhibitors may act on the receptor itself or on its the downstream signalling pathways in order to inhibit FGFR3 signalling. Suitable examples of FGFR3 inhibitors therefore include tyrosine kinase inhibitors (such as infigratinib, pemigatinib, futibatinib, erdafitinib or TYRA-300), C-type natriuretic peptide (CNP), FGFR3 siRNA and FGFR3 antisense oligonucleotides.

Infigratinib is a FGFR selective tyrosine kinase inhibitor which has been approved for the treatment of certain cancers (Truseltiq), and is in clinical development for the increasing height in children with achondroplasia (Savarirayan et al., Ther Adv Musculoskelet Dis 2022 Mar. 21; 14).

TYRA-300 is an oral, potent FGFR 3-selective tyrosine kinase inhibitor that has activity in the presence of mutations including the FGFR3 V555 mutation, and which has selectivity for FGFR3 over FGF1 and other FGFR isoforms. TYRA-300 was developed by Tyra Biosciences.

FGFR3 signals through several intracellular pathways, including signal transducer and activator of transcription (STAT) and mitogen-activated protein kinase (MAPK) and constitutive activation of FGFR3 is associated with the achondroplasia phenotype. FGFR3 activation is associated with increased phosphorylation of the STAT and MAPK pathways. The MAPK pathway can be regulated by C-type natriuretic peptide (CNP). CNP binding to its receptor, natriuretic-peptide receptor B (NPR-B), gives rise to cGMP production which activates different signaling mediators, including cyclic nucleotide phosphodiesterases (PDEs), cGMP-regulated ion channels (cGICs), and cGMP-dependent protein kinases (cGKI and cGKII). The CNP/NPR-B effect on FGFR3 is mediated by activation of cGKII, which inhibits the activation of RAF-1, and consequently, the activation of MEK1/2 and ERK1/2 in the MAPK pathway. An example of an FGFR3 inhibitor that acts downstream of FGFR3 includes molecules that activate the NPR-B receptor, as activation of this receptor inhibits FGFR3 downstream signaling by inhibiting the pathway of mitogen-activated protein kinase (MAPK).

Vosoritide is a C-type natriuretic peptide (CNP) variant which has been approved for the treatment of achondroplasia in pediatric patients whose bones are still growing, and works directly on the growth plates of bones to promote new bone growth (https://www.ema.europa.eu/en/medicines/human/EPAR/voxzogo).

TransCon™ CNP is an investigational long acting CNP prodrug which is in clinical development for treatment of achondroplasia in pediatric patients with open bone epiphysis (Breinholt et al., Br J Clin Pharmacol. 2022 November; 88(11):4763-4772). In a phase 2 clinical trial (ACcomplisH), TransCon™ CNP demonstrated superiority compared to placebo in change in annualized ACH-specific height SDS, and a reduction in achondroplasia-related adverse events was reported.

CNP is a potent regulator of growth plate chondrogenesis, and CNP binding to the natriuretic peptide binding receptor-2 (NPR-B) on the chondrocyte cell surface induces intracellular synthesis of cyclic guanosine monophosphate (cGMP) and activating cGMP-dependent signal transduction (Potter et al., Handb Exp Pharmacol. 2009; (191): 341-366). CNP is indicated in cardiac remodeling and acute myocardial infarction, relaxation of smooth muscle and treatment of hypertension (Nakagawa and Saito, Biology 2022, 11, 1017). Perez-Ternero et al., PNAS 2022, vol 119/13 e2116470119, reports that C-type natriuretic peptide is a pivotal regulator of metabolic homeostasis. CNP exerts these metabolic regulatory actions by inhibiting sympathetic thermogenic programming via Gi-coupled natriuretic peptide receptor (NPR)-C and reducing peroxisome proliferator-activated receptor-γ coactivator-1α expression, while concomitantly driving adipogenesis via NPR-B/protein kinase-G. Perez-Ternero et al., Front. Mol. Neurosci. 15:991112. doi: 10.3389/fnmol.2022.991112 reports from CNP deletion mice that CNP has a role in regulation of the blood brain barrier and modulation of locomotor reactivity to novel environments, and notably that CNP deletion in gbCNP−/− mice results in reduced body weight, but unchanged muscle mass.

Another example of an FGFR3 inhibitor which acts on the pathway downstream of FGFR3 itself is the molecule Meclizine which attenuates the MAPK signalling pathway at the level of ERK phosphorylation (see e.g. Kitoh et al 2020. PLoS ONE 15(4): e0229639. https://doi.org/10.1371/journal.pone.0229639).

Mackler et al., 1973 (Arch. Biochem and Biophys 159, 885-888) reports the oxidative energy production is decreased in adult achondroplasia (ACH). Domondon et al., Am J Physiol Renal Physiol 2019 317: F1164-F1168, reviews the regulation of mitochondrial function by natriuretic peptides, highlighting the role of ANP and BNP/NPR-A activation in mitochondria. Miyashita et al., Diabetes 2009 58, 2880-2892 reported that BNP/cGK cascades can promote muscle mitochondrial biogenesis and fat oxidation and prevent obesity BNP-TG mice. In contrast, Perez-Ternero et al., PNAS 2022 Vol. 119 No. 13 e2116470119 reports that CNP has a preservative function in coordinating metabolic homeostasis, inhibiting sympathetic thermogenic programming via NPR-C, whilst concomitantly driving adipogenesis via NPR-B/protein kinase-G, indicating that CNP has an opposing action to NPR-A ANP/BNP activation. PGC-1alpha is a a key regulator of mitochondrial biogenesis, and as reported in Domondon 2018, PGC-1alpha is positively regulated via ANP and BNP.

### STATEMENT OF THE INVENTION

Treatment of achondroplasia has been focused on promoting endochondrial bone growth and therapeutics have been selected for their ability to improve annualized growth/height velocity of achondroplasia in children. The inventors of the present invention have surprisingly found that treatment of children with achondroplasia with a CNP drug, TransCon CNP, resulted in an improvement of physical function, as well as a reduction of achondroplasia disease related adverse events. Based on the studies reported below, these improvements in physical function are believed to result from improvements in muscular function, independent of bone growth. Children on placebo showed deteriorated physical performance and a much higher incidence of achondroplasia disease related adverse events. Furthermore, in an achondroplasia mouse model, treatment with TransCon CNP was shown to increase survival of pups within the first 15 days of life. This finding correlated with a reduced incidence of material infanticide, a phenomenon associated with muscle weakness in pups, and occurred over a time frame that is inconsistent with effects relating to bone growth. These findings, that CNP, an inhibitor of FGFR3 signalling (as discussed above CNP inhibits FGFR3 mediated signalling), is effective in enhancing physical performance and muscle function, independent of the enhanced endochondrial bone growth illustrates that CNP and other FGFR3 inhibition strategies are suitable therapeutics for the improvement of muscle function, for example in the treatment of hypotonia and related disorders, both in children and in adults. The binding of CNP to the NPR-B receptor may contribute to the mechanism of action and as such NPR-B receptor agonists are also contemplated for this use. The binding of CNP to the NPR-C receptor may contribute to the mechanism of action and as such NPR-C receptor agonists are also contemplated for this use.

The invention provides for a method of improving muscle function in a subject suffering from a disease or condition in which muscle function is impaired, the method comprising administering a therapeutically effective amount of an inhibitor of FGFR3 signaling to said subject.

The invention provides for a method of improving muscle function in a subject suffering from a disease or condition in which muscle function is impaired, the method comprising administering a therapeutically effective amount of an NPR-B agonist to said subject.

The invention provides for a method of improving muscle function in a subject suffering from a disease or condition in which muscle function is impaired, the method comprising administering a therapeutically effective amount of an an NPR-C agonist to said subject.

The invention provides for a method of improving muscle function in a subject suffering from a disease or condition in which muscle function is impaired, the method comprising administering a therapeutically effective amount of an inhibitor of FGFR3 signaling, an NPR-B agonist or an NPR-C agonist to said subject.

The invention provides for a method of improving muscle function in a subject suffering from a disease or condition in which muscle function is impaired, the method comprising administering a therapeutically effective amount of a C-type natriuretic peptide (CNP) to said subject. In certain embodiments the NPR-B agonist or CNP is vosoritide (SEQ ID NO:30).

The invention provides for a method of improving muscle function in a subject suffering from a disease or condition in which muscle function is impaired, the method comprising administering a therapeutically effective amount of a C-type natriuretic peptide (CNP) to said subject, wherein the CNP is administered as a CNP conjugate and/or as a prodrug of CNP; or a pharmaceutically acceptable salt thereof.

The invention provides for a method of improving muscle function in a subject suffering from a disease or condition in which muscle function is impaired, the method comprising administering a therapeutically effective amount of a C-type natriuretic peptide (CNP) to said subject, wherein the CNP is administered in as a CNP conjugate and/or as a prodrug of CNP; or a pharmaceutically acceptable salt thereof; wherein said method comprises administering successive doses of a therapeutically effective amount of the CNP conjugate and/or prodrug of CNP to the subject, wherein the sustained exposure of free CNP in the blood plasma of the patient between successive doses of the therapeutically effective amount of the CNP conjugate and/or prodrug of CNP is at least about 1 pmol/L. In certain embodiments the successive doses are administered, for example daily or weekly or biweekly, or monthly. In certain embodiments time interval between successive doses is at least about 24 hours, such as at least about one week. In certain embodiments the CNP conjugate or prodrug of CNP is a compound of formula (IIf′), formula (IIf), compound (1), or a pharmaceutically acceptable salt thereof.

The invention provides for a method of improving muscle function in a subject suffering from a disease or condition in which muscle function is impaired, the method comprising administering a therapeutically effective amount of a C-type natriuretic peptide (CNP) to said subject, wherein the CNP is administered in as a CNP conjugate and/or as a prodrug of CNP; or a pharmaceutically acceptable salt thereof, wherein the CNP conjugate or prodrug of CNP is a compound of formula (IIf′), formula (IIf), compound (1), or a pharmaceutically acceptable salt thereof.

In certain embodiments, the subject is a human subject. In certain embodiments, the subject is a human subject less than 18 years of age. In certain embodiments, the subject is a human subject at least 18 years of age. In certain embodiments, the subject has closed bone epiphysis.

The invention provides for a method of improving muscle function in a subject suffering from a disease or condition in which muscle function is impaired, the method comprising administering a therapeutically effective amount of a C-type natriuretic peptide (CNP) to said subject, wherein said method comprises an infusion, such as a i.v. or s.c. infusion, of the CNP to the subject, wherein said infusion results in a sustained exposure of CNP (free CNP) in the blood plasma of the patient over a period of at least about 1 hour of at least 1 pmol/L. In certain embodiments the sustained exposure of CNP (free CNP) in the blood plasma of the patient over a period of at least about 2 hours, such as at least about 4 hours, such as at least about 6 hours.

The invention provides for a method of improving muscle function in a subject suffering from a disease or condition in which muscle function is impaired, the method comprising administering a therapeutically effective amount of an inhibitor of FGFR3 signaling to said subject, wherein the inhibitor of FGFR3 signaling (a FGFR3 antagonist) is an FGFR3 tyrosine kinase inhibitor, such as a FGFR3 tyrosine kinase inhibitor is selected from the group consisting of infigratinib, pemigatinib, futibatinib, erdafitinib and TYRA-300.

The invention provides for a method (such as a therapeutic method) for improving muscle function in a subject.

The invention provides for a method (such as a therapeutic method) for improving skeletal muscle function in a subject.

The invention provides for a method (such as a therapeutic method) for improving muscle strength and/or improving muscle stamina in a subject (e.g. skeletal muscle strength and/or skeletal muscle stamina).

The invention provides for a method (such as a therapeutic method) for improving muscle tonicity in a subject (e.g. skeletal muscle tonicity).

In each case the method comprises administering an effective amount of an inhibitor of FGFR3 signalling or an effective amount of an NPR-C agonist or an effective amount of an NPR-B agonist to said subject. In certain embodiments, the method comprises administering an effective amount of an NPR-B/NPR-C agonist (e.g a CNP).

The subject may be suffering from a disease or a disorder which causes an impairment of muscle function (e.g. muscle strength, stamina and/or tonicity). The disease or a disorder may be one which causes an impairment of skeletal muscle function.

Optionally, the inhibitors of FGFR3 signalling or NPR-B agonists or NPR-C agonists, or CNP, such as CNP conjugate or prodrug of CNP or pharmaceutically acceptable salt thereof, may be used in combination with a growth hormone, such as human growth hormone.

The invention provides an inhibitor of FGFR3 signalling or NPR-B agonists or an NPR-C agonist for use in a method of improving muscle function in a subject, optionally where the subject is suffering from a disease or a disorder which causes an impairment in muscle function.

The invention provides an inhibitor of FGFR3 signalling or NPR-B agonists or an NPR-C agonist for use in a method of improving skeletal muscle function in a subject, optionally wherein the subject is suffering from a disease or a disorder which causes an impairment in skeletal muscle function.

In certain embodiments the subject has a chondroplasia disease, such as a disease selected from the group consisting of achondroplasia, hypochondroplasia, and thanatophoric dysplasia. The subject is preferably a human subject. The subject may be less than 18 years of age or at least 18 years of age. The subject may have closed bone epiphysis.

In certain embodiments the inhibitor of FGFR3 signalling is an FGFR3 antagonist or an NPR-B agonist.

In certain embodiments NPR-C agonists may be used in the methods or uses of the invention.

FGFR3 antagonists may include a fibroblast growth factor receptor (FGFR) 3 tyrosine kinase inhibitor, an anti-FGFR3 antibody, an anti-FGFR3 antisense oligonucleotide or an anti-FGFR3 siRNA. A suitable NPR-B agonist is a C-type natriuretic peptide, or conjugate or prodrug thereof. A suitable NPR-C agonist is a C-type natriuretic peptide, or conjugate or prodrug thereof.

Examples of FGFR3 antagonists include fibroblast growth factor receptor (FGFR) 3 tyrosine kinase inhibitor, such as infigratinib, pemigatinib, futibatinib, erdafitinib or TYRA-300. WO2022147246A1 discloses such FGFR3 antagonists, which is hereby incorporated by reference in its entirety.

The invention provides a method of improving muscle function (e.g. skeletal muscle function) in a subject suffering from a disease or condition in which muscle function (e.g skeletal muscle function) is impaired, said method comprising administering a therapeutically effective amount of a CNP.

The invention provides a method of improving muscle function (e.g skeletal muscle function) in a subject suffering from a disease or condition in which muscle function (e.g skeletal muscle function) is impaired, said method comprising administering a therapeutically effective amount of vosoritide.

In certain embodiments, the CNP comprises or consists of SEQ ID NO: 24. In certain embodiments, the CNP comprises or consists of a peptide selected from the group consisting of SEQ ID NO:30, SEQ ID NO:98, SEQ ID NO:99, SEQ ID NO:100, SEQ ID NO:101, SEQ ID NO:102, SEQ ID NO:103, SEQ ID NO:104, SEQ ID NO: 105 and SEQ ID NO:90. In some embodiments, the CNP is a CNP conjugate or pharmaceutically acceptable salt thereof, comprising a CNP selected from the group consisting of SEQ ID NO:30, SEQ ID NO:98, SEQ ID NO:99, SEQ ID NO:100, SEQ ID NO:101, SEQ ID NO:102, SEQ ID NO: 103, SEQ ID NO:104 or SEQ ID NO: 105 and SEQ ID NO:90.

The improvement in muscle function can be for example, a) increased muscle (e.g. skeletal muscle) strength, b) increased muscle (e.g. skeletal muscle) tone, c) increased muscle (e.g. skeletal muscle) stamina, d) increased muscle (e.g. skeletal muscle) mass, e) decreased muscle (e.g. skeletal muscle) fatigue, f) increased cardiovascular endurance, g) increased cardiovascular fitness, h) decreased exercise intolerance, i) increased exercise capacity, j) decreased exercise induced fatigue, or k) increased hypertonia.

The administration of the effective amount of the inhibitor of FGFR3 signalling, or the effective amount of the NPR-B agonist, or the effective amount of the NPR-C agonist may give rise to increased muscle (e.g. skeletal muscle) mass and/or skeletal muscle/fat ratio in the subject.

The administration of the effective amount of the inhibitor of FGFR3 signalling, or the effective amount of the NPR-B agonist, or the effective amount of the NPR-C agonist may give rise to treatment or prevention of musculo-skeletal pain in the subject, an improvement in posture or a reduction in an abnormal curvature of the spine, an improvement in kyphosis, lordosis, spinal stenosis or scoliosis, improvement in sleep apnea, obstructive sleep apnea, otitis media, or a reduction in obesity. These improvements may arise as a result of the improvement(s) in muscle function (e.g. skeletal muscle function). Thus, the treatment, prevention or improvement in any condition referred to above is preferably by improvement of muscle function (e.g. skeletal muscle function).

In certain embodiments the CNP is administered as a prodrug. In certain embodiments, the CNP prodrug provides a sustained exposure of active CNP peptide after administration to the subject.

The prodrug is optionally a compound of formula (IIf′) or formula (IIf) or compound (1), or a pharmaceutically acceptable salt thereof.

In some embodiments, wherein the subject is an adult human being (i.e. at least 18 years of age), the subject may have been treated with the inhibitor of FGFR3 signalling, or NPR-B agonist (e.g. CNP) or NPR-C agonist before the age of 18. The invention therefore provides for a therapeutic treatment which is initiated before the age of 18 years and is continued into beyond 18 years of age.

Administration of the the inhibitor of FGFR3 signalling, or NPR-B agonist, or NPR-C agonist may give rise to one or more of improvement in muscle function, selected from the group consisting of:
a) increased skeletal muscle strength,
b) increased skeletal muscle tone,
c) increased skeletal muscle stamina,
d) increased skeletal muscle mass,
e) decreased skeletal muscle fatigue,
f) increased cardiovascular endurance,
g) increased cardiovascular fitness,
h) decreased exercise intolerance,
i) increased exercise capacity, or
j) decreased exercise induced fatigue, and
k) decreased hypotonia.

As such, the method can alternatively be described as a method of increasing skeletal muscle strength, in a subject (optionally wherein the subject is suffering from a disease or condition in which muscle e.g. skeletal muscle function is impaired), the method comprising administering an effective amount of an inhibitor of FGFR3 signalling, or an effective amount of an NPR-B agonist, or an effective amount of an NPR-C agonist to said subject.

The method can also be described as a method of increasing skeletal muscle tone in a subject (optionally wherein the subject is suffering from a disease or condition in which muscle e.g. skeletal muscle function is impaired), the method comprising administering an effective amount of an inhibitor of FGFR3 signalling or an effective amount of an NPR-B agonist, or an effective amount of an NPR-C agonist to said subject.

The method can also be described as a method of increasing skeletal muscle stamina in a subject (optionally wherein the subject is suffering from a disease or condition in which muscle e.g. skeletal muscle function is impaired), the method comprising administering an effective amount of an inhibitor of FGFR3 signalling or an effective amount of an NPR-B agonist, or an effective amount of an NPR-C agonist to said subject.

The method can also be described as a method of increasing skeletal muscle mass in a subject (optionally wherein the subject is suffering from a disease or condition in which muscle e.g. skeletal muscle function is impaired), the method comprising administering an effective amount of an inhibitor of FGFR3 signalling or an effective amount of an NPR-B agonist, or an effective amount of an NPR-C agonist to said subject.

The method can also be described as a method of decreasing skeletal muscle fatigue in a subject (optionally wherein the subject is suffering from a disease or condition in which muscle e.g. skeletal muscle function is impaired), the method comprising administering an effective amount of an inhibitor of FGFR3 signalling or an effective amount of an NPR-B agonist, or an effective amount of an NPR-C agonist to said subject.

The method can also be described as a method of increasing cardiovascular endurance in a subject (optionally wherein the subject is suffering from a disease or condition in which muscle e.g. skeletal muscle function is impaired), the method comprising administering an effective amount of an inhibitor of FGFR3 signalling or an effective amount of an NPR-B agonist, or an effective amount of an NPR-C agonist to said subject.

The method can also be described as a method of increasing cardiovascular fitness in a subject (optionally wherein the subject is suffering from a disease or condition in which muscle e.g. skeletal muscle function is impaired), the method comprising administering an effective amount of an inhibitor of FGFR3 signalling or an effective amount of an NPR-B agonist, or an effective amount of an NPR-C agonist to said subject.

The method can also be described as a method of decreasing exercise intolerance in a subject (optionally wherein the subject is suffering from a disease or condition in which muscle e.g. skeletal muscle function is impaired), the method comprising administering an effective amount of an inhibitor of FGFR3 signalling or an effective amount of an NPR-B agonist, or an effective amount of an NPR-C agonist to said subject.

The method can also be described as a method of increasing exercise capacity in a subject (optionally wherein the subject is suffering from a disease or condition in which muscle e.g. skeletal muscle function is impaired), the method comprising administering an effective amount of an inhibitor of FGFR3 signalling or an effective amount of an NPR-B agonist, or an effective amount of an NPR-C agonist to said subject.

The method can also be described as a method of decreasing exercise induced fatigue in a subject (optionally wherein the subject is suffering from a disease or condition in which muscle e.g. skeletal muscle function is impaired), the method comprising administering an effective amount of an inhibitor of FGFR3 signalling or an effective amount of an NPR-B agonist, or an effective amount of an NPR-C agonist to said subject.

The method can also be described as a method of decreasing hypotonia in a subject (optionally wherein the subject is suffering from a disease or condition in which muscle e.g. skeletal muscle function is impaired), the method comprising administering an effective amount of an inhibitor of FGFR3 signalling or an effective amount of an NPR-B agonist, or an effective amount of an NPR-C agonist to said subject.

Administration of the inhibitor of FGFR3 signalling or an effective amount of an NPR-B agonist, or NPR-C agonist may give rise to an increase in the skeletal muscle/fat ratio of the subject and the method may alternatively be framed as a method of increasing the skeletal muscle/fat ratio in a subject (optionally wherein the subject is suffering from a disease or condition in which muscle e.g. skeletal muscle function is impaired), the method comprising administering an effective amount of an inhibitor of FGFR3 signalling, or an effective amount of an NPR-B agonist, or an effective amount of an NPR-C agonist to said subject.

Administration of the inhibitor of FGFR3 signalling or an effective amount of an NPR-B agonist, or NPR-C agonist may give rise to a decrease in hypotonia in the subject and the method can therefore be framed as a method of decreasing hypotonia in a subject (optionally wherein the subject is suffering from a disease or condition in which muscle e.g. skeletal muscle function is impaired), the method comprising administering an effective amount of an inhibitor of FGFR3 signalling, or an effective amount of an NPR-B agonist, or an effective amount of an NPR-C agonist to said subject. The method thus may be described as a method of treating hypotonia in the subject.

Administration of the inhibitor of FGFR3 signalling or an effective amount of an NPR-B agonist, or NPR-C agonist may give rise to a decrease in musculoskeletal pain in the subject and the method can therefore be framed as a method of treating, preventing or reducing decreasing musculoskeletal pain in a subject (optionally wherein the subject is suffering from a disease or condition in which muscle e.g. skeletal muscle function is impaired), the method comprising administering an effective amount of an inhibitor of FGFR3 signalling, or an effective amount of an NPR-B agonist, or an effective amount of an NPR-C agonist to said subject.

The method of also gives rise to an improvement in posture, an improvement in an abnormal curvature of the spine and the method can therefore be framed as a method of improving posture or for treatment of an abnormal curvature of the spine in a subject (optionally wherein the subject is suffering from a disease or condition in which muscle e.g. skeletal muscle function is impaired), the method comprising administering an effective amount of an inhibitor of FGFR3 signalling or an effective amount of an NPR-B agonist, or an effective amount of an NPR-C agonist to said subject. The method may give rise to an improvement in kyphosis, lordosis or scoliosis and as such can be defined as a method of treating one or more of these conditions in a subject (optionally wherein the subject is suffering from a disease or condition in which muscle e.g. skeletal muscle function is impaired), the method comprising administering an effective amount of an inhibitor of FGFR3 signalling, or an effective amount of an NPR-B agonist, or an effective amount of an NPR-C agonist to said subject.

The method may give rise to an improvement in sleep apnea, snoring, obstructive sleep apnea, otitis media and as such can be defined as a method of treating one or more of these conditions in a subject (optionally wherein the subject is suffering from a disease or condition in which muscle e.g. skeletal muscle function is impaired), the method comprising administering an effective amount of an inhibitor of FGFR3 signalling or an effective amount of an NPR-B agonist, or an effective amount of an NPR-C agonist to said subject. A reduction in the frequency of occurrence, incidence and/or severity of the sleep apnea, snoring, obstructive sleep apnea, otitis media may result.

The method may give rise to a reduction in obesity and as such can be defined as a method of reducing obesity in a subject (optionally wherein the subject is suffering from a disease or condition in which muscle e.g. skeletal muscle function is impaired), the method comprising administering an effective amount of an inhibitor of FGFR3 signalling or an effective amount of an NPR-B agonist, or an effective amount of an NPR-C agonist to said subject.

The present invention also provides for a method for treating kyphosis in a patient in need of said treatment, said method comprising administering an effective amount of CNP, CNP conjugate or pharmaceutically acceptable salt thereof or unit dosage form to the patient thereby treating kyphosis.

In certain embodiments, the patient has been diagnosed with a bone dysplasia or bone disorder, such as a disorder selected from the group consisting of achondroplasia, hypochondroplasia, short stature, Noonan syndrome and SHOX deficiency.

The present invention also provides for a method for treating foramen magnum stenosis in a patient in need of said treatment, said method comprising administering an effective amount of CNP, CNP conjugate or pharmaceutically acceptable salt thereof or unit dosage form to the patient thereby treating foramen magnum stenosis. In some embodiments the patient has achondroplsia.

The present invention also provides for a method for treating otitis media or ear infection in a patient or for reducing incidence of ear infection in a patient in need of said treatment, said method comprising administering a therapeutically effective amount of a CNP, CNP conjugate or pharmaceutically acceptable salt thereof or unit dosage form to the patient thereby treating otitis media, or ear infection or reducing incidence of ear infection. In some embodiments the patient has achondroplsia.

The present invention also provides for a method for treating sleep apnea syndrome in a patient in need to said treatment, said method comprising administering a therapeutically effective amount of a CNP, CNP conjugate or pharmaceutically acceptable salt thereof or unit dosage form to the patient. By way of example, the treatment may reduce the incidence of sleep apnea or reduce the severity of sleep apnea. In some embodiments the patient has achondroplsia.

The present invention also provides for a method for reducing the frequency of achondroplasia related adverse events in a patient diagnosed with achondroplasia, said method comprising administering a therapeutically effective amount of a CNP, CNP conjugate or pharmaceutically acceptable salt thereof or unit dosage form, wherein optionally the patient may be a pediatric patient, and/or a patient with open bone epiphysis

In any of the methods described above the subject may have a chondrodysplasia disease, such as a disease selected from the group consisting of achondroplasia, hypochondroplasia, and thanatophoric dysplasia.

In the methods described above the subject may have achondroplasia. In some embodiments the subject does not have achondroplsia.

In any of the methods described above the subject may have achondroplasia wherein the subject is a human subject of at least 18 years of age, or a human subject whose bone epiphesis have closed.

To the extent that a method is referred to herein, the invention also provides an inhibitor of FGFR3 signalling, or an effective amount of an NPR-B agonist, or an effective amount of an NPR-C agonist for use in that method and use of an inhibitor of FGFR3 signalling, or an effective amount of an NPR-B agonist, or an effective amount of an NPR-C agonist in the manufacture of a medicament for use in that method.

In certain embodiments, the CNP is in the form of a unit dosage forms comprising a CNP conjugate or pharmaceutically acceptable salt thereof.

In certain embodiments, the unit dosage form of CNP comprises a therapeutically effective amount of a CNP conjugate or pharmaceutically acceptable salt thereof in which a CNP moiety is reversibly conjugated to a polymeric moiety.

### FIGURES

FIG. 1: Percentage survival of Fgfr3 Y367C/+ pups over study period, comparing groups treated with Compound (1) at 5.6 mg/kg/day (solid line), Compound (1) at 1.2 mg/kg/every third day (dashed line) or Vehicle alone (dotted line).

FIG. 2: The change in pedicular width after 52 weeks on treatment (cohorts 3 and 4), as compared to placebo, as detailed in Example 14. The data illustrates a trend for increased pedicular width on treatment as compared to placebo.

FIG. 3: The change in mean hand length after 52 weeks on treatment (Cohorts 3 and 4) as compared to placebo, as detailed in Example 15. The results indicate a clear trend for dose dependent increase in the hand length on treatment.

### DEFINITIONS

As used herein, the term “about” in combination with a numerical value is used to indicate a range ranging from and including the numerical value plus and minus no more than 10% of said numerical value, in certain embodiments, no more than 8% of said numerical value, in certain embodiments, no more than 5% of said numerical value and in certain embodiments, no more than 2% of said numerical value. For example, the phrase “about 200” is used to mean a range ranging from and including 200+/−10%, i.e. ranging from and including 180 to 220; in certain embodiments, 200+/−8%, i.e. ranging from and including 184 to 216; in certain embodiments, ranging from and including 200+/−5%, i.e. ranging from and including 190 to 210; and in certain embodiments 200+/−2%, i.e. ranging from and including 196 to 204. It is understood that a percentage given as “about 20%” does not mean “20%+/−10%”, i.e. ranging from and including 10 to 30%, but “about 20%” means ranging from and including 18 to 22%, i.e. plus and minus 10% of the numerical value which is 20.

As used herein, the term “antimicrobial” refers to a chemical substance, such as a chemical substance that kills or inhibits the growth of microorganisms, such as bacteria, fungi, yeasts, protozoans, molds and/or destroys viruses.

As used herein, the term “buffer” or “buffering agent” refers to a chemical compound that maintains the pH in a desired range. Physiologically tolerated buffers are, for example, sodium phosphate, succinate, histidine, bicarbonate, citrate, acetate, sulfate, nitrate, chloride and pyruvate. Antacids such as Mg(OH)₂ or ZnCO₃ may be also used.

As used herein, the term “CNP” refers to all CNP polypeptides, in certain embodiments from mammalian species, such as from human and mammalian species, in particular from human and murine species, as well as their variants, analogs, orthologs, homologs and derivatives and fragments thereof, that are characterized by regulating the growth, proliferation and differentiation of cartilaginous growth plate chondrocytes. The human prepro-CNP which comprises 126 amino acids is further cleaved to yield CNP-53 and CNP-22. The term “CNP” also includes all CNP variants, analogs, orthologs, homologs, derivatives and fragments thereof. The CNP variants, analogs, orthologs, homologs, derivatives and fragments thereof as disclosed in WO 2009/067639 A2 and WO 2010/135541 A2 are herewith incorporated by reference. CNP peptides and pharmaceutical compositions comprising CNP peptides are disclosed in WO2009/067639, WO2010/135541, WO2017/020034, WO2017/100400, WO2021055497, WO2021/030411, WO2023/283657, WO2022/115563 which are all incorporated by reference herein. Exemplary CNP peptides are provided herein, and include vosoritide.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₅₀ carbon may optionally be replaced by a substituent as defined above. Optionally, a C₁₋₁₀, C₁₋₂₀ alkyl or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example of such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the term “C₂₋₁₀ alkenyl”, “C₂₋₂₀ alkenyl” or “C₂₋₅₀ alkenyl” alone or in combination means a straight-chain or branched hydrocarbon moiety comprising at least one carbon-carbon double bond having 2 to 10, 2 to 20 or 2 to 50 carbon atoms. Each hydrogen atom of a C₂₋₁₀ alkenyl, C₂₋₂₀ alkenyl or C₂₋₅₀ alkenyl group may optionally be replaced by a substituent as defined above. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C≡CH, —CH₂—C≡CH, —CH₂—CH₂—C≡CH and —CH₂—C≡C—CH₃. When two moieties of a molecule are linked by the alkynyl group, then an example is —C≡C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined above. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which are in certain embodiments, selected from the group consisting of

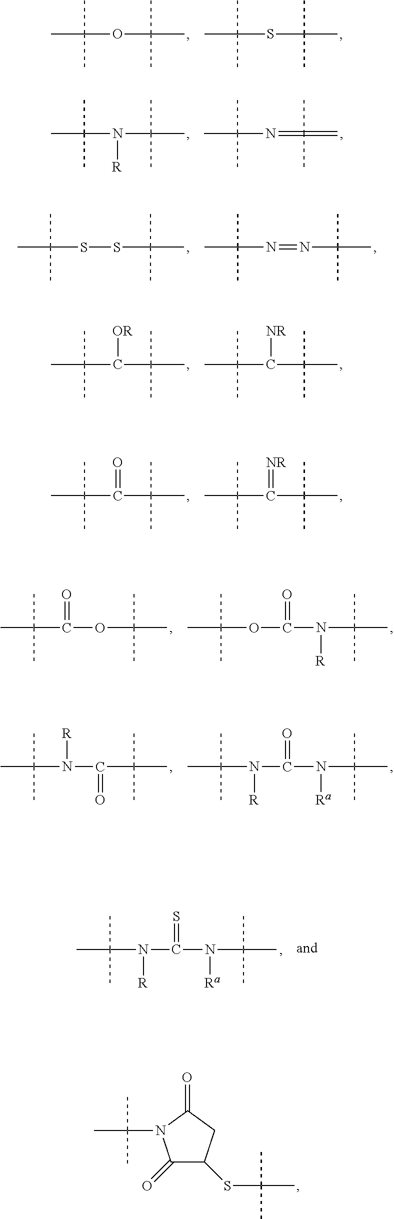

wherein
dashed lines indicate attachment to the remainder of the moiety or reagent; and
—R and —R^{a} are independently of each other selected from the group consisting of —H, methyl, ethyl, propyl, butyl, pentyl and hexyl.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined above. The term “C₃₋₁₀ cycloalkyl” also includes bridged bicycles like norbornane or norbornene.

As used herein, the term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In certain embodiments, an 8- to 30-membered carbopolycyclyl means a cyclic moiety of two, three, four or five rings, in certain embodiments of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3-to 10-membered heterocyclyl or 3- to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8-to 11-membered heterobicycle carbon may be replaced by a substituent as defined below.

Similarly, the term “8- to 30-membered heteropolycyclyl” or “8- to 30-membered heteropolycycle” means a heterocyclic moiety of more than two rings with 8 to 30 ring atoms, in certain embodiments of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

It is understood that the phrase “the pair R^{x}/R^{y} is joined together with the atom to which they are attached to form a C₃₋₁₀ cycloalkyl or a 3- to 10-membered heterocyclyl” in relation with a moiety of the structure:

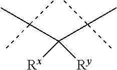

means that R^{x} and R^{y} form the following structure:

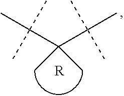

wherein R is C₃₋₁₀ cycloalkyl or 3- to 10-membered heterocyclyl.

It is also understood that the phrase “the pair R^{x}/R^{y} is joined together with the atoms to which they are attached to form a ring A” in relation with a moiety of the structure:

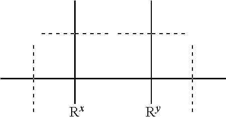

means that R^{x} and R^{y} form the following structure:

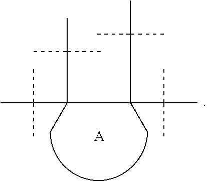

As used herein, the term “CNP polypeptide variant” refers to a polypeptide from the same species that differs from a reference CNP polypeptide. Generally, differences are limited so that the amino acid sequence of the reference and the variant are closely similar overall and, in many regions, identical. In certain embodiments, CNP polypeptide variants are at least 70%, 80%, 90%, or 95% identical to a reference CNP polypeptide. By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. These alterations of the reference sequence may occur at the amino (N-terminal) or carboxy terminal (C-terminal) positions of the reference amino acid sequence or anywhere between those terminal positions, interspersed either individually among residues in the reference sequence or in one or more contiguous groups within the reference sequence. The query sequence may be an entire amino acid sequence of the reference sequence or any fragment specified as described herein. Such CNP polypeptide variants may be naturally occurring variants, such as naturally occurring allelic variants encoded by one of several alternate forms of a CNP occupying a given locus on a chromosome or an organism, or isoforms encoded by naturally occurring splice variants originating from a single primary transcript. Alternatively, a CNP polypeptide variant may be a variant that is not known to occur naturally and that can be made by mutagenesis techniques known in the art. It is known in the art that one or more amino acids may be deleted from the N-terminus or C-terminus of a bioactive peptide or protein without substantial loss of biological function. Such N- and/or C-terminal deletions are also encompassed by the term CNP polypeptide variant.

As used herein, the term “dose” or “unit dose” refers to the predetermined amount of the drug, such as CNP, administered at one time to produce a certain degree of biological response in a patient. The dose of a drug is governed by its inherent potency and in this case, it is a therapeutic dose or therapeutic unit dose.

As used herein, the term “dosage form” refers to the physical form that comprises the active pharmaceutical ingredient in combination with selected additional ingredients or excipients and which is intended to be delivered to sites of action within the body by various routes of drug administration. It also refers to the physical form in which a precise mixture of active pharmaceutical ingredients and excipients are presented to help administration and delivery to the sites of action, achieve rapid onset of action and improve bioavailability. As used herein, the term “unit dosage form” refers to a dosage form configured for a single administration to a patient. For example, a unit dosage form can be a single vial or the container containing an amount of drug suitable for a single administration.

As used herein, the term “dosage regimen” is the combination of dose, and frequency with which a drug is administered. Dosage regimen can also include a route of administration (e.g., subcutaneous) and/or duration of administration (e.g., until a patient reaches 18 years old or ephiphyseal closure). Administration of a dosage regimen may maintain a steady-state serum concentration of CNP, in which peaks, troughs and area under the curve over a defined interval remain with defined margins of fluctuation and/or the ratio of peaks to troughs does not exceed a defined threshold.

As used herein, the term “drug” refers to a substance used in the treatment, cure, prevention, or diagnosis of a disease or used to otherwise enhance physical or mental well-being. If a drug, such as CNP, is conjugated to another moiety, the moiety of the resulting product that originated from the drug is referred to as “drug moiety”.

As used herein, the term “excipient” refers to compounds administered together with the drug or drug conjugate, for example, buffering agents, isotonicity modifiers, preservatives, stabilizers, anti-adsorption agents, oxidation protection agents, or other auxiliary agents. However, in some cases, one excipient may have dual or triple functions. The term “excipient” may also refer to a diluent, adjuvant, or vehicle with which the drug or drug conjugate, is administered. Such pharmaceutical excipient can be sterile liquids, such as water and oils, including those of petroleum, animal, vegetable or synthetic origin, including, but not limited to peanut oil, soybean oil, mineral oil, sesame oil and the like. Water is a preferred excipient when the pharmaceutical formulation is administered orally. Saline and aqueous dextrose are preferred excipients when the pharmaceutical formulation is administered intravenously or subcutaneously. Saline solutions and aqueous dextrose and glycerol solutions are in certain embodiments, employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, water, ethanol and the like. The pharmaceutical formulation can also contain minor amounts of wetting or emulsifying agents, pH buffering agents, like, for example, acetate, succinate, Tris (tris(hydroxymethyl)aminomethane), carbonate, phosphate, HEPES (4-(2-hydroxyethyl)-1-piperazineethanesulfonic acid), MES (2-(N-morpholino)ethanesulfonic acid), or can contain detergents, like Tween®, poloxamers, poloxamines, CHAPS, Igepal®, or amino acids like, for example, glycine, lysine, or histidine. These pharmaceutical formulations can take the form of solutions, suspensions, emulsions, tablets, pills, capsules, powders, sustained-release formulations and the like. The pharmaceutical formulation can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such formulations will contain a therapeutically effective amount of the drug or drug moiety, together with a suitable amount of excipient so as to provide the form for proper administration to the patient. The formulation should suit the mode of administration.

As used herein, the term “formulation” or “pharmaceutical formulation” refers to a formulation containing one or more CNP conjugates and one or more excipients, as well as any product which results, directly or indirectly, from combination, complexation or aggregation of any two or more of the ingredients of the composition, or from dissociation of one or more of the ingredients, or from other types of reactions or interactions of one or more of the ingredients. Accordingly, the pharmaceutical formulations of the present invention encompass any formulation or composition made by admixing one or more CNP conjugates and a pharmaceutically acceptable excipient such as a buffering agent and bulking agent.

As used herein, the term “free form” of a drug refers to the drug in its unmodified, pharmacologically fully active form, e.g. after being released from the CNP conjugate or pharmaceutically acceptable salt thereof.

As used herein, the term “functional group” means a group of atoms which can react with other groups of atoms. Functional groups include, but are not limited, to the following groups: carboxylic acid (—(C═O)OH), primary or secondary amine (—NH₂, —NH—), maleimide, thiol (—SH), sulfonic acid (—(O═S=O)OH), carbonate, carbamate (—O(C═O)N<), hydroxyl (—OH), aldehyde (—(C═O)H), ketone (—(C═O)—), hydrazine (>N—N<), isocyanate, isothiocyanate, phosphoric acid (—O(P═O)OHOH), phosphonic acid (—O(P═O)OHH), haloacetyl, alkyl halide, acryloyl, aryl fluoride, hydroxylamine, disulfide, sulfonamides, sulfuric acid, vinyl sulfone, vinyl ketone, diazoalkane, oxirane and aziridine.

As used herein, the term “halogen” means fluoro, chloro, bromo or iodo. It is generally preferred that halogen is fluoro or chloro.

As used herein, the term “interrupted” means that a moiety is inserted in between two carbon atoms or—if the insertion is at one of the moiety's ends—between a carbon or heteroatom and a hydrogen atom, in certain embodiments between a carbon and a hydrogen atom.

As used herein, the term “isotonicity agent” refers to a compound that minimizes pain, irritation and tissue damage that can result from cell damage due to osmotic pressure differences between the injected solution and plasma.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “-” indicates attachment to another moiety. Accordingly, a drug moiety, such as a CNP moiety, is released from a conjugate as a drug, such as CNP.

It is understood that if the sequence or chemical structure of a group of atoms is provided which group of atoms is attached to two moieties or is interrupting a moiety, said sequence or chemical structure can be attached to the two moieties in either orientation, unless explicitly stated otherwise. For example, a moiety “—C(O)N(R′)—” can be attached to two moieties or interrupting a moiety either as “—C(O)N(R¹)—” or as “—N(R¹)C(O)—”. Similarly, a moiety:

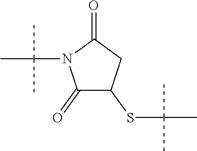

can be attached to two moieties or can interrupt a moiety either as:

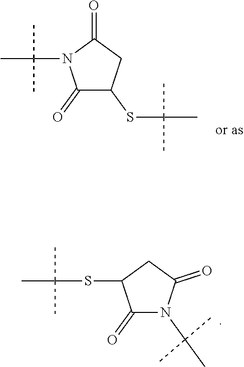

In case the CNP moiety comprises one or more acidic or basic groups, the unit dosage form comprises also their corresponding pharmaceutically or toxicologically acceptable salts, in particular their pharmaceutically utilizable salts. Thus, the CNP moieties comprising one or more acidic groups can be present and used, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids, and other salts or amines known to the person skilled in the art. CNP moieties comprising one or more basic groups, i.e. groups which can be protonated, can be present and can be used in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. For the person skilled in the art further methods are known for converting the basic group into a cation like the alkylation of an amine group resulting in a positively charged ammonium group and an appropriate counterion of the salt. If the CNP moieties simultaneously comprise acidic and basic groups, the pharmaceutical formulations according to the present invention also include, in addition to the salt forms mentioned, inner salts or betaines (zwitterions). The respective salts can be obtained by customary methods which are known to the person skilled in the art like, for example by contacting these conjugates with an organic or inorganic acid or base in a solvent or dispersant, or by anion exchange or cation exchange with other salts. The unit dosage form according to the present invention also includes all salts of the CNP conjugates which, owing to low physiological compatibility, are not directly suitable for use in pharmaceuticals but which can be used, for example, as intermediates for chemical reactions or for the preparation of pharmaceutically acceptable salts.

As used herein, the term “patient” refers to a subject amenable to treatment or prophylaxis according to the invention, particularly a human subject.

As used herein, the term “pharmaceutically acceptable” means a substance that does not cause harm when administered to a patient and preferably means approved by a regulatory agency, such as the EMA (Europe) and/or the FDA (US) and/or any other national regulatory agency for use in animals, preferably for use in humans.

As used herein, the term “physiological conditions” refers to an aqueous buffer at pH 7.4 and 37° C.

The term “polypeptide” as used herein refers to a chain of at least 2 and up to and including 50 amino acid monomer moieties linked by peptide (amide) linkages. Only for CNP drugs and CNP moieties also the sequences having more than 50 amino acids will be referred to as “polypeptide” for simplification.

As used herein, the term “preservative” refers to a chemical substance that has antimicrobial effects and prevents chemical degradation.

As used herein, the term “protein” refers to a chain of more than 50 amino acid monomer moieties linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. It is understood that a polymer may also comprise one or more other chemical groups and/or moieties, such as, for example, one or more functional groups. In certain embodiments, a soluble polymer has a molecular weight of at least 0.5 kDa, e.g. a molecular weight of at least 1 kDa, a molecular weight of at least 2 kDa, a molecular weight of at least 3 kDa or a molecular weight of at least 5 kDa. If the polymer is soluble, in certain embodiments it has a molecular weight of at most 1000 kDa, such as at most 750 kDa, such as at most 500 kDa, such as at most 300 kDa, such as at most 200 kDa, such as at most 100 kDa.

It is understood that also a protein or a polypeptide is a polymer in which the amino acids are the repeating structural units, even though the side chains of each amino acid may be different.

As used herein, the term “polymeric” or “polymeric moiety” means a reagent or a moiety comprising one or more polymers or polymer moieties. A polymeric reagent or moiety may optionally also comprise one or more other moiety/moieties, which are in certain embodiments selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

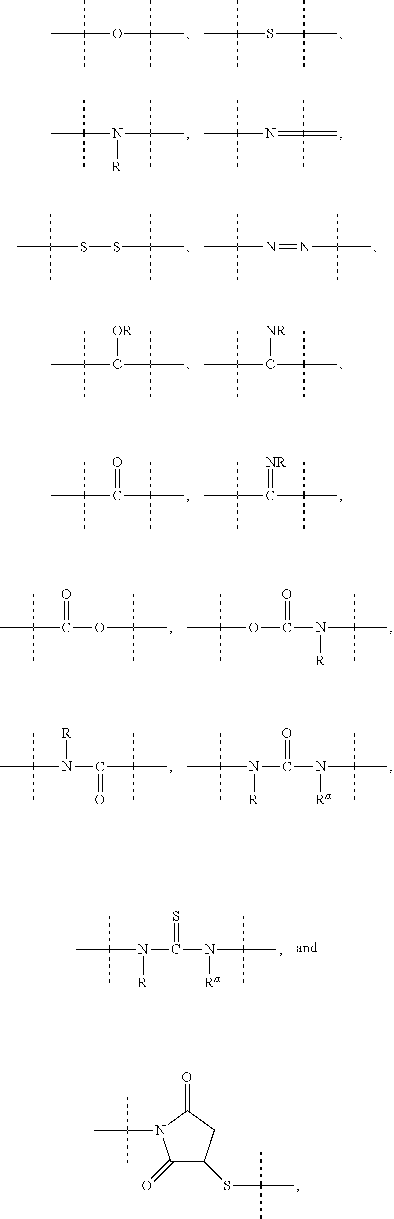

wherein
dashed lines indicate attachment to the remainder of the moiety or reagent, and
—R and —R^{a} are independently of each other selected from the group consisting of —H, methyl, ethyl, propyl, butyl, pentyl and hexyl.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lies. An integer for “x” given as “about x” means that the arithmetic mean numbers of monomers lies in a range of integers of x+/−10%, in certain embodiments lies in a range of integers x+/−8%, in certain embodiments lies in a range of integers x+/−5% and in certain embodiments lies in a range of integers x+/−2%.

As used herein, the term “PEG-based” in relation to a moiety or reagent means that said moiety or reagent comprises PEG. In certain embodiments, a PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60% (w/w) PEG, such as at least 70% (w/w) PEG, such as at least 80% (w/w) PEG, such as at least 90% (w/w) PEG, such as at least 95% (w/w) PEG. The remaining weight percentage of the PEG-based moiety or reagent are other moieties selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

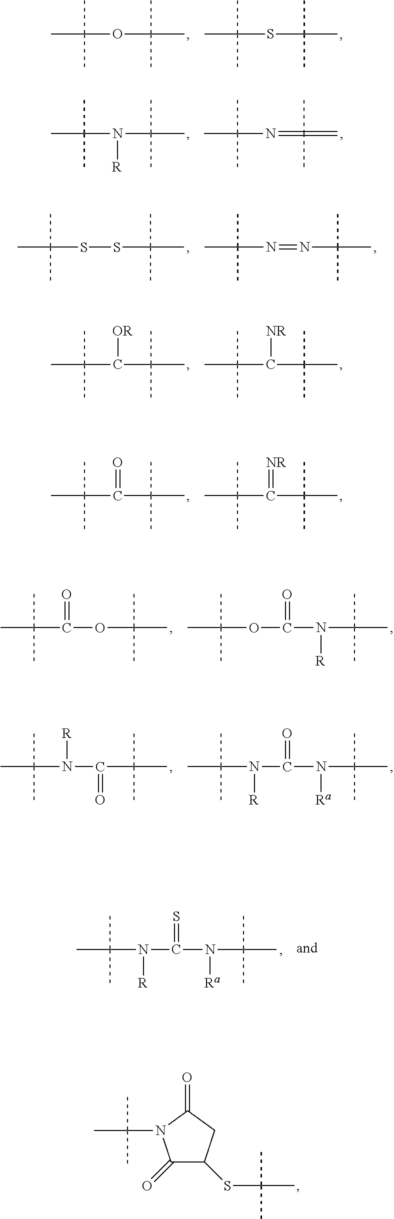

wherein
dashed lines indicate attachment to the remainder of the moiety or reagent, and
—R and —R^{a} are independently of each other selected from the group consisting of —H, methyl, ethyl, propyl, butyl, pentyl and hexyl.

As used herein, the term “PEG-based comprising at least X % PEG” in relation to a moiety or reagent means that said moiety or reagent comprises at least X % (w/w) ethylene glycol units (—CH₂CH₂O—), wherein the ethylene glycol units may be arranged blockwise, alternating or may be randomly distributed within the moiety or reagent and in certain embodiments, all ethylene glycol units of said moiety or reagent are present in one block; the remaining weight percentage of the PEG-based moiety or reagent are other moieties in certain embodiments selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

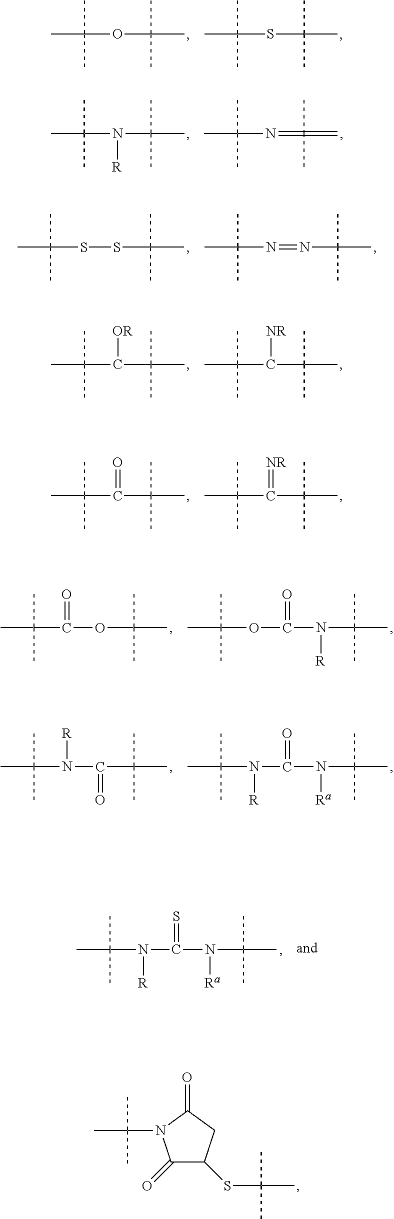

wherein
dashed lines indicate attachment to the remainder of the moiety or reagent, and
—R and —R^{a} are independently of each other selected from the group consisting of —H, methyl, ethyl, propyl, butyl, pentyl and hexyl.

As used herein, the term “hyaluronic acid-based comprising at least X % hyaluronic acid” is used accordingly.

As used herein, the term “prodrug” refers to a drug moiety, such as a CNP moiety, reversibly and covalently conjugated to a polymeric moiety, such as —Z, through a reversible linker moiety. A prodrug releases the reversibly and covalently bound drug moiety in the form of its corresponding drug. In other words, a prodrug is a conjugate comprising a drug moiety, such as a CNP moiety, which is covalently and reversibly conjugated to a polymeric moiety via a reversible linker moiety, which covalent and reversible conjugation of the polymeric moiety to the reversible linker moiety is either direct or through a spacer. Such prodrugs or conjugates release the formerly conjugated drug moiety in the form of a free drug.

As used herein, the term “random coil” refers to a peptide or protein adopting/having/forming, in certain embodiments having, a conformation which substantially lacks a defined secondary and tertiary structure as determined by circular dichroism spectroscopy performed in aqueous buffer at ambient temperature, and pH 7.4. In certain embodiments, the ambient temperature is about 20° C., i.e. between 18° C. and 22° C., while in certain embodiments the ambient temperature is 20° C.

As used herein, the term “reversible linkage” is a linkage that is cleavable, in the absence of enzymes under physiological conditions (aqueous buffer at pH 7.4, 37° C.) with a half-life ranging from one hour to six months, such as from one hour to four months, such as from one hour to three months, from one hour to two months or from one hour to one month. Accordingly, a stable linkage is a linkage having a half-life under physiological conditions (aqueous buffer at pH 7.4, 37° C.) of more than six months.

As used herein, the term “reagent” means a chemical compound which comprises at least one functional group for reaction with the functional group of another chemical compound or drug. It is understood that a drug comprising a functional group (such as a primary or secondary amine or hydroxyl functional group) is also a reagent.

As used herein, the term “reversible linker moiety” is a moiety which is covalently conjugated to a drug moiety, such as a CNP moiety, through a reversible linkage and is also covalently conjugated to a polymeric moiety, such as —Z, wherein the covalent conjugation to said polymeric moiety is either direct or through a spacer moiety, such as -L²-. In certain embodiments, the linkage between —Z and -L²- is a stable linkage. A conjugate comprising a reversible linker moiety can be referred to as a reversible conjugate.

As used herein, the term “spacer” or “spacer moiety” refers to a moiety suitable for connecting two moieties. Suitable spacers may be selected from the group consisting of C₁₋₅₀ alkyl, C₂₋₅₀ alkenyl or C₂₋₅₀ alkynyl, which C₁₋₅₀ so alkyl, C₂₋₅₀ alkenyl or C₂₋₅₀ alkynyl is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4- to 7-membered heterocyclyl, phenyl and naphthyl.

As used herein, the term “substituted” means that one or more —H atom(s) of a molecule or moiety are replaced by a different atom or a group of atoms, which are referred to as “substituent”.

In certain embodiments, such one or more substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b})—SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

—R^{x1}—R^{x1a}, —R^{x1b} are independently of each other selected from the group consisting of —H, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3}); —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different; each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;
each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments, the one or more substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₁₀ alkyl, C₂₋₁₀ alkenyl, and C₂₋₁₀ alkynyl; wherein -T⁰, C₁₋₁₀ alkyl, C₂₋₁₀ alkenyl, and C_{2 -10} alkynyl are optionally substituted with one or more —R^{x2}, which are the same or different and wherein C₁₋₁₀ alkyl, C₂₋₁₀ alkenyl, and C₂₋₁₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

each —R^{x1}, —R^{x1a}, —R^{x1b}, —R^{x3}, —R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T° is independently optionally substituted with one or more —R^{x2}, which are the same or different;
each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4}R^{x4b}), —SR^{x4}, —N(R^{x4}R^{x4}), —NO₂, —OC(O)R^{x4}, —N(R^{x4})C(O)R^{x4a}—N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}) and C₁₋₆ alkyl; wherein C₁₋₆ alkyl is optionally substituted with one or more halogen, which are the same or different;
each —R^{x4}, —R^{x4a}, —R^{x4b} is independently selected from the group consisting of —H, halogen, C₁₋₆ alkyl, C₂₋₆ alkenyl, and C₂₋₆ alkynyl.

In certain embodiments, the one or more substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{X1b}), —OC(O)N(R^{x1}R^{x1a}), -T⁰, C₁₋₆ alkyl, C₂₋₆ alkenyl, and C₂₋₆ alkynyl; wherein -T⁰, C₁₋₆ alkyl, C₂₋₆ alkenyl, and C₂₋₆ alkynyl are optionally substituted with one or more —R^{x2}, which are the same or different and wherein C₁₋₆ alkyl, C₂₋₆ alkenyl, and C₂₋₆ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a}) and —OC(O)N(R^{x3})—;
each —R^{x1}, —R^{x1a}, —R^{x1b}, —R^{x2}, —R^{x3}—R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different.

In certain embodiments, a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

As used herein, the term “therapeutically effective amount” means an amount sufficient to cure, alleviate or partially arrest the clinical manifestations of a given disease and its complications. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician. Within the scope of this invention, therapeutically effective amount relates to dosages that aim to achieve therapeutic effect for an extended period of time, i.e. for at least one day, such as for two days, such as for three days, such as for four days, such as for five days, such as for six days, such as for one week or such as for two weeks.

As used herein, the term “traceless linker” means a reversible linker which upon cleavage releases the drug in its free form.

As used herein, the term “water-soluble” with reference to a polymeric moiety means that when such polymeric moiety is part of the CNP conjugate, at least 1 g of the CNP conjugate comprising such water-soluble polymeric moiety can be dissolved in one liter of water at 20° C. to form a homogeneous solution.

In general, the term “comprise” or “comprising” also encompasses “consist of” or “consisting of”.

The amino acid sequences of CNP polypeptides can be varied without significant effect on the structure or function of the peptide. Such mutants include deletions, insertions, inversions, repeats, and substitutions selected according to general rules known in the art so as to have little effect on activity. For example, guidance concerning how to make phenotypically silent amino acid substitutions is provided in Bowie et al. (1990), Science 247:1306-1310, which is hereby incorporated by reference in its entirety, wherein the authors indicate that there are two main approaches for studying the tolerance of the amino acid sequence to change.

As used herein, the term “CNP analog” refers to CNP of different and unrelated organisms which perform the same functions in each organism, but which did not originate from an ancestral structure that the organisms' ancestors had in common. Instead, analogous CNPs arose separately and then later evolved to perform the same or similar functions. In other words, analogous CNP polypeptides are polypeptides with quite different amino acid sequences that perform the same biological activity, namely regulating the growth, proliferation and differentiation of cartilaginous growth plate chondrocytes.

As used herein, the term “CNP ortholog” refers to CNP within two different species which sequences are related to each other via a common homologous CNP in an ancestral species, but which have evolved to become different from each other.

As used herein, the term “CNP homolog” refers to CNP of different organisms which perform the same functions in each organism, and which originate from an ancestral structure that the organisms' ancestors had in common. In other words, homologous CNP polypeptides are polypeptides with quite similar amino acid sequences that perform the same biological activity, namely regulating the growth, proliferation and differentiation of cartilaginous growth plate chondrocytes. In certain embodiments, CNP polypeptide homologs may be defined as polypeptides exhibiting at least 40%, 50%, 60%, 70%, 80%, 90% or 95% identity to a reference CNP polypeptide.

### DETAILED DESCRIPTION OF THE INVENTION

The invention relates to a method of improving muscle function, such as skeletal muscle function, in a subject suffering from a disease or condition in which muscle function is impaired, the method comprising administering an effective amount of an inhibitor of FGFR3 signalling, or an effective amount of an NPR-B agonist, or an effective amount of an NPR-C agonist to said subject.

### Inhibitors of FGFR3 Signalling

The FGFR3 signalling pathway is well understood and involves the MAPK and the STET pathways. In non pathological states, the receptor is activated by ligand (e.g. FGF1, FGF2 or FGF9) binding and receptor dimerization which brings tyrosine kinase domains of each member of the receptor dimer pair into close proximity, allowing them to cross-phosphorylate each other on tyrosines in their activation loops. This activates the kinases, which then bind adaptor proteins and phosphorylate cytoplasmic substrates, triggering downstream signalling cascades that control cell growth and differentiation. Certain pathological states are associated with constitutive FGFR3 activation, and in these states receptor dimerization and phosphorylation may occur without ligand binding.

Inhibition of FGFR3 signalling can occur by reducing or preventing the activity of the signalling cascade at any point in the cascade. For example, FGFR3 antagonists may bind to the receptor itself to reduce or prevent binding of the activating ligand and such molecules may be defined as direct FGFR3 antagonists. Examples include anti FGFR3 antibodies, e.g. FGFR3 monoclonal antibodies, Suitable examples are disclosed in WO2022/040560 which discloses anti-FGFR3 monoclonal antibodies and their use in treatment of achondroplasia and is hereby incorporated by reference in its entirety. Likewise WO2018/145120 and WO2020/180898 both hereby incorporated by reference in their entirety disclose anti-FGFR3 monoclonal antibodies and their use in treatment. Vofatamab (B-701) is a monoclonal antibody specific for fibroblast growth factor receptor 3 in clinical development.

Inhibitors of FGFR3 signalling may also act to prevent FGFR3 signalling by preventing or reducing ligand binding to FGFR3. Decoy molecules for the FGFR3 ligand have been developed for this purpose, By providing an alternative molecule to which the ligand can bind, signalling through the FGFR3 molecule is reduced or inhibited. An example of a molecule that has been used to prevent excessive intracellular signaling through FGFR3, and rescue the symptoms of achondroplasia, is a soluble form of human FGFR3 (sFGFR3). This has been shown to acts as a decoy receptor and prevents FGF from binding to FGFR3. FGFR3 soluble decoys/sFGFR3 polypeptide are disclosed in WO16110786, WO2022/106976 and WO2018/007597, which are all hereby incorporated by reference in their entirety. Recifercept is a FGFR3 soluble decoy in clinical development for treatment of achondroplsia in children (Gongalves et al., PLoS One. 2020; 15(12): e0244368).

Inhibitors of FGFR3 signalling may also act to reduce or prevent FGFR3 signalling, by acting on (e.g. binding to or otherwise preventing the activity of) the intracellular portion of the FGFR3 molecule itself, for example by preventing or reducing phosphorylation of the FGFR3 molecule. Tyrosine kinase inhibitors, e.g. infigratinib or TYR 300, such as FGFR3 selective tyrosine kinase inhibitors can be used for this purpose. WO 2022/187443 discloses FGFR3 selective FGFR3 inhibitors (tyrosine kinase inhibitors) for use in treatment of achondroplasia and other disorders and is hereby incorporated by reference in its entirety. LY3866288, also referred to as LOXO-435 (also referred to as LOX-24350) is a FGFR3 inhibitor in clinical development.

Alternatively inhibitors of FGFR3 signalling may perform this function by acting on (e.g. binding to or otherwise preventing the activity of) molecules that are downstream of the receptor in one or more of its signalling pathways. Such inhibitors of the FGFR3 signalling pathway may act to reduce or prevent the activity of the MAPK signalling pathway or the STAT signalling pathway downstream from FGFR3. Examples of targets for inhibitors of FGFR3 signalling in the MAPK pathway are the proteins ras, raf, mek and erk. CNP acts in this way as an inhibitor of the FGFR3 signalling pathway; activation of the CNP receptor NPR-B gives rise to cGMP production and activation of PKG which inhibits the raf kinase. CNP, and other NPR-B agonists can therefore also be inhibitors of FGFR3 signalling. Inhibitors of FGFR3 signalling therefore may act directly or indirectly on one or more of FGFR3 itself, ras, raf, mek and erk, or STAT.

Similarly, preventing or reducing the expression of the FGFR3 protein itself is another possible way to inhibit FGFR3 signalling. An inhibitor of FGFR3 signalling may thus act to reduce or decrease the amount of FGFR3 protein in the cell. Examples of suitable strategies include antisense molecules and siRNA e.g. directed to the FGFR3 protein itself. In such cases the amount of FGFR3 in the relevant cell may be less than 90. 80. 70. 60, 50, 40, 30, 20, 10% of the amount of FGFR3 in the cell in the absence of the siRNA molecule or antisense molecule, or before the siRNA molecule or antisense molecule is administered.

Suitable examples of inhibitors of FGFR3 signalling therefore includes antibodies to FGFR3 (e.g. antagonistic antibodies to FGFR3) tyrosine kinase inhibitors (such as infigratinib, pemigatinib, futibatinib, erdafitinib or TYRA-300), molecules that prevent or reduce FGFR3-ligand binding, molecules that inhibit FGFR3 signalling via activation of the NPR-B receptor (e.g. C-type natriuretic peptide (CNP) and variants thereof), FGFR3 siRNAs and FGFR3 antisense oligonucleotides.

In certain embodiments, the fibroblast growth factor receptor 3 (FGFR3) antagonist is infigatinib which has the structure below, or a pharmaceutically acceptable salt thereof:

### Infigratinib

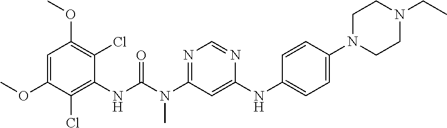 [SMILES: CCN1CCN(c2ccc(Nc3cc(N(C)C(=O)Nc4c(Cl)c(OC)cc(OC)c4Cl)ncn3)cc2)CC1]

Infigratinib has been approved for the treatment of certain cancers, and is presently in clinical development for the treatment of achondroplasia in children 3-11 years of age, who receive upto 0.25 mg/kg Infigatinib daily in tablet form (https://clinicaltrials.gov/ct2/show/NCT04265651). Infigratinib at high doses used in treatment of cancer is associated with side effects which may include muscle weakness and muscle cramps, it may therefore desirable to limit the daily dose in the context of the present invention to about 3 mg/kg or below, such as about 0.25 mg/kg or below.

Other inhibitors of FGFR3 signalling include pemigatinib, futibatinib, erdafitinib or TYRA-300. Inhibitors of FGFR3 signalling, including TYRA-300 are disclosed in WO2023/279041, WO2021/138392, WO2022/147246 and WO2021/138391 which are all hereby incorporated by reference in their entirety.

Inhibitors of FGFR3 signalling can be identified and characterized by standard methods, such as those described e.g. in WO2023/279041 WO2021/138392, WO2022/147246 and WO2021/138391.

### CNP Drugs

In an advantageous embodiment, C-type natriuretic peptide (CNP) drugs are used. As discussed above, in vivo CNP binds NPR-B to exert its effect on the FGFR3 signalling pathway and can thus also be described as a NPR-B agonist. Other NPR-B agonists may also be used according to the invention, including small molecule NPR-B receptor agonists.

CNP drugs are molecules which include a CNP peptide as defined above. The CNP drugs and CNP peptides are preferably either administered in a form in which they are NPR-B agonists as defined above, or they are administered in a form that gives rise to the production of NPR-B agonists in vivo (e.g. as a result of in vivo processing which releases a CNP peptide which is an NPR-B agonist in vivo).

By way of example, the CNP drug may be administered in the form of a CNP peptide, or a CNP peptide conjugate or CNP prodrug. The methods of the invention may also employ NPR-C ligands; CNP is an example of an NPR-C ligand.

### FGFR3 Inhibition Assay

Guagnana et al., J. Med. Chem. 2011, 54, 7066-7083, hereby incorporated by reference in its entirety provides a radiometric kinase assay which may be used to identify a FGFR3 tyrosine kinase inhibitor: The enzymatic kinase activity measures the phosphorylation of a synthetic substrate by the purified GST-fusion FGFR3-K650E kinase domain, in the presence of radiolabeled ATP. Also disclosed in Guagnana are BaF3 Cell Lines Proliferation Assays and FGFR1-4 Cellular Autophosphorylation Assay which may also be used to identify FGFR3 inhibitors.

### an NPR-B Activity Assay May be Used to Identify NPR-B Agonists

Functional CNP peptides, and free CNP released from CNP prodrug, and other NPR-B agonists may be identified using a NPR-B assay, such as the NPR-B assay reported in Breinholt et al., 2019 J Pharmacol Exp Ther 370:459-471, which is hereby incorporated by reference in its entirety.

The activity of CNP to elicit an intracellular cyclic guanosine monophosphate (cGMP) response may be determined in NIH3T3 cells. These cells express NPR-B on the cell surface (Abbey and Potter, 2003 Endocrinology, Volume 144, Issue 1, 1 Jan. 2003, Pages 240-246), and stimulation of this receptor with CNP leads to intracellular production of the secondary messenger cGMP. In brief, NIH3T3 cells are cultured in Dulbecco's modified Eagle's medium F-12 medium with 5% FBS and 5 mM glutamine at 37° C. and 5% CO₂. For each assay, cells are resuspended in stimulation buffer (Dulbecco's PBS with 0.5 mM 3-isobutyl-l-methylxanthine), seeded in a 96-well plate (5×10⁴/well), and incubated in duplicate with the CNP at different concentrations. After an incubation of 30 minutes at 37° C. and 5% CO₂. cells were lysed in the supplied lysis buffer, and the cGMP level is measured using a commercially available cGMP assay based on time-resolved fluorescence energy transfer (cGMP kit, catalogue 62GM2PEB; Cisbio, Codolet, France). Activity is determined using a four-parameter logistic curve fit and by parallel line analysis of the sample in comparison with CNP-38 (SEQ ID NO:24 (CNP-38): LQEHPNARKYKGANKKGLSKGCFGLKLDRIGSMSGLGC, wherein the cysteines at position 22 and 38 are connected through a disulfide-bridge) as a reference standard (PLA 2.0 software; Stegmann Systems, Rodgau, Germany).

In certain embodiments, the NPR-B agonist (e.g. C-type natriuretic peptide) has at least about 25% of the NPR-B activity (activity to elicit intracellular cGMP response in a NIH3T3 cell assay) of a CNP-38 reference standard, such as at least about 50%, 75%, 80%, 85%, 90% of the activity of the CNP-38 reference standard in the NPR-B activity assay as described above.

It will be understood that for CNP prodrugs, the activity of the prodrug in the CNP assay will need to be assessed on the CNP released (free CNP) from the prodrug.

An NPR-C Affinity & Agonist Assays which May be Used to Identify NPR-C Agonists

Functional CNP peptides, and free CNP released from CNP prodrug, and other NPR-C ligands may be identified using a NPR-C affinity assay, such as the NPR-C affinity assay reported in Breinholt et al., 2019 J Pharmacol Exp Ther 370:459-471.

This NPR-C affinity assay, uses a HEK293 cell line, stably overexpressing human NPR-C. This NPR-C affinity assay which may be used to assess the relative NPR-C affinity of a CNP or another NPR-C ligand versus a CNP-38 standard. In certain embodiments, the C-type natriuretic peptide has at least about 25% of the NPR-C affinity of the CNP-38 reference standard, such as at least about 50% of the affinity of the CNP-38 reference standard, such as at least about 75% affinity to NPR-C of the CNP-38 reference standard, in the NPR-C affinity assay.

Zhou and Murthy, Am J Physiol Cell Physiol 284: C1255-C1261, 2003, which is hereby incorporated by reference in its entirety, reports on the G-protein-activating activity of NPRC, and provides an assay for the identification of receptor-activated G proteins by [35S]GTPγS binding assay, and an assay for PLC-beta activity, either of which may be used to identify NPR-C ligands with NPR-C agonist activity (NPR-C agonists: for example a standard to use when assessing NPR-C agonists in a NPR-C activity assay is cANP4-23). Zhou and Murthery further discloses a [125I] ANP binding assay, which may be used to identify NPR-C ligands.

### Exemplary CNPs (Including CNP Peptides)

Naturally occurring human CNP-22 (SEQ ID NO: 1) has the following sequence:

GLSKGCFGLKLDRIGSMSGLGC,

wherein the cysteines at position 6 and 22 are connected through a disulfide-bridge.

In certain embodiments, the term “CNP” also refers to the following peptide sequences:

SEQ ID NO: 2 (CNP-53):
DLRVDTKSRAAWARLLQEHPNARKYKGANKKGLSKGCFGLKLDRIGSMS
GLGC;
SEQ ID NO: 3 (G-CNP-53):
GDLRVDTKSRAAWARLLQEHPNARKYKGANKKGLSKGCFGLKLDRIGSM
SGLGC;
SEQ ID NO: 4 (M-CNP-53):
MDLRVDTKSRAAWARLLQEHPNARKYKGANKKGLSKGCFGLKLDRIGSM
SGLGC;
SEQ ID NO: 5 (P-CNP-53):
PDLRVDTKSRAAWARLLQEHPNARKYKGANKKGLSKGCFGLKLDRIGSM
SGLGC;
SEQ ID NO: 6 (CNP-53 M48N):
DLRVDTKSRAAWARLLQEHPNARKYKGANKKGLSKGCFGLKLDRIGSNS
GLGC;
SEQ ID NO: 7 (CNP-53 Δ15-31):
DLRVDTKSRAAWARGLSKGCFGLKLDRIGSMSGLGC;
SEQ ID NO: 8 (CNP-52):
LRVDTKSRAAWARLLQEHPNARKYKGANKKGLSKGCFGLKLDRIGSMSG
LGC;
SEQ ID NO: 9 (CNP-51):
RVDTKSRAAWARLLQEHPNARKYKGANKKGLSKGCFGLKLDRIGSMSGL
GC;
SEQ ID NO: 10 (CNP-50):
VDTKSRAAWARLLQEHPNARKYKGANKKGLSKGCFGLKLDRIGSMSGLG
C;
SEQ ID NO: 11 (CNP-49):
DTKSRAAWARLLQEHPNARKYKGANKKGLSKGCFGLKLDRIGSMSGLGC;
SEQ ID NO: 12 (CNP-48):
TKSRAAWARLLQEHPNARKYKGANKKGLSKGCFGLKLDRIGSMSGLGC;
SEQ ID NO: 13 (CNP-47):
KSRAAWARLLQEHPNARKYKGANKKGLSKGCFGLKLDRIGSMSGLGC;
SEQ ID NO: 14 (CNP-46):
SRAAWARLLQEHPNARKYKGANKKGLSKGCFGLKLDRIGSMSGLGC;
SEQ ID NO: 15 (CNP-45):
RAAWARLLQEHPNARKYKGANKKGLSKGCFGLKLDRIGSMSGLGC;
SEQ ID NO: 16 (CNP-44):
AAWARLLQEHPNARKYKGANKKGLSKGCFGLKLDRIGSMSGLGC;
SEQ ID NO: 17 (CNP-44 Δ14-22):
AAWARLLQEHPNAGLSKGCFGLKLDRIGSMSGLGC;
SEQ ID NO: 18 (CNP-44 Δ15-22):
AAWARLLQEHPNARGLSKGCFGLKLDRIGSMSGLGC;
SEQ ID NO: 19 (CNP-43):
AWARLLQEHPNARKYKGANKKGLSKGCFGLKLDRIGSMSGLGC;
SEQ ID NO: 20 (CNP-42):
WARLLQEHPNARKYKGANKKGLSKGCFGLKLDRIGSMSGLGC;
SEQ ID NO: 21 (CNP-41):
ARLLQEHPNARKYKGANKKGLSKGCFGLKLDRIGSMSGLGC;
SEQ ID NO: 22 (CNP-40):
RLLQEHPNARKYKGANKKGLSKGCFGLKLDRIGSMSGLGC;
SEQ ID NO: 23 (CNP-39):
LLQEHPNARKYKGANKKGLSKGCFGLKLDRIGSMSGLGC;
SEQ ID NO: 24 (CNP-38):
LQEHPNARKYKGANKKGLSKGCFGLKLDRIGSMSGLGC,

wherein the cysteines at position 22 and 38 are connected through a disulfide-bridge;

SEQ ID NO: 25 (CNP-37):
QEHPNARKYKGANKKGLSKGCFGLKLDRIGSMSGLGC;
SEQ ID NO: 26 (CNP-37 Q1pQ, wherein pQ =
pyroglutamate):
pQEHPNARKYKGANKKGLSKGCFGLKLDRIGSMSGLGC;
SEQ ID NO: 27 (G-CNP-37):
GQEHPNARKYKGANKKGLSKGCFGLKLDRIGSMSGLGC;
SEQ ID NO: 28 (P-CNP-37):
PQEHPNARKYKGANKKGLSKGCFGLKLDRIGSMSGLGC;
SEQ ID NO: 29 (M-CNP-37):
MQEHPNARKYKGANKKGLSKGCFGLKLDRIGSMSGLGC;
SEQ ID NO: 30 (PG-CNP-37) (Vosoritide peptide
sequence):
PGQEHPNARKYKGANKKGLSKGCFGLKLDRIGSMSGLGC;
SEQ ID NO: 31 (MG-CNP-37):
MGQEHPNARKYKGANKKGLSKGCFGLKLDRIGSMSGLGC;
SEQ ID NO: 32 (CNP-37 M32N):
QEHPNARKYKGANKKGLSKGCFGLKLDRIGSNSGLGC;
SEQ ID NO: 33 (G-CNP-37 M32N):
GQEHPNARKYKGANKKGLSKGCFGLKLDRIGSNSGLGC;
SEQ ID NO: 34 (G-CNP-37 K14Q):
GQEHPNARKYKGANQKGLSKGCFGLKLDRIGSMSGLGC;
SEQ ID NO: 35 (G-CNP-37 K14P):
GQEHPNARKYKGANPKGLSKGCFGLKLDRIGSMSGLGC;
SEQ ID NO: 36 (G-CNP-37 K14Q, Δ15):
GQEHPNARKYKGANQGLSKGCFGLKLDRIGSMSGLGC;
SEQ ID NO: 37 (G-CNP-37 K14Q, K15Q):
GQEHPNARKYKGANQQGLSKGCFGLKLDRIGSMSGLGC;
SEQ ID NO: 38 (CNP-36):
EHPNARKYKGANKKGLSKGCFGLKLDRIGSMSGLGC;
SEQ ID NO: 39 (CNP-35):
HPNARKYKGANKKGLSKGCFGLKLDRIGSMSGLGC;
SEQ ID NO: 40 (CNP-34):
PNARKYKGANKKGLSKGCFGLKLDRIGSMSGLGC;
SEQ ID NO: 41 (CNP-33):
NARKYKGANKKGLSKGCFGLKLDRIGSMSGLGC;
SEQ ID NO: 42 (CNP-32):
ARKYKGANKKGLSKGCFGLKLDRIGSMSGLGC;
SEQ ID NO: 43 (CNP-31):
RKYKGANKKGLSKGCFGLKLDRIGSMSGLGC;
SEQ ID NO: 44 (CNP-30):
KYKGANKKGLSKGCFGLKLDRIGSMSGLGC;
SEQ ID NO: 45 (CNP-29):
YKGANKKGLSKGCFGLKLDRIGSMSGLGC;
SEQ ID NO: 46 (CNP-28):
KGANKKGLSKGCFGLKLDRIGSMSGLGC;
SEQ ID NO: 47 (GHKSEVAHRF-CNP-28):
GHKSEVAHRFKGANKKGLSKGCFGLKLDRIGSMSGLGC;
SEQ ID NO: 48 (CNP-27):
GANKKGLSKGCFGLKLDRIGSMSGLGC;
SEQ ID NO: 49 (CNP-27 K4Q, K5Q):
GANQQGLSKGCFGLKLDRIGSMSGLGC;
SEQ ID NO: 50 (CNP-27 K4R, K5R):
GANRRGLSKGCFGLKLDRIGSMSGLGC;
SEQ ID NO: 51 (CNP-27 K4P, K5R):
GANPRGLSKGCFGLKLDRIGSMSGLGC;
SEQ ID NO: 52 (CNP-27 K4S, K5S):
GANSSGLSKGCFGLKLDRIGSMSGLGC;
SEQ ID NO: 53 (CNP-27 K4P, K5R):
GANGANPRGLSRGCFGLKLDRIGSMSGLGC;
SEQ ID NO: 54 (CNP-27 K4R, K5R, K9R):
GANRRGLSRGCFGLKLDRIGSMSGLGC;
SEQ ID NO: 55 (CNP-27 K4R, K5R, K9R, M22N):
GANRRGLSRGCFGLKLDRIGSNSGLGC;
SEQ ID NO: 56 (P-CNP-27 K4R, K5R, K9R):
PGANRRGLSRGCFGLKLDRIGSMSGLGC;
SEQ ID NO: 57 (M-CNP-27 K4R, K5R, K9R):
MGANRRGLSRGCFGLKLDRIGSMSGLGC;
SEQ ID NO: 58 (HSA fragment-CNP-27):
GHKSEVAHRFKGANKKGLSKGCFGLKLDRIGSMSGLG;
SEQ ID NO: 59 (HSA fragment-CNP-27 M22N):
GHKSEVAHRFKGANKKGLSKGCFGLKLDRIGSNSGLGC;
SEQ ID NO: 60 (M-HSA fragment-CNP-27):
MGHKSEVAHRFKGANKKGLSKGCFGLKLDRIGSMSGLGC;
SEQ ID NO: 61 (P-HSA fragment-CNP-27):
PGHKSEVAHRFKGANKKGLSKGCFGLKLDRIGSMSGLGC;
SEQ ID NO: 62 (CNP-26):
ANKKGLSKGCFGLKLDRIGSMSGLGC;
SEQ ID NO: 63 (CNP-25):
NKKGLSKGCFGLKLDRIGSMSGLGC;
SEQ ID NO: 64 (CNP-24):
KKGLSKGCFGLKLDRIGSMSGLGC;
SEQ ID NO: 65 (CNP-23):
KGLSKGCFGLKLDRIGSMSGLGC;
SEQ ID NO: 66 (R-CNP-22):
RGLSKGCFGLKLDRIGSMSGLGC;
SEQ ID NO: 67 (ER-CNP-22):
ERGLSKGCFGLKLDRIGSMSGLGC;
SEQ ID NO: 68 (R-CNP-22 K4R):
RGLSRGCFGLKLDRIGSMSGLGC;
SEQ ID NO: 69 (ER-CNP-22 4KR):
ERGLSRGCFGLKLDRIGSMSGLGC;
SEQ ID NO: 70 (RR-CNP-22):
RRGLSRGCFGLKLDRIGSMSGLGC;
SEQ ID NO: 71 (HRGP fragment-CNP-22):
GHHSHEQHPHGANQQGLSKGCFGLKLDRIGSMSGLGC;
SEQ ID NO: 72 (HRGP fragment-CNP-22):
GAHHPHEHDTHGANQQGLSKGCFGLKLDRIGSMSGLGC;
SEQ ID NO: 73 (HRGP fragment-CNP-22):
GHHSHEQHPHGANPRGLSKGCFGLKLDRIGSMSGLGC;
SEQ ID NO: 74 (IgG1(Fc) fragment-CNP-22):
GQPREPQVYTLPPSGLSKGCFGLKLDRIGSMSGLGC;
SEQ ID NO: 75 (HSA fragment-CNP-22):
GQHKDDNPNLPRGANPRGLSKGCFGLKLDRIGSMSGLGC;
SEQ ID NO: 76 (HSA fragment-CNP-22):
GERAFKAWAVARLSQGLSKGCFGLKLDRIGSMSGLGC;
SEQ ID NO: 77 (osteocrin NPR C inhibitor
fragment-CNP22):
FGIPMDRIGRNPRGLSKGCFGLKLDRIGSMSGLGC;
SEQ ID NO: 78 (FGF2 heparin-binding domain
fragment-CNP22):
GKRTGQYKLGSKTGPGPKGLSKGCFGLKLDRIGSMSGLGC;
SEQ ID NO: 79 (IgG1(Fc) fragment-CNP-22 K4R):
GQPREPQVYTGANQQGLSRGCFGLKLDRIGSMSGLGC;
SEQ ID NO: 80 (HSA fragment-CNP-22 K4R):
GVPQVSTSTGANQQGLSRGCFGLKLDRIGSMSGLGC;
SEQ ID NO: 81 (fibronectin fragment-CNP-22 K4R):
GQPSSSSQSTGANQQGLSRGCFGLKLDRIGSMSGLGC;
SEQ ID NO: 82 (fibronectin fragment-CNP-22 K4R):
GQTHSSGTQSGANQQGLSRGCFGLKLDRIGSMSGLGC;
SEQ ID NO: 83 (fibronectin fragment-CNP-22 K4R):
GSTGQWHSESGANQQGLSRGCFGLKLDRIGSMSGLGC;
SEQ ID NO: 84 (zinc finger fragment-CNP-22 K4R):
GSSSSSSSSSGANQQGL SRGCFGLKLDRIGSMSGLGC;
SEQ ID NO: 85 (CNP-21):
LSKGCFGLKLDRIGSMSGLGC;
SEQ ID NO: 86 (CNP-20):
SKGCFGLKLDRIGSMSGLGC;
SEQ ID NO: 87 (CNP-19):
KGCFGLKLDRIGSMSGLGC;
SEQ ID NO: 88 (CNP-18):
GCFGLKLDRIGSMSGLGC;
SEQ ID NO: 89 (CNP-17):
CFGLKLDRIGSMSGLGC;
SEQ ID NO: 90 (BNP fragment-CNP-17-BNP fragment):
SPKMVQGSGCFGLKLDRIGSMSGLGCKVLRRH;
SEQ ID NO: 91 (CNP-38 L1G):
GQEHPNARKYKGANKKGLSKGCFGLKLDRIGSMSGLGC;
SEQ ID NO: 92 (Ac-CNP-37; wherein Ac = acetyl):
Ac-QEHPNARKYKGANKKGLSKGCFGLKLDRIGSMSGLGC;
SEQ ID NO: 93:
QEHPNARX₁YX₂GANX₃X₄GLSX₅GCFGLX₆LDRIGSMSGLGC,

wherein X₁, X₂, X₃, X₄, X₅ and X₆ are independently of each other selected from the group consisting of K, R, P. S and Q, with the provision that at least one of X₁, X₂, X₃, X₄, X₅ and X₆ is selected from the group consisting of R, P, S and Q; in certain embodiments, X₁, X₂, X₃, X₄, X₅ and X₆ are selected from the group consisting of K and R, with the provision that at least one of X₁, X₂, X₃, X₄, X₅ and X₆ is R;

SEQ ID NO: 94:
QEHPNARKYKGANX₁X₂GLSX₃GCFGLXALDRIGSMSGLGC,

wherein X₁, X₂, X₃ and X₄ are independently of each other selected from the group consisting of K, R, P. S and Q, with the provision that at least one of X₁, X₂, X₃ and X₄ is selected from the group consisting of R, P. S and Q; in certain embodiments, X₁, X₂, X₃ and X₄ are selected from K and R, with the provision that at least one of X₁, X₂, X₃ and X₄ is R;

SEQ ID NO: 95:
QEHPNARKYKGANX₁X₂GLSKGCFGLKLDRIGSMSGLGC,

wherein X₁X₂ are selected from the group consisting of KR, RK, KP, PK, SS, RS, SR, QK, QR, KQ, RQ, RR and QQ.

It is understood that also the equivalents of the cysteines in positions 22 and 38 of SEQ ID NO:24 are connected through a disulfide-bridge in SEQ ID NOs: 2 to 95.

It is also recognized by one of ordinary skill in the art that the conjugates of the present invention may be prodrugs.

The unit dose comprised within the unit dosage form of the present invention depends on the patient's actual body weight.

Vosoritide is approved for daily subcutanteous administration, and is dosed at or about 15 ug/kg, although may be dosed higher in infants (e.g. about 30 ug/kg). Vosoritide is presently available in 0.4 mg, 0.56 mg and 1.2 mg vials, and the current recommended daily dose based on actual body weight (ABW) is as follows.
10-11 kg: 0.24 mg SC qDay
12-16 kg: 0.28 mg SC qDay
17-21 kg: 0.32 mg SC qDay
22-32 kg: 0.4 mg SC qDay
33-43 kg: 0.5 mg SC qDay
44-59 kg: 0.6 mg SC qDay
60-89 kg: 0.7 mg SC qDay
≥90 kg: 0.8 mg SC qDay

For younger children it has been reported that a high dose is more efficacious.

In certain embodiments, the unit dose ranges from 50 μg to 7000 μg of CNP. In certain embodiments, the unit dose ranges from 100 μg to 5000 μg of CNP. In certain embodiments, the unit dose ranges from 100 μg to 3000 μg of CNP. In certain embodiments, the unit dose ranges from 100 μg to 2000 μg of CNP. In certain embodiments, the unit dose ranges from 100 μg to 1000 μg of CNP. In certain embodiments, the unit dose ranges from 150 μg to 750 μg of CNP. In certain embodiments, the unit dose ranges from 150 μg to 500 μg of CNP. In certain embodiments, the unit dose ranges from 150 μg to 350 μg of CNP. In certain embodiments, the unit dose is about 700 μg CNP. In certain embodiments, the unit dose is about 600 μg CNP. In certain embodiments, the unit dose is about 500 μg CNP. In certain embodiments, the unit dose is about 400 μg CNP. In certain embodiments, the unit dose is about 300 μg CNP.

In certain embodiments, the unit dose is about 6 μg CNP/kg. In certain embodiments, the unit dose is about 20 μg CNP/kg. In certain embodiments, the unit dose is about 50 μg CNP/kg. In certain embodiments, the unit dose is about 75 μg CNP/kg. In certain embodiments, the unit dose is about 100 μg CNP/kg. In certain embodiments, the unit dose is about 125 μg CNP/kg. In certain embodiments, the unit dose is about 150 μg CNP/kg.

In certain embodiments, the unit dose is 6 μg CNP/kg. In certain embodiments, the unit dose is 20 μg CNP/kg. In certain embodiments, the unit dose is 50 μg CNP/kg. In certain embodiments, the unit dose is 75 μg CNP/kg. In certain embodiments, the unit dose is 100 μg CNP/kg. In certain embodiments, the unit dose is 125 μg CNP/kg. In certain embodiments, the unit dose is 150 μg CNP/kg.

In certain embodiments, the unit dosage form is liquid. In certain embodiments, the unit dosage form is solid.

In certain embodiments, such as for compound of formula (IIf′) or formula (IIf) or compound (1), the unit dose ranges from about 12.3 nmol CNP/kg to at least about 37 nmol CNP/kg. In certain embodiments, the unit dose ranges from 12.3 nmol CNP/kg to 36.9 nmol CNP/kg. In certain embodiments, the unit dose ranges is at least 24.6 nmol CNP/kg. In certain embodiments, the unit dose ranges is about 24.6 nmol CNP/kg. In certain embodiments, the unit dose ranges is 24.6 nmol CNP/kg.

In certain embodiments, such as for compound of formula (IIf′) or formula (IIf) or compound (1), the unit dose ranges from about 6 μg CNP/kg to at least about 100 μg CNP/kg. In certain embodiments, the unit dose ranges from about 6 μg CNP/kg to about 150 μg CNP/kg.

In certain embodiments, such as for compound of formula (IIf′) or formula (IIf) or compound (1), the unit dose comprised within the unit dosage form of the present invention ranges from 6 μg CNP/kg to at least 100 μg CNP/kg. In certain embodiments, the unit dose ranges from 6 μg CNP/kg to 150 μg CNP/kg.

The unit dose is preferably administered weekly.

For CNP conjugates or CNP prodrugs, such as such as for compound of formula (IIf′) or formula (IIf) or compound (1), it is understood that “x” μg CNP/kg refers to “x” μg of CNP, i.e. of the CNP moiety comprised within the CNP conjugate, per kilogram of patient's body weight. Similarly, it is understood that “y” nmol CNP/kg refers to “y” nmol of CNP, i.e. of the CNP moiety comprised within the CNP conjugate, per kilogram of patient's body weight.

### The Subject/Patient

In certain embodiments, the subject is an adult. Adult humans are 18 years of age and above. In certain embodiments the subject is at least 19 years of age, such as at least 20 or 25 years of age.

In certain embodiments, the subject is a pediatric patient, i.e. below 18 years of age, such as below 16 years or below 14 years, or 5 years of age. In certain embodiments, the subject is an infant (e.g. less than 1 year or less than 9 or 6 months).

The subject is also referred to as the patient.

In certain embodiments, the patient's body weight ranges from about 2 kg to about 80 kg. In certain embodiments, the patient's body weight ranges from about 4 kg to about 60 kg. In certain embodiment, the patient's body weight is about 5 kg. In certain embodiment, the patient's body weight is about 9 kg. In certain embodiment, the patient's body weight is about 10 kg. In certain embodiment, the patient's body weight is about 11 kg. In certain embodiment, the patient's body weight is about 12 kg. In certain embodiment, the patient's body weight is about 15 kg. In certain embodiment, the patient's body weight is about 20 kg. In certain embodiment, the patient's body weight is about (at least about) 30 kg. In certain embodiment, the patient's body weight is about (at least about) 40 kg. In certain embodiment, the patient's body weight is about (at least about) 50 kg. In certain embodiment, the patient's body weight is about (at least about) 60 kg. In certain embodiment, the patient's body weight is about (at least about) 70 kg. In certain embodiment, the patient's body weight is about (at least about) 80 kg.

Diseases or Conditions in which Muscle Function is Impaired

In certain embodiments the subject is suffering from a disease or condition in which muscle function (e.g. skeletal muscle) is impaired. By this it is meant that the normal muscle function is reduced or absent. The reduction in muscle function can be assessed relative to threshold levels or by reference to an individual without the disease or condition. Loss or reduction in muscle function could arise as a result of the disease or condition acting directly on the muscle (e.g. a myopathy) or may be a disease affecting the neuromuscular junction or a disease affecting the nervous system.

The disease or condition in which muscle function (e.g. skeletal muscle) is impaired may include a chondrodysplasia disease, such as a disease selected from the group consisting of achondroplasia, hypochondroplasia, thanatophoric dysplasia. In certain embodiments the subject has achondroplasia.

In certain embodiments the subject has a Rasopathy.

In certain embodiments the subject has a chondrodysplasia disease, such as a disease selected from the group consisting of achondroplasia, hypochondroplasia, thanatophoric dysplasia. In certain embodiments the subject has achondroplasia.

In certain embodiments the subject does not have a chondrodysplasia disease, such as a disease selected from the group consisting of achondroplasia, hypochondroplasia, thanatophoric dysplasia. In certain embodiments the subject does not have achondroplasia. In certain embodiments the subject does not have a chondrodysplasia disease.

Skeletal muscle is one of the three main types of muscle in the body, the others being cardiac muscle and smooth muscle. Skeletal muscle is muscle which is attached to the bone via tendons. Skeletal muscle may also be referred to a striated muscle. Administering the inhibitor of FGFR3 signalling or NPR-B agonist or NPR-C agonist has been shown to give rise to an improvement in muscle function, particularly skeletal muscle function.

In certain embodiments the subject has a myopathy. Myopathies are a heterogeneous group of disorders primarily affecting the skeletal muscle structure, metabolism, or channel function, which typically result in muscle weakness, stiffness, cramps and spasms, and may result in or contribute to skeletal deformities, such as abnormal curvature of the spine. Myopathies usually present with muscle weakness interfering in daily life activities. In some embodiments, the myopathy or impaired muscle function may be a mitochondrial myopathy, i.e. a myopathy caused by a defect in the mitochondria. Myopathy may be inherited or acquired. Inherited myopathies may be congenital myopathy, i.e. myopathy symptoms start at birth or early childhood. In certain embodiments the subject has a disease or condition associated with an impairment in neuromuscular function, such as a neuromuscular or a neurodegenerative disease.

In some embodiments therefore the methods are carried out on a subject having a disease or condition associated with an impairment in neuromuscular function, such as a neuromuscular or a neurodegenerative disease.

The invention also provides a method of treating or preventing a disease or condition associated with an impairment in neuromuscular function, such as a neuromuscular or neurodegenerative disease, the method comprising administering a therapeutically effective amount of an inhibitor of FGFR3 signaling, an NPR-B agonist or an NPR-C agonist (e.g. an NPR-B agonist, such as a C type natriuretic peptide (such as conjugates of CNP)), to a subject who is suffering from the neuromuscular or or neurodegenerative disease. In some embodiments, the neuromuscular or neurodegenerative disease is is one in which mitochondrial dysfunction is present.

The said administration of the effective amount of the inhibitor of FGFR3 signaling, NPR-B agonist or NPR-C agonist (e.g. NPR-B agonist) to the subject who has a disease or condition associated with an impairment in neuromuscular function may result in improved mitochondrial function in said subject. This may, for example be observed (or monitored) via improved muscle function such as improved skeletal muscle function or improved neuromuscular function; or via a slowing, delaying or reduction of disease progression, such as a reduced rate of loss or decline of muscle function, or reduced rate of loss or decline of neuromuscular function (e.g. compared to a reference, or to the subject prior to, or in the absence of the treatment). In some embodiments, the method of treatment may be initiated after diagnosis of the disease or condition associated with an impairment in neuromuscular function, and optionally prior to significant or noticeable loss in muscle or neuromuscular function.

Impaired mitochondrial function, or mitochondrial dysfunction, is indicated in many neurodegenerative disease, including Parkinson's diseases (PD), Alzheimer's (AD), Huntington's disease (HD), ataxias, such as Friedreich's ataxia (FRDA), and amyotrophic lateral sclerosis (ALS). Mitochondrial dysfunction may be identified by the diagnosis of a disease or disorder which is associated with mitochondrial dysfunction, via genetic testing, i.e. identification of a genetic polymorphism which is associated with or causes mitochondria dysfunction, via biochemical analysis from a biopsy of the affected tissue, or via biochemical markers in blood or urine (See for example Muraresku et al., Curr Genet Med Rep. 2018 June; 6(2): 62-72).

The disease or condition associated with an impairment in neuromuscular function may include a neuromuscular disorder/neuromuscular disease (NMDs). Suitable NMD disorders and diseases may be associated with mitochondrial impairment and dysfunction (see for example Marra et al., Biomolecules 2021, vol 11(11); 1633. The methods of the present invention may therefore be used to improve mitochondrial function, or slow the decline in mitochondrial function, in subject having a disease or condition associated with an impairment in neuromuscular function (e.g. a NMD) (e.g. compared to a reference, or to the subject prior to or in the absence of the treatment).

The methods of the present invention may therefore be used to improve muscle function, or slow the decline in muscle function, in these diseases or conditions associated with an impairment in neuromuscular function. Examples of neuromuscular diseases include genetically acquired diseases, including muscular dystrophies and myopathies as well as neuromuscular diseases. Neuromuscular diseases include neurodegenerative diseases and disorders associated with loss of mitochondrial function, and may for example, be selected from the group consisting of: Motor neuron disease (MND, also known as Amyotrophic Lateral Sclerosis, ALS); Parkinson's Disease (PD), Multiple Sclerosis (MS, including progressive MS or relapsing remitting MS); Alzheimer's Disease (AD); ataxia's such as Friedreich's ataxia (FRDA), and Huntington's disease (HD).

Parkinson's disease (PD) is associated with mitochondrial dysfunction and the pathogenesis in PD leads to muscular weakness and fatigue (Borsche et al., J Parkinsons Dis. 2021; 11(1):45-60). Timmer et al., J Neurosci. 2007 Jan. 17; 27(3): 459-471 indicates aberrant FGFR3/FGF-2 signaling in animal models of PD. Central and systemic CNP (amino-terminal proCNP) levels are both reduced in Parkinson's Disease, and can be restored by treatment with monoamine oxidase inhibitors (Espiner et al., J Neural Transm (Vienna). 2014 April; 121(4):371-8; Woodward, Parkinsonism Relat Disord. 2017 October; 43:15-19). Muscle weakness and fatigue, which may significantly influence gait, are commonly reported by patients with PD. Zheng et al., Sci Transl Med. 2010 Oct. 6; 2(52): 52ra73 reports PGC-1alpha as a therapeutic target in Parkinson's disease. Mitochondrial dysfunction is a central dysfunction in the pathology of Parkinson's Disease (PD), and PD-associated genes, including PGC-1α, are strictly connected with mitochondrial integrity (Piccinin, Int. J. Mol. Sci 2001 22(7) 3487. Low expression of PGC-1α is indicated in PD, and there is a need for therapeutic agents which elevate expression of PGC-1α and enhance mitochondrial function in PD patients.

Muscle function may be routinely monitored in PD patients using physical functioning tests such as sit to stand tests and six-minute walk tests (see e.g. Clael et al, Neurosci J 2918 8507018). Suitable physical functioning tests used in monitoring of PD patients may include speech, facial expression, rising from a chair, gait, or postural stability (see e.g. Brusse et al., Physical Therapy, Volume 85, Issue 2, 1 Feb. 2005, Pages 134-141). Recently, the FDA has approved a PD monitoring app (Rube Labs) for patient monitoring via a smart device (e.g. a phone or smart watch), which can provide all day monitoring of physical activities such as walking, as well as other PD symptoms such as tremors and dyskinesia, it is therefore envisaged that such smart device apps be used to monitor the effective treatment according to the present invention.

Reduced expression of PGC-la contributes to mitochondrial dysfunction and correlates with neuronal loss in multiple sclerosis (Witte et al., Acta Neuropathol. 2013 February; 125(2):231-43; Peixoto de Barcelos Biology (Basel) 2019 8(2):37). Rosenkranz et al., eLife 2021; 10:e61798 reports that enhancing mitochondrial activity in neurons protects against neurodegeneration in a mouse model of multiple sclerosis (MS) and proposes that boosting activity of mitochondria in neurons may be a therapeutic strategy for MS. Rajendran et al., Cells 2021 reports on FGF/FGFR signaling in MS and FGFR inhibition as a therapeutic option to reduce inflammation and induce remyelination. Multiple sclerosis causes muscular fatigue, pain, imbalance, and a reduction in physical activity which may further contribute to muscle weakness. Indeed, muscular fatigue is considered to be the most common MS symptom. Monitoring of muscle function in MS may be via self-report questionnaires, or via the use of smart devices, which may provide remote monitoring of fatigue and activity (e.g. Block Front. Neurol. 2022 13 https://doi.org/10.3389/fneur.2022.878313, Stuart et al., Mult Scler J Exp Transl Clin. 2020 Dec. 7; 6(4):2055217320975185.). Witte et al., reports that reduced expression of PGC-1alpha is correlated with neuronal loss in MS.

Zhao et al., Cells 2002 July; 11(13) 2049 reports that mitochondria dysfunction is a key contributing factor in amyotrophic lateral sclerosis (ALS) and that mitochondria disfunction is associated with and involved in the pathogenesis of the disease. Zhao et al., Molecular Neurodegeneration 6, Article number: 51 (2011) reports that PGC-1alpha plays a protective role in ALS.

Impairment of PGC-la mediated mitochondrial biogenesis is indicated in Alzheimer's disease pathology, and precedes mitochondrial dysfunction associated with progression of AD (Bhatia, Curr. Neuropharmacol. 2002 20(4): 675-692. Physical functioning is affected in people living with dementia, such as Alzheimer's Disease (AD), and can lead to slow reaction time, muscle weakness, poor coordination and impaired balance, and along with cognitive impairment is a major contributor to events such as falls and fractures. In later stages of dementia, physical ability may be significantly compromised, severely limiting walking, gait and motion (see e.g. Taraldsen et al., BMC Geriatrics volume 21, Article number: 670 (2021) which reports on the use of physical accelerometer sensors to monitor daily physical activity in dementia patients).

Various changes in muscle function may arise as a result of administration of the inhibitor of FGFR3 signalling or or NPR-B agonist or NPR-C agonist.

For example there may be an improvement in muscle strength. Muscle strength is the maximal ability to exert force for a short period of time. This may be assessed for example using the Oxford Scale which involves testing key muscles from the upper nd lower extremities against the examiner's resistance and grading the patient's strength on a 0 to 5 scale accordingly (Naqvi U. Muscle strength grading. InStatpearls [Internet] 2019 May 29. StatPearls Publishing. Available from: https://www.ncbi.nlm.nih.gov/books/NBK436008/):
0 No muscle activation
1 Trace muscle activation, such as a twitch, without achieving full range of motion
2 Muscle activation with gravity eliminated, achieving full range of motion
3 Muscle activation against gravity, full range of motion
4 Muscle activation against some resistance, full range of motion
5 Muscle activation against examiner's full resistance, full range of motion

Commonly tested muscles include the shoulder abductors, elbow flexors, elbow extensors, wrist extensors, finger flexors, hand intrinsics, hip flexors, knee extensors, dorsiflexors, great toe extensor, and plantar flexors. These muscle groups are commonly chosen, so that important spinal nerve roots are assessed systematically eg testing the strength of the elbow flexors, elbow extensors, wrist extensors, finger flexors, and hand intrinsics allow for a methodical evaluation of the C5 to T1 nerve roots. Alternatively, or additionally, distal strength can be semiquantitatively measured with a handgrip ergometer (or with an inflated BP cuff squeezed by the patient) to record grip strength, or dynamometry, which is a more precise measurement of the force that a muscle can exert and can allow for differences in strength to be recorded over time. An increase in muscle strength therefore may be observed as an increase in the Oxford score, or an increase in distal strength as assessed by a handgrip ergometer (or with an inflated BP cuff squeezed by the patient) to record grip strength, or dynamometry.

For example there may be an improvement in muscle tone. Muscle tone is also referred to as residual muscle tension or tonus and is the continuous and passive partial contraction of the muscles, or the muscle's resistance to passive stretch during resting state. It helps to maintain posture and declines during REM sleep. It is distinct from muscle strength. Muscle tone is regulated by the activity of the motor neurons and can be affected by various factors, including age, disease, and nerve damagehypotonia. Hypotonia refers to a decreased resting tone of the muscle and decreased resistance to passive movement. Hypotonia is rarely seen in isolation without some degree of weakness. Hypotonus (low muscle tone) describes a reduced stiffness of muscle that does not effectively support upright posture against gravity or to produce adequate force during contraction; as a result, hypotonic muscles are typically more compliant than they are stiff. Hypotonia is observed for example in subjects having various conditions such as Down syndrome, Muscular dystrophy, Cerebral palsy, Prader-Willi syndrome, Myotonic dystrophy, Marfan syndrome, Tay-Sachs disease. Hypotonia is in general assessed by medical observation and an improvement in hypotonia will result in increased resting tone and resistance to passive movement, as assessed by a medical professional.

For example there may be an improvement in muscular endurance (or muscle stamina). This is the ability of a muscle's or group of muscles' resistance to fatigue during repetitive muscle contractions against and external force. This is generally assessed observationally, e,g, by carrying out a repetitive exercise over a period of time, for example counting the number of push ups a patient can carry out in a given period, e.g. 60 seconds, or determining how long an individual takes to carry out a set number of repeats of an exercise. An improvement in muscular endurance would manifest in an improvement in the number of repeptive exercises in the given period of time or in a reduction in the amount of time required to carry out a set number of repeats of the exercise.

For example there may be an improvement in muscle mass. Muscle mass is the amount of muscle in the subject's body, including skeletal muscles, smooth muscles, and cardiac muscles. In preferred embodiments there is an improvement in skeletal muscle mass. Muscle mass can be measured e.g. by carrying out Dual energy X-ray absorptiometry, a CT scan, or an MRI scan. Muscle mass may be increased by the methods of the invention, and an increase in muscle mass may also give rise to an increase in muscle to fat ratio (e.g skeletal muscle to fat ratio). Fat mass can be measured using DEXA and Bioelectric Impedance Analysis (BIA). Additionally or alternatively there may also be an increase in one or more of muscle volume, muscle density and muscle length of the subject.

For example there may be a decrease in muscle fatigue. Muscle fatigue is used to describe the decline in force produced by a muscle or group of muscles during repetitive contractions against an external force. An example of a test to measure muscle fatigue is the Biering-Sorenson test which has been used to assess fatigue of the trunk extensor muscle group and which is also used as a test for back pain. The test as described by Sorenson (Biering-Sorensen F. Physical measurements as risk indicators for low-back trouble over a one-year period. Spine. 1984; 9:106-119.) is “measuring how many seconds the subject is able to keep the unsupported upper body (from the upper border of the iliac crest) horizontal, while placed prone with the buttocks and legs fixed to the couch by three wide canvas straps and the arms folded across the chest. A decrease in muscle fatigue would manifest in an improvement in the amount of time the subject is able to remain in the required position.

For example there may be an increase in cardiovascular endurance, which is the ability of the subject's heart and lungs to fuel their body with oxygen. This could also be defined the ability to perform moderate to high intensity exercise (at a percentage of VO2max) for a prolonged period. Cardiovascular endurance can be measured by standard techniques including measuring V02 max through indirect calorimetry. VO2max is the maximum amount of oxygen that an individual can utilize during intense or maximal exercises and is typically measured by determining volume and gas concentrations of inspired and expired air while a person performs maximal, graded exercise on a treadmill or cycle ergometer. VO2max can be expressed in terms of liters of oxygen consumed per minute (1/min), or the values can be normalized for differences in body size and expressed as milliliters of oxygen consumed per kilogram of body weight per minute (ml/kg/min). An increase in cardiovascular endurance may therefore be observed as an increase in VO2max (1/min), or (ml/kg/min) using this assay.

For example there may be an increase in cardiovascular fitness. Cardiovascular fitness is the the body's maximal ability to take up oxygen. This is also measured as VO2max (L O2/Min). An increase in cardiovascular fitness may therefore also be observed as an increase in VO2max (1/min), or (ml/kg/min).

For example there may be a decrease in exercise intolerance. Exercise intolerance is the reduced ability of the body to perform activities that involve strenuous movement, and may result in inability or decreased ability to perform physical exercise at a normal level or duration. Objective tests for exercise intolerance include moderate activity, e.g. stair climbing, walking for six minutes, a shuttle-walk test, a cardiac stress test, and the cardiopulmonary exercise test (CPET). In the six-minute walk test, the goal is to see how far the person can walk, with approximately 600 meters being a reasonable outcome for an average person without exercise intolerance. A decrease in exercise intolerance may therefore be observed as an increase in the distance a person can walk in the six-minute test.

For example there may be an increase in exercise capacity, which is the maximum amount of physical exertion that a patient can sustain. This can be assessed by maximal exercise tests (e.g. ESTs which are symptom-limited tests performed with a 12-lead ECG used to diagnose exercise-induced myocardial ischaemia, arrhythmias or an abnormal blood pressure response, or a cardiopulmonary exercise test), submaximal exercise tests, six minute walk tests and incremental shuttle walk tests, with the appropriate test being selected depending on the subject's physical state. An increase in exercise capacity may therefore be observed as an increase in the score obtained in any of these assessments.

For example there may be a decrease in exercise-induced fatigue (EF), which is a reduction in maximal voluntary muscle force that results from intense and prolonged exercise. In general this is measured by observation of fatigue state after intense and prolonged exercise.

Many of the improvements in muscle function are based on observation by a medical professional and are not quantified, however the increase or improvement may be one which is observed by the medical professional on the basis of the standard tests for that muscle function that are used in the field. Any such increases or improvements that can be quantified may be an increase or improvement in the performance as determined by a score or value that is at least 1, 2, 3, 5, 10, 15, 20, 25, 30, 35%, improved (whether the actual value increases or decreases, depending on the nature of the test) relative to that value without or before administration of the FGFR3 signalling inhibitor or or NPR-B agonist or NPR-C agonist, for example after at least 1, 2, 3, 4, 5, 6, 12, 18, 24, 36 months of administration of the FGFR3 signalling inhibitor or NPR-B agonist or NPR-C agonist. Optionally, biochemical assays on muscle biopsy or for products of muscle metabolism released to body fluids may be used in these methods, and may give rise to quantitative outcomes.

As noted above, an improvement in muscle function may be defined as an improvement relative to muscle function without or before administration of FGFR3 signalling inhibitor or or NPR-B agonist or NPR-C agonist. In certain embodiments therefore, a subject treated with an FGFR3 signalling inhibitor or or NPR-B agonist or NPR-C agonist may still have an overall decline in muscle function (e.g. over time), but this decline in muscle function may in these embodiments be less than the decline that would have occurred in the absence of the treatment. In such optional embodiments, an improvement in muscle function may be manifest as a reduction in decline of muscle function (e.g. as compared to the decline in muscle function in the absence of treatment). This is particularly the case for those conditions referred to herein as a disease or condition associated with an impairment in neuromuscular function, such as a neuromuscular or a neurodegenerative disease. This may mean that a subject takes longer to reach a certain level of muscle function than would be expected, based on the subject's diagnosis. Alternatively stated. the progression of the disease (e.g. in terms of muscle function) is slowed, e.g. so that it takes at least 1, 2, 3, 6, months or at least 1, 2, 3 years longer to reach a given point in disease progression than in the absence of treatment.

There may also be an improvement in Musculo-skeletal pain in the subject. Musculoskeletal pain is defined as acute or chronic pain that affects bones, muscles, ligaments, tendons, and even nerves. The pain may comprise a number of different pain syndromes, which range from local pain to neuropathic pain. Musculoskeletal pain is primarily somatic in nature. The most prevalent forms of musculoskeletal pain are chronic low back pain, neck pain.

Musculo skeletal pain, for example, may be pain in the musculoskeletal system including joints, ligaments, muscles, nerves, or tendons, In some embodiments the musculo-skeletal pain may be chronic pain, typically a pain that carries on for longer than 12 weeks. In some embodiments the musculo-skeletal pain may be muscle pain, such as skeletal muscle pain, such as chronic skeletal muscle pain. In some embodiments the musculo-skeletal pain may be pain associated with abnormal curvature of the spine. In some embodiments the pain may not be associated with arthritis, such as osteoarthritis. In some embodiments the musculo-skeletal pain may be myalgia. In some embodiments, the musculo-skeletal pain may be associated with or caused by muscle cramps or spasms. Musculo-skeletal pain may be diagnosed or monitored via, for example, patient reported pain (See for example Nielsen and Arendt-Nielsen, Curr Pain Headache Rep. 2003 December; 7(6):443-51); MRI or CT scan; electromyography e.g. to measure electrical activity in nerves and muscles.

There may also be an improvement in posture, or a reduction in an abnormal curvature of the spine. Poor or bad posture refers to postural dysfunction, and may be defined as when a subject's spine is positioned in unnatural positions, in which the curves are emphasized. Complications of poor posture include back pain, spinal dysfunction, joint degeneration, rounded shoulders and a potbelly. The improvements associated with posture and spine curvature after administration of the inhibitor of FGFR3 signalling, or NPR-B agonist, or NPR-C agonist, may result from the improvements in muscle function as described above. This is consistent with the observations in Example 1 that treatment with the test Compound (1) had an impact on survival after only 15 days of treatment which is a time frame that is consistent with effects on muscle historic survival and increased incidence of maternal infanticide has been described in mouse models with muscle weakness as a major phenotypical trait (e.g., Sullivan 2014).

The improvement may be in kyphosis, lordosis, or scoliosis, or in spinal stenosis, which may e.g. arise from any of the above spinal deformities. Kyphosis is an exaggerated, forward rounding of the upper part of the spine. Lordosis is an exaggerated inward curve of the spine. Scoliosis is an abnormal lateral curvature of the spine. Spinal stenosis may for example arise from any of the above spinal deformities and is a narrowing of the spinal canal in the spine. Optionally the improvement in kyphosis, lordosis, spinal stenosis or scoliosis is by improving muscle function. These spinal abnormalities may occur in chondrodysplasia disease and preferably the subject with kyphosis, lordosis, spinal stenosis or scoliosis has a chondrodysplasia disease, such as a disease selected from the group consisting of achondroplasia, hypochondroplasia, and thanatophoric dysplasia.

Abnormal curvature of the spine may, for example, be monitored via physical examination, or an imaging method (e.g. x-ray examination, spinal radiography, CT scan or MRI scan). Signs of abnormal curvature of the spine may include, for example, uneven shoulders, head not cantered above the pelvis, one or both hips raised or unusually high, uneven waist, body lean. Curvature of the spine may for example be measured using the Cobb method.

### Further Embodiments Relating to Treatment of Abnormal Curvature of the Spine.

In some embodiments the invention relates to the treatment of a spinal deformity in a subject who has a chondrodysplasia disease, such as a chondrodysplasia disease disclosed herein, such as achondroplasia. Spinal deformities in chondrodysplasias such as achondroplasia can lead to pain and disability. Congenital narrowing of spinal canal is common in achondroplasia and is associated with neurological symptoms that worsen with age and can cause premature or sudden death, and may require corrective surgery or repeated corrective surgeries during the lifetime of the patient.

The spinal deformity may include an abnormal curvature of the spine which can be corrected, at least partially by the strengthening of muscles in the back, suitably via the therapeutic uses disclosed or claimed herein. The spinal deformity may be an abnormal spinal pedicle morphology or spinal stenosis, which is typical in achondroplasia, which may be further compounded by an abnormal curvature of the spine. The abnormal spinal pedicle morphology may be a reduction in the interpedicular distance or reduced pedicular width. Inter-pediculate narrowing and thickened pedicles are common on achondroplasia and the resulting spinal canal stenosis often requires surgical intervention, for example to correct narrowing of the foramen magnum (FM).

The invention provides for the use of the inhibitor of FGFR3 signaling or the NPR-B agonist for use according to the invention or as according to any one of the claims, wherein said use gives rise to an increase spine height, such as increased thoracic height; increased interpedicular distance or increased pedicular width.

The invention provides for the use of the NPR-B agonist for use according to the invention or as according to any one of the claims, wherein said use gives rise to an increase spine height, such as increased thoracic height; increased interpedicular distance or increased pedicular width. The invention provides for a method of treating a spinal pedicular deformity in a subject in need of treatment, said method comprising administering a therapeutically effective amount of an inhibitor of FGFR3 signaling, or an NPR-B agonist to said subject. Suitably said method may comprise an initial step of (i) diagnosing or measuring the spinal pedicular deformity in the subject, and then (ii) administering a therapeutically effective amount of an inhibitor of FGFR3 signaling, an NPR-B agonist, to the subject. Suitably the measurement step may involve one or more x-rays, such as an anteroposterior (AP) X-ray, and or a lateral x-ray. Lateral x-rays are useful to measure e.g. the pedicular width, and the AP X-ray is useful to measure e.g. the interpedicular distance. X-rays of the spine or spinal deformity taken prior to and after the administration may be used to determine the effective treatment. The diagnosis or measurement step may be the measurement of one or more of spine height, thoracic height; interpedicular distance and pedicular width. The diagnosis measurement step may be or include the measurement of spinal pedicle morphology or spinal stenosism/spinal canal stenosis.

In certain embodiments the administration gives rise to an improvement in sleep apnea, obstructive sleep apnea, or otitis media. Sleep apnea is a condition in which the subject's breathing stops and restarts many times during sleep. This can prevent the subject's body from getting enough oxygen. Obstructive sleep apnea (OSA), is a form of sleep apnea that occurs when throat muscles relax and block the flow of air into the lungs. Otitis media is an infection of the middle ear that causes inflammation (redness and swelling) and a build-up of fluid behind the eardrum, and is associated with mid face hypoplasia. Otitis media can result in hearing loss. Optionally the improvement in sleep apnea, obstructive sleep apnea, or otitis media is by improving muscle function. These conditions may occur in chondrodysplasia disease and preferably the subject with sleep apnea, obstructive sleep apnea, or otitis media has a chondrodysplasia disease, such as a disease selected from the group consisting of achondroplasia, hypochondroplasia, and thanatophoric dysplasia. It is believed that an improvement in muscle function will give rise to a benefit in these conditions in view of an association between sleep-disordered breathing, and upper airway (e.g. laryngeal and phalinguial) muscle weakness.

In some embodiments the invention relates to a method of treating obstructive sleep apnea. Sleep apnea such as obstructive sleep apnea may be monitored for example via nocturnal polysomnography/using breathing monitor.

Otitis media is defined as an infection of the middle ear space, and is typically diagnosed and monitored via a physical exam, and several diagnostic tools are available such as a pneumatic otoscope, tympanometry, and acoustic reflectometry. Otitis media is often associated with ear pain, reduced hearing and fever, which may also be monitored. In some embodiments, the otitis media is acute otitis media, and the present invention provides effective treatment which may reduce the frequency of acute otitis media events, reduce the severity of acute otitis media, or both. In some embodiments, the invention relates to a prophylactic treatment of otitis media. The monitoring of otitis media may therefore be assessing the frequency or severity of acute otitis media events, or both.

In certain embodiments the administration gives rise to a reduction in obesity. Where subjects prior to treatment are suffering from low muscle function, this makes it harder for them to exercise and to exercise well and effectively for weight control and general physical health. By improving muscle function in a subject, a reduction in obesity can also be obtained. This problem has been observed in patients having chondrodysplasia disease and obesity is noted as a major health problem in achondroplasia necessitating an early yet complex clinical management (see Saint-Laurent, C., Garde-Etayo, L. & Gouze, E. Obesity in achondroplasia patients: from evidence to medical monitoring. Orphanet J Rare Dis 14, 253 (2019). https://doi.org/10.1186/s13023-019-1247-6) which notes that children with achondroplasia are limited by their psychomotor development and their physical condition, and the early appearance of overweight and obesity contributes to worsen a sedentary lifestyle and/or to exclude these children from the sports practiced by children their age. Optionally the reduction in obesity is by improving muscle function. Obesity may occur in chondrodysplasia disease and preferably the subject with obesity has a chondrodysplasia disease, such as a disease selected from the group consisting of achondroplasia, hypochondroplasia, and thanatophoric dysplasia.

Obesity is defined as a body mass index (BMI) of over 30. Thus a reduction in obesity may be a reduction in BMI. The subject may have a BMI of over 30 at initiation of administration, e.g. a BMI of over 30, 32, 35, 38, 40 before administration. In certain embodiments the has a BMI of 25-30 before administration, e.g. over 26, 27, 28, 29.

### Embodiments Relating to Hand Length

Hand deformities are a characteristic in achondroplasia, and include short finger length, trident hand deformity, and an inability to fully extend fingers.

The invention provides for an inhibitor of FGFR3 signaling or an NPR-B agonist, for use in promoting hand length growth or finger length growth in a human subject who is less than 18 years of age or whose bone epiphysis have not closed.

The invention provides for an inhibitor of FGFR3 signaling or an NPR-B agonist, for use in correcting a hand deformity in a human subject who is less than 18 years of age or whose bone epiphysis have not closed.

The invention provides for an inhibitor of FGFR3 signaling or an NPR-B agonist, for use according to the invention or according to any one of the claims, wherein said use increases hand length growth or finger length growth in the human subject, such as a human subject who is less than 18 years of age or whose bone epiphysis have not closed.

The invention provides for a method of increasing the rate of hand or finger length in a subject in need of treatment, said method comprising administering a therapeutically effective amount of an inhibitor of FGFR3 signaling, or an NPR-B agonist to said subject.

Suitably the method of the invention may comprise the steps of (i) assessing hand length or finger length(s) in the subject in need of treatment, and (ii) administering a therapeutically effective amount of an inhibitor of FGFR3 signaling, or an NPR-B agonist, to the subject, and (iii) optionally measuring the increase in hand length or finger length(s) after said administration.

The invention provides for a method of correcting hand deformity in a subject in need of treatment, said method comprising administering a therapeutically effective amount of an inhibitor of FGFR3 signaling, or an NPR-B agonist to said subject.

Suitably the method of the invention may comprise the steps of (i) assessing hand deformity, in the subject in need of treatment, and (ii) administering a therapeutically effective amount of an inhibitor of FGFR3 signaling, or an NPR-B agonist, to the subject, and (iii) optionally assessing the change in hand deformity in the subject after said administration.

### Methods Encompassing Assessment Steps

In certain embodiments, the invention provides methods of improving muscle function in a subject suffering from a disease or condition in which muscle function is impaired. The methods include assessing muscle function at least once in the subject suffering from a disease or condition in which muscle function is impaired, and administering a therapeutically effective amount or regimen of an inhibitor of FGFR3 signaling, an NPR-B agonist or an NPR-C agonist to the subject.

This may be described as a method of improving muscle function in a subject suffering from a disease or condition in which muscle function is impaired, the method comprising:
(i) Assessing muscle function, at least once, in the subject suffering from a disease or condition in which muscle function is impaired, and
(ii) administering a therapeutically effective amount of an inhibitor of FGFR3 signaling, an NPR-B agonist or an NPR-C agonist, to the subject.

The assessing step can be performed before the administering step, e.g. to determine a baseline value or determination of muscle function before administration begins. This may be useful e.g. for comparison with subsequent values or determination of muscle function, which may be assessed after administration. For example, in such case, the assessing step can be performed, e.g., up to a week, a month or three months before initiating administration. The assessment step before administration can be used as a comparison, e.g. to determine the effectiveness of the treatment. The administering step may be initiated responsive to the assessing step.

The administering step will in general comprise administering the inhibitor of FGFR3 signaling, the NPR-B agonist or the NPR-C agonist a plurality of times. Assessment of muscle function can also be performed at least twice, or a plurality of times. Optionally it is performed once or or at least once before initiating the administering step (e.g. to determine a baseline) and once or at least once after initiating the administering step. The assessing step may also be performed at least three times, once or at least once before initiating the administering step (e.g. to determine a baseline) and twice or at least twice after initiating the administering step.

It may be informative to compare muscle function determined before initiating the administering step to muscle function determined after initiating the administering step, or to compare successive determinations of muscle function, or to use regular determinations of muscle function e.g. to observe trends in muscle function, e.g. in response to treatment. This can be used to determine treatment outcomes, or to inform modifications to a treatment protocol.

Assessment of muscle function before initiating administration can also be used, optionally in combination with other signs or symptoms of a subject, to diagnose a subject as suffering from a disease or condition in which muscle function is impaired. The methods referred to herein may thus additionally comprise diagnosing the subject as suffering from an impairment of muscle function and/or from a disease or condition in which muscle function is impaired. Administration can begin responsive to the assessment step and/or to diagnosis of the disease or condition in the subject. Administration can also begin in response to assessment of impaired muscle function relative to reference values, e.g. which may be obtained by assessment carried out on normal or control subjects (e.g. subjects known not to be suffering from a disease or condition in which muscle function is impaired).

An assessing step can additionally or alternatively be performed after initiating the administration, e.g., between successive doses of drug. The assessing step performed after initiating the administration can be performed once or a plurality of times. The assessing step may be performed at least 4, 5, 6, 7, 8, 9, 10 times, e.g weekly, monthly, annually, e.g. at regular intervals. The assessing step may be performed for the duration of the treatment.

Assessment of muscle function can include any of a) skeletal muscle strength, b) skeletal muscle tone c) skeletal muscle stamina, d) skeletal muscle mass, e) skeletal muscle fatigue, f) cardiovascular endurance, g) cardiovascular fitness, h) exercise intolerance, i) exercise capacity, j) exercise induced fatigue, k) hypotonia, l) skeletal muscle mass and/or muscle/fat ratio (such as skeletal muscle/fat ratio), m) musculo-skeletal pain, n) posture or curvature of the spine, optionally kyphosis, lordosis, spinal stenosis or scoliosis o) sleep apnea, obstructive sleep apnea, or otitis media, p) obesity.

Such assessments include those described elsewhere herein. Examples include measures of muscle weight, muscle length, muscle density, muscle size and/or volume. The assessments may including subject imaging, observing subject performance. The assessments may be with or without quantitative metrics in physical testing, such as for muscle fatigue and cardiovascular testing or by biochemical assays on muscle biopsy or for products of muscle metabolism released to body fluids.

The assessment may comprise imaging, e.g. at least one imaging step (such as X-ray examination, spinal radiography, CT scan or MRI scan).

The assessment may comprise determining a performance metric of the subject, which may for example be a measurement or an observation of the subject's ability to carry out certain tasks, e.g. with reference to physical functioning tests such as those described elsewhere herein (e.g. to determine muscle fatigue and endurance levels such as sit to stand tests and six-minute walk tests).

If more than one assessment is carried out, a composite index may be generated. In certain embodiments a composite index is generated for at least 2, 3, 4, 5, 6, 7 (or 1-6, 2-5, 3-4) of any assessments carried out, e.g. for at least 2, 3, 4, 5, 6, 7 (or 1-6, 2-5, 3-4) of the assessments a) to p) set out above. If a composite index is generated, this may form the basis of any comparisons referred to elsewhere herein.

An improvement in muscle function can be any improvement in muscle function as defined elsewhere herein.

In some methods, an assessing step is performed at least once before initiating the administering step e.g. to determine a baseline value or determination for muscle function and once, twice or more times after initiating administration, e.g. to allow determination of changes of muscle function responsive to administration.

Comparison of values of muscle function before and after administration can indicate increased muscle function, unchanged muscle function or decreased muscle function. Increased muscle function following and responsive to initiation of administration may be one indicator of a positive response to treatment, i.e., that the treatment is successful.

In some embodiments, an increase in muscle function or progressive increases with further dosing, or an increase in muscle function when muscle function was declining in the subject before treatment or shows decline in historical control subjects are indicators of a positive response to treatment.

A finding of unchanged muscle function or decreased muscle function may indicate a positive response to treatment or may indicate a need for a change in the treatment protocol (e.g., to increase dosage and/or frequency thereof), and this will depend on the particular condition and expected outcome. For example, if muscle function was impaired but stable in a subject before initiating administration of drug, unchanged or decreased muscle function after administration of drug may be a negative indicator of response to treatment. But if muscle function was impaired and declining in a subject before initiating administration of drug, then unchanged muscle function following and response to administration of drug may indicate a positive response to treatment. Likewise, even decreased muscle function following initiating administration of drug is less than expected based on a decline in the subject before initiating administration or in untreated historical control subjects can indicate a positive response to treatment. Decreased muscle function beyond any expectations based on a decline in the subject before initiating administration of drug or in untreated historical control subjects may indicate a negative response to drug administration. The expectations may depend on comparison with control value(s) determined in the same subject before initiating drug administration, reference subjects or normal and/or control subjects.

Assessment of changes in muscle function in response to administration can be used to guide treatment decisions whether to continue administration of the drug, modify the regimen of the drug (e.g., change dosage or frequency) or discontinue administration of the drug. For example, if the assessment provides an indication of a positive response to administration, the same treatment can continue to be administered, e.g. under the same regimen. Variation of the dosage and/or frequency up or down can also be considered depending on the magnitude of the response and any side effects the subject is experiencing. For example, an increased dose and/or frequency can be administered for an increased response or decreased dose and/or frequency to reduce any side effects. If the assessment indicates a negative response to treatment (or no positive response to treatment) and the subject is tolerating the administered drug without unacceptable side effects, then a higher dose or frequency of the same drug can be administered to attempt to elicit a positive treatment response. If the assessment continues to indicate a negative response to treatment (or no positive response to treatment) or the subject cannot tolerate a higher dose or frequency due to side effects, then administration of the drug can be discontinued.

An assessment step may follow any method of treatment referred to herein. For example, the invention provides the method according to any one of Items 34-114.

Methods relating to treating or preventing a disease or condition associated with an impairment in neuromuscular function, such as a neurodegenerative disease in a subject may likewise incorporate assessment steps relating to determination of muscle (e.g. skeletal muscle function, e.g as referred to above). Alternatively or additionally such methods may incorporate assessment steps relating to determination of mitochondrial dysfunction, neuromuscular function, e.g. to establish whether the treatment gives rise to a slowing, or delaying or reduction of disease progression, such as a reduced rate of loss or decline of muscle function, or reduced rate of loss or decline of neuromuscular function.

Combination with Growth Hormone

Human growth hormone is approved for pediatric treatment of achondroplasia in Japan, and combination treatment with CNP is under clinical investigation. The combination treatment of growth hormone in the methods and uses of the present invention are expected to be advantageous.

In some embodiments the growth hormone is or comprises somatropin, or is a somatropin conjugate such as a PEGylated somatropin, or a fatty acid growth hormone conjugate.

In some embodiments the growth hormone is a conjugate of growth hormone such as a conjugate of human growth hormone. The conjugate moiety may, for example comprise a PEG moiety, a fatty acid moiety, a serum albumin binding moiety, an antibody moiety, or an antibody fragment moiety.

In some embodiments the growth hormone is a long-acting growth hormone, such as a growth hormone for weekly administration. By way of example long-acting growth hormone may be administered weekly or less frequently than weekly.

Suitable, the growth hormone may be a controlled-release hGH. Long acting human growth hormone and controlled-release hGH are disclosed in WO 2018/060314 A1, which is hereby incorporated by reference in its entirety.

In some embodiments the growth hormone is lonapegsomatropin (lonapegsomatropin-tcgd). In some embodiments, the growth hormone conjugate, such as human growth hormone conjugate, long acting human growth hormone or controlled-release hGH, such as lonapegsomatropin, is administered to a subject in a dose ranging from about 0.021 mg/kg/week to about 0.7 mg/kg/week such as at or about 0.21 mg/kg/week (mg/kg refers to the mass of growth hormone polypeptide without the conjugate moiety administered per week).

In some embodiments, the growth hormone is selected from the group consisting of somapacitan or somapacitan-beco (marketed as SOGROYA® Novo Nordisk), somatrogon (marketed as NGENLA™ by (Pfizer/OPKO), eftansomatropin alfa (also known as eftansomatropin), efpegsomatropin, albusomatropin, somavaratan, ibutamoren, and lonapegsomatropin-tcgd.

### Exemplary Administration Protocol for CNP Drugs, Such as Drugs Comprising CNP Conjugate, Such as CNP of Formula (IIf′) or Formula (III) or Compound (1).

Prior to a e.g. subcutaneous administration to a patient in need thereof, the solid unit dosage form is reconstituted. Reconstitution of the solid unit dosage form into a reconstituted formulation is done by adding a predefined amount of reconstitution solution to the solid unit dosage form. Therefore, a further aspect of the present invention is a method of reconstituting the solid unit dosage form of the present invention wherein the method comprises the step of
(a) contacting the solid unit dosage form of the present invention with a reconstitution solution.

Reconstitution may take place in the container in which the solid unit dosage form is provided, such as in a vial; syringe such as a dual-chamber syringe; ampoule; cartridge, such as a dual-chamber cartridge; or the solid unit dosage form may be transferred to a different container where is then reconstituted. In certain embodiments, the container in which the reconstitution of the solid unit dosage form takes place is a vial. In certain embodiments, the container in which the reconstitution of the solid unit dosage form takes place is a syringe. In certain embodiments, the container in which the reconstitution of the solid unit dosage form takes place is a dual-chamber syringe. In certain embodiments, the container in which the reconstitution of the solid unit dosage form takes place is a cartridge. In certain embodiments, the container in which the reconstitution of the solid unit dosage form takes place is a dual-chamber cartridge.

In certain embodiments, the solid unit dosage form according to the present invention is provided in a first chamber of the dual-chamber syringe and the reconstitution solution is provided in a second chamber of the dual-chamber syringe.

The reconstitution solution is a sterile liquid, such as water or buffer, which may comprise further additives, such as preservatives and/or antimicrobials.

In certain embodiments, the reconstituted solution comprises one or more preservative and/or antimicrobial and/or antioxidant.

In certain embodiments, the reconstituted solution comprises one or more preservative.

The preservative may be selected from the group consisting of m-cresol, benzoic acid, phenol, methylparaben, ethylparaben, propylparaben, butylparaben, potassium sorbate, chlorobutanol, benzyl alcohol, phenylmercuric nitrate, thimerosal, sorbic acid, potassium sorbate, chlorocresol, benzalkonium chloride, 2-ethoxyethanol, chlorhexidine, chlorobutanol, phenylethyl alcohol, phenylmercuric acetate and mixtures thereof.

In certain embodiments, the preservative is m-cresol. In certain embodiments, the preservative is benzylalcohol. In certain embodiments, the preservative is benzoic acid. In certain embodiments, the preservative is phenol. In certain embodiments, the preservative is methylparaben. In certain embodiments, the preservative is ethylparaben. In certain embodiments, the preservative is propylparaben. In certain embodiments, the preservative is butylparaben. In certain embodiments, the preservative is potassium sorbate. In certain embodiments, the preservative is benzyl alcohol. In certain embodiments, the preservative is phenylmercuric nitrate. In certain embodiments, the preservative is thimerosal. In certain embodiments, the preservative is sorbic acid. In certain embodiments, the preservative is potassium sorbate. In certain embodiments, the preservative is chlorocresol. In certain embodiments, the preservative is benzalkonium chloride. In certain embodiments, the preservative is 2-ethoxyethanol. In certain embodiments, the preservative is chlorhexidine. In certain embodiments, the preservative is chlorbutanol. In certain embodiments, the preservative is phenylethyl alcohol. In certain embodiments, the preservative is phenylmercuric acetate.

In certain embodiments, the preservative has a concentration ranging from 1 to 10 mg/ml. In certain embodiments, the preservative has a concentration ranging from 1.5 to 3.5 mg/ml. In certain embodiments, the preservative has a concentration ranging from 2 to 3 mg/ml.

The antioxidant may be selected from the group consisting of methionine, butylhydroxytoluene, butylhydroxyanisol, tocopherol, propylgallate, ascorbic acid, ethylenediaminetetraacetic acid (EDTA), poly(ethylenimine), vitamin E and mixtures thereof.

In certain embodiments, the preservative is methionine. In certain embodiments, the preservative is butylhydroxytoluene. In certain embodiments, the preservative is butylhydroxyanisol. In certain embodiments, the preservative is tocopherol. In certain embodiments, the preservative is propylgallate. In certain embodiments, the preservative is ethylenediaminetetraacetic acid. In certain embodiments, the preservative is poly(ethylenimine). In certain embodiments, the preservative is vitamin E.

As defined herein, the term “methionine” is intended to encompass both D-methionine and L-methionine, and mixtures thereof. In certain embodiments, the term “methionine” refers to L-methionine. In certain embodiments, the term “methionine” refers to D-methionine. In certain embodiments, the term “methionine” refers to a mixture of D-methionine or L-methionine. In certain embodiments, the term “methionine” refers to L-methionine hydrochloride salt.

As defined herein, the term “EDTA” is intended to encompass all EDTA forms that are known in the art such as EDTA salts, including EDTA metal salts, such as EDTA disodium salt, EDTA dipotassium salt, EDTA calcium salt, EDTA dimagnesium salt or mixtures thereof. In certain embodiments, EDTA refers to EDTA disodium salt. In certain embodiments, the term “EDTA” refers to EDTA dicalcium salt. In certain embodiments, the term “EDTA” refers to EDTA anhydrous.

In certain embodiments, the molar ratio of antioxidant to CNP moiety is from about 0.1:1 to about 100:1. In certain embodiments, the molar ratio of antioxidant to CNP moiety is from about 0.1:1 to about 70:1. In certain embodiments, the molar ratio of antioxidant to CNP moiety is from about 0.1:1 to about 15:1. In certain embodiments, the molar ratio of antioxidant to CNP moiety is from about 1:1 to about 10:1. In certain embodiments, the molar ratio of antioxidant to CNP moiety is from about 3:1 to about 7:1.

In certain embodiments, the reconstituted solution does not comprise an antimicrobial. In certain embodiments, the reconstituted solution comprises one or more excipient.

In certain embodiments, the reconstitution solution is sterile water. In certain embodiments, the reconstitution solution is sterile water comprising 0.7-1.1% benzylalcohol. In certain embodiments, the reconstitution solution is sterile water comprising 0.9% benzylalcohol.

In certain embodiments, the reconstituted solution comprises a pH-modifying agent.

As used herein, the term “pH-modifying agent” refers to a chemical compound that is used to modify the pH of the reconstitution solution.

In certain embodiments, the pH-modifying agent may be an acid or acidic salt thereof. The acid may be selected from the group consisting of acetic acid, citric acid, succinic acid, hydrochloric acid, phosphoric acid, carbonic acid, nitric acid and mixtures thereof.

In certain embodiments, the pH-modifying agent may be a base or basic salt thereof. The base may be selected from the group consisting of Tris (tris(hydroxymethyl)aminomethane), sodium hydroxide, potassium hydroxide, lysine and mixtures thereof.

In certain embodiments, the volume of the reconstitution solution ranges from about 0.1 ml to about 4 ml. In certain embodiments, the volume of the reconstitution solution is about 1 ml, such as about 2 ml, such as about 3 ml or such as about 4 ml.

In certain embodiments, the volume of the reconstitution solution is about 0.79 ml. In certain embodiments, the volume of the reconstitution solution is 0.79 ml. In certain embodiments, the volume of the reconstitution solution is about 1 ml. In certain embodiments, the volume of the reconstitution solution is 1 ml. In certain embodiments, the volume of the reconstitution solution is about 1.1 ml. In certain embodiments, the volume of the reconstitution solution is 1.1 ml. In certain embodiments, the volume of the reconstitution solution is about 1.25 ml. In certain embodiments, the volume of the reconstitution solution is 1.25 ml

It is understood that the volume of the unit dose or injection volume is based on the patient's actual body weight and the concentration of the reconstituted solution. In certain embodiments, the concentration of CNP within the reconstituted solution is not more than 7 mg/ml. In certain embodiments, the concentration of CNP within the reconstituted solution is not less than 0.5 mg/ml. In certain embodiments, the concentration of CNP within the reconstituted solution is 0.75 mg/ml. In certain embodiments, the concentration of CNP within the reconstituted solution is 1 mg/ml. In certain embodiments, the concentration of CNP within the reconstituted solution is 2.2 mg/ml. In certain embodiments, the concentration of CNP within the reconstituted solution is 3.6 mg/ml. In certain embodiments, the concentration of CNP within the reconstituted solution is 4.6 mg/ml. In certain embodiments, the concentration of CNP within the reconstituted solution is 5 mg/ml. In certain embodiments, the concentration of CNP within the reconstituted solution is 5.5 mg/ml.

After reconstitution, a unit dose has a volume of not more than 4 ml. In certain embodiments, the volume of the unit dose ranges from about 0.01 ml to about 1.1 ml. In certain embodiments, the volume of the unit dose ranges from 0.01 ml to 0.75 ml. In certain embodiments, the volume of the unit dose ranges from 0.01 ml to 0.50 ml.

In certain embodiments, the volume of the unit dose is about 0.03 ml. In certain embodiments, the volume of the unit dose is about 0.05 ml. In certain embodiments, the volume of the unit dose is about 0.1 ml. In certain embodiments, the volume of the unit dose is about 0.2 ml. In certain embodiments, the volume of the unit dose is about 0.25 ml. In certain embodiments, the volume of the unit dose is about 0.3 ml. In certain embodiments, the volume of the unit dose is about 0.35 ml. In certain embodiments, the volume of the unit dose is about 0.4 ml. In certain embodiments, the volume of the unit dose is about 0.5 ml. In certain embodiments, the volume of the unit dose is about 0.6 ml. In certain embodiments, the volume of the unit dose is about 0.75 ml. In certain embodiments, the volume of the unit dose is about 1 ml.

In certain embodiments, the patient is an infant and the volume of the unit dose ranges from about 10 μl to 100 μl. In certain embodiments, the patient is an infant and the volume of the unit dose ranges from about 10 μl to 50 μl. In certain embodiments, the patient is an infant and the volume of the unit dose ranges from about 10 μl to 30 μl.

In certain embodiments, the patient is an infant and the volume of the unit dose is about 10 μl. In certain embodiments, the patient is an infant and the volume of the unit dose is about 15 μl. In certain embodiments, the patient is an infant and the volume of the unit dose is about 20 μl.

In certain embodiments, the patient is an infant and the volume of the unit dose is 10 μl. In certain embodiments, the patient is an infant and the volume of the unit dose is 15 μl. In certain embodiments, the patient is an infant and the volume of the unit dose is 20 μl.

In certain embodiments, the patient is an infant, the unit dose is 20 μg CNP/kg and the volume of the unit dose is about 10 μl. In certain embodiments, the patient is an infant, the unit dose is 20 μg CNP/kg and the volume of the unit dose is about 15 μl. In certain embodiments, the patient is an infant, the unit dose is 20 μg CNP/kg and the volume of the unit dose is about 20 μl.

In certain embodiments, the unit dose is 6 μg CNP/kg and the volume of the unit dose is 0.06 ml. In certain embodiments, the unit dose is 20 μg CNP/kg and the volume of the unit dose 0.3 ml. In certain embodiments, the unit dose is 50 μg CNP/kg and the volume of the unit dose is 0.2 ml. In certain embodiments, the unit dose is 75 μg CNP/kg and the volume of the unit dose is 0.4 ml. In certain embodiments, the unit dose is 100 μg CNP/kg and the volume of the unit dose is 0.5 ml. In certain embodiments, the unit dose is 150 μg CNP/kg and the volume of the unit dose is 0.5 ml.

In certain embodiments, the pH of the liquid unit dosage form is from about pH 4 to about pH 6. In certain embodiments, the pH of the liquid unit dosage form is from about pH 4.5 to about pH 5.5. In certain embodiments, the pH of the liquid unit dosage form is about 5. In certain embodiments, the pH of the liquid unit dosage form is 5.

In certain embodiments, the unit dosage form of the present invention further comprises a buffering agent, an isotonicity agent and a pH-modifying agent.

In certain embodiments, the buffering agent has a concentration ranging from 1.3 to 57.6 mM in the unit dosage form. In certain embodiments, the buffering agent has a concentration ranging from 1.7 to 33 mM in the unit dosage form. In certain embodiments, the buffering agent has a concentration ranging from 5.1 to 20.3 mM in the unit dosage form. In certain embodiments, the buffering agent has a concentration of about 10 mM in the unit dosage form.

Exemplary buffering agents may be selected from the group consisting of succinic acid, citric acid, lactic acid, acetic acid, glutamic acid, fumaric acid, aspartic acid, glutaric acid, phosphoric acid, histidine, gluconic acid, tartaric acid, malic acid and mixtures thereof. It is clear to the person skilled in the art that the corresponding conjugate bases or salts of the buffering agents such as succinate, citrate, lactate, acetate, glutamate, fumarate, aspartate, glutarate, phosphate, gluconate, tartrate, malate and mixtures thereof, respectively, may also be included.

In certain embodiments, the buffering agent is succinic acid. In certain embodiments, the buffering agent is citric acid. In certain embodiments, the buffering agent is lactic acid. In certain embodiments, the buffering agent is acetic acid. In certain embodiments, the buffering agent is glutamic acid. In certain embodiments, the buffering agent is fumaric acid. In certain embodiments, the buffering agent is aspartic acid. In certain embodiments, the buffering agent is glutaric acid. In certain embodiments, the buffering agent is phosphoric acid. In certain embodiments, the buffering agent is histidine. In certain embodiments, the buffering agent is gluconic acid. In certain embodiments, the buffering agent is tartaric acid. In certain embodiments, the buffering agent is malic acid.

The isotonicity agent may be selected from the group consisting of trehalose, mannitol, sucrose, raffinose, gelatin, lactose, dibasic calcium phosphate, sorbitol, xylitol, glycine, histidine, hydroxyethylstarch, dextrose, dextran, Ficoll®, propylene glycol and mixtures thereof.

In certain embodiments, the isotonicity agent may be selected from the group consisting of trehalose, mannitol, sucrose, raffinose, gelatin, lactose, dibasic calcium phosphate, sorbitol, xylitol, glycine, histidine, hydroxyethylstarch, dextrose, dextran, propylene glycol and mixtures thereof.

In certain embodiments, the isotonicity agent is selected from the group consisting of trehalose, sucrose and glycine. In certain embodiments, the isotonicity agent is a non-reducing sugar such as trehalose or sucrose.

In certain embodiments, the isotonicity agent is trehalose.

As defined herein, the term “trehalose” is intended to encompass all salts and hydration states of trehalose, such as trehalose anhydrous or trehalose dihydrate. In certain embodiments, the term “trehalose” refers to trehalose anhydrous. In certain embodiments, the term “trehalose” refers to trehalose dihydrate.

In certain embodiments, the unit dosage form comprises succinic acid and trehalose.

In certain embodiments, the unit dosage form comprises

| | | |
|---|---|---|
| CNP conjugate | 0.9-82.1 | mg/ml |
| succinic acid | 1.3-57.6 | mM |
| trehalose dihydrate | 67-111.6 | mg/ml, |

and has a pH ranging from pH 4.0 to pH 6.0.

In certain embodiments, the unit dosage form comprises:

| | | |
|---|---|---|
| CNP conjugate | 19.8-73.6 | mg/ml |
| succinic acid | 1.7-50 | mM |
| trehalose dihydrate | 63-100 | mg/ml |

and has a pH ranging from pH 4.0 to pH 6.0.

In certain embodiments, the unit dosage form comprises:

| | | |
|---|---|---|
| CNP conjugate | 27.5-50.5 | mg/ml |
| succinic acid | 5.1-20.3 | mM |
| trehalose dihydrate | 67-95 | mg/ml |

and has a pH ranging from pH 4.0 to pH 6.0.

In certain embodiments, the unit dosage form comprises about 8.2 mg/ml CNP conjugate, about 10 mM succinic acid, about 89 mg/ml trehalose dihydrate and optionally Tris and/or hydrochloric acid and has a pH of about 5.

In certain embodiments, the unit dosage form comprises 8.2 mg/ml CNP conjugate, 10 mM succinic acid, 89 mg/ml trehalose dihydrate and optionally Tris and/or hydrochloric acid and has a pH of 5.

In certain embodiments, the unit dosage form comprises about 11 mg/ml CNP conjugate, about 10 mM succinic acid, about 88.5 mg/ml trehalose dihydrate and optionally Tris and/or hydrochloric acid and has a pH of about 5.

In certain embodiments, the unit dosage form comprises 11 mg/ml CNP conjugate, 10 mM succinic acid, 88.5 mg/ml trehalose dihydrate and optionally Tris and/or hydrochloric acid and has a pH of 5.

In certain embodiments, the unit dosage form comprises about 24.2 mg/ml CNP conjugate, about 10 mM succinic acid, about 85 mg/ml trehalose dihydrate and optionally Tris and/or hydrochloric acid and has a pH of about 5.

In certain embodiments, the unit dosage form comprises 24.2 mg/ml CNP conjugate, 10 mM succinic acid, 85 mg/ml trehalose dihydrate and optionally Tris and/or hydrochloric acid and has a pH of 5.

In certain embodiments, the unit dosage form comprises about 39.6 mg/ml CNP conjugate, about 10 mM succinic acid, about 80 mg/ml trehalose dihydrate and optionally Tris and/or hydrochloric acid and has a pH of about 5.

In certain embodiments, the unit dosage form comprises 39.6 mg/ml CNP conjugate, 10 mM succinic acid, 80 mg/ml trehalose dihydrate and optionally Tris and/or hydrochloric acid and has a pH of 5.

In certain embodiments, the unit dosage form comprises about 50.5 mg/ml CNP conjugate, about 10 mM succinic acid, about 77 mg/ml trehalose dihydrate and optionally Tris and/or hydrochloric acid and has a pH of about 5.

In certain embodiments, the unit dosage form comprises 50.5 mg/ml CNP conjugate, 10 mM succinic acid, 77 mg/ml trehalose dihydrate and optionally Tris and/or hydrochloric acid and has a pH of 5.

In certain embodiments, the unit dosage form comprises about 54.9 mg/ml CNP conjugate, about 10 mM succinic acid, about 75 mg/ml trehalose dihydrate and optionally Tris and/or hydrochloric acid and has a pH of about 5.

In certain embodiments, the unit dosage form comprises 54.9 mg/ml CNP conjugate, 10 mM succinic acid, 75 mg/ml trehalose dihydrate and optionally Tris and/or hydrochloric acid and has a pH of about 5.

In certain embodiments, the unit dosage form comprises about 60.4 mg/ml CNP conjugate, about 10 mM succinic acid, about 73 mg/ml trehalose dihydrate and optionally Tris and/or hydrochloric acid and has a pH of about 5.

In certain embodiments, the unit dosage form comprises 60.4 mg/ml CNP conjugate, 10 mM succinic acid, 73 mg/ml trehalose dihydrate and optionally Tris and/or hydrochloric acid and has a pH of 5.

In certain embodiments, the unit dosage form comprises based on the total weight of the solid unit dosage form:

| | | |
|---|---|---|
| CNP conjugate | 8.2-44.4% | (w/w) |
| succinic acid | 0.9-1.2% | (w/w) |
| trehalose dihydrate | 53.7-89.1% | (w/w) |
| Tris | 1.0-1.5% | (w/w). |

In certain embodiments, the unit dosage form comprises, based on the total weight of the solid unit dosage form, about 8.2% (w/w) CNP conjugate, about 1.2% (w/w) succinic acid, about 89.1% (w/w) trehalose dihydrate and about 1.5% (w/w) Tris.

In certain embodiments, the unit dosage form comprises, based on the total weight of the solid unit dosage form, 8.2% (w/w) CNP conjugate, 1.2% (w/w) succinic acid, 89.1% (w/w) trehalose dihydrate and 1.5% (w/w) Tris.

In certain embodiments, the unit dosage form comprises, based on the total weight of the solid unit dosage form, about 10.7% (w/w) CNP conjugate, about 1.2% (w/w) succinic acid, about 86.8% (w/w) trehalose dihydrate and about 1.3% (w/w) Tris.

In certain embodiments, the unit dosage form comprises, based on the total weight of the solid unit dosage form, 10.7% (w/w) CNP conjugate, 1.2% (w/w) succinic acid, 86.8% (w/w) trehalose dihydrate and 1.3% (w/w) Tris.

In certain embodiments, the unit dosage form comprises, based on the total weight of the solid unit dosage form, about 21.6% (w/w) CNP conjugate, about 1.1% (w/w) succinic acid, about 76.1% (w/w) trehalose dihydrate and about 1.2% (w/w) Tris.

In certain embodiments, the unit dosage form comprises, based on the total weight of the solid unit dosage form, 21.6% (w/w) CNP conjugate, 1.1% (w/w) succinic acid, 76.1% (w/w) trehalose dihydrate and 1.2% (w/w) Tris.

In certain embodiments, the unit dosage form comprises, based on the total weight of the solid unit dosage form, about 32.4% (w/w) CNP conjugate, about 1.0% (w/w) succinic acid, about 65.4% (w/w) trehalose dihydrate and about 1.2% (w/w) Tris.

In certain embodiments, the unit dosage form comprises, based on the total weight of the solid unit dosage form, 32.4% (w/w) CNP conjugate, 1.0% (w/w) succinic acid, 65.4% (w/w) trehalose dihydrate and 1.2% (w/w) Tris.

In certain embodiments, the unit dosage form comprises, based on the total weight of the solid unit dosage form, about 38.9% (w/w) CNP conjugate, about 0.9% (w/w) succinic acid, about 59.2% (w/w) trehalose dihydrate and about 1% (w/w) Tris.

In certain embodiments, the unit dosage form comprises, based on the total weight of the solid unit dosage form, 38.9% (w/w) CNP conjugate, 0.9% (w/w) succinic acid, 59.2% (w/w) trehalose dihydrate and 1% (w/w) Tris.

In certain embodiments, the unit dosage form comprises, based on the total weight of the solid unit dosage form, about 41.5% (w/w) CNP conjugate, about 0.9% (w/w) succinic acid, about 56.6% (w/w) trehalose dihydrate and about 1% (w/w) Tris.

In certain embodiments, the unit dosage form comprises, based on the total weight of the solid unit dosage form, 41.5% (w/w) CNP conjugate, 0.9% (w/w) succinic acid, 56.6% (w/w) trehalose dihydrate and 1% (w/w) Tris.

In certain embodiments, the unit dosage form comprises, based on the total weight of the solid unit dosage form, about 44.4% (w/w) CNP conjugate, about 0.9% (w/w) succinic acid, about 53.7% (w/w) trehalose dihydrate and about 1% (w/w) Tris.

In certain embodiments, the unit dosage form comprises, based on the total weight of the solid unit dosage form, 44.4% (w/w) CNP conjugate, 0.9% (w/w) succinic acid, 53.7% (w/w) trehalose dihydrate and 1% (w/w) Tris.

Applicant surprisingly found that upon administration to a patient in need thereof of the unit dosage form of CNP conjugate such as a compound of formula (IIf′) or formula (IIf) or compound (1), the incidence of hypotension is less than 10%, preferably less than 8%, most preferably less than 5%, even more preferably is less than 3%. In certain embodiments, upon administration to a patient in need thereof of the unit dosage form of the present invention, the incidence of hypotension is less than 1%. In certain embodiments, there is no incidence of hypotension.

Also, it was surprisingly found that no treatment emergent anti-CNP antibodies were detected upon treatment with the CNP conjugate (CNP conjugate such as a compound of formula (IIf′) or formula (IIf) or compound). In certain embodiments, no anti-CNP binding antibodies have been detected upon 1 to 9 months of repeated weekly exposure to the conjugate of the present invention. In certain embodiments, no anti-CNP binding antibodies have been detected upon 52 weeks of repeated weekly exposure to the conjugate of the present invention.

Moreover, it was surprisingly found that the administration of 100 μg CNP/kg (CNP conjugate such as a compound of formula (IIf′) or formula (IIf) or compound) per week to pediatric patients aged 2 to 10 years, such as 2 to 5 years or such as 5 to 10 years, in need of CNP treatment resulted in similar responses measured as annualized growth velocity.

In certain embodiments, the CNP moiety of the CNP conjugate has the sequence of SEQ ID NO:9, SEQ ID NO: 10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25 or SEQ ID NO:30. In certain embodiments, the CNP moiety has the sequence of SEQ ID NO:20, SEQ ID NO:21, SEQ ID NO:22, SEQ ID NO:23, SEQ ID NO:24 or SEQ ID NO:25. In certain embodiments, the CNP moiety has the sequence of SEQ ID NO:20. In certain embodiments, the CNP moiety has the sequence of SEQ ID NO:21. In certain embodiments, the CNP moiety has the sequence of SEQ ID NO:22. In certain embodiments, the CNP moiety has the sequence of SEQ ID NO:23. In certain embodiments, the CNP moiety has the sequence of SEQ ID NO:24. In certain embodiments, the CNP moiety has the sequence of SEQ ID NO:25.

### Exemplary CNP Conjugates and CNP Prodrugs

In certain embodiments, the CNP conjugate is of formula (Ia) or (Ib):

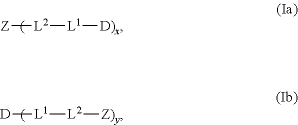

wherein
   -D is a CNP moiety;
   -L¹- is a reversible linker moiety;
   -L²- is a single chemical bond or a spacer moiety;
   —Z is a polymeric moiety;
   x is an integer selected from the group consisting of 1, 2, 3, 4, 5, 6, 7, 8, 9, 10, 11, 12, 13, 14, 15 and 16; and
y is an integer selected from the group consisting of 1, 2, 3, 4 and 5.
   -D of formula (Ia) or (Ib) is covalently and reversibly conjugated to -L¹-.

In certain embodiments, x of formula (Ia) is an integer selected from the group consisting of 1, 2, 3, 4, 6 and 8. In certain embodiments, x of formula (Ia) is an integer selected from the group consisting of 1, 2, 4 and 6. In certain embodiments, x of formula (Ia) is an integer selected from the group consisting of 1, 4 and 6 and in certain embodiments, x of formula (Ia) is 1.

In certain embodiments, y of formula (Ib) is an integer selected from the group consisting of 2, 3, 4 and 5. In certain embodiments, y of formula (Ib) is an integer selected from the group consisting of 2, 3 and 4. In certain embodiments, y of formula (Ib) is an integer selected from the group consisting of 2 and 3. In certain embodiments, y of formula (Ib) is an integer selected from the group consisting of 1, 2 and 3. In certain embodiments, y of formula (Ib) is 1. In certain embodiments, y of formula (Ib) is 2.

In certain embodiments, the CNP conjugate is of formula (Ia) with x=1.

In certain embodiments, -D of formula (Ia) or (Ib) has the sequence of SEQ ID NO:9, SEQ ID NO: 10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25 or SEQ ID NO:30. In certain embodiments, -D of formula (Ia) or (Ib) has the sequence of SEQ ID NO:20, SEQ ID NO:21, SEQ ID NO:22, SEQ ID NO:23, SEQ ID NO:24 or SEQ ID NO:25.

In certain embodiments, -D of formula (Ia) or (Ib) has the sequence of SEQ ID NO:20. In certain embodiments, -D of formula (Ia) or (Ib) has the sequence of SEQ ID NO:21. In certain embodiments, -D of formula (Ia) or (Ib) has the sequence of SEQ ID NO:22. In certain embodiments, -D of formula (Ia) or (Ib) has the sequence of SEQ ID NO:23. In certain embodiments, -D of formula (Ia) or (Ib) has the sequence of SEQ ID NO:24. In certain embodiments, -D of formula (Ia) or (Ib) has the sequence of SEQ ID NO:25.

In certain embodiments, -D of formula (Ia) or (Ib) has the sequence of SEQ ID NO:30, SEQ ID NO:98, SEQ ID NO:99 or SEQ ID NO 90.

The moiety -L¹- of formula (Ia) or (Ib) is either conjugated to a functional group of the side chain of an amino acid residue of -D, to the N-terminal amine functional group or to the C-terminal carboxyl functional group of -D or to a nitrogen atom in the backbone polypeptide chain of -D. Attachment to either the N-terminus or C-terminus can either be direct through the corresponding amine or carboxyl functional group, respectively, or indirect wherein a spacer moiety is first conjugated to the amine or carboxyl functional group to which spacer moiety -L¹- is conjugated.

The moiety -L¹- of formula (Ia) or (Ib) is a reversible linker from which the drug, i.e. D-H is released in its free form, i.e. -L¹- is a traceless linker. Suitable reversible linkers are known in the art, such as for example the reversible linker moieties disclosed in WO 2005/099768 A2, WO 2006/136586 A2, WO 2011/089216 A1 and WO 2013/024053 A1, which are incorporated by reference herewith.

In certain embodiments, -L¹- is a reversible linker as described in WO 2011/012722 A1, WO 2011/089214 A1, WO 2011/089215 A1, WO 2013/024052 A1 and WO 2013/160340 A1 which are incorporated by reference herewith.

The moiety -L¹- can be connected to -D through any type of linkage, provided that it is reversible. In certain embodiments, -L¹- is connected to -D through a linkage selected from the group consisting of amide, ester, carbamate, acetal, aminal, imine, oxime, hydrazone, disulfide and acylguanidine. In certain embodiments, -L¹- is connected to -D through a linkage selected from the group consisting of amide, ester, carbamate and acylguanidine. It is understood that these linkages may not per se be reversible, but that neighboring groups comprised in -L¹- may render the linkage reversible.

In certain embodiments, the moiety -L¹- is connected to -D through an amide linkage.

A moiety -L¹- is disclosed in WO 2009/095479 A2. Accordingly, in certain embodiments, the moiety -L¹- is of formula (II):

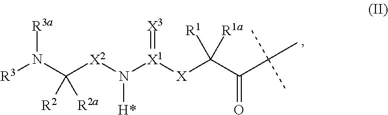

wherein the dashed line indicates the attachment to a nitrogen of -D which is a CNP moiety by forming an amide bond;
—X— is —C(R⁴R^{4a})—; —O—; —C(R⁴R^{4a})—C(R⁵R^{5a})—; —C(R⁵R^{5a})—C(R⁴R^{4a}); —C(R⁴R^{4a})—N(R⁶)—; —N(R⁶)—C(R⁴R^{4a})—; —C(R⁴R^{4a})—O—; —O—C(R⁴R^{4a}); or
X¹ is C; or S(O);
—X²— is —C(R⁸R^{8a})—; or —C(R⁸R^{8a})—C(R⁹R^{9a})—;
—X³ is ═O; ═S; or ═N—CN;
—R¹, —R^{1a}, —R², —R^{2a}, —R⁴, —R^{4a}, —R⁵, —R^{5a}, —R⁶, —R⁸, —R^{8a}, —R⁹, —R^{9a} are independently selected from the group consisting of —H; and C₁₋₆ alkyl;
—R³, —R^{3a} are independently selected from the group consisting of —H; and C₁₋₆ alkyl, provided that in case one of —R³, —R^{3a} or both are other than —H they are connected to the N to which they are attached through an spa-hybridized carbon atom;
—R⁷ is —N(R¹⁰R^{10a}); or —NR¹⁰—(C═O)—R¹¹;
—R^{7a}, —R¹⁰, —R^{10a}—R¹¹ are independently of each other —H; or C₁₋₆ alkyl;
optionally, one or more of the pairs -R^{1a}/—R^{4a}, -R^{1a}/—R^{5a}, —R^{1a}/_R^{7a}, —R^{4a}/—R^{5a}, —R^{8a}/—R^{9a} form a chemical bond;
optionally, one or more of the pairs -R¹/—R^{1a}, -R²/—R^{2a}, —R⁴/—R^{4a}, —R⁵/—R^{5a}, —R⁸/—R^{8a}, —R⁹/—R^{9a} are joined together with the atom to which they are attached to form a C₃₋₁₀ cycloalkyl; or 3- to 10-membered heterocyclyl;
optionally, one or more of the pairs -R¹/—R⁴, -R¹/—R⁵, -R¹/—R⁶, -R¹/—R^{7a}, -R⁴/—R⁵, -R⁴/—R⁶, —R⁸/—R⁹, -R²/—R³ are joined together with the atoms to which they are attached to form a ring A;
optionally, —R³/—R^{3a} are joined together with the nitrogen atom to which they are attached to form a 3- to 10-membered heterocycle;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein -L¹- is substituted with -L²-Z and wherein -L¹- is optionally further substituted, provided that the hydrogen marked with the asterisk in formula (II) is not replaced by -L²-Z or a substituent;
   wherein
   -L²- is a single chemical bond or a spacer; and
   —Z is a water-soluble polymeric moiety;

In certain embodiments, -L¹- of formula (II) is substituted with one moiety -L²-Z. In certain embodiments, -L¹- of formula (II) is not further substituted.

It is understood that if -R³/—R^{3a} of formula (II) are joined together with the nitrogen atom to which they are attached to form a 3- to 10-membered heterocycle, only such 3- to 10-membered heterocycles, in which the atoms directly attached to the nitrogen are sp³-hybridized carbon atoms may be formed. In other words, such 3- to 10-membered heterocycle formed by -R³/—R^{3a} together with the nitrogen atom to which they are attached has the following structure:

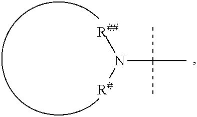

wherein
the dashed line indicates attachment to the rest of -L¹-;
the ring comprises 3 to 10 atoms comprising at least one nitrogen; and
R^{#} and R^{##} represent a spa-hydridized carbon atom.

It is also understood that the 3- to 10-membered heterocycle may be further substituted.

Exemplary embodiments of suitable 3- to 10-membered heterocycles formed by -R³/—R^{3a} of formula (II) together with the nitrogen atom to which they are attached are the following:

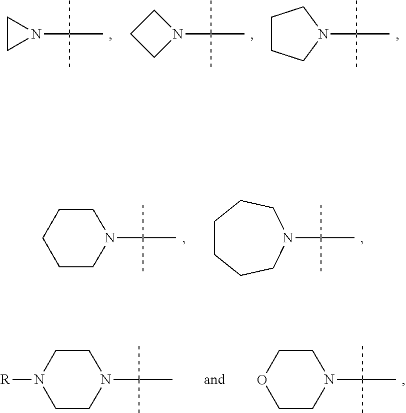

wherein
dashed lines indicate attachment to the rest of the molecule; and
—R is selected from the group consisting of —H and C₁₋₆ alkyl.

-L¹- of formula (II) may optionally be further substituted. In general, any substituent may be used as far as the cleavage principle is not affected, i.e. the hydrogen marked with the asterisk in formula (II) is not replaced and the nitrogen of the moiety

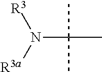

of formula (II) remains part of a primary, secondary or tertiary amine, i.e. —R³ and —R^{3a} are independently of each other —H or are connected to —N< through a sp³-hybridized carbon atom.

In certain embodiments, —R¹ or —R^{1a} of formula (II) is substituted with -L²-Z. In certain embodiments, —R² or —R^{2a} of formula (II) is substituted with -L²-Z. In certain embodiments, —R³ or -R^{3a} of formula (II) is substituted with -L²-Z. In certain embodiments, —R⁴ of formula (II) is substituted with -L²-Z. In certain embodiments, —R⁵ or -R^{5a} of formula (II) is substituted with -L²-Z. In certain embodiments, —R⁶ of formula (II) is substituted with -L²-Z. In certain embodiments, —R⁷ or —R^{7a} of formula (II) is substituted with -L²-Z. In certain embodiments, —R⁸ or -R^{8a} of formula (II) is substituted with -L²-Z. In certain embodiments, —R⁹ or —R^{9a} of formula (II) is substituted with -L²-Z.

In certain embodiments, —R⁴ of formula (II) is substituted with -L²-Z.

In certain embodiments, —X— of formula (II) is —C(R⁴R^{4a})— or —N(R⁴)—. In certain embodiments, —X— of formula (II) is —C(R⁴R^{4a})—.

In certain embodiments, X¹ of formula (II) is C.

In certain embodiments, ═X³ of formula (II) is ═O.

In certain embodiments, —X²— of formula (II) is —C(R⁸R^{8a})—.

In certain embodiments, —R⁸ and —R^{8a} of formula (II) are independently selected from the group consisting of —H, methyl and ethyl. In certain embodiments, at least one of —R⁸ and —R^{8a} of formula (II) is —H. In certain embodiments, both —R⁸ and —R^{8a} of formula (II) are —H.

In certain embodiments, —R¹ and —R^{1a} of formula (II) are independently selected from the group consisting of —H, methyl and ethyl. In certain embodiments, at least one of —R¹ and —R^{1a} of formula (II) is —H. In certain embodiments, both —R¹ and —R^{1a} of formula (II) are —H.

In certain embodiments, —R² and -R^{2a} of formula (II) are independently selected from the group consisting of —H, methyl and ethyl. In certain embodiments, at least one of —R² and -R^{2a} of formula (II) is —H. In certain embodiments, both —R² and —R^{2a} of formula (II) are H.

In certain embodiments, —R³ and —R^{3a} of formula (II) are independently selected from the group consisting of —H, methyl, ethyl, propyl and butyl. In certain embodiments, at least one of —R³ and -R^{3a} of formula (II) is methyl. In certain embodiments, —R³ and -R^{3a} of formula (II) are both —H. In certain embodiments, —R³ and -R^{3a} of formula (II) are both methyl. In certain embodiments, —R³ of formula (II) is —H and -R^{3a} of formula (II) is methyl.

In certain embodiments, —R⁴ and -R^{4a} of formula (II) are independently selected from the group consisting of —H, methyl and ethyl. In certain embodiments, at least one of —R⁴ and -R^{4a} of formula (II) is —H. In certain embodiments, both —R⁴ and —R^{4a} of formula (II) are —H.

In certain embodiments, the moiety -L¹- is of formula (IIa):

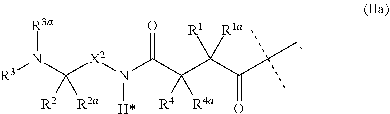

wherein the dashed line indicates the attachment to a nitrogen of -D which is a CNP moiety by forming an amide bond;
—R¹, —R^{1a}, —R², —R^{2a}, —R³, —R^{3a}, —R⁴, —R^{4a} and —X²— are used as defined in formula (II); and wherein -L¹- is substituted with -L²-Z and wherein -L¹- is optionally further substituted, provided that the hydrogen marked with the asterisk in formula (IIa) is not replaced by -L²-Z or a substituent.

In certain embodiments, -L¹- of formula (IIa) is substituted with one moiety -L²-Z. In certain embodiments, the moiety -L¹- of formula (IIa) is not further substituted.

In certain embodiments, —R¹ and —R^{1a} of formula (IIa) are independently selected from the group consisting of —H, methyl and ethyl. In certain embodiments, at least one of —R¹ and —R^{1a} of formula (IIa) is —H. In certain embodiments, both —R¹ and —R^{1a} of formula (IIa) are —H.

In certain embodiments, —R⁴ and -R^{4a} of formula (IIa) are independently selected from the group consisting of —H, methyl and ethyl. In certain embodiments, at least one of —R⁴ and -R^{4a} of formula (IIa) is —H. In certain embodiments, both —R⁴ and —R^{4a} of formula (IIa) are —H.

In certain embodiments, —X²— of formula (IIa) is —C(R⁸R^{8a})—.

In certain embodiments, —R⁸ and -R^{8a} of formula (IIa) are independently selected from the group consisting of —H, methyl and ethyl. In certain embodiments, at least one of —R⁸ and -R^{8a} of formula (IIa) is —H. In certain embodiments, both —R⁸ and -R^{8a} of formula (IIa) are —H.

In certain embodiments, —R² and -R^{2a} of formula (IIa) are independently selected from the group consisting of —H, methyl and ethyl. In certain embodiments, at least one of —R² and —R^{2a} of formula (IIa) is —H. In certain embodiments, both —R² and —R^{2a} of formula (IIa) are H.

In certain embodiments, —R³ and -R^{3a} of formula (IIa) are independently selected from the group consisting of —H, methyl, ethyl, propyl and butyl. In certain embodiments, at least one of —R³ and -R^{3a} of formula (IIa) is methyl. In certain embodiments, —R³ and -R^{3a} of formula (IIa) are both —H. In certain embodiments, —R³ and -R^{3a} of formula (IIa) are both methyl. In certain embodiments, —R³ of formula (IIa) is —H and -R^{3a} of formula (IIa) is methyl. In certain embodiments, the moiety -L¹- is of formula (IIb):

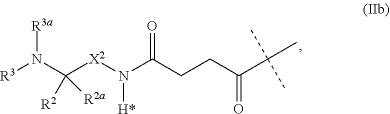

wherein the dashed line indicates the attachment to a nitrogen of -D which is a CNP moiety by forming an amide bond;
—R², —R^{2a}, —R³, —R^{3a} and —X²— are used as defined in formula (II); and
wherein -L¹- is substituted with -L²-Z and wherein -L¹- is optionally further substituted, provided that the hydrogen marked with the asterisk in formula (IIb) is not replaced by -L²-Z or a substituent.

In certain embodiments, -L¹- of formula (IIb) is substituted with one moiety -L²-Z. In certain embodiments, the moiety -L¹- of formula (IIb) is not further substituted.

In certain embodiments, —X²— of formula (IIb) is —C(R⁸R^{8a})—.

In certain embodiments, —R⁸ and —R^{8a} of formula (IIb) are independently selected from the group consisting of —H, methyl and ethyl. In certain embodiments, at least one of —R⁸ and —R^{8a} of formula (IIb) is —H. In certain embodiments, both —R⁸ and —R^{8a} of formula (IIb) are —H.

In certain embodiments, —R² and -R^{2a} of formula (IIb) are independently selected from the group consisting of —H, methyl and ethyl. In certain embodiments, at least one of —R² and —R^{2a} of formula (IIb) is —H. In certain embodiments, both —R² and —R^{2a} of formula (IIb) are H.

In certain embodiments, —R³ and -R^{3a} of formula (IIb) are independently selected from the group consisting of —H, methyl, ethyl, propyl and butyl. In certain embodiments, at least one of —R³ and -R^{3a} of formula (IIb) is methyl. In certain embodiments, —R³ and -R^{3a} of formula (IIb) are both —H. In certain embodiments, —R³ and —R^{3a} of formula (IIb) are both methyl. In certain embodiments, —R³ of formula (IIb) is —H and -R^{3a} of formula (IIb) is methyl.

In certain embodiments, the moiety -L¹- is of formula (IIb′):

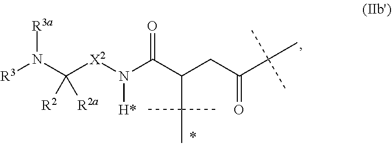

wherein
wherein the dashed line indicates the attachment to a nitrogen of -D which is a CNP moiety by forming an amide bond;
the dashed line marked with the asterisk indicates attachment to -L²-;
—R², —R^{2a}, —R³, —R^{3a} and —X²— are used as defined in formula (II); and
wherein -L¹- is optionally further substituted, provided that the hydrogen marked with the asterisk in formula (IIb′) is not replaced by a substituent.

In certain embodiments, the moiety -L¹- of formula (IIb′) is not further substituted.

In certain embodiments, —X²— of formula (IIb′) is —C(R⁸R^{8a})—.

In certain embodiments, —R⁸ and -R^{8a} of formula (IIb′) are independently selected from the group consisting of —H, methyl and ethyl. In certain embodiments, at least one of —R⁸ and -R^{8a} of formula (IIb′) is —H. In certain embodiments, both —R⁸ and -R^{8a} of formula (IIb′) are —H.

In certain embodiments, —R² and -R^{2a} of formula (IIb′) are independently selected from the group consisting of —H, methyl and ethyl. In certain embodiments, at least one of —R² and —R^{2a} of formula (IIb′) is —H. In certain embodiments, both —R² and —R^{2a} of formula (IIb′) are H.

In certain embodiments, —R³ and -R^{3a} of formula (IIb′) are independently selected from the group consisting of —H, methyl, ethyl, propyl and butyl. In certain embodiments, at least one of —R³ and -R^{3a} of formula (IIb′) is methyl. In certain embodiments, —R³ and -R^{3a} of formula (IIb′) are both —H. In certain embodiments, —R³ and —R^{3′} of formula (IIb′) are both methyl. In certain embodiments, —R³ of formula (IIb′) is —H and -R^{3a} of formula (IIb′) is methyl.

In certain embodiments, the moiety -L¹- is of formula (IIc):

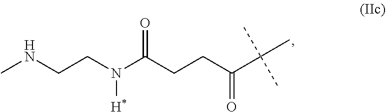

wherein the dashed line indicates the attachment to a nitrogen of -D which is a CNP moiety by forming an amide bond; and
wherein -L¹- is substituted with -L²-Z and wherein -L¹- is optionally further substituted, provided that the hydrogen marked with the asterisk in formula (IIc) is not replaced by -L²-Z or a substituent.

In certain embodiments, -L¹- of formula (IIc) is substituted with one moiety -L²-Z. In certain embodiments, the moiety -L¹- of formula (IIc) is not further substituted.

In certain embodiments, the moiety -L¹- is selected from the group consisting of formula (IIc-i), (IIc-ii), (IIc-iii), (IIc-iv) and (IIc-v):

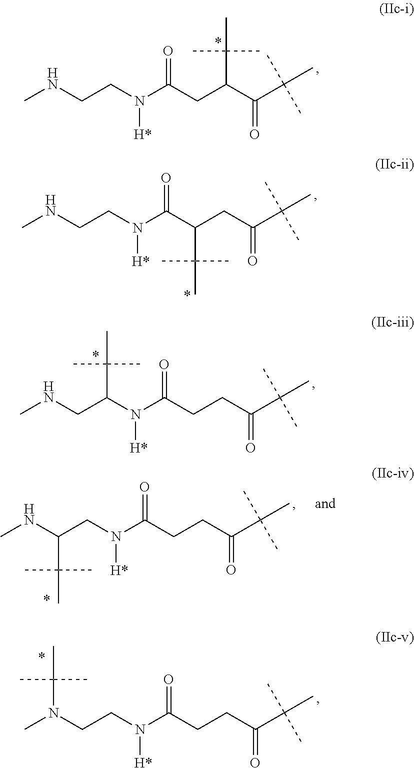

wherein
the unmarked dashed line indicates the attachment to a nitrogen of -D which is a CNP moiety by forming an amide bond; and
the dashed line marked with the asterisk indicates attachment to -L²-Z; and
-L¹- is optionally further substituted, provided that the hydrogen marked with the asterisk in formula (IIc-i), (IIc-ii), (IIc-iii), (IIc-iv) and (IIc-v) is not replaced by a substituent.

In certain embodiments, the moiety -L¹- of formula (IIc-i), (IIc-ii), (IIc-iii), (IIc-iv) and (IIc-v) is not further substituted.

In certain embodiments, the moiety -L¹- is of formula (IIc-ii):

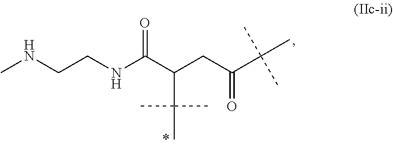

wherein
the unmarked dashed line indicates the attachment to a nitrogen of -D which is a CNP moiety by forming an amide bond; and
the dashed line marked with the asterisk indicates attachment to -L²-Z.

In certain embodiments, -L¹- of formula (IIc-ii) is substituted with one moiety -L²-Z.

The optional further substituents of -L¹- of formula (II), (IIa), (IIb), (IIb′), (IIc), (IIc-a), (IIc-b), (IIc-i), (IIc-ii), (IIc-iii), (IIc-iv), (IIc-v) are in certain embodiments as described above.

Another moiety -L¹- is disclosed in WO2016/020373A1. Accordingly, in certain embodiments, the moiety -L¹- is of formula (III):

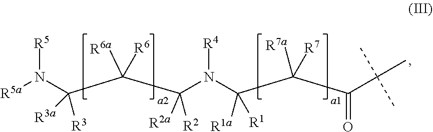

wherein
the dashed line indicates attachment to a primary or secondary amine or hydroxyl of -D which is a CNP moiety by forming an amide or ester linkage, respectively;
—R¹, —R^{1a}, —R², —R^{2a}, —R³ and —R^{3a} are independently of each other selected from the group consisting of —H, —C(R⁸R^{8a}R^{8b}), —C(═O)R⁸, —C≡N, —C(═NR⁸)R^{8a}, —CR⁸(═CR^{8a}R^{8b}), —C≡CR⁸ and -T;
—R⁴, —R⁵ and -R^{5a} are independently of each other selected from the group consisting of —H, —C(R⁹R^{9a}R^{9b}) and -T;
a1 and a2 are independently of each other 0 or 1;
each —R⁶, -R^{6a}, —R⁷, -R^{7a}, —R⁸, —R^{8a}, —R^{8b}, —R⁹, —R^{9a}, —R^{9b} are independently of each other selected from the group consisting of —H, halogen, —CN, —COOR¹⁰, —OR¹⁰, —C(O)R¹⁰, —C(O)N(R¹⁰R^{10a}), —S(O)₂N(R¹⁰R^{10a}), —S(O)N(R¹⁰R^{10a}), —S(O)₂R¹⁰—S(O)R¹⁰, —N(R¹⁰)S(O)₂N(R^{10a}R^{10b}), —SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O)OR^{10a}, —N(R¹⁰)C(O)N(R^{10a}R^{10b}), —OC(O)N(R¹⁰R^{10a}), -T C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each —R¹⁰, —R^{10a}, —R^{10b} is independently selected from the group consisting of —H, -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a}), —N(R¹²)C(O)N(R^{12a}) and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 1 1-membered heterobicyclyl; wherein each T is independently optionally substituted with one or more —R¹¹, which are the same or different;
each —R¹¹ is independently of each other selected from halogen, —CN, oxo (═O), —COOR¹³, —OR¹³, —C(O)R¹³, —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{13a}, —N(R¹³)C(O)OR^{13a}, —N(R¹³)C(O)N(R^{13a}R^{13b}), —OC(O)N(R¹³R^{13a}) and C₁₋₆ alkyl; wherein C₁₋₆ alkyl is optionally substituted with one or more halogen, which are the same or different;
each —R¹², —R^{12a}, —R¹³, —R^{13a}, —R^{13b} is independently selected from the group consisting of —H, and C₁₋₆ alkyl; wherein C₁₋₆ alkyl is optionally substituted with one or more halogen, which are the same or different;
optionally, one or more of the pairs -R¹/—R^{1a}, —R²/—R^{2a}, -R³/—R^{3a}, -R⁶/—R^{6a}, -R⁷/—R^{7a} are joined together with the atom to which they are attached to form a C₃₋₁₀ cycloalkyl or a 3- to 10-membered heterocyclyl;
optionally, one or more of the pairs -R¹/—R², —R¹/—R³, —R¹/—R⁴, —R¹/—R⁵, —R¹/—R⁶, -R¹/—R⁷, -R²/—R³, -R²/—R⁴, -R²/—R⁵, -R²/—R⁶, -R²/—R⁷, -R³/—R⁴, -R³/—R⁵, —R³/—R⁶, -R³/—R⁷, -R⁴/—R⁵, -R⁴/—R⁶, -R⁴/—R⁷, -R⁵/—R⁶, -R⁵/—R⁷, -R⁶/—R⁷ are joined together with the atoms to which they are attached to form a ring A;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl;
wherein -L¹- is substituted with -L²-Z and wherein -L¹- is optionally further substituted;
   wherein
   -L²- is a single chemical bond or a spacer; and
   —Z is a water-soluble polymeric moiety.

The optional further substituents of -L¹- of formula (III) are in certain embodiments as described above. In certain embodiments, -L¹- of formula (III) is substituted with one moiety -L²-Z. In certain embodiments, -L¹- of formula (III) is not further substituted.

Additional embodiments for -L¹- are disclosed in EP1536334B1, WO2009/009712A1, WO2008/034122A1, WO2009/143412A2, WO2011/082368A2, and US8618124B2, which are herewith incorporated by reference in their entirety.

Additional embodiments for -L¹- are disclosed in U.S. Pat. No. 8,946,405B2 and U.S. Pat. No. 8,754,190B2, which are herewith incorporated by reference in their entirety. Accordingly, a moiety -L¹- is of formula (IV)

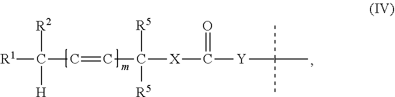

wherein
   the dashed line indicates attachment to -D which is a CNP moiety and wherein attachment is through a functional group of -D selected from the group consisting of —OH, —SH and —NH₂;
   m is 0 or 1;
   at least one or both of —R¹ and —R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴,
   one and only one of —R¹ and —R² is selected from the group consisting of —H, optionally substituted alkyl, optionally substituted arylalkyl, and optionally substituted heteroarylalkyl;
   —R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
   —R⁴ is selected from the group consisting of optionally substituted alkyl, optionally substituted aryl, optionally substituted arylalkyl, optionally substituted heteroaryl, and optionally substituted heteroarylalkyl;
   each —R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
   —R⁹ is selected from the group consisting of —H and optionally substituted alkyl;
   —Y— is absent and —X— is —O— or —S—; or
   —Y— is —N(Q)CH₂— and —X— is —O—;
   Q is selected from the group consisting of optionally substituted alkyl, optionally substituted aryl, optionally substituted arylalkyl, optionally substituted heteroaryl and optionally substituted heteroarylalkyl;
   optionally, —R¹ and —R² may be joined to form a 3 to 8-membered ring; and
   optionally, both —R⁹ together with the nitrogen to which they are attached form a heterocyclic ring;
   wherein -L¹- is substituted with -L²-Z and wherein -L¹- is optionally further substituted;
      wherein
      -L²- is a single chemical bond or a spacer; and
      —Z is a water-soluble polymeric moiety.

The optional further substituents of -L¹- of formula (IV) are in certain embodiments, as described above. In certain embodiments, -L¹- of formula (IV) is substituted with one moiety -L²-Z. In certain embodiments, -L¹- of formula (IV) is not further substituted.

Only in the context of formula (IV) the terms used have the following meaning:

The term “alkyl” as used herein includes linear, branched or cyclic saturated hydrocarbon groups of 1 to 8 carbons, or in certain embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, in certain embodiments, 6 to 10 carbons, including groups such as phenyl, naphthyl, and anthracenyl. The term “heteroaryl” includes aromatic rings comprising 3 to 15 carbons containing at least one N, O or S atom, in certain embodiments, 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In certain embodiments, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted. Optional substituents on any group, including the above, include halo, nitro, cyano, —OR, —SR, —NR₂, —OCOR, —NRCOR, —COOR, —CONR₂, —SOR, —SO₂R, —SONR₂, —SO₂NR₂, wherein each R is independently alkyl, alkenyl, alkynyl, aryl or heteroaryl, or two R groups taken together with the atoms to which they are attached form a ring.

An additional embodiment for -L¹- is disclosed in WO2013/036857A1, which is herewith incorporated by reference in its entirety. Accordingly, in certain embodiments, moiety -L¹- is of formula (V):

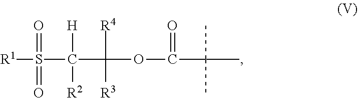

wherein
the dashed line indicates attachment to -D which is a CNP moiety and wherein attachment is through an amine functional group of -D;
—R¹ is selected from the group consisting of optionally substituted C₁-C₆ linear, branched, or cyclic alkyl; optionally substituted aryl; optionally substituted heteroaryl; alkoxy; and —NR⁵₂;
—R² is selected from the group consisting of —H; optionally substituted C₁-C₆ alkyl; optionally substituted aryl; and optionally substituted heteroaryl;
—R³ is selected from the group consisting of —H; optionally substituted C₁-C₆ alkyl; optionally substituted aryl; and optionally substituted heteroaryl;
—R⁴ is selected from the group consisting of —H; optionally substituted C₁-C₆ alkyl; optionally substituted aryl; and optionally substituted heteroaryl;
each —R⁵ is independently of each other selected from the group consisting of —H; optionally substituted C₁-C₆ alkyl; optionally substituted aryl; and optionally substituted heteroaryl; or when taken together two —R⁵ can be cycloalkyl or cycloheteroalkyl;
wherein -L¹- is substituted with -L²-Z and wherein -L¹- is optionally further substituted;
   wherein
   -L²- is a single chemical bond or a spacer; and
   —Z is a water-soluble polymeric.

The optional further substituents of -L¹- of formula (V) are, in certain embodiments as described above.

In certain embodiments, -L¹- of formula (V) is substituted with one moiety -L²-Z.

In certain embodiments, -L¹- of formula (V) is not further substituted.

Only in the context of formula (V) the terms used have the following meaning:

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 C.

“Aryl” includes aromatic hydrocarbon groups of 6-18 carbons, in certain embodiments, 6-10 carbons, including groups such as phenyl, naphthyl, and anthracene “Heteroaryl” includes aromatic rings comprising 3-15 carbons containing at least one N, O or S atom, in certain embodiments, 3-7 carbons containing at least one N, O or S atom, including groups such as pyrrolyl, pyridyl, pyrimidinyl, imidazolyl, oxazolyl, isoxazolyl, thiszolyl, isothiazolyl, quinolyl, indolyl, indenyl, and similar.

The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketone; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

Another embodiment for -L¹- is disclosed in WO 2022/115563 A1, which is herewith incorporated by reference in its entirety. Accordingly, in certain embodiments, -L¹- is of formula (Va):

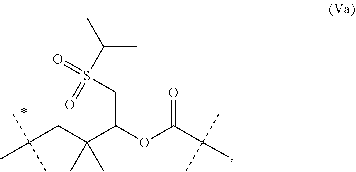

wherein the dashed line marked with the asterisk indicates the attachment to -L²-Z and the unmarked dashed line indicates the attachment to -D.

In certain embodiments, -L¹- is of formula (Va), the dashed line marked with the asterisk indicates the attachment to -L²-Z and the unmarked dashed line indicates the attachment to -D, wherein -D is a CNP moiety of the following amino acid sequence:

### SEQ ID NO:97 (CNP-38 N6Q, N14Q):

### LQEHPQARKYKGAQKKGLSKGCFGLKLDRIGSMSGLGC,

wherein the cysteines at position 22 and 38 are connected through a disulfide-bridge; and
wherein the attachment to -L¹- takes place either at the N-terminal or ring of the peptide.

Another embodiment for -L¹- is disclosed in U.S. Pat. No. 7,585,837B2, which is herewith incorporated by reference in its entirety. Accordingly, in certain embodiments, a moiety -L¹- is of formula (VI):

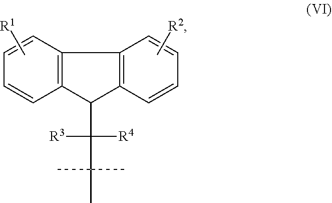

wherein
the dashed line indicates attachment to -D which is a CNP moiety and wherein attachment is through an amine functional group of -D;
R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl;
wherein -L¹- is substituted with -L²-Z and wherein -L¹- is optionally further substituted;
   wherein
   -L²- is a single chemical bond or a spacer; and
   —Z is a water-soluble polymeric moiety.

Suitable substituents for formulas (VI) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

In certain embodiments, -L¹- of formula (VI) is substituted with one moiety -L²-Z. The optional further substituents of -L¹- of formula (VI) are in certain embodiments as described above.

In certain embodiments, -L¹- of formula (VI) is not further substituted.

Only in the context of formula (VI) the terms used have the following meaning:

The terms “alkyl”, “alkoxy”, “alkoxyalkyl”, “aryl”, “alkaryl” and “aralkyl” mean alkyl radicals of 1-8, in certain embodiments, 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

A further embodiment for -L¹- is disclosed in WO2002/089789A1, which is herewith incorporated by reference in its entirety. Accordingly, a moiety -L¹- is of formula (VII):

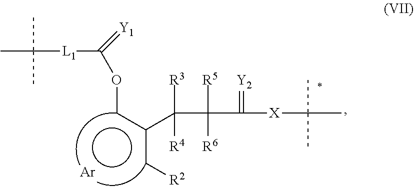

wherein
the dashed line indicates attachment to -D which is a CNP moiety and wherein attachment is through an amine functional group of -D;
L₁ is a bifunctional linking group,
Y₁ and Y₂ are independently O, S or NR⁷;
R², R³, R⁴, R⁵, R⁶ and R⁷ are independently selected from the group consisting of hydrogen, C₁₋₆ alkyls, C_{3 -12} branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
Ar is a moiety which when included in formula (VII) forms a multisubstituted aromatic hydrocarbon or a multi-substituted heterocyclic group;
X is a chemical bond or a moiety that is actively transported into a target cell, a hydrophobic moiety, or a combination thereof,
y is 0 or 1;
wherein -L¹- is substituted with -L²-Z and wherein -L¹- is optionally further substituted;
   wherein
   -L²- is a single chemical bond or a spacer; and
   —Z is a water-soluble polymeric moiety.

In certain embodiments, -L¹- of formula (VII) is substituted with one moiety -L²-Z. The optional further substituents of -L¹- of formula (VII) are in certain embodiments, as described above.

In certain embodiments, -L¹- of formula (VII) is not further substituted.

Only in the context of formula (VII) the terms used have the following meaning:

The term “alkyl” shall be understood to include, e.g. straight, branched, substituted C₁₋₁₂ alkyls, including alkoxy, C₃₋₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substituted cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophone; alkoxy includes moieities such as methoxy; and phenoxy includes moieties such as 3-nitrophenoxy. Halo- shall be understood to include fluoro, chloro, iodo and bromo.

In certain embodiments, -L¹- comprises a substructure of formula (VIII):

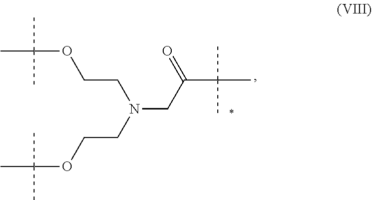

wherein
the dashed line marked with the asterisk indicates attachment to a nitrogen of -D which is a CNP moiety by forming an amide bond;
the unmarked dashed lines indicate attachment to the remainder of -L¹-; and
wherein -L¹- is substituted with -L²-Z and wherein -L¹- is optionally further substituted;
   wherein
   -L²- is a single chemical bond or a spacer; and
   —Z is a water-soluble polymeric moiety.

In certain embodiments, -L¹- of formula (VIII) is substituted with one moiety -L²-Z. The optional further substituents of -L¹- of formula (VIII) are as described above.

In certain embodiments, -L¹- of formula (VIII) is not further substituted.

In certain embodiments, -L¹- comprises a substructure of formula (IX):

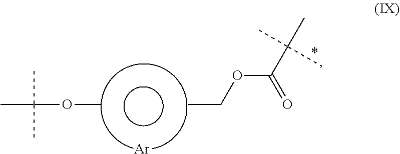

wherein
the dashed line marked with the asterisk indicates attachment to a nitrogen of -D which is a CNP moiety by forming a carbamate bond;
the unmarked dashed lines indicate attachment to the remainder of -L¹-; and
wherein -L¹- is substituted with -L²-Z and wherein -L¹- is optionally further substituted;
   wherein
   -L²- is a single chemical bond or a spacer; and
   —Z is a water-soluble polymeric moiety.

The optional further substituents of -L¹- of formula (IX) are as described above. In certain embodiments, -L¹- of formula (IX) is substituted with one moiety -L²-Z. In certain embodiments, -L¹- of formula (IX) is not further substituted.

The moiety -D may be connected to -L¹- through any functional group of D-H and is connected to -L¹- through an amine functional group of D-H. This may be the N-terminal amine functional group or an amine functional group provided by a lysine side chain, i.e. by the lysines at position 9, 11, 15, 16, 20 and 26, if the CNP has the sequence of SEQ ID NO:24.

Attachment of -L¹- to the ring of a CNP moiety significantly reduces the CNP conjugate's affinity to NPR-B compared to attachment at the N-terminus or to the non-ring part of CNP, which reduced affinity to NPR-B in turn reduces the risk of cardiovascular side effects, such as hypotension.

Accordingly, in certain embodiments, -L¹- is conjugated to the side chain of an amino acid residue of said ring moiety of -D or to the backbone of said ring moiety of -D. In certain embodiments, -L¹- is covalently and reversibly conjugated to the side chain of an amino acid residue of said ring moiety of -D. If -D is a CNP moiety with the sequence of SEQ ID NO:24, -L¹- is, in certain embodiments, conjugated to the amine functional group provided by the lysine at position 26 of the corresponding drug D-H.

The moiety -L²- is a chemical bond or a spacer moiety. In certain embodiments, -L²- is a chemical bond. In certain embodiments, -L²- is a spacer moiety.

The moiety -L²- can be attached to -L¹- by replacing any —H present, except where explicitly excluded.

When -L²- is other than a single chemical bond, -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—,
—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—,
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

When -L²- is other than a single chemical bond, -L²- is selected from the group consisting of -T-, —C(O)O—, —O—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₂₀ alkyl, C₂₋₂₀ alkenyl, and C₂₋₂₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a}) and —OC(O)N(R^{y3})—;
—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a}) and —OC(O)N(R^{y4})—,
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
—R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

When -L²- is other than a single chemical bond, -L²- is selected from the group consisting of -T-, —C(O)O—, —O—, —C(O)N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —O—, —C(O)—, —C(O)N(R^{y3})—, —S(O)₂N(R^{y3})—, —S(O)N(R^{y3})—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
R^{y1} and —R^{y1a} are independently selected from the group consisting of —H, -T, C₁₋₁₀ alkyl, C₂₋₁₀ alkenyl, and C₂₋₁₀ alkynyl;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;
each —R^{y2} is independently selected from the group consisting of halogen, and C₁₋₆ alkyl; and each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments, -L²- is a C₁₋₂₀ alkyl chain, which is optionally interrupted by one or more groups independently selected from —O—, -T- and —C(O)N(R^{y1})—; and which C₁₋₂₀ alkyl chain is optionally substituted with one or more groups independently selected from —OH, -T and —C(O)N(R^{y6}R^{y6a}); wherein —R^{y1}, —R^{y6}, —R^{y6a} are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments, -L²- has a molecular weight in the range of from 14 g/mol to 750 g/mol.

In certain embodiments, -L²- has a chain length of 1 to 20 atoms.

As used herein, the term “chain length” with regard to the moiety -L²- refers to the number of atoms of -L²- present in the shortest connection between -L¹- and —Z.

In certain embodiments, -L²- is of formula (i):

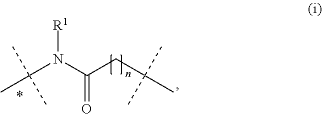

wherein
the dashed line marked with the asterisk indicates attachment to -L¹-;
the unmarked dashed line indicates attachment to —Z;
—R¹ is selected from the group consisting of —H, C₁₋₆ alkyl, C₂₋₆ alkenyl and C₂₋₆ alkynyl;
n is selected from the group consisting of 0, 1, 2, 3, 4, 5, 6, 7, 8, 9, 10, 11, 12, 13, 14, 15, 16, 17 and 18; and
wherein the moiety of formula (i) is optionally further substituted.

In certain embodiments, —R¹ of formula (i) is selected from the group consisting of —H, methyl, ethyl, propyl, and butyl. In certain embodiments, —R¹ of formula (i) is selected from the group consisting of —H, methyl, ethyl and propyl. In certain embodiments, —R¹ of formula (i) is selected from the group consisting of —H and methyl. In certain embodiments, —R¹ of formula (i) is methyl.

In certain embodiments, n of formula (i) is selected from the group consisting of 0, 1, 2, 3, 4, 5, 6, 7, 8, 9 and 10. In certain embodiments, n of formula (i) is selected from the group consisting of 0, 1, 2, 3, 4 and 5. In certain embodiments, n of formula (i) is selected from the group consisting of 0, 1, 2 and 3. In certain embodiments, n of formula (i) is selected from the group consisting of 0 and 1. In certain embodiments, n of formula (i) is 0.

In certain embodiments, -L²- is a moiety selected from the group consisting of:

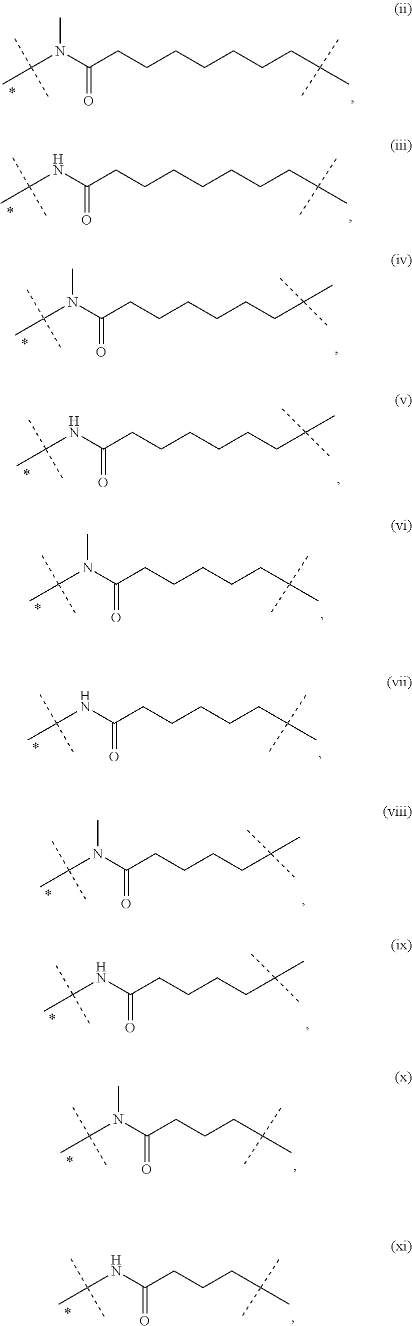

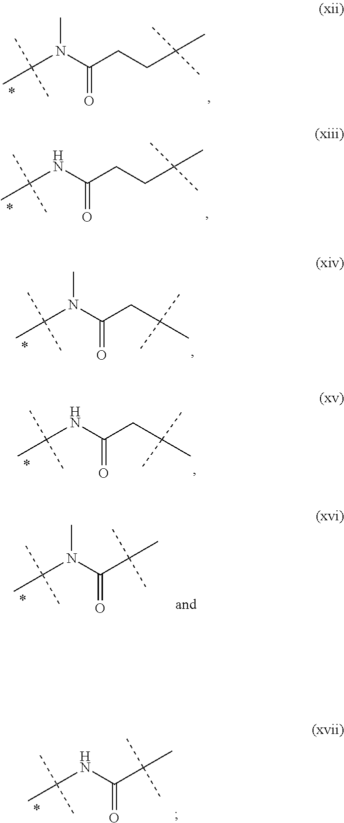

wherein
the dashed line marked with the asterisk indicates attachment to -L¹-;
the unmarked dashed line indicates attachment to —Z and
wherein the moieties (ii), (iii), (iv), (v), (vi), (vii), (viii), (ix), (x), (xi), (xii), (xiii), (xiv), (xv), (xvi) and (xvii) are optionally further substituted.

In certain embodiments, -L²- is selected from the group consisting of

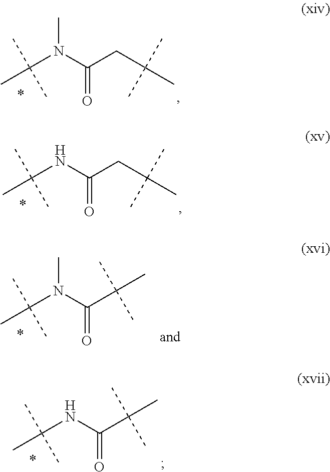

wherein
the dashed line marked with the asterisk indicates attachment to -L¹-; and
the unmarked dashed line indicates attachment to —Z.

In certain embodiments, -L²- is selected from the group consisting of

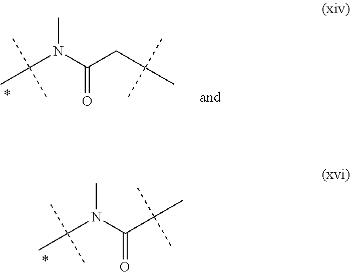

wherein
the dashed line marked with the asterisk indicates attachment to -L¹-; and
the unmarked dashed line indicates attachment to —Z.

In certain embodiments, -L²- is of formula (xvi):

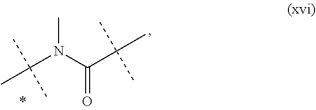

wherein
the dashed line marked with the asterisk indicates attachment to -L¹-; and
the unmarked dashed line indicates attachment to —Z.

In certain embodiments, the moiety -L¹-L²- is selected from the group consisting of

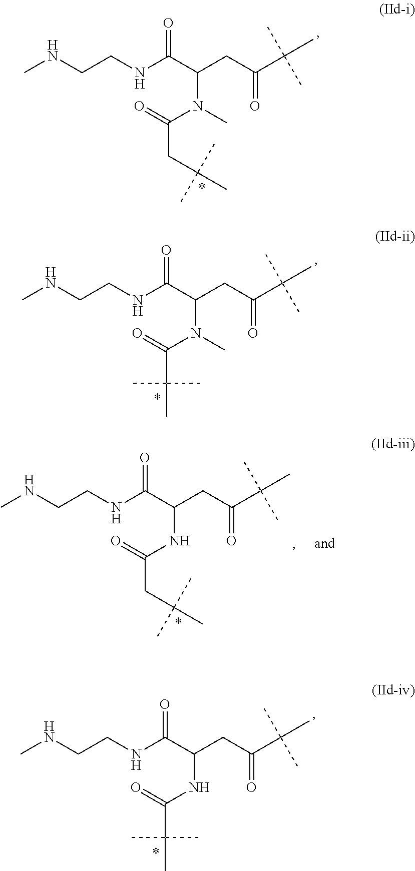

wherein
the unmarked dashed line indicates the attachment to a nitrogen of -D which is a CNP moiety by forming
an amide bond; and
the dashed line marked with the asterisk indicates attachment to —Z.

In certain embodiments, the moiety -L¹-L²- is of formula (IId-ii):

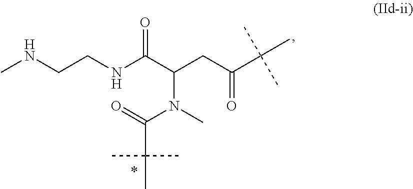

wherein
the unmarked dashed line indicates the attachment to a nitrogen of -D which is a CNP moiety by forming an amide bond; and
the dashed line marked with the asterisk indicates attachment to —Z.

In certain embodiments, the moiety -L¹-L²- is of formula (IId-ii′):

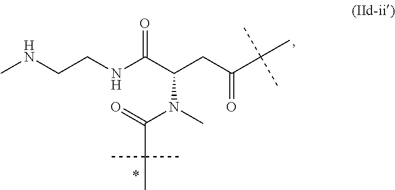

wherein
the unmarked dashed line indicates the attachment to a nitrogen of -D which is a CNP moiety by forming an amide bond; and
the dashed line marked with the asterisk indicates attachment to —Z.

In certain embodiments, the moiety -L¹-L²- is selected from the group consisting of

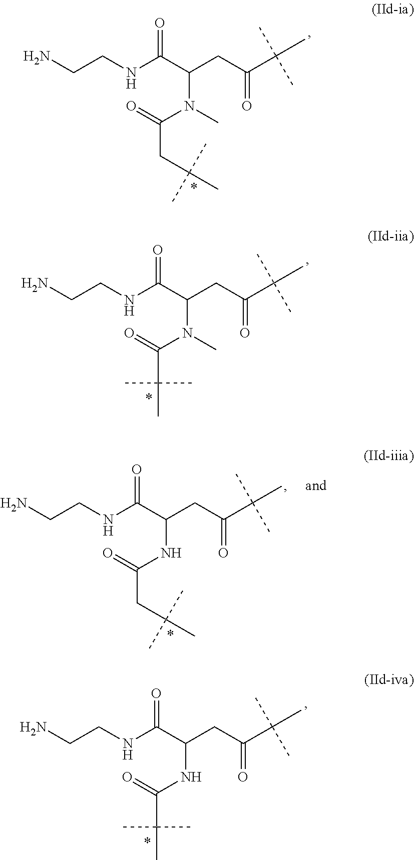

wherein
the unmarked dashed line indicates the attachment to a nitrogen of -D which is a CNP moiety by forming an amide bond; and
the dashed line marked with the asterisk indicates attachment to —Z.

In certain embodiments, —Z of formula (Ia) or (Ib) has a molecular weight ranging from 5 to 200 kDa. In certain embodiments, —Z of formula (Ia) or (Ib) has a molecular weight ranging from 8 to 100 kDa. In certain embodiments, —Z of formula (Ia) or (Ib) has a molecular weight ranging from 10 to 80 kDa. In certain embodiments, —Z of formula (Ia) or (Ib) has a molecular weight ranging from 12 to 60 kDa. In certain embodiments, —Z of formula (Ia) or (Ib) has a molecular weight ranging from 15 to 40 kDa. In certain embodiments, —Z of formula (Ia) or (Ib) has a molecular weight of about 20 kDa. In certain embodiments, —Z of formula (Ia) or (Ib) has a molecular weight of about 40 kDa.

The polymeric moiety —Z of formula (Ia) or (Ib) comprises a polymer. In certain embodiments, —Z of formula (Ia) or (Ib) comprises a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments, —Z of formula (Ia) or (Ib) comprises a protein. Preferred proteins are selected from the group consisting of carboxyl-terminal peptide of the chorionic gonadotropin as described in US 2012/0035101 A1 which are herewith incorporated by reference; albumin; XTEN sequences as described in WO 2011123813 A2 which are herewith incorporated by reference; proline/alanine random coil sequences as described in WO 2011/144756 A1 which are herewith incorporated by reference; proline/alanine/serine random coil sequences as described in WO 2008/155134 A1 and WO 2013/024049 A1 which are herewith incorporated by reference; and Fc-fusion proteins.

In certain embodiments, —Z of formula (Ia) or (Ib) is a polysarcosine. In certain embodiments, —Z of formula (Ia) or (Ib) comprises poly(N-methylglycine). In certain embodiments, —Z of formula (Ia) or (Ib) comprises a random coil protein moiety. In certain embodiments, —Z of formula (Ia) or (Ib) comprises one random coil protein moiety. In certain embodiments, —Z of formula (Ia) or (Ib) comprises two random coil protein moieties. In certain embodiments, —Z of formula (Ia) or (Ib) comprises three random coil protein moieties. In certain embodiments, —Z of formula (Ia) or (Ib) comprises four random coil protein moieties. In certain embodiments, —Z of formula (Ia) or (Ib) comprises five random coil protein moieties. In certain embodiments, —Z of formula (Ia) or (Ib) comprises six random coil protein moieties. In certain embodiments, —Z of formula (Ia) or (Ib) comprises seven random coil protein moieties. In certain embodiments, —Z of formula (Ia) or (Ib) comprises eight random coil protein moieties.

In certain embodiments, such random coil protein moiety comprises at least 25 amino acid residues and at most 2000 amino acids. In certain embodiments, such random coil protein moiety comprises at least 30 amino acid residues and at most 1500 amino acid residues. In certain embodiments, such random coil protein moiety comprises at least 50 amino acid residues and at most 500 amino acid residues.

In certain embodiments, —Z of formula (Ia) or (Ib) comprises a fatty acid derivative. In certain embodiments, —Z of formula (Ia) or (Ib) is a fatty acid derivative. In certain embodiments, —Z of formula (Ia) is a fatty acid derivative and x is 1.

In certain embodiments, —Z of formula (Ia) or (Ib) is a fatty acid derivative as disclosed in WO 2006/097537 A2 which is herewith incorporated by reference.

In certain embodiments, —Z of formula (Ia) or (Ib) comprises a fatty acid derivative as disclosed in WO 2021/055497 A1 which is herewith incorporated by reference. Accordingly, in certain embodiments, —Z of formula (Ia) or (Ib) has the following structure (w):

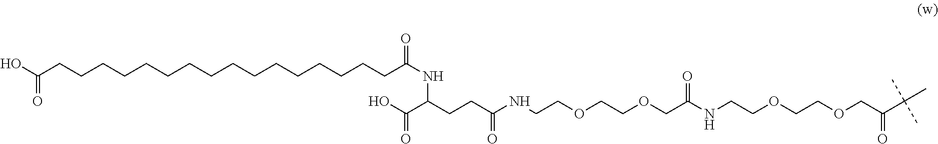

wherein the dashed line indicates the attachment to -L²- or -L¹- in formula (Ia) or (Ib).

In certain embodiments, —Z is of formula (w) and -L¹- is of formula (V).

In certain embodiments, —Z-L²-L¹- is of formula (w-a):

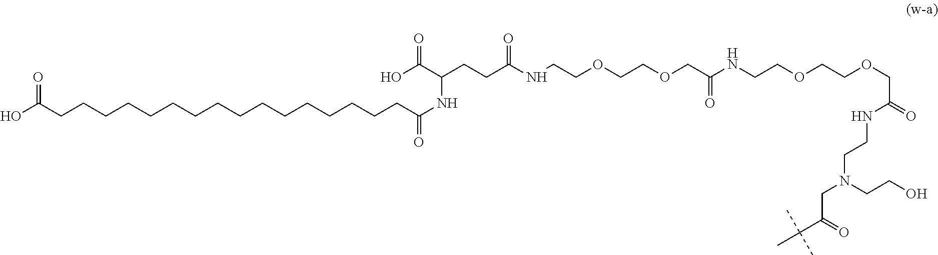

wherein the dashed line indicates the attachment to -D of formula (Ia) or (Ib).

In certain embodiments, CNP has the sequence selected from the group consisting of:

(SEQ ID NO: 98)
PGQEHPQARRYRGAQRRGLSRGCFGLKLDRIGSMSGLGC;
(SEQ ID NO: 30)
PGQEHPNARKYKGANKKGLSKGCFGLKLDRIGSMSGLGC;
(SEQ ID NO: 99)
PGQEHPNARRYRGANRRGLSRGCFGLKLDRIGSMSGLGC;
and
(SEQ ID NO: 100)
PGQEHPQARKYKGAQKKGLSKGCFGLKLDRIGSMSGLGC

In certain embodiments, CNP has the sequence selected from the group consisting of SEQ ID NO:98, SEQ ID NO:30, SEQ ID NO:99 and SEQ ID NO:100, —Z is of formula (w) and -L¹- is a reversible linker moiety. In certain embodiments, CNP has the sequence selected from the group consisting of SEQ ID NO:98, SEQ ID NO:30, SEQ ID NO:99 and SEQ ID NO: 100, —Z is of formula (w) and -L¹- is of formula (V). In certain embodiments, CNP has the sequence selected from the group consisting of SEQ ID NO:98, SEQ ID NO:30, SEQ ID NO:99 and SEQ ID NO:100, —Z-L²-L¹- is of formula (w-a). Said -L¹- may be attached to said CNP via a lysine other than the lysine within the ring structure or it may be attached to the N-terminus.

In certain embodiments, CNP of SEQ ID NO:98, SEQ ID NO:30, SEQ ID NO:99 and SEQ ID NO:100 further comprises an acetyl group, such as an acetyl group at the N-terminus of the peptide. In certain embodiments, CNP of SEQ ID NO:98, SEQ ID NO:30, SEQ ID NO:99 and SEQ ID NO:100 further comprises an —OH or —NH₂ group at the C-terminus. In certain embodiments, CNP of SEQ ID NO:98, SEQ ID NO:30, SEQ ID NO:99 and SEQ ID NO:100 and -L¹- is attached to a residue of the CNP ring moiety or at a site other than the CNP moiety.

In certain embodiments, -L¹- is attached at a lysine residue, such as the lysine residue in bold in SEQ ID NO:98, SEQ ID NO:30, SEQ ID NO:99 and SEQ ID NO: 100:

(SEQ ID NO: 98)
PGQEHPQARRYRGAQRRGLSRGCFGLKLDRIGSMSGLGC;
(SEQ ID NO: 30)
PGQEHPNARKYKGANKKGLSKGCFGLKLDRIGSMSGLGC;
(SEQ ID NO: 99)
PGQEHPNARRYRGANRRGLSRGCFGLKLDRIGSMSGLGC;
and
(SEQ ID NO: 100)
PGQEHPQARKYKGAQKKGLSKGCFGLKLDRIGSMSGLGC

In certain embodiments, CNP is selected from the group consisting of:

(SEQ ID NO: 101)
Ac-PGQEHPQARRYRGAQRRGLSRGCFGLKLDRIGSMSGLGC;
(SEQ ID NO: 102)
Ac-PGQEHPNARKYKGANKKGLSKGCFGLKLDRIGSMSGLGC-NH₂;
(SEQ ID NO: 103)
Ac-PGQEHPNARRYRGANRRGLSRGCFGLKLDRIGSMSGLGC;
(SEQ ID NO: 104)
Ac-PGQEHPNARRYRGANRRGLSRGCFGLKLDRIGSMSGLGC-NH₂;
and
(SEQ ID NO: 105)
Ac-PGQEHPQARRYRGAQRRGLSRGCFGLKLDRIGSMSGLGC-NH₂.

In certain embodiments, —Z of formula (Ia) or (Ib) is a hyaluronic acid-based polymer.

In certain embodiments, —Z of formula (Ia) or (Ib) is a polymeric moiety as disclosed in WO 2013/024047 A1 which is herewith incorporated by reference.

In certain embodiments, —Z of formula (Ia) or (Ib) is a polymeric moiety as disclosed in WO 2013/024048 A1 which is herewith incorporated by reference.

In certain embodiments, —Z of formula (Ia) or (Ib) is a PEG-based polymer. In certain embodiments, —Z is a branched or multi-arm PEG-based polymer.

In certain embodiments, —Z of formula (Ia) or (Ib) is a branched polymer. In certain embodiments, —Z of formula (Ia) or (Ib) is a branched polymer having one, two, three, four, five or six branching points. In certain embodiments, —Z of formula (Ia) or (Ib) is a branched polymer having one, two or three branching points. In certain embodiments, —Z of formula (Ia) or (Ib) is a branched polymer having one branching point. In certain embodiments, —Z of formula (Ia) or (Ib) is a branched polymer having two branching points. In certain embodiments, —Z of formula (Ia) or (Ib) is a branched polymer having three branching points. In certain embodiments, a branching point is selected from the group consisting of —N<, —CH< and >C<.

In certain embodiments, such branched moiety —Z of formula (Ia) or (Ib) is PEG-based.

In certain embodiments, such branched moiety —Z of formula (Ia) or (Ib) has a molecular weight ranging from and including 5 kDa to 500 kDa. In certain embodiments, such branched moiety —Z of formula (Ia) or (Ib) has a molecular weight ranging from and including 10 kDa to 250 kDa. In certain embodiments, such branched moiety —Z of formula (Ia) or (Ib) has a molecular weight ranging from and including 10 kDa to 150 kDa. In certain embodiments, such branched moiety —Z of formula (Ia) or (Ib) has a molecular weight ranging from and including 12 kDa to 100 kDa. In certain embodiments, such branched moiety —Z of formula (Ia) or (Ib) has a molecular weight ranging from and including 15 kDa to 80 kDa. In certain embodiments, such branched moiety —Z of formula (Ia) or (Ib) has a molecular weight ranging from and including 10 kDa to 80 kDa. In certain embodiments, the molecular weight is about 10 kDa. In certain embodiments, the molecular weight of such branched moiety —Z of formula (Ia) or (Ib) is about 20 kDa. In certain embodiments, the molecular weight of such branched moiety —Z of formula (Ia) or (Ib) is about 30 kDa. In certain embodiments, the molecular weight of such a branched moiety —Z of formula (Ia) or (Ib) is about 40 kDa. In certain embodiments, the molecular weight of such a branched moiety —Z of formula (Ia) or (Ib) is about 50 kDa. In certain embodiments, the molecular weight of such a branched moiety —Z of formula (Ia) or (Ib) is about 60 kDa. In certain embodiments, the molecular weight of such a branched moiety —Z of formula (Ia) or (Ib) is about 70 kDa. In certain embodiments, the molecular weight of such a branched moiety —Z of formula (Ia) or (Ib) is about 80 kDa. In certain embodiments, such branched moiety —Z of formula (Ia) or (Ib) has a molecular weight of about 40 kDa.

In certain embodiments, —Z comprises a moiety

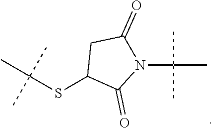

In certain embodiments, —Z comprises an amide bond.

In certain embodiments, —Z of formula (Ia) or (Ib) comprises a moiety of formula (a):

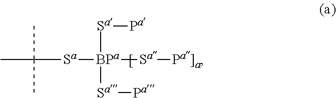

wherein
the dashed line indicates attachment to -L²- or to the remainder of —Z;
BPⁿ is a branching point selected from the group consisting of —N<, —CR< and >C<;
—R is selected from the group consisting of —H and C₁₋₆ alkyl;
a is 0 if BP^{a} is —N< or —CR< and a is 1 if BP^{a} is >C<;
—S^{a}—, —S^{a′}—, —S^{a″}— and —S^{a′″}— are independently of each other a chemical bond or are selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, N(R²)S(O)₂N(R^{2a})—, —S—, —N(R²)—, —OC(OR²)(R^{2a})—, —N(R²)C(O)N(R^{2a})—, and —OC(O)N(R²)—;
each -T- is independently selected from the group consisting of phenyl, naphthyl, indenyl, indanyl, tetralinyl, C₃₋₁₀ cycloalkyl, 3- to 10-membered heterocyclyl, 8- to 1 1-membered heterobicyclyl, 8- to 30-membered carbopolycyclyl, and 8- to 30-membered heteropolycyclyl; wherein each -T- is independently optionally substituted with one or more —R¹, which are the same or different;
each —R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
each —R², —R^{2a}, —R³, —R^{3a} and —R^{3b} is independently selected from the group consisting of —H, and C₁₋₆ alkyl,
wherein C₁₋₆ alkyl is optionally substituted with one or more halogen, which are the same or different;
and
—P^{a′}, —P^{a″} and —P^{a′″} are independently a polymeric moiety.

Optionally, the moiety of formula (a) is substituted with one or more substituents.

In certain embodiments, BP^{a} of formula (a) is —N<. In certain embodiments, BP^{a} of formula (a) is —CR<. In certain embodiments, —R is —H.

Accordingly, in certain embodiments, a of formula (a) is 0.

In certain embodiments, BP^{a} of formula (a) is >C<.

In certain embodiments, —S^{a}— of formula (a) is a chemical bond.

In certain embodiments, —S^{a}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a}), and —OC(O)N(R⁴)—; wherein —R⁴ and —R^{4a} are independently selected from the group consisting of —H, methyl, ethyl, propyl and butyl. In certain embodiments, —S^{a}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments, —S^{a′}— of formula (a) is a chemical bond.

In certain embodiments, —S^{a′}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a}) and —OC(O)N(R⁴)—; wherein —R⁴ and —R^{4a} are independently selected from the group consisting of —H, methyl, ethyl, propyl and butyl. In certain embodiments, —S^{a′}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments, —S^{a″}— of formula (a) is a chemical bond.

In certain embodiments, —S^{a″}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a}) and —OC(O)N(R⁴)—; wherein —R⁴ and —R^{4a} are independently selected from the group consisting of —H, methyl, ethyl, propyl and butyl. In certain embodiments, —S^{a″}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments, —S^{a″}— of formula (a) is a chemical bond.

In certain embodiments, —S^{a″}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a}) and —OC(O)N(R⁴)—; wherein —R⁴ and —R^{4a} are independently selected from the group consisting of —H, methyl, ethyl, propyl and butyl. In certain embodiments, —S^{a′″}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments, —P^{a′}, —P^{a″} and —P^{a′″} of formula (a) independently comprise a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments, —P^{a′}, —P^{a″} and —P^{a′″} of formula (a) independently have a molecular weight ranging from and including 5 kDa to 50 kDa, in certain embodiments ranging from and including 5 kDa to 40 kDa, in certain embodiments ranging from and including 7.5 kDa to 35 kDa, in certain embodiments ranging from and 7.5 to 30 kDa, in certain embodiments ranging from and including 10 to 30 kDa.

In certain embodiments, —P^{a′}, —P^{a″} and —P^{a′″} of formula (a) have a molecular weight of about 5 kDa. In certain embodiments, —P^{a′}, —P^{a″} and —P^{a′″} of formula (a) have a molecular weight of about 7.5 kDa. In certain embodiments, —P^{a′}, —P^{a″} and —P^{a′″} of formula (a) have a molecular weight of about 10 kDa. In certain embodiments, —P^{a′}, —P^{a″} and —P^{a′″} of formula (a) have a molecular weight of about 12.5 kDa. In certain embodiments, —P^{a′}, —P^{a″} and —P^{a′″} of formula (a) have a molecular weight of about 15 kDa. In certain embodiments, —P^{a′}, —P^{a″} and —P^{a′″} of formula (a) have a molecular weight of about 20 kDa.

In certain embodiments, —P^{a′}, —P^{a″} and —P^{a′″} of formula (a) independently comprise a PEG-based moiety. In certain embodiments, —P^{a′}, —P^{a″} and —P^{a′″} of formula (a) independently comprise a PEG-based moiety comprising at least 20% PEG, in certain embodiments at least 30% PEG, in certain embodiments at least 40% PEG, in certain embodiments at least 50% PEG, in certain embodiments at least 60% PEG, in certain embodiments at least 70% PEG, in certain embodiments at least 80% PEG and in certain embodiments at least 90% PEG. In certain embodiments, —P^{a′}, —P^{a″} and —P^{a′″} of formula (a) independently comprise a protein moiety, in certain embodiments a random coil protein moiety and in certain embodiments a random coil protein moiety selected from the group consisting of PA, PAS, PAG, PG and XTEN moieties.

In certain embodiments, —P^{a′}, —P^{a″} and —P^{a′″} of formula (a) are a PA moiety. In certain embodiments, —P^{a′}, —P^{a″} and —P^{a′″} of formula (a) are a PAS moiety. In certain embodiments, —P^{a′}, —P^{a″} and —P^{a′″} of formula (a) are a PAG moiety. In certain embodiments, —P^{a′}, —P^{a″} and —P^{a′″} of formula (a) are a PG moiety. In certain embodiments, —P^{a′}, —P^{a″} and —P^{a′″} of formula (a) are an XTEN moiety.

In certain embodiments, —Z comprises one moiety of formula (a). In certain embodiments, —Z comprises two moieties of formula (a). In another embodiment, —Z comprises three moieties of formula (a). In certain embodiments, —Z comprises four moieties of formula (a). In certain embodiments, —Z comprises five moieties of formula (a). In certain embodiments, —Z comprises six moieties of formula (a).

In certain embodiments, —Z comprises a moiety of formula (b):

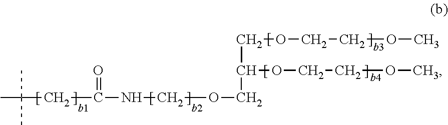

wherein
the dashed line indicates attachment to -L²- or to the remainder of —Z;
b1 is selected from the group consisting of 0, 1, 2, 3, 4, 5, 6, 7 and 8;
b2 is selected from the group consisting of 1, 2, 3, 4, 5, 6, 7 and 8;
b3 is an integer ranging from and including 150 to 1000; in certain embodiments, ranging from and including 150 to 500; and in certain embodiments, ranging from and including 200 to 460; and
b4 is an integer ranging from and including 150 to 1000; in certain embodiments, ranging from and including 150 to 500; and in certain embodiments, ranging from and including 200 to 460.

Optionally, the moiety of formula (b) is substituted with one or more substituents.

In certain embodiments, b3 and b4 of formula (b) are the same integer. In certain embodiments, b3 and b4 of formula (b) are both an integer ranging from 200 to 250 and in certain embodiments, b3 and b4 of formula (b) are about 225. In certain embodiments, b3 and b4 of formula (b) are both an integer ranging from 400 to 500 and in certain embodiments, b3 and b4 of formula (b) are about 450.

In certain embodiments, b1 of formula (b) is selected from the group consisting of 0, 1, 2, 3 and 4. In certain embodiments, b1 of formula (b) is selected from the group consisting of 1, 2 and 3. In certain embodiments, b1 of formula (b) is 2.

In certain embodiments, b2 of formula (b) is selected from the group consisting of 1, 2, 3, 4 and 5. In certain embodiments, b2 of formula (b) is selected from the group consisting of 2, 3 and 4. In certain embodiments, b2 of formula (b) is 3.

In certain embodiments, b1 of formula (b) is 2, b2 of formula (b) is 3, and b3 and b4 are both about 450. In certain embodiments, b1 of formula (b) is 2, b2 of formula (b) is 3, and b3 and b4 are both about 225.

In certain embodiments, —Z comprises one moiety of formula (b). In certain embodiments, —Z comprises two moieties of formula (b). In certain embodiments, —Z comprises three moieties of formula (b). In certain embodiments, —Z comprises four moieties of formula (b). In certain embodiments, —Z comprises five moieties of formula (b). In certain embodiments, —Z comprises six moieties of formula (b).

In certain embodiments, —Z comprises a moiety of formula (c):

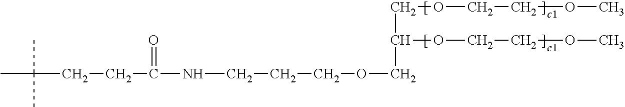

wherein
the dashed line indicates attachment to -L²- or to the remainder of —Z;
c1 and c2 are independently an integer ranging from and including 150 to 500; in certain embodiments, ranging from and including 200 to 460.

Optionally, the moiety of formula (c) is substituted with one or more substituents.

In certain embodiments, both c1 and c2 of formula (c) are the same integer.

In certain embodiments, c1 and c2 of formula (c) range from and include 200 to 250 and in certain embodiments, are about 225. In certain embodiments, c1 and c2 of formula (c) range from and include 400 to 500 and in certain embodiments, c1 and c2 of formula (c) are about 450.

In certain embodiments, the moiety —Z is a branched PEG-based polymer comprising at least 10% PEG, has one branching point and two PEG-based polymer arms and has a molecular weight of about 40 kDa. Accordingly, each of the two PEG-based polymer arms has a molecular weight of about 20 kDa. In certain embodiments, the branching point is —CH<.

In certain embodiments, —Z comprises one moiety of formula (c). In certain embodiments, —Z comprises two moieties of formula (c). In certain embodiments, —Z comprises three moieties of formula (c). In certain embodiments, —Z comprises four moieties of formula (c). In certain embodiments, —Z comprises five moieties of formula (c). In certain embodiments, —Z comprises six moieties of formula (c).

In certain embodiments, the moiety —Z is of formula (d):

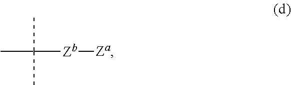

wherein
the dashed line indicates attachment to -L²-;
—Z^{b}— is selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
   each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8-to 30-membered carbopolycyclyl, and 8- to 30-membered heteropolycyclyl; wherein each -T- is independently optionally substituted with one or more —R¹, which are the same or different; each —R¹ is independently selected from the group consisting of halogen, —CN, oxo (═O), —COOR³, —OR³—C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
   each —R², —R^{2a}, —R³, —R^{3a} and —R^{3b} is independently selected from the group consisting of —H, and C₁₋₆ alkyl, wherein C₁₋₆ alkyl is optionally substituted with one or more halogen, which are the same or different;
and
—Z^{a} is

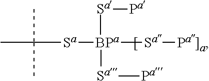

wherein
BP^{a}, —S^{a}—, —S^{a′}—, —S^{a″}—, —S^{a′″}—, —P^{a′}, —P^{a″}, —P^{a′″} and a are used as defined for formula (a).

Optionally, the moiety of formula (d) is substituted with one or more substituents.

In certain embodiments, BP^{a}, —S^{a}—, —S^{a′}—, —S^{a″}—, —S^{a′″}—, —P^{a′}, —P^{a″}, —P^{a′″} of formula (d) are as defined above for formula (a).

In certain embodiments, —Z^{a} of formula (d) is of formula (b). In certain embodiments, b1, b2, b3 and b4 are as described for formula (b).

In certain embodiments, the moiety —Z of formula (Ia) or (Ib) is of formula (e):

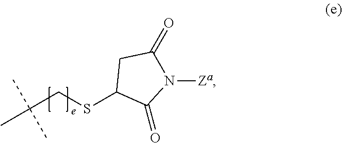

wherein
the dashed line indicates attachment to -L²-;
e is selected from the group consisting of 1, 2, 3, 4, 5, 6, 7, 8, 9, 10, 11, 12, 13, 14 and 15; and
—Z^{a} is

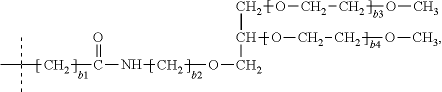

wherein
b1, b2, b3 and b4 are used as defined for formula (b).

Optionally, the moiety of formula (e) is substituted with one or more substituents.

In certain embodiments, for b1, b2, b3 and b4 of formula (e) are as defined above for formula (b).

In certain embodiments, e of formula (e) is 1. In certain embodiments, e of formula (e) is 2. In certain embodiments, e of formula (e) is 3. In certain embodiments, e of formula (e) is 4. In certain embodiments, e of formula (e) is 5. In certain embodiments, e of formula (e) is 6. In certain embodiments, e of formula (e) is 7. In certain embodiments, e of formula (e) is 8. In certain embodiments, e of formula (e) is 9. In certain embodiments, e of formula (e) is 10. In certain embodiments, e of formula (e) is 11. In certain embodiments, e of formula (e) is 12. In certain embodiments, e of formula (e) is 13. In certain embodiments, e of formula (e) is 14. In certain embodiments, e of formula (e) is 15.

In certain embodiments, e of formula (e) is selected from the group consisting of 2, 3, 4, 5, 6, 7, 8 and 9. In certain embodiments, e of formula (e) is selected from 3, 4, 5 and 6. In certain embodiments, e of formula (e) is 5.

In certain embodiments, e of formula (e) is 5, b1 of formula (e) is 2, b2 of formula (e) is 3 and b3 and b4 of formula (e) are both about 450.

In certain embodiments, the moiety —Z of formula (Ia) or (Ib) is of formula (e-i) or (e-i′):

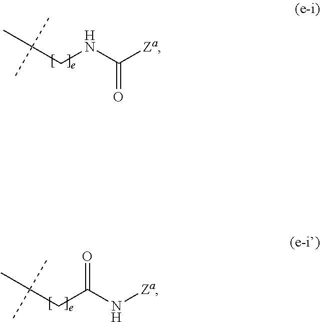

wherein
the dashed line indicates attachment to -L²-,
e is selected from the group consisting of 1, 2, 3, 4, 5, 6, 7, 8, 9, 10, 11, 12, 13, 14 and 15;
—Z^{a} is

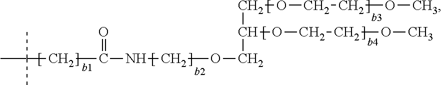

wherein
b1, b2, b3 and b4 are used as defined for formula (b).

In certain embodiments, for b1, b2, b3 and b4 of formula (e-i) and (e-i′) are as defined above for formula (b). In certain embodiments, e of formula (e-i) and (e-i′) are as described for formula (e). In certain embodiments, b1 of formula (e-i) and (e-i′) is 2, b2 of formula (e-i) and (e-i′) is 3 and b3 and b4 of formula (e-i) and (e-i′) are both about 450.

In certain embodiments, —Z of formula (Ia) or (Ib) is of formula (e-i).

In certain embodiments, the moiety —Z is a branched PEG-based polymer comprising at least 10% PEG, has three branching points and four PEG-based polymer arms and has a molecular weight of about 40 kDa. Accordingly, each of the four PEG-based polymer arms has a molecular weight of about 10 kDa. In certain embodiments, each of the three branching points is —CH<.

In certain embodiments, the moiety —Z is of formula (f):

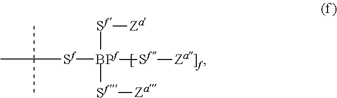

wherein
the dashed line indicates attachment to -L²-;
BP^{f} is a branching point selected from the group consisting of —N<, —CR< and >C<;
—R is selected from the group consisting of —H and C₁₋₆ alkyl;
f is 0 if BP^{f} is —N< or —CR< and f is 1 if BP^{f} is >C<;
—S^{f}—, —S^{f′}—, —S^{f″}— and —S^{f′″}— are independently either a chemical bond or are independently selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²);
   each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8-to 30-membered carbopolycyclyl, and 8- to 30-membered heteropolycyclyl; wherein each -T- is independently optionally substituted with one or more —R¹, which are the same or different; each R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
   each —R², —R^{2a}, —R³, —R^{3a} and —R^{3b} is independently selected from the group consisting of —H, and C₁₋₆ alkyl, wherein C₁₋₆ alkyl is optionally substituted with one or more halogen, which are the same or different;
and
—Z^{a′}, —Z^{a″} and —Z^{a′″} are independently

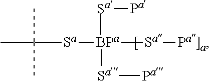

wherein
BP^{a}, —S^{a}—, —S^{a′}—, —S^{a″}—, —S^{a′″}—, —P^{a′}, —P^{a″}, —P^{a′″} and a are used as defined for formula (a).

Optionally, the moiety of formula (f) is substituted with one or more substituents.

In certain embodiments, BP^{a}—S^{a}—, —S^{a′}—, —S^{a″}—, —S^{a′″}—, —P^{a′}, —P^{a″}, —P^{a′″} of formula (f) are as defined above for formula (a).

In certain embodiments, BP^{f} of formula (f) is —CR< and r is 0. In certain embodiments, —R is —H.

In certain embodiments, —S^{f}— of formula (f) is a chemical bond.

In certain embodiments, —Z^{a′}, —Z^{a″} and —Z^{a′″} of formula (f) have the same structure. In certain embodiments, —Z^{a′}, —Z^{a″} and —Z^{a′″} of formula (f) are of formula (b).

In certain embodiments, b1, b2, b3 and b4 are as described for formula (b).

In certain embodiments, —S^{f}— of formula (f) is a chemical bond, BP^{a} of formula (f) is —CR< with —R being —H. In certain embodiments, —S^{f}— of formula (f) is a chemical bond, BP^{a} of formula (f) is —CR< with —R being —H and —Z^{a′}, —Z^{a″} and —Z^{a′″} of formula (f) are of formula (b).

In certain embodiments, —Z is of formula (g):

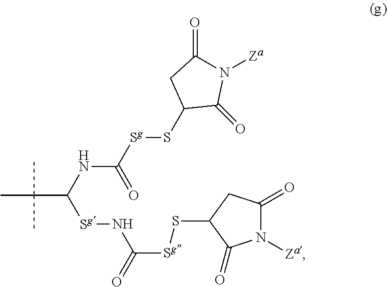

wherein
the dashed line indicates attachment to -L²-;
—S^{g}—, —S^{g′}— and —S^{g″}— are independently selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a}) and —OC(O)N(R²);
   each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8-to 30-membered carbopolycyclyl, and 8- to 30-membered heteropolycyclyl; wherein each -T- is independently optionally substituted with one or more —R¹, which are the same or different;
   each R¹ is independently selected from the group consisting of halogen, —CN, oxo (═O), —COOR³, —OR³, —C(O)R³, —C(O)N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
   each —R², —R^{2a}, —R³, —R^{3a}, and —R^{3b} is independently selected from the group consisting of —H, and C₁₋₆ alkyl, wherein C₁₋₆ alkyl is optionally substituted with one or more halogen, which are the same or different;
and
—Z^{a} and —Z^{a′} are independently

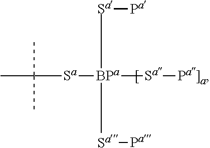

wherein
BP^{a}, —S^{a}, —S^{a′}, —S^{a″}, —S^{a′″}, —P^{a′}, —P^{a″}, —P^{a′″} and a are used as defined for formula (a).

Optionally, the moiety of formula (g) is substituted with one or more substituents.

In certain embodiments, BP^{a}, —S^{a}, —S^{a′}, —S^{a″}, —S^{a′″}, —P^{a′}, —P^{a″}, and —P^{a′″} of formula (g) are as defined above for formula (a).

In certain embodiments, —S^{g}— of formula (g) is selected from the group consisting of C₁₋₆ alkyl, C₂₋₆ alkenyl and C₂₋₆ alkynyl, which are optionally substituted with one or more —R¹, which is the same or different,
wherein
—R¹ is selected from the group consisting of halogen, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different; and
—R³, —R^{3a} and —R^{3b} are independently selected from —H, methyl, ethyl, propyl and butyl.

In certain embodiments, —S^{g}— of formula (g) is selected from C₁₋₆ alkyl.

In certain embodiments, —S^{g′}— of formula (g) is selected from the group consisting of C₁₋₆ alkyl, C₂₋₆ alkenyl and C₂₋₆ alkynyl, which are optionally substituted with one or more —R¹, which is the same or different,
wherein
—R¹ is selected from the group consisting of halogen, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different; and
—R³, —R^{3a} and —R^{3b} are independently selected from —H, methyl, ethyl, propyl, and butyl.

In certain embodiments, —S^{g′}— of formula (g) is C₁₋₆ alkyl.

In certain embodiments, —S^{g″}— of formula (g) is selected from the group consisting of C₁₋₆ alkyl, C₂₋₆ alkenyl and C₂₋₆ alkynyl, which are optionally substituted with one or more —R¹, which is the same or different,
wherein
—R¹ is selected from the group consisting of halogen, oxo (═O), —COOR³, —OR³—C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different; and
—R³, —R^{3a} and —R^{3b} are independently selected from —H, methyl, ethyl, propyl and butyl.

In certain embodiments, —S^{g″}— of formula (g) is C₁₋₆ alkyl.

In certain embodiments, —Z^{a} and —Z^{a′} of formula (g) have the same structure. In certain embodiments, —Z^{a} and —Z^{a′} of formula (g) are of formula (b).

In certain embodiments, of BP^{a}, —S^{a}—, —S^{a′}—, —S^{a″}—, —S^{a′″}—, —P^{a′}, —P^{a″} and —P^{a′″} of formula (g-i) are as defined above for formula (a).

In certain embodiments, of —S^{g}—, —S^{g′}— and —S^{g″}— of formula (g-i) are as defined for formula (g).

In certain embodiments, —Z^{a} and —Z^{a′} of formula (g-i) have the same structure. In certain embodiments, —Z^{a} and —Z^{a′} of formula (g-i) are of formula (b). In certain embodiments, for b1, b2, b3 and b4 are as described for formula (b).

In certain embodiments, —Z is of formula (h):

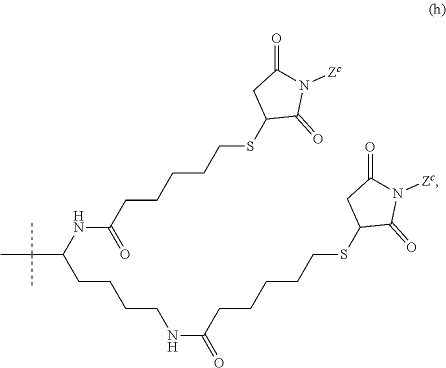

wherein
the dashed line indicates attachment to -L²-; and
each —Z^{c} is a moiety

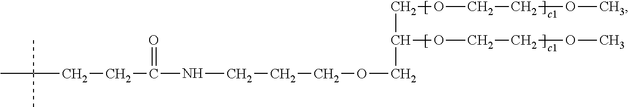

wherein
each c1 is an integer independently ranging from about 200 to 250.

Optionally, the moiety of formula (h) is substituted with one or more substituents.

In certain embodiments, both c1 of formula (h) are the same. In certain embodiments, both c1 of formula (h) are about 225.

In certain embodiments, the moiety —Z is of formula (h-i):

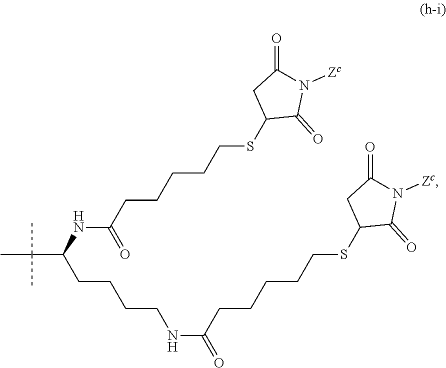

wherein
the dashed line indicates attachment to -L²-; and
each —Z^{c} is a moiety

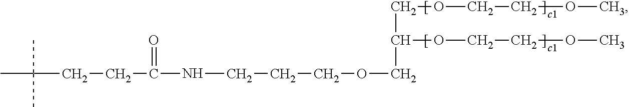

each c1 is an integer independently ranging from 200 to 250.

Optionally, the moiety of formula (h-i) is substituted with one or more substituents.

In certain embodiments, both c1 of formula (h-i) are the same. In certain embodiments, both c1 of formula (h-i) are about 225.

In certain embodiments, the CNP conjugate is of formula (IIf):

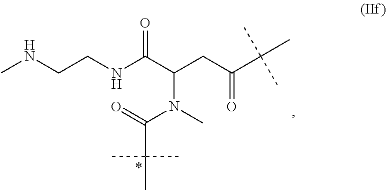

wherein
the unmarked dashed line indicates the attachment to a nitrogen of -D which is a CNP moiety by forming an amide bond; and
the dashed line marked with the asterisk indicates attachment to —Z having the structure

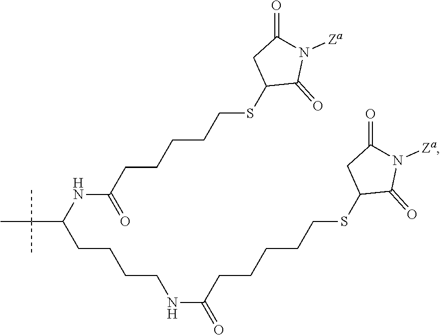

wherein
each —Z^{a} is

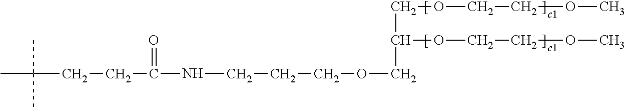

wherein
each c1 is an integer independently ranging from 200 to 250.

In certain embodiments, each c1 of formula (III) is about 225.

In certain embodiments, -D of formula (IIf) is a CNP moiety, i.e. the conjugate of formula (IIf) is a CNP conjugate.

In certain embodiments, -D of formula (IIf) is a CNP moiety having the sequence of SEQ ID NO:24, SEQ ID NO:25 or SEQ ID NO:30. In certain embodiments, -D of formula (IIf) is a CNP moiety having the sequence of SEQ ID NO:24. In certain embodiments, -D of formula (IIf) is a CNP moiety having the sequence of SEQ ID NO:20. In certain embodiments, -D of formula (IIf) is a CNP moiety having the sequence of SEQ ID NO:21. In certain embodiments, -D of formula (IIf) is a CNP moiety having the sequence of SEQ ID NO:22. In certain embodiments, -D of formula (IIf) is a CNP moiety having the sequence of SEQ ID NO:23. In certain embodiments, -D of formula (IIf) is a CNP moiety having the sequence of SEQ ID NO:30.

In certain embodiments, -D of formula (IIf) is a CNP moiety which is attached to -L¹- through the nitrogen of the N-terminal amine functional group of CNP.

In certain embodiments, -D of formula (III) is a CNP moiety which is attached to -L¹- through a nitrogen provided by the amine functional group of a lysine side chain of the CNP moiety.

In certain embodiments, said lysine side chain is not part of the ring formed by the disulfide bridge between the cysteine residues at positions 22 and 38, if the CNP moiety is of SEQ ID NO:24.

Accordingly, in certain embodiments, the CNP moiety is connected to -L¹- in the CNP conjugate of formula (III) through the amine functional group provided by the side chain of the lysine at position 9, if the CNP has the sequence of SEQ ID NO:24.

In certain embodiments, the CNP moiety is connected to -L¹- in the CNP conjugate of formula (III) through the amine functional group provided by the side chain of the lysine at position 11, if the CNP has the sequence of SEQ ID NO:24.

In certain embodiments, the CNP moiety is connected to -L¹- in the CNP conjugate of formula (III) through the amine functional group provided by the side chain of the lysine at position 15, if the CNP has the sequence of SEQ ID NO: 24.

In certain embodiments, the CNP moiety is connected to -L¹- in the CNP conjugate of formula (III) through the amine functional group provided by the side chain of the lysine at position 16, if the CNP has the sequence of SEQ ID NO:24.

In certain embodiments, the CNP moiety is connected to -L¹- in the CNP conjugate of formula (III) through the amine functional group provided by the side chain of the lysine at position 20, if the CNP has the sequence of SEQ ID NO:24.

In certain embodiments, said lysine side chain is part of the ring formed by the disulfide bridge between the cysteine residues at positions 22 and 38, if the CNP moiety is of SEQ ID NO:24.

Accordingly, in certain embodiments, the CNP moiety is connected to -L¹- in the CNP conjugate of formula (III) through the amine functional group provided by the side chain of the lysine at position 26, if the CNP has the sequence of SEQ ID NO:24.

In certain embodiments, the CNP conjugate is of formula (III), wherein c1 is about 225, -D is a CNP moiety having the sequence of SEQ ID NO:20 and is attached to -L¹- through the amine functional group provided by the side chain of the lysine at position 30.

In certain embodiments, the CNP conjugate is of formula (III), wherein c1 is about 225, -D is a CNP moiety having the sequence of SEQ ID NO:21 and is attached to -L¹- through the amine functional group provided by the side chain of the lysine at position 29.

In certain embodiments, the CNP conjugate is of formula (III), wherein c1 is about 225, -D is a CNP moiety having the sequence of SEQ ID NO:22 and is attached to -L¹- through the amine functional group provided by the side chain of the lysine at position 28.

In certain embodiments, the CNP conjugate is of formula (III), wherein c1 is about 225, -D is a CNP moiety having the sequence of SEQ ID NO:23 and is attached to -L¹- through the amine functional group provided by the side chain of the lysine at position 27.

In certain embodiments, the CNP conjugate is of formula (III), wherein c1 is about 225, -D is a CNP moiety having the sequence of SEQ ID NO:30 and is attached to -L¹- through the amine functional group provided by the side chain of the lysine at position 27.

It is understood that the positions of the cysteines and lysines mentioned above vary depending on the lengths of the CNP moiety and that the person skilled in the art will have no difficulty identifying the corresponding cysteines and lysines in longer or shorter versions of the CNP moiety and also understands that for example some lysines may not be present in shorter CNP moieties. It is further understood that as a result of for example site-directed mutagenesis there might be more lysine residues in the non-ring forming part and/or ring forming part of the CNP moiety.

In certain embodiments, the CNP conjugate is of formula (III), wherein c1 is about 225, -D is a CNP moiety having the sequence of SEQ ID NO:24 and is attached to -L¹- through the amine functional group provided by the side chain of the lysine at position 26.

In certain embodiments, the CNP conjugate is of formula (IIf′)

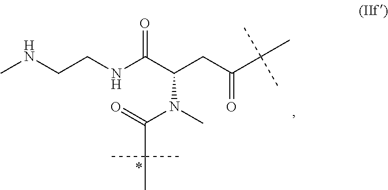

wherein
the unmarked dashed line indicates the attachment to a nitrogen provided by the side chain of the lysine at position 26 of the CNP moiety of SEQ ID NO:24 by forming an amide bond; and
the dashed line marked with the asterisk indicates attachment to —Z having the structure

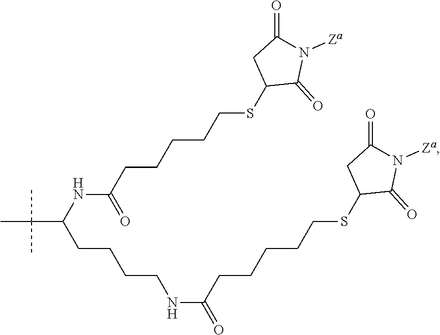

wherein
each Z^{a} is

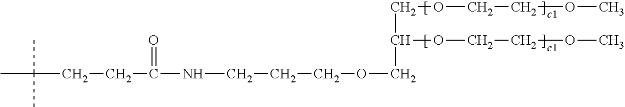

wherein
each c1 is an integer independently ranging from 200 to 250.

In certain embodiments, each c1 of formula (IIf′) is about 225.

In certain embodiments, the unit dosage form of the present invention comprises a CNP conjugate or pharmaceutically acceptable salt thereof of formula (IIf):

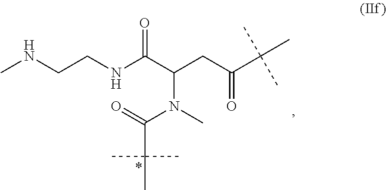

wherein
the unmarked dashed line indicates the attachment to a nitrogen provided by the side chain of the lysine at position 26 of a CNP moiety of SEQ ID NO:24 by forming an amide bond;
the dashed line marked with the asterisk indicates attachment to —Z having the structure

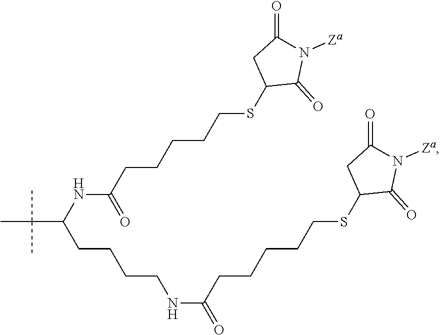

wherein
each Z^{a} is

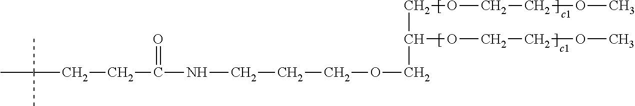

wherein
each c1 is an integer independently ranging from 200 to 250,
   and wherein the unit dose is about 50 μg CNP/kg.

In certain embodiments, the unit dosage form of the present invention comprises a CNP conjugate or pharmaceutically acceptable salt thereof of formula (IIf):

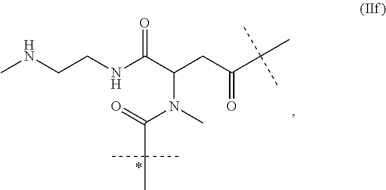

wherein
the unmarked dashed line indicates the attachment to a nitrogen provided by the side chain of the lysine at position 26 of a CNP moiety of SEQ ID NO:24 by forming an amide bond;
the dashed line marked with the asterisk indicates attachment to —Z having the structure

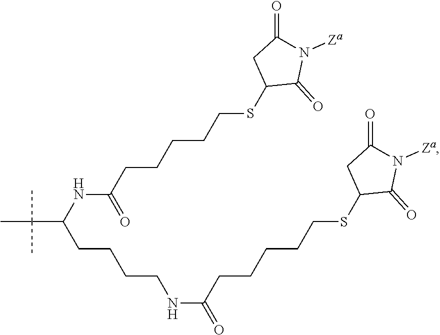

wherein
each Z^{a} is

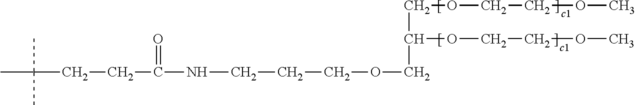

wherein
each c1 is an integer independently ranging from 200 to 250,
   and wherein the unit dose is about 75 μg CNP/kg.

In certain embodiments, the unit dosage form of the present invention comprises a CNP conjugate or pharmaceutically acceptable salt thereof of formula (IIf):

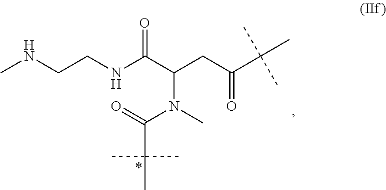

wherein
the unmarked dashed line indicates the attachment to a nitrogen provided by the side chain of the lysine at position 26 of a CNP moiety of SEQ ID NO:24 by forming an amide bond;
the dashed line marked with the asterisk indicates attachment to —Z having the structure

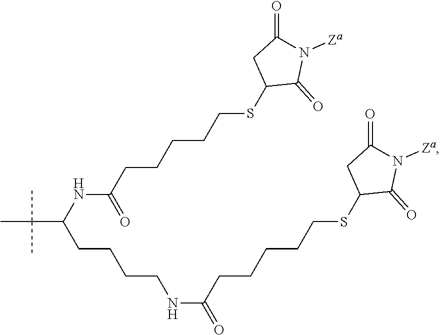

wherein
each Z^{c} is

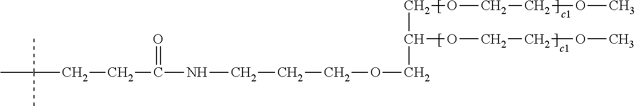

wherein
each c1 is an integer independently ranging from 200 to 250,
   and wherein the unit dose is about 100 μg CNP/kg.

In certain embodiments, the unit dosage form of the present invention comprises a CNP conjugate or pharmaceutically acceptable salt thereof of formula (IIf′):

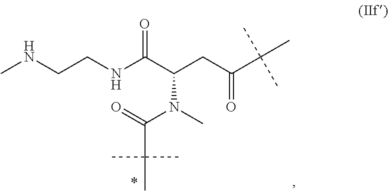

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

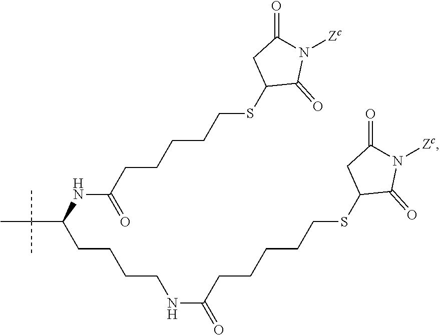

wherein
each Z^{c} is

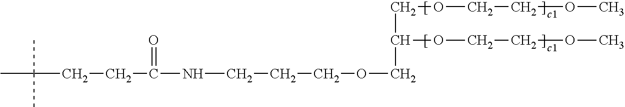

wherein
each c1 is an integer independently ranging from 200 to 250,
   and wherein the unit dose is 50 μg CNP/kg.

In certain embodiments, the unit dosage form of the present invention comprises a CNP conjugate or pharmaceutically acceptable salt thereof of formula (IIf′)

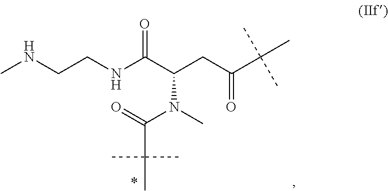

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

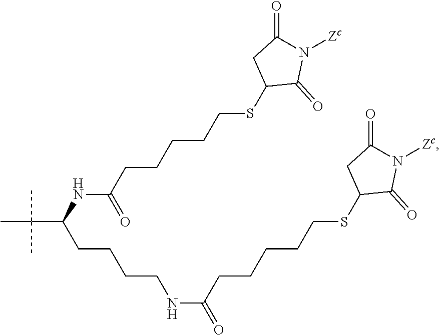

wherein
each Z^{a} is

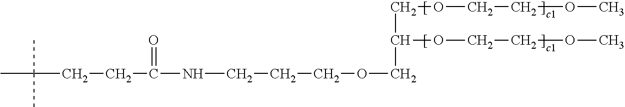

wherein
each c1 is an integer independently ranging from 200 to 250,
   and wherein the unit dose is 75 μg CNP/kg.

In certain embodiments, the unit dosage form of the present invention comprises a CNP conjugate or pharmaceutically acceptable salt thereof of formula (IIf′)

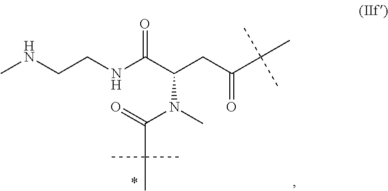

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

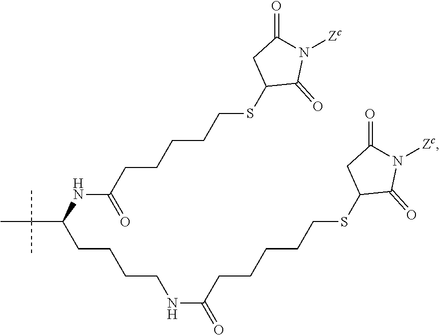

wherein
each Z^{a} is

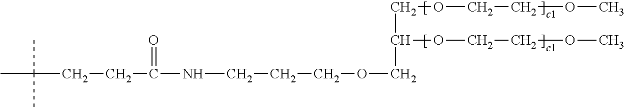

wherein
each c1 is an integer independently ranging from 200 to 250,
   and wherein the unit dose is 100 μg CNP/kg.

In some embodiments, with reference to the —Z^{c} or —Z^{a} moiety, such as in the CNP conjugate of formula (III) or (IIf′), c1 is an integer independently from about 100 to about 500, such as from about 150 to about 300, such as from about 200 to about 250.

In some embodiments, with reference to the —Z^{c} or —Z^{a} moiety, —Z^{c} or —Z^{a} is a branched PEG moiety which comprises 2 linear PEG arms, e.g. annotated as —[—O—CH₂—CH₂)_{cl}—O—CH₃ herein, which are independently at least about 5 kDa, such as from about 5 kDa to about 20 kDa, such as from about 7 kDa to about 15 kDa, such as about 8 kDa to about 12 kDa, such as about 10 kDa.

In some embodiments, with reference to the —Z^{c} or —Z^{a} moiety, such as in the CNP conjugate of formula (III) or (IIf′), each c1 present in the CNP conjugate is on average from about 100 to about 500, such as from about 150 to about 300, such as from about 200 to about 250.

In some embodiments, each PEG arm present in the CNP conjugate, e.g. annotated as —[—O—CH₂—CH₂)_{c1}—O—CH₃ herein, is on average at least about 5 kDa, such as from about 5 kDa to about 20 kDa, such as from about 7 kDa to about 15 kDa, such as about 8 kDa to about 12 kDa, such as about 10 kDa.

A further aspect of the present invention is a method of improving muscle function in a human patient having a disease treatable by CNP, the method comprising the step of administering an inhibitor of FGFR3 signalling, or an effective amount of an NPR-B agonist or an effective amount of an NPR-C agonist to said subject optionally wherein said inhibitor of FGFR3 signalling or NPR-B agonist and/or NPR-C agonist is a CNP conjugate or pharmaceutically acceptable salt thereof and said CNP conjugate or pharmaceutically acceptable salt thereof is administered at a unit dose from about 6 μg CNP/kg to at least about 150 μg CNP/kg.

A further aspect of the present invention is a method of improving skeletal muscle function in a human patient having a disease treatable by CNP, the method comprising the step of administering an inhibitor of FGFR3 signalling, or an effective amount of an NPR-B agonist or an effective amount of an NPR-C agonist to said subject optionally wherein said inhibitor of FGFR3 signalling or NPR-B agonist and/or NPR-C agonist is a CNP conjugate or pharmaceutically acceptable salt thereof and said CNP conjugate or pharmaceutically acceptable salt thereof is administered at a unit dose from about 6 μg CNP/kg to at least about 150 μg CNP/kg.

In certain embodiments, the present invention is a method of improving muscle function in a human patient having a disease treatable by CNP, the method comprising the step of administering an effective amount of inhibitor of FGFR3 signalling, or an effective amount of an NPR-B agonist, or an effective amount of an NPR-C agonist to said subject optionally wherein said inhibitor of FGFR3 signalling or NPR-B agonist or NPR-C agonist is a CNP conjugate or pharmaceutically acceptable salt thereof at a unit dose from about 6 μg CNP/kg to at least about 100 μg CNP/kg.

In certain embodiments, the subject has a disease or condition treatable by CNP optionally wherein the disease or condition is selected from the group consisting of bone-related disorders such as skeletal dysplasias; cancer; autoimmune diseases; fibrotic diseases; inflammatory diseases; central nervous system diseases such as neurodegenerative diseases; infectious diseases; lung diseases; heart and vascular diseases; metabolic diseases and ophthalmic diseases.

In certain embodiments, the subject has a Rasopathy.

In certain embodiments, said disease treatable by the methods or uses of the present invention include RASopathies: RASopathies result from genetic abnormalities of the Ras/MAPK pathway (and include pathways activated by FGFR3) (for a review Rauen, Annu Rev Genomics Hum Genet. 2013; 14: 355-369. doi:10.1146/annurev-genom-091212-153523), and include neurofibromatosis type 1, Noonan syndrome, Noonan syndrome with multiple lentigines, capillary malformation-arteriovenous malformation syndrome, Costello syndrome, cardio-facio-cutaneous syndrome, and Legius syndrome.

In certain embodiments, the invention relates to treatment of a RASopathies using a CNP, or an NPR-B agonist and/or NPR-C agonist.

In certain embodiments, said disease treatable by CNP is selected from the group consisting of achondroplasia, hypochondroplasia, short stature, dwarfism, osteochondrodysplasias, thanatophoric dysplasia, osteogenesis imperfecta, achondrogenesis, chondrodysplasia punctata, homozygous achondroplasia, camptomelic dysplasia, congenital lethal hypophosphatasia, perinatal lethal type of osteogenesis imperfecta, short-rib polydactyly syndromes, rhizomelic type of chondrodysplasia punctata, Jansen-type metaphyseal dysplasia, spondyloepiphyseal dysplasia congenita, atelosteogenesis, diastrophic dysplasia, congenital short femur, Langer-type mesomelic dysplasia, Nievergelt-type mesomelic dysplasia, Robinow syndrome, Reinhardt syndrome, acrodysostosis, peripheral dysostosis, Kniest dysplasia, fibrochondrogenesis, Roberts syndrome, acromesomelic dysplasia, micromelia, Morquio syndrome, Kniest syndrome, metatrophic dysplasia, spondyloepimetaphy seal dysplasia, neurofibromatosis, Legius syndrome, LEOPARD syndrome, Noonan syndrome, hereditary gingival fibromatosis, neurofibromatosis type 1, Legius syndrome, cardiofaciocutaneous syndrome, Costello syndrome, SHOX deficiency, idiopathic short stature, growth hormone deficiency, osteoarthritis, cleidocranial dysostosis, craniosynostosis (e.g., Muenke syndrome, Crouzon syndrome, Apert syndrome, Jackson-Weiss syndrome, Pfeiffer syndrome, or Crouzonodermoskeletal syndrome), dactyly, brachydactyly, camptodactyly, polydactyly, syndactyly, dyssegmental dysplasia, enchondromatosis, fibrous dysplasia, hereditary multiple exostoses, hypophosphatemic rickets, Jaffe-Lichtenstein syndrome, Marfan syndrome, McCune-Albright syndrome, osteopetrosis, osteopoikilosis, hemorrhagic shock, hypertension, restenosis, arteriosclerosis, acute decompensated heart failure, congestive heart failure, cardiac edema, nephredema, hepatic edema, acute renal insufficiency, chronic renal insufficiency, glaucoma, elevated intraocular pressure, multiple myeloma, myeloproliferative syndrome, leukemia, plasma cell leukemia, lymphoma, glioblastoma, prostate cancer, bladder cancer, mammary cancer, growth retardation, skull deformities, orthodontic defects, cervical cord compression, spinal stenosis, hydrocephalus, hearing loss due to chronic otitis, cardiovascular disease, neurological disease and obesity.

In certain embodiments, said disease treatable by CNP is selected from the group consisting of achondroplasia such as homozygous achondroplasia, hypochondroplasia, short stature, dwarfism, osteochondrodysplasias, thanatophoric dysplasia, osteogenesis imperfecta, achondrogenesis, chondrodysplasia punctata, camptomelic dysplasia, congenital lethal hypophosphatasia, perinatal lethal type of osteogenesis imperfecta, short-rib polydactyly syndromes, rhizomelic type of chondrodysplasia punctata, Jansen-type metaphyseal dysplasia, spondyloepiphyseal dysplasia congenita, atelosteogenesis, diastrophic dysplasia, congenital short femur, Langer-type mesomelic dysplasia, Nievergelt-type mesomelic dysplasia, Robinow syndrome, Reinhardt syndrome, acrodysostosis, peripheral dysostosis, Kniest dysplasia, fibrochondrogenesis, Roberts syndrome, acromesomelic dysplasia, micromelia, Morquio syndrome, Kniest syndrome, metatrophic dysplasia, spondyloepimetaphy seal dysplasia, neurofibromatosis, Legius syndrome, LEOPARD syndrome, Noonan syndrome, hereditary gingival fibromatosis, neurofibromatosis type 1, Legius syndrome, cardiofaciocutaneous syndrome, Costello syndrome, SHOX deficiency, idiopathic short stature, growth hormone deficiency, osteoarthritis, cleidocranial dysostosis, craniosynostosis (e.g., Muenke syndrome, Crouzon syndrome, Apert syndrome, Jackson-Weiss syndrome, Pfeiffer syndrome, or Crouzonodermoskeletal syndrome), dactyly, brachydactyly, camptodactyly, polydactyly, syndactyly, dyssegmental dysplasia, enchondromatosis, fibrous dysplasia, hereditary multiple exostoses, hypophosphatemic rickets, Jaffe-Lichtenstein syndrome, Marfan syndrome, McCune-Albright syndrome, osteopetrosis, osteopoikilosis, hemorrhagic shock, hypertension, restenosis, arteriosclerosis, acute decompensated heart failure, congestive heart failure, cardiac edema, nephredema, hepatic edema, acute renal insufficiency, chronic renal insufficiency, glaucoma, elevated intraocular pressure, multiple myeloma, myeloproliferative syndrome, leukemia, plasma cell leukemia, lymphoma, glioblastoma, prostate cancer, bladder cancer, mammary cancer, growth retardation, skull deformities, orthodontic defects, cervical cord compression, spinal stenosis, hydrocephalus, hearing loss due to chronic otitis, obesity, disorders involving abnormal RAS-mitogen-activated protein kinase signaling, pulmonary hypertension, vasculopathy, endothelial dysfunction, liver cirrhosis, liver ascites, liver fibrosis, hepatorenal syndrome, asthma, pulmonary fibrosis, chronic kidney diseases, cardiorenal syndrome, dyspnea and lysosomal storage diseases such as mucopolysaccharidosis.

In certain embodiments, said disease treatable by CNP is one or more cardiovascular diseases selected from the group consisting of arrhythmia such as cardiac or sinus arrhythmia; atrial fibrillation; atrial flutter; bradycardia; Brugada syndrome; premature cardiac complexes; commotio cordis; heart block; long QT syndrome; parasystole; pre-excitation syndrome; tachycardia; ventricular fibrillation; ventricular flutter; cardiac conduction system disease; low cardiac output; cardiomegaly; dilated cardiomyopathy; hypertrophy such as left ventricular hypertrophy or right ventricularhyperthrophy; cardiomyopathy such as alcoholic, dilated, hypertrophic, restrictive, diabetic or Chagas cardiomyopathy; arrhythmogenic right ventricular dysplasia; endocardial fibroelastosis; endomyocardial fibrosis; glycogen storage disease type IIb; Kearns-Sayre syndrome; myocardial reperfusion injury; myocarditis; sarcoglycanophaties; endocarditis such as bacterial or non-infective endocarditis; heart arrest; sudden cardiac death; out-of-hospital cardiat arrest; cardio-renal syndrome; paroxysmal dyspnea; cardiac edema, heart failure such as diastolic or systolic heart failure; heart valve disease; aortic valve insufficiency; aortic valve stenosis; heart valve prolapse; mitral valve insufficiency; mitral valve stenosis; pulmonary atresia; pulmonary valve insufficiency; pulmonary valve stenosis; tricuspid atresia; tricuspid valve insufficiency; tricuspid valve stenosis; myocardial ischemia; acute coronary syndrome; angina pectoris; coronary disease; Kounis syndrome; myocardial infarction; pulmonary heart disease; ventricular dysfunction such as left or right ventricular dysfunction; ventricular outflow obstruction; aortic valve stenosis, pulmonary valve stenosis; hypertension; atherosclerosis; restenosis; critical limb ischaemia; peripheral arterial disease; ischemia such as ischemia-reperfusion injury or ischemic injuries; abnormal fluid accumulation in the heart and myocardial edema.

In certain embodiments, said disease treatable by CNP is selected from the group consisting of ischemic heart disease such as myocardial infarction; congestive heart failure; arrhythmia and atherosclerosis.

In certain embodiments, said disease treatable by CNP is one or more central nervous system diseases selected from the group consisting of brain ischemia such as ischemic hypoxia; brain infarction; transient ischemic attack; vertebrobasilar insufficiency; cerebrovascular disorders; stroke; intracranial hemorrhages; corneal neovascularization; corneal transplantation; gragft-versus-host disease; graft rejection; glaucoma such as angle-closure, neovascular, open-angle or low tension glaucoma; ischemic optic neuropathy; central serous chorioretinopathy; retinopathy such as diabetic or hypertensive retinopathy; retinal degeneration; macular degeneration; geographic atrophy; macular edema; Stargardt disease; vitelliform macular dystrophy; wet macular degeneration; retinoschisis; retinal detachment; retinal perforations; retinal haemorrhage; retinal neovascularization; retinal vein occlusion; retinal artery occlusion; retinopathy of prematurity and proliferative vitreoretinopathy.

In certain embodiments, said disease treatable by CNP is selected from the group consisting of hypophosphatasia, hypochondroplasia, Muenke syndrome, hypertension, osteogenesis imperfecta and achondroplasia.

In certain embodiments, said disease treatable by CNP is selected from the group consisting of achondroplasia, hypochondroplasia, short stature, Noonan syndrome or SHOX deficiency.

In certain embodiments, said disease treatable by CNP is hypophosphatasia. In certain embodiments, said disease treatable by CNP is hypochondroplasia. In certain embodiments, said disease treatable by CNP is Muenke syndrome. In certain embodiments, said disease treatable by CNP is hypertension. In certain embodiments, said disease treatable by CNP is osteogenesis imperfecta. In certain embodiments, said disease treatable by CNP is achondroplasia.

In certain embodiments, the present invention relates to a unit dosage form for use in improving muscle function in a patient having skeletal dysplasia, such as achondroplasia, the unit dosage form comprising a CNP conjugate or pharmaceutically acceptable salt thereof at a unit dose of at least 24 nmol CNP/kg and is administered to a human patient.

In certain embodiments, the present invention relates to a unit dosage form for use in improving muscle function in a patient having skeletal dysplasia, such as achondroplasia, the unit dosage form comprising a CNP conjugate or pharmaceutically acceptable salt thereof at a unit dose of about 24 nmol CNP/kg and is administered to a human patient.

In certain embodiments, the present invention relates to a unit dosage form for use in improving muscle function in a patient having skeletal dysplasia, such as achondroplasia, the unit dosage form comprising a CNP conjugate or pharmaceutically acceptable salt thereof at a unit dose of about 100 μg CNP/kg and is administered to a human patient.

In certain embodiments, the present invention relates to a unit dosage form for use in improving muscle function in a patient having of skeletal dysplasia, such as achondroplasia, the unit dosage form comprising a CNP conjugate or pharmaceutically acceptable salt thereof at a unit dose of 100 μg CNP/kg and is administered to a human patient.

In certain embodiments, the present invention relates to a unit dosage form for use in improving muscle function in a patient having skeletal dysplasia, such as achondroplasia, the unit dosage form comprising a CNP conjugate or pharmaceutically acceptable salt thereof of formula (IIf) or (IIf′) at a unit dose of 100 μg CNP/kg and is administered to a human patient aged upto 18 years of age or is a human patient with open bone epipheses.

In certain embodiments, the present invention relates to a unit dosage form for use in improving muscle function in a patient having skeletal dysplasia, such as achondroplasia, the unit dosage form comprising a CNP conjugate or pharmaceutically acceptable salt thereof of formula (IIf) or (IIf′) at a unit dose of 100 μg CNP/kg and is administered to a human patient aged 18 years of age or older.

In certain embodiments, the present invention relates to a unit dosage form for use in improving muscle function in a patient having skeletal dysplasia, such as achondroplasia, the unit dosage form comprising a CNP conjugate or pharmaceutically acceptable salt thereof of formula (IIf) or (IIf′) at a unit dose of 100 μg CNP/kg and is administered to a human patient aged 2 to 10 years.

In certain embodiments, the present invention relates to a unit dosage form for use in improving muscle function in a patient having skeletal dysplasia, such as achondroplasia, the unit dosage form comprising a CNP conjugate or pharmaceutically acceptable salt thereof of formula (IIf) or (IIf′) at a unit dose of 100 μg CNP/kg and is administered to a human patient aged 2 to 5 years.

In certain embodiments, the present invention relates to a unit dosage form for use in improving muscle function in a patient having skeletal dysplasia, such as achondroplasia, the unit dosage form comprising a CNP conjugate or pharmaceutically acceptable salt thereof of formula (IIf) or (IIf′) at a unit dose of 100 μg CNP/kg and is administered to a human patient aged 2 to 5 years.

In certain embodiments, the present invention relates to a unit dosage form for use in improving muscle function in a patient having skeletal dysplasia, such as achondroplasia, the unit dosage form comprising a CNP conjugate or pharmaceutically acceptable salt thereof at a unit dose ranging from 12.3 nmol CNP/kg to 36.9 nmol CNP/kg, wherein said unit dosage form is administered to a human patient with open epiphysis and said treatment reduces the incidence of achondroplasia-related adverse events in the human patient.

In certain embodiments, the present invention relates to a unit dosage form for use in improving muscle function in a patient having skeletal dysplasia, such as achondroplasia, the unit dosage form comprising a CNP conjugate or pharmaceutically acceptable salt thereof at a unit dose of 24.6 nmol CNP/kg, wherein said unit dosage form is administered to a human patient with open epiphysis and said treatment reduces the incidence of achondroplasia-related adverse events in the human patient.

In certain embodiments, the present invention relates to a unit dosage form for use in improving muscle function in a patient having skeletal dysplasia, such as achondroplasia, the unit dosage form comprising a CNP conjugate or pharmaceutically acceptable salt thereof at a unit dose of 100 μg CNP/kg, wherein said unit dosage form is administered to a human patient with open epiphysis and said treatment reduces the incidence of achondroplasia-related adverse events in the human patient.

In certain embodiments, the present invention relates to a unit dosage form for use in improving muscle function in a patient having skeletal dysplasia, such as achondroplasia, the unit dosage form comprising a CNP conjugate or pharmaceutically acceptable salt thereof of formula (IIf) or (IIf′) at a unit dose of 100 μg CNP/kg, wherein said unit dosage form is administered to a human patient with open epiphysis and said treatment reduces the incidence of achondroplasia-related adverse events in the human patient.

In certain embodiments, the present invention relates to a unit dosage form for use in improving muscle function in a patient having skeletal dysplasia, such as achondroplasia, the unit dosage form comprising a CNP conjugate or pharmaceutically acceptable salt thereof at a unit dose of 24.6 nmol CNP/kg, wherein said unit dosage form is administered weekly to a human patient with open epiphysis via subcutaneous injection and wherein each administration is associated with a frequency of injection site reaction of less than 3%, such as less than 2%, such as less than 1% or absence of injection site reaction.

In certain embodiments, the present invention relates to a unit dosage form for use in improving muscle function in a patient having skeletal dysplasia, such as achondroplasia, the unit dosage form comprising a CNP conjugate or pharmaceutically acceptable salt thereof of formula (IIf) or (IIf′) at a unit dose of 100 μg CNP/kg, wherein said unit dosage form is administered weekly to a human patient with open epiphysis via subcutaneous injection and wherein each administration is associated with a frequency of injection site reaction of less than 3%, such as less than 2%, such as less than 1% or absence of injection site reaction.

A further aspect of the present invention relates to a method of improving muscle function in a human patient having achondroplasia, the method comprising the step of administering a CNP conjugate or pharmaceutically acceptable salt thereof at a unit dose from about 6 μg CNP/kg to at least about 150 μg CNP/kg.

Optionally, the unit dose is about 50 μg CNP/kg to about 150 μg CNP/kg, about 75 μg CNP/kg to about 125 μg CNP/kg, about 90 μg CNP/kg to about 110 μg CNP/kg or about 100 ug CNP/kg or 100 ug/CNP/kg. Such dosages are preferably administered weekly. Such dosages are preferably administered by weekly subcutaneous injection. A preferred compound in such methods is a compound of formula (IIf) or (IIf′) with dosages referring to the mass of the CNP1-38 moiety of said compound. Thus, a preferred regimen is a compound of formula (IIf) or (IIf′) administered subcutaneously weekly at unit doses of about 100 ug CNP/kg of the CNP1-38 moiety of the compound of formula (IIf) or (IIf′). Dosages of other compounds can be adjusted for equimolar delivery of the CNP moiety of the relevant compound as for the CNP moiety of a compound of formula (IIf) or (IIf′) within a tolerance of +/−20% or +/−10%. Dosages regiments of other compounds or the compound of formula (III) or (IIf′) can be adjusted to deliver the same area under the curve+/−20% or +/−10% on a molar basis of CNP moiety, as a compound of formula (IIf) or (IIf′) administered weekly subcutaneously. The same regimens can be used in treating other disease treatable with CNP disclosed herein.

In certain embodiments, the present invention relates to a method of improving muscle function in a human patient having achondroplasia, the method comprising the step of administering a CNP conjugate or pharmaceutically acceptable salt thereof at a unit dose of 6 μg CNP/kg, 20 μg CNP/kg, 50 μg CNP/kg, 75 μg CNP/kg, 100 μg CNP/kg or 125 μg CNP/kg.

In certain embodiments, the present invention relates to a method of improving muscle function in a human patient having achondroplasia, the method comprising the step of administering a CNP conjugate or pharmaceutically acceptable salt thereof at a unit dose of 50 μg CNP/kg to 100 μg CNP/kg.

In certain embodiments, the present invention relates to a method of improving muscle function in a human patient having achondroplasia the method comprising the step of administering a CNP conjugate or pharmaceutically acceptable salt thereof at a unit dose of 50 μg CNP/kg.

In certain embodiments, the present invention relates to a method of improving muscle function in a human patient having achondroplasia, the method comprising the step of administering a CNP conjugate or pharmaceutically acceptable salt thereof at a unit dose of 100 μg CNP/kg.

In certain embodiments, the present invention relates to a method of improving muscle function in a human patient having achondroplasia, the method comprising the step of administering a CNP conjugate or pharmaceutically acceptable salt thereof at a unit dose of 50 μg CNP/kg to 100 μg CNP/kg, wherein the CNP conjugate is a compound of formula (IIf′):

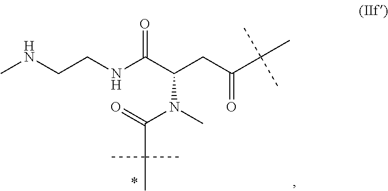

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

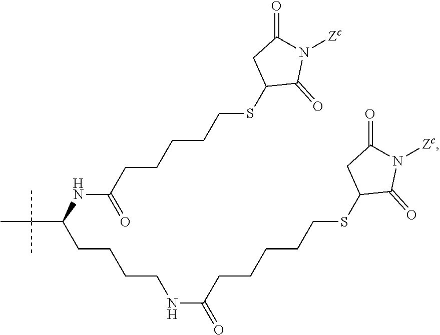

wherein
each Z^{c} is

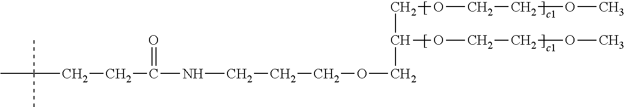

and wherein
each c1 is an integer independently ranging from 200 to 250.

In certain embodiments, the present invention relates to a method of improving muscle function in a human patient having achondroplasia, the method comprising the step of administering a CNP conjugate or pharmaceutically acceptable salt thereof at a unit dose of 50 μg CNP/kg, wherein the CNP conjugate is a compound of formula (IIf′):

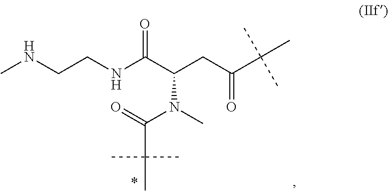

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

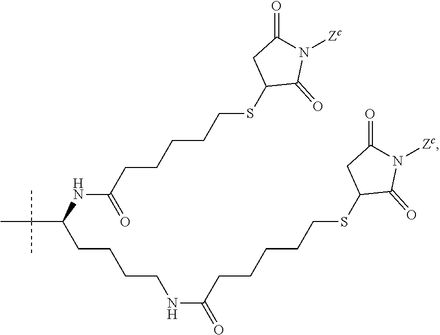

wherein
each Z^{c} is

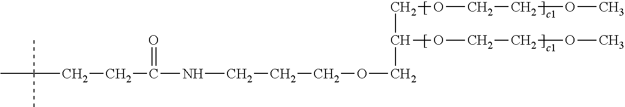

and wherein
each c1 is an integer independently ranging from 200 to 250.

In certain embodiments, the present invention relates to a method of improving muscle function in a human patient having achondroplasia, the method comprising the step of administering a CNP conjugate or pharmaceutically acceptable salt thereof at a unit dose of 100 μg CNP/kg, wherein the CNP conjugate is a compound of formula (IIf′):

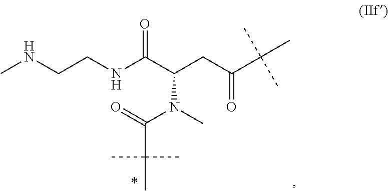

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

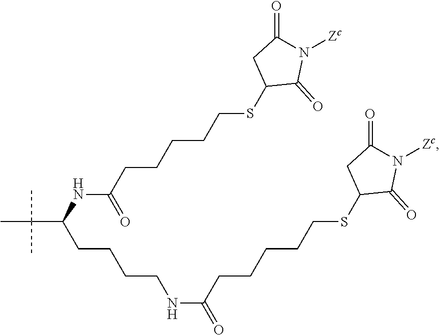

wherein
each Z^{c} is

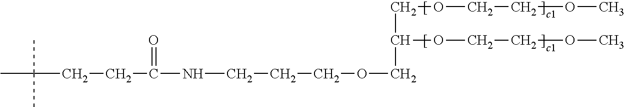

and wherein
each c1 is an integer independently ranging from 200 to 250.

In certain embodiments, the method or use of the invention comprises the step of administering a unit dosage comprising a unit dose of 24.6 nmol CNP/kg, wherein the unit dosage comprises a CNP conjugate or pharmaceutically acceptable salt thereof of formula (IIf) or (IIf′). In certain embodiments, the age of the patient ranges from 2 to 5 years old or is less than 5 years old. In certain embodiments, the age of the patient is less than 14 or 16 years of age, or is less than 18 years, or is up to 18 years of age. In certain embodiments, the patient is 18 years or older or has closed bone epipheses.

In certain embodiments, the present invention relates to a method of improving muscle function in a human patient having achondroplasia, the method comprising the step of administering a unit dosage comprising a unit dose of 100 μg CNP/kg, wherein the unit dosage comprises a CNP conjugate or pharmaceutically acceptable salt thereof of formula (IIf) or (IIf′). In certain embodiments, the age of the patient ranges from 2 to 5 years old or is less than 5 years old. In certain embodiments, the age of the patient is less than 14 or 16 years of age, or is less than 18 years, or is up to 18 years of age. In certain embodiments, the patient is 18 years or older or has closed bone epipheses.

In certain embodiments, the present invention relates to a method of of improving muscle function in a human patient having achondroplasia, the method comprising the step of administering a unit dosage comprising a unit dose of 100 μg CNP/kg, wherein the unit dosage comprises a CNP conjugate or pharmaceutically acceptable salt thereof of formula (IIf) or (If′). In certain embodiments, the age of the patient ranges from 2 to 5 years old or is less than 5 years old. In certain embodiments, the age of the patient is less than 14 or 16 years of age, or is less than 18 years, or is up to 18 years of age. In certain embodiments, the patient is 18 years or older or has closed bone epipheses.

In certain embodiments, the present invention relates to a method of improving muscle function in a human patient having achondroplasia, the method comprising the step of administering a unit dosage comprising a unit dose of 100 μg CNP/kg, wherein the unit dosage comprises a CNP conjugate or pharmaceutically acceptable salt thereof of formula (IIf) or (IIf′). In certain embodiments, the age of the patient ranges from 2 to 5 years old or is less than 5 years old. In certain embodiments, the age of the patient is less than 14 or 16 years of age, or is less than 18 years, or is up to 18 years of age. In certain embodiments, the patient is 18 years or older or has closed bone epipheses.

In certain embodiments, the present invention relates to a method of improving muscle function in a human patient having achondroplasia, the method comprising the step of administering a unit dosage comprising a unit dose of 100 μg CNP/kg, wherein the unit dosage comprises a CNP conjugate or pharmaceutically acceptable salt thereof of formula (III) or (If′). In certain embodiments, the age of the patient ranges from 2 to 5 years old or is less than 5 years old. In certain embodiments, the age of the patient is less than 14 or 16 years of age, or is less than 18 years, or is up to 18 years of age. In certain embodiments, the patient is 18 years or older or has closed bone epipheses.

Unit doses may be administered once or multiple times. For multiple administrations, the interval is preferably weekly, but can be twice a week, biweekly, or monthly among others. In certain embodiments, one unit dose is administered weekly via one subcutaneous injection. In certain embodiments, one unit dose is administered monthly via one subcutaneous injection. In certain embodiments, for patient groups weighing 55 kg or above, one unit dose may be split and administered via two simultaneous or successive injections.

In certain embodiments, the subject is a human, such as an adult human, with a weight of at least about 40 kg, or at least about 50 kg.

The CNP conjugate or pharmaceutically acceptable salt thereof may be administered for at least six months, a year, five years, ten years, until a patient is 18 years old, until patient's epiphyseal closure or indefinitely. In certain embodiments, the CNP conjugate or pharmaceutically acceptable salt thereof may be administered until a patient is 18 years old. In certain embodiments, the CNP conjugate or pharmaceutically acceptable salt thereof may be administered until a patient's ephiphysis is closed.

Treatment may start antenatally, at birth or on diagnosis of a deficit or risk relating to CNP. In certain embodiments, treatment may be continued from childhood (younger than 18 years in humans) through and into adulthood (over 18 years of age in humans). In some embodiments treatment is initiated in adulthood (18 years and older for human subjects).

In some embodiments, for example when the subject has a chondrodysplasia disease, such as achondroplasia, the subject is treated via administering a therapeutically effective amount of, an inhibitor of FGFR3 signaling, an NPR-B agonist or an NPR-C agonist when less than 18 years of age, and as an adult. In some embodiments the treatment is continued from childhood (less than 18 years of age) into adulthood (18 years and above). In some embodiments, the subject has had effective treatment for at least 1 year, such as at least 2 years, such as at least 3 years, such as at least 5 years, such as at least 10 years prior to their 18th birthday. In some embodiments, the subject has had effective treatment for at least 1 year, such as at least 2 years, such as at least 3 years, such as at least 5 years, such as at least 10 years after their 18th birthday.

If a unit dose is determined for a particular drug, such as as a reversible CNP conjugate of formula (IIf) or (IIf′), then the unit dose can be used as a guide for other reversible CNP conjugates, such that the unit dose of other reversible conjugates is the same by moles of CNP as that for CNP of formula (IIf) or (IIf′). Such guidance is particularly useful when the other conjugate releases a CNP moiety with a release half-life within plus or minus 20% of that of the conjugate of CNP of formula (IIf) or (IIf′). Thus, for example, if a unit dose is determined for a reversible conjugate of CNP-38, then an otherwise similar reversible conjugate of CNP-53 can be administered by multiplying the dose for CNP-38 by the ratio of molecular weights of CNP-53/CNP-38.

In certain embodiments, administration of the unit dose takes place by subcutaneous injection with a syringe, needle, pen injector or auto-injector. In certain embodiments, administration of the unit dose takes place by subcutaneous injection with a syringe. In certain embodiments, administration of the unit dose takes place by subcutaneous injection with a pen injector. In certain embodiments, administration of the unit dose takes place by subcutaneous injection with an auto-injector.

The present methods can be used for of improving muscle function in a patient having or at risk of (e.g., genetic risk) of a disease treatable with CNP. The present methods can also be used for treating a population of patients having or at risk of such a disease. Such a population can include at least 10, 100 or 1000 patients or may represent all patients at a particular institution.

Although clinical trials can be useful for determining doses and dosage regimens de novo, the present methods can also be performed not in the course of a clinical trial. Preferably the regimen used results in a statistically significant improvement (p≤0.05) in at least one aspect of muscle function or other aspect referred to above in such a population relative to a contemporary or historical control population receiving placebo. Improvement in an aspect of muscle function or other aspect referred to above can also be monitored in an individual patient compared with baseline measurement(s) before treatment or comparison with mean values from a historical control population. For example, improvement in an aspect of muscle function or other aspect referred to above can be deemed significant if beyond the mean and one or two standard deviations of the mean value in such a control population in a direction indicating improvement. Although clinical trials can be useful for determining doses and dosage regimens de novo, the present methods can also be performed not in the course of a clinical trial.

Treating refers to curing, reducing or inhibit further deterioration of at least one sign or symptom of a disease or stabilizing at least one sign or symptom of disease. Treating can be determined by comparing sign(s) and symptom(s) in an individual patient before (baseline) and after receiving treatment or by comparing a population of treated patients to a control population as in a clinical trial or trial with an animal model.

Prophylaxis refers to preventing, reducing risk or delaying onset of at least one sign or symptom of disease in a population of patients (or animal models) at risk of the disease administered a drug or CNP conjugate according to the invention compared to a control population of patients (or animal models) at risk of the disease not treated with a drug or CNP conjugate according to the invention. The amount is also considered prophylactically effective if an individual treated patient achieves an outcome more favorable than the mean outcome in a control population of comparable patients not treated by methods of the invention.

In certain embodiments, the present invention is a kit of parts comprising the unit dosage form of the present invention in a vial, a syringe prefilled with a reconstitution solution, a needle for transferring the reconstitution solution from the syringe into the vial, an administration syringe and an injection needle.

The reconstitution solution is a sterile liquid, such as sterile water.

In certain embodiments, a vial adapter could be used instead of the needle for transferring the reconstitution solution from the syringe into the vial and for transferring the reconstituted unit dosage form from the vial to the administration syringe. In certain embodiments, the vial adaptor is a one-piece moulded plastic part with a plastic spike or needle for penetration.

The needle for transferring the reconstitution solution into the vial is a large-bore transfer needle that ensures transfer of the reconstitution solution from the syringe into the vial comprising the unit dosage form of the present invention. The large inner diameter of the needle cannula ensures an increased flow, thereby decreasing the required injection force. In certain embodiments, said needle is a 21 G×25 mm needle.

The injection needle should ensure a comfortable subcutaneous injection, preferably with an injection time of the reconstituted unit dosage form of below 10 s. In other words, the injection needle should have a certain needle gauge and needle length to ensure that the drug is administered in the subcutaneous layer with limited pain. Various injection needles were tested and it was found that the needle length may range from about 3 mm to 13 mm. Also, it was found that injection needles such as a 29 G×8 mm needle, 30 G×12 mm needle or 30 G×4 mm needle meet the requirements above.

In certain embodiments, injection of a highly concentrated reconstituted unit dosage form, such as of a unit dosage form comprising about 60.4 mg/ml CNP conjugate may be challenging due to the high viscosity of the CNP conjugate. Applicant surprisingly found that by using a 30 G×12 mm or 30 G×4 mm needle even highly concentrated unit dosage forms can be administered.

In certain embodiments, the present invention is a kit of parts comprising a pen injector or auto-injector and a vial comprising the unit dosage of the present invention, said vial being in the form of a pre-filled cartridge for use in the pen injector or auto-injector.

In certain embodiments, the present invention is a pen injector or auto-injector comprising the unit dosage form of the present invention.

The present invention also provides for a CNP, CNP conjugate or pharmaceutically acceptable salt thereof (such as a CNP or CNP conjugate or pharmaceutically acceptable salt thereof as described herein), or unit dosage form (as described herein), which is administered to a patient to provide a sustained exposure of free plasma CNP (free CNP) at an efficacious level between administrations for use in the therapeutic methods disclosed herein. Suitably, said CNP, CNP conjugate or pharmaceutically acceptable salt thereof or unit dosage form is administered via a dosing frequency which sustains plasma free CNP concentration at a therapeutic level between successive doses, such as a plasma free CNP concentration of at least about 1 pmol/L. It will be understood that a sustained-release may be obtained by treatment with repeat dosing of a therapeutically effective amount of a CNP conjugate or pharmaceutically acceptable salt thereof.

Suitably, via repeated administrations of the CNP or CNP conjugate or pharmaceutically acceptable salt thereof or unit dosage form, e.g. daily, at least daily, weekly, at least weekly, monthly or at least monthly, the sustained exposure of free CNP is for a period of at least 6 months, such as at least 9 months, such as at least 1 year.

It is recognized that during the initial treatment period, for example the first 1, 2 or 3 months from initiation of treatment (in treatment naïve patients), the minimum free CNP concentration in plasma may gradually increase after each successive dose (which may be referred to as a run in period), until a sustained therapeutically effective exposure level is maintained, that is, trough and peak values of CNP are at a steady state as a result of administration and elimination amounts being about equal through repeated cycles of administration.

A sustained exposure may be obtained when the minimum free CNP concentration in plasma between successive administrations, also referred to as trough concentration, remains at a level which is at least about 1 pmol/L, or at least about 1.5 pmol/L, or about at least 5 pmol/L, or about 8 pmol/L, or about 15 pmol/L. Alternatively, a sustained exposure may be obtained when the minimum free CNP concentration in plasma between successive administrations, also referred to as trough concentration, remains at a level which is at least about 1.4 pmol/L, or at least about 1.6 pmol/L, or about at least 6.5 pmol/L, or about 9.4 pmol/L, or about 19.7 pmol/L.

As illustrated by the data from the clinical trial reported herein, such levels of sustained free CNP-38 exposure were found to be therapeutically effective.

In certain embodiments, a sustained exposure may be obtained when the minimum free CNP concentration in plasma between successive administrations, also referred to as trough concentration, such as at least about 1.4 pmol/L, or at least about 1.6 pmol/L, or about at least 6.5 pmol/L, or about 9.4 pmol/L, or about 19.7 pmol/L.

In certain embodiments, the minimum free CNP concentration in plasma between successive administrations, also referred to as trough concentration, remains at a level of at least about 1.8 pmol/L, or at least about 2.3 pmol/L, or about at least 8.4 pmol/L, or about 12 pmol/L, or about 25 pmol/l. In certain embodiments, the minimum free CNP concentration in plasma between successive administrations, also referred to as trough concentration, remains at a level of at least about 2.7 pmol/L, or at least about 2.4 pmol/L, or about at least 9.7 pmol/L, or about 14.1 pmol/L, or about 29 pmol/L. In certain embodiments, the minimum free CNP concentration in plasma between successive administrations, also referred to as trough concentration, remains at a level of at least about 2.7 pmol/L, or at least about 2.4 pmol/L, or about at least 9.7 pmol/L, or about 14.1 pmol/L, or about 29 pmol/L.

In certain embodiments, the CNP, CNP conjugate or pharmaceutically acceptable salt thereof or unit dosage form is administered in a regimen (route of administration, frequency and amount) to achieve plasma levels of free CNP in which at steady state, troughs range from 1.8 pmol/L to about 29 pmol/L and peaks range from about 30 pmol/L to about 100 pmol/L. In certain embodiments, troughs range from about 8 pmol/L to about 29 pmol/L and peaks range from about 30 pmol/L to about 50 pmol/L. In certain embodiments, troughs range from about 18 pmol/L to about 29 pmol/L and peaks range from about 30 pmol/L to about 50 pmol/L. In certain embodiments, the ratio of peaks to troughs is no more than 1.5:1, 2:1 or 3:1. Optionally administration is weekly and by a subcutaneous route. In certain embodiments, free CNP concentrations are measured in a patient. Levels can be measured at baseline before treatment and one or more times after treatment commences.

In certain embodiments the method comprises administering successive doses of a therapeutically effective amount of the CNP conjugate and/or prodrug of CNP to the subject, wherein the sustained exposure of free CNP in the blood plasma of the patient between successive doses of the therapeutically effective amount of the CNP conjugate and/or prodrug of CNP is at least about 1 pmol/L, such as at least about 4 pmol/L, such as between about 4 pmol/L to about 30 pmol/L. In certain embodiments said method comprises administering successive doses of a therapeutically effective amount of the CNP conjugate and/or prodrug of CNP to the subject, wherein the sustained exposure of free CNP in the blood plasma of the patient between successive doses of the therapeutically effective amount of the CNP conjugate and/or prodrug of CNP is at least about 10 pmol/L

Administration in certain embodiments may be via an infusion, such as a i.v. or s.c. infusion, of the CNP to the subject, for example wherein said infusion results in a sustained exposure of free CNP in the blood plasma of the patient over a period of at least 1 hour of at least 1 pmol/L, such as at least about 4 pmol/L, such as at least about 9 pmol/L or about 4 pmol/L to about 30 pmol/L.

In addition to the injection methods described elsewhere herein, in some embodiments administration may be by an infusion, for example into the blood stream e.g. via in intravenous (i.v.) infusion, or via subcutaneous infusion, so that the drug slowly drips (delivered) into the bloodstream. Infusion may be administered, for example, via a drip (e.g. a i.v. drip) or via a a drug pump such as an osmotic pump. A drug infusion may therefore be used to maintain a sustained exposure of free CNP in the blood plasma, even with regards CNP peptides which have a short plasma half-life (e.g. a t½ of less than 6 yours, such as less than 3 hours, such as less than 1 hour, such as less than 30 minutes). By way of example vosoritide as a reported t½ of about 28 minutes (see for example Chan, Clin Pharmacokinet. 2022; 61(2): 263-280).

The present invention also provides for a method for reducing the frequency of achondroplasia related adverse events in a patient diagnosed with achondroplasia, said method comprising administering a therapeutically effective amount of a CNP, CNP conjugate or pharmaceutically acceptable salt thereof or unit dosage form, wherein optionally the patient may be a pediatric patient, and/or a patient with open bone epiphysis.

In certain embodiments, the achondroplasia-related adverse event is selected from the group consisting of sleep apnea syndrome, ear infection, foramen magnum stenosis and kyphosis.

In certain embodiments, the achondroplasia-related adverse event is selected from the group consisting of sleep apnea syndrome, ear infection, foramen magnum stenosis and kyphosis.

The present invention also provides for a method for treating sleep apnea syndrome in a patient in need to said treatment, said method comprising administering a therapeutically effective amount of a CNP, CNP conjugate or pharmaceutically acceptable salt thereof or unit dosage form to the patient. By way of example, the treatment may reduce the incidence of sleep apnea or reduce the severity of sleep apnea.

The present invention also provides for a method for treating ear infection in a patient or for reducing incidence of ear infection in a patient, in a patient in need of said treatment, said method comprising administering a therapeutically effective amount of a CNP, CNP conjugate or pharmaceutically acceptable salt thereof or unit dosage form to the patient thereby treating ear infection or reducing incidence of ear infection.

The present invention also provides for a method for treating foramen magnum stenosis in a patient in need of said treatment, said method comprising administering an effective amount of CNP, CNP conjugate or pharmaceutically acceptable salt thereof or unit dosage form to the patient thereby treating foramen magnum stenosis.

The present invention also provides for a method for treating kyphosis in a patient in need of said treatment, said method comprising administering an effective amount of CNP, CNP conjugate or pharmaceutically acceptable salt thereof or unit dosage form to the patient thereby treating kyphosis. In certain embodiments, the patient has been diagnosed with a bone dysplasia or bone disorder, such as a disorder selected from the group consisting of achondroplasia, hypochondroplasia, short stature, Noonan syndrome and SHOX deficiency.

In certain embodiments, the patient has been diagnosed with achondroplasia. In certain embodiments, the patient is a pediatric patient with open bone epiphysis.

In certain embodiments, the patient is aged up to 18 years of age or is less than 18 years of age. In certain embodiments, the patient is aged up to 16 years of age or is less than 16 years of age. In certain embodiments, the patient is aged up to 14 years of age or is less than 14 years of age. In certain embodiments, the patient is aged up to 10 years of age or is less than 10 years of age. In certain embodiments, the patient is aged up to 5 years of age or is less than 5 years of age. In certain embodiments, the patient is an achondroplasia patient aged up to 2-5 years of age, such as 2, 3 or 4 years of age. In certain embodiments, the patient is an achondroplasia patient at least 6 months of age, such as at least 1 year of age or at least 2 years of age. In certain embodiments, the patient is an achondroplasia patient with open bone epiphysis. In certain embodiments, the patient is an achondroplasia patient aged up to 18 years of age or is less than 18 years of age. In certain embodiments, the patient is an achondroplasia patient aged up to 16 years of age or is less than 16 years of age. In certain embodiments, the patient is an achondroplasia patient aged up to 14 years of age or is less than 14 years of age. In certain embodiments, the patient is an achondroplasia patient aged up to 10 years of age or is less than 10 years of age. In certain embodiments, the patient is an achondroplasia patient aged up to 5 years of age or is less than 5 years of age. In certain embodiments, the patient is an achondroplasia patient up to 2-5 years of age, such as 2, 3 or 4 years of age. In certain embodiments, the patient is an achondroplasia patient at least 6 months of age, such as at least 1 year of age or at least 2 years of age.

In some embodiments, for example when the subject has a chondrodysplasia disease, such as achondroplasia, the subject is treated via administering a therapeutically effective amount of, an inhibitor of FGFR3 signaling, an NPR-B agonist or an NPR-C agonist when less than 18 years of age, and as an adult. In some embodiments the treatment is continued from childhood (less than 18 years of age) into adulthood (18 years and above), e.g over a period of at least 1, 3, 5, 10, 15 years, starting when the patient is a child, optionally wherein the treatment continues for at least 1, 3, 5, 10, 15 years after the subject is 18 years of age. In some embodiments, the subject has had effective treatment for at least 1 year, such as at least 2 years, such as at least 3 years, such as at least 5 years, such as at least 10 years prior to their 18th birthday. In some embodiments, the subject has had effective treatment for at least 1 year, such as at least 2 years, such as at least 3 years, such as at least 5 years, such as at least 10 years after their 18th birthday. In further embodiments, the subject has had effective treatment for at least 1 year, such as at least 2 years, such as at least 3 years, such as at least 5 years, such as at least 10 years, prior to their 18th birthday and at least 1 year, such as at least 2 years, such as at least 3 years, such as at least 5 years, such as at least 10 years after their 18th birthday.

In some embodiments, for example when the subject has a chondrodysplasia disease, such as achondroplasia, the subject is treated via administering a therapeutically effective amount of, an inhibitor of FGFR3 signaling, an NPR-B agonist or an NPR-C agonist when the subject has open bone epiphysis and when the subject has closed bone epiphysis. In some embodiments the treatment is continued from a period where the bone epiphysis is open into the period in which it is closed, e.g over a period of at least 1, 3, 5, 10, 15 years, starting when the patient has open bone epiphysis, optionally wherein the treatment continues for at least 1, 3, 5, 10, 15 years after closure of the bone epiphysis. In some embodiments, the subject has had effective treatment for at least 1 year, such as at least 2 years, such as at least 3 years, such as at least 5 years, such as at least 10 years prior to closure of the bone epiphysis. In some embodiments, the subject has had effective treatment for at least 1 year, such as at least 2 years, such as at least 3 years, such as at least 5 years, such as at least 10 years after closure of the bone epiphysis. In further embodiments, the subject has had effective treatment for at least 1 year, such as at least 2 years, such as at least 3 years, such as at least 5 years, such as at least 10 years, prior to closure of the bone epiphysis and at least 1 year, such as at least 2 years, such as at least 3 years, such as at least 5 years, such as at least 10 years after closure of the bone epiphysis. The treatment is therefore optionally initiated before closure of the bone epiphysis and continued after closure of the bone epiphysis.

The invention is further described by the following non-limiting items.

### Items

1. A method of improving muscle function in a subject suffering from a disease or condition in which muscle function is impaired, the method comprising administering a therapeutically effective amount of, an inhibitor of FGFR3 signaling, an NPR-B agonist or an NPR-C agonist, to said subject. 35
2. The method of item 1, wherein the inhibitor of FGFR3 signaling is an FGFR3 antagonist or an NPR-B agonist.
3. The method of item 1 or 2, wherein the NPR-B agonist is or comprises a C-type natriuretic peptide (CNP).
4. The method of any one of items 1 to 3, wherein the NPR-C agonist is or comprises a C-type natriuretic peptide (CNP).
5. The method of any one of items 1 to 4, wherein the NPR-B agonist or NPR-C agonist is administered as a CNP conjugate or a pharmaceutically acceptable salt thereof.
6. The method of any one of items 1 to 5, wherein the NPR-B agonist or NPR-C agonist is a prodrug of CNP.
7. The method of item 5 or 6, wherein the CNP conjugate or prodrug of CNP is a compound of formula (IIf′), formula (IIf), compound (1), or a pharmaceutically acceptable salt thereof.
8. The method according to any one of items 5 to 7, wherein said method comprises administering successive doses of a therapeutically effective amount of the CNP conjugate and/or prodrug of CNP to the subject, wherein the sustained exposure of free CNP in the blood plasma of the patient between successive doses of the therapeutically effective amount of the CNP conjugate and/or prodrug of CNP is at least about 1 pmol/L, such as at least about 4 pmol/L, or such as between about 4 pmol/L to about 30 pmol/L.
9. The method according to item 8, wherein said method comprises administering successive doses of a therapeutically effective amount of the CNP conjugate and/or prodrug of CNP to the subject, wherein the sustained exposure of free CNP in the blood plasma of the patient between successive doses of the therapeutically effective amount of the CNP conjugate and/or prodrug of CNP is at least about 9 pmol/L.
10. The method according to item 8 or 9, wherein the therapeutically effective amount of the CNP conjugate and/or prodrug of are administered daily or weekly.
11. The method according to any one of items 3 to 7, wherein said method comprises an infusion, such as a i.v. or s.c. infusion, of the CNP to the subject, wherein said infusion results in a sustained exposure of free CNP in the blood plasma of the patient over a period of at least 1 hour of at least 1 pmol/L, such as at least about 4 pmol/L, such as at least about 9 pmol/L or about 4 pmol/L to about 30 pmol/L.
12. The method of any one of items 1 to 11, wherein the NPR-B agonist is or comprises vosoritide (SEQ ID NO:30).
13. The method of item 2, wherein the FGFR3 antagonist is an FGFR3 tyrosine kinase inhibitor.
14. The method of item 13, wherein the FGFR3 tyrosine kinase inhibitor is selected from the group consisting of infigratinib, pemigatinib, futibatinib, erdafitinib and TYRA-300.
15. The method of any one of the preceding items, wherein the subject is a human subject.
16. The method of item 15, wherein the subject is less than 18 years of age.
17. The method of item 15, wherein the subject is at least 18 years of age.
18. The method of any one of the preceding items, wherein the subject has closed bone epiphysis.
19. The method of any one of the preceding items, wherein the improvement in muscle function is one or more of:
a) increased skeletal muscle strength,
b) increased skeletal muscle tone,
c) increased skeletal muscle stamina,
d) increased skeletal muscle mass,
e) decreased skeletal muscle fatigue,
f) increased cardiovascular endurance,
g) increased cardiovascular fitness,
h) decreased exercise intolerance,
i) increased exercise capacity,
j) decreased exercise induced fatigue, and
k) decreased hypotonia.
20. The method of any one of the preceding items, wherein the administration gives rise to increased skeletal muscle mass and/or muscle/fat ratio (such as skeletal muscle/fat ratio), in the subject.
21. The method of any one of the preceding items, wherein said subject has hypotonia.
22. The method of any one of the preceding items, wherein said administration gives rise to treatment or prevention of musculo-skeletal pain in the subject.
23. The method of any one of the preceding items, wherein said administration gives rise to an improvement in posture or a reduction in an abnormal curvature of the spine.
24. The method of any one of the preceding items, wherein said administration gives rise to an improvement in kyphosis, lordosis, spinal stenosis or scoliosis.
25. The method of any one of the preceding items, wherein said administration gives rise to an improvement in sleep apnea, obstructive sleep apnea, or otitis media (e.g. acute otitis media).
26. The method of any one of the preceding items, wherein said administration gives rise to a reduction in obesity.
27. The method of any one of the preceding items, wherein the subject has a chondrodysplasia disease, such as a disease selected from the group consisting of achondroplasia, hypochondroplasia, and thanatophoric dysplasia.
28. The method of any one of items 1 to 27, wherein the subject has a RASopathy, such as a RASopathy selected from the group consisting of neurofibromatosis type 1 (NF 1), Noonan syndrome, Noonan syndrome with multiple lentigines, capillary malformation-arteriovenous malformation syndrome, Costello syndrome, cardio-facio-cutaneous syndrome, and Legius syndrome.
29. The method of any one of items 1 to 28, wherein the subject has a disease or condition associated with an impairment in neuromuscular function, such as a neuromuscular or a neurodegenerative disease.
30. The method of item 29, wherein the neurodegenerative disease is selected from the group consisting of Parkinson's Disease, Alzheimer's Disease, Multiple Sclerosis, Amyotrophic lateral sclerosis (ALS), ataxias such as Friedreich's ataxia (FRDA), and Huntington's disease (HD).
31. The method of item 29 or 30, wherein said disease or condition associated with an impairment in neuromuscular function is a disease or condition in which mitochondrial dysfunction is present.
32. The method of any one of items 29 to 31 wherein said treatment gives rise to a reduction or decline of muscle function.
33. The method of any one of the preceding items, further comprising the administration of a growth hormone, such as a human growth hormone, or prodrug thereof.
34. A method of improving muscle function in a subject suffering from a disease or condition in which muscle function is impaired, the method comprising:
(i) Assessing muscle function, at least once, in the subject suffering from a disease or condition in which muscle function is impaired, and
(ii) administering a therapeutically effective amount of an inhibitor of FGFR3 signaling, an NPR-B agonist or an NPR-C agonist, to the subject.
35. The method of item 34, wherein the assessment of muscle function provides a baseline value or determination of muscle function, optionally wherein said assessment is carried out within a month before initiating the administering step.
36. The method of item 34 or 35, wherein the administering step is initiated responsive to the assessing step.
37. The method of any one of items 34 to 36, further comprising diagnosing the subject as suffering from an impairment of muscle function and/or from a disease or condition in which muscle function is impaired from the assessing step.
38. The method of item 37, wherein the administering step is initiated responsive to diagnosing the subject as suffering from an impairment of muscle function and/or from a disease or condition in which muscle function is impaired.
39. The method of any of items 34 to 37, wherein the assessing step is performed at least twice, once or at least once to determine a baseline before initiating the administering step and once or at least once after initiating the administering step.
40. The method of item 39, wherein the assessing step is performed at least three times, once or at least once to determine a baseline before initiating the administering step and twice or at least twice after initiating the administering step.
41. The method of item 40, wherein the assessing step is performed at least twice before initiating the administering step for determining whether there is a trend in muscle function before treatment and at least twice after initiating the administering step for determining whether there is a response to treatment.
42. The method of any one of items 39-41, further comprising comparing muscle function determined before initiating the administering step to muscle function determined after initiating the administering step.
43. The method of item 42, wherein the comparison indicates an improvement in muscle function which is an improvement as defined in any of items 19 to 26.
44. The method of item 42, wherein the comparison indicates an improvement in muscle function which is a reduction in decline in muscle function responsive to the administering step.
45. The method of item 43 or 44, wherein (i) the dose of and/or (ii) the frequency of, the administration of the inhibitor of FGFR3 signaling, an NPR-B agonist or an NPR-C agonist is maintained responsive to the comparison.
46. The method of item 42, wherein the comparison indicates unchanged or decreased muscle function and (i) the dose of and/or (ii) the frequency of, the administration of the inhibitor of FGFR3 signaling, an NPR-B agonist or an NPR-C agonist is increased in response to the comparison.
47. The method of any one of items 34 to 46, wherein the muscle function is skeletal muscle function.
48. The method of any one of items 34 to 47, wherein muscle function in the subject is assessed by assessing one or more of:
a) skeletal muscle strength,
b) skeletal muscle tone,
c) skeletal muscle stamina,
d) skeletal muscle mass,
e) skeletal muscle fatigue,
f) cardiovascular endurance,
g) cardiovascular fitness,
h) exercise intolerance,
i) exercise capacity,
j) exercise induced fatigue,
k) hypotonia
l) skeletal muscle mass and/or muscle/fat ratio (such as skeletal muscle/fat ratio)
m) musculo-skeletal pain,
n) posture or curvature of the spine, optionally kyphosis, lordosis, spinal stenosis or scoliosis,
o) sleep apnea, obstructive sleep apnea, or otitis media (e.g. acute otitis media),
p) obesity.
49. The method of item 48, further comprising determining a composite index for outcome of the assessment of at least two of a) to p) values for at least two of a)-p).
50. The method of item 48, wherein the assessment comprises a) imaging, b) determining a performance metric of the subject and/or c) a measurement of one or more of muscle mass, muscle volume, muscle density or muscle length of the subject.
51. The method of any one of items 34 to 50, wherein the subject has a disease or condition associated with an impairment in neuromuscular function, such as a neuromuscular or a neurodegenerative disease, optionally wherein the neurodegenerative disease is selected from the group consisting of Parkinson's Disease, Alzheimer's Disease, Multiple Sclerosis, Amyotrophic lateral sclerosis (ALS), ataxias such as Friedreich's ataxia (FRDA), and Huntington's disease (HD).
52. The method of item 51, wherein said disease or condition associated with an impairment in neuromuscular function is a disease or condition in which mitochondrial dysfunction is present.
53. A method of increasing skeletal muscle strength, optionally in a subject diagnosed with a disease or condition in which muscle e.g. skeletal muscle function is impaired, the method comprising administering an effective amount of an inhibitor of FGFR3 signalling, or an effective amount of an NPR-B agonist, or an effective amount of an NPR-C agonist to said subject thereby increasing skeletal muscle strength.
54. The method of item 53, further comprising detecting the increased muscle strength.
55. A method of increasing muscle tone in a subject, optionally in a subject diagnosed with impaired muscle tone, the method comprising administering an effective amount of an inhibitor of FGFR3 signalling or an effective amount of an NPR-B agonist, or an effective amount of an NPR-C agonist to said subject, thereby increasing muscle tone.
56. The method of item 55 further comprising detecting the increased muscle tone.
57. A method of increasing skeletal muscle stamina in a subject, optionally in a subject diagnosed with impaired skeletal muscle stamina, the method comprising administering an effective amount of an inhibitor of FGFR3 signalling or an effective amount of an NPR-B agonist, or an effective amount of an NPR-C agonist to said subject thereby increasing skeletal muscle stamina.
58. The method of item 57, further comprising detecting the increased skeletal muscle stamina.
59. A method of increasing skeletal muscle mass in a subject, optionally in a subject diagnosed with impaired skeletal muscle mass, the method comprising administering an effective amount of an inhibitor of FGFR3 signalling or an effective amount of an NPR-B agonist, or an effective amount of an NPR-C agonist to said subject thereby increasing skeletal muscle mass.
60. The method of item 59, further comprising detecting the increased muscle mass.
61. A method of decreasing skeletal muscle fatigue in a subject, optionally in a subject diagnosed with skeletal muscle fatigue, the method comprising administering an effective amount of an inhibitor of FGFR3 signalling or an effective amount of an NPR-B agonist, or an effective amount of an NPR-C agonist to said subject thereby decreasing skeletal muscle fatigue.
62. The method of item 61, further comprising detecting the decrease skeletal muscle fatigue.
63. A method of increasing cardiovascular endurance in a subject, optionally wherein the subject has been diagnosed with impaired cardiovascular endurance, the method comprising administering an effective amount of an inhibitor of FGFR3 signalling or an effective amount of an NPR-B agonist, or an effective amount of an NPR-C agonist to said subject thereby increasing cardiovascular endurance.
64. The method of item 63 further comprising detecting the increased cardiovascular endurance.
65. A method of increasing cardiovascular fitness in a subject, (optionally wherein the subject has been diagnosed with impaired cardiovascular fitness), the method comprising administering an effective amount of an inhibitor of FGFR3 signalling or an effective amount of an NPR-B agonist, or an effective amount of an NPR-C agonist to said subject thereby increasing cardiovascular endurance.
66. The method of item 65 further comprising detecting the increased cardiovascular endurance.
67. A method of decreasing exercise intolerance in a subject, optionally wherein the subject has been diagnosed with impaired tolerance to exercise, the method comprising administering an effective amount of an inhibitor of FGFR3 signalling or an effective amount of an NPR-B agonist, or an effective amount of an NPR-C agonist to said subject thereby decreasing exercise intolerance.
68. The method of item 67, further comprising detecting the decreased exercise intolerance.
69. A method of increasing exercise capacity in a subject, optionally wherein the subject has been diagnosed with impaired exercise capacity, the method comprising administering an effective amount of an inhibitor of FGFR3 signalling or an effective amount of an NPR-B agonist, or an effective amount of an NPR-C agonist to said subject thereby increasing exercise capacity.
70. The method of item 69, further comprising detecting the increased exercise intolerance.
71. A method of decreasing exercise induced fatigue in a subject, optionally wherein the subject has been diagnosed with excessive fatigue, the method comprising administering an effective amount of an inhibitor of FGFR3 signalling or an effective amount of an NPR-B agonist, or an effective amount of an NPR-C agonist to said subject thereby reducing fatigue.
72. The method of item 71 further comprising detecting the reduced fatigue
73. A method of decreasing hypotonia in a subject, optionally wherein the subject has been diagnosed with hypotonia, the method comprising administering an effective amount of an inhibitor of FGFR3 signalling or an effective amount of an NPR-B agonist, or an effective amount of an NPR-C agonist to said subject thereby decreasing hypertonia.
74. The method of item 73, further comprising detecting the decreased hypertonia.
75. A method of increasing the skeletal muscle/fat ratio in a subject, optionally wherein the subject has been diagnosed with a disease or condition in which muscle function is impaired, the method comprising administering an effective amount of an inhibitor of FGFR3 signalling, or an effective amount of an NPR-B agonist, or an effective amount of an NPR-C agonist to said subject thereby increasing the skeletal muscle/fat ratio.
76. The method of item 75 further comprising detecting the increased skeletal muscle/fat ratio.
77. A method of treating, preventing or reducing decreasing musculoskeletal pain in a subject, optionally wherein the subject is experiencing musculoskeletal pain, the method comprising administering an effective amount of an inhibitor of FGFR3 signalling, or an effective amount of an NPR-B agonist, or an effective amount of an NPR-C agonist to said subject thereby reducing musculoskeletal pain.
78. The method of item 77, further comprising detecting the reduced musculoskeletal pain.
79. A method of improving posture or treating an abnormal curvature of the spine in a subject, optionally wherein the subject has been diagnosed with abnormal curvature of the spine, the method comprising administering an effective amount of an inhibitor of FGFR3 signalling or an effective amount of an NPR-B agonist, or an effective amount of an NPR-C agonist to said subject, thereby reducing abnormal curvature and optionally improving kyphosis, lordosis or scoliosis.
80. The method of item 79 further comprising detecting the reduced abnormal curvature and optionally the improved kyphosis, lordosis or scoliosis.
81. A method of treating sleep apnea, snoring, obstructive sleep apnea, or otitis media (e.g. acute otitis media) in a subject, optionally diagnosed with one or more of these conditions, the method comprising administering an effective amount of an inhibitor of FGFR3 signalling or an effective amount of an NPR-B agonist, or an effective amount of an NPR-C agonist to said subject, thereby reducing frequency of occurrence, incidence and/or severity of the sleep apnea, snoring, obstructive sleep apnea, otitis media (e.g. acute otitis media).
82. The method of item 81 further comprising detecting the reduced frequency of occurrence, incidence and/or severity of the sleep apnea, snoring, obstructive sleep apnea, or otitis media (e.g. acute otitis media).
83. A method of reducing obesity in a subject, optionally wherein the subject has been diagnosed with obesity, the method comprising administering an effective amount of an inhibitor of FGFR3 signalling or an effective amount of an NPR-B agonist, or an effective amount of an NPR-C agonist to said subject, thereby reducing obesity.
84. The method of item 83, further comprising detecting the reduced obesity.
85. A method for treating kyphosis in a subject, optionally, wherein the subject has been diagnosed with kyphosis, said method comprising administering an effective amount of CNP, CNP conjugate or pharmaceutically acceptable salt thereof or unit dosage form to the patient, thereby reducing kyphosis.
86. The method of item 85, further comprising detecting the reduced kyphosis.
87. A method of treating foramen magnum stenosis in a subject, optionally wherein the subject has been diagnosed with foramen magnum stenosis, said method comprising administering an effective amount of CNP, CNP conjugate or pharmaceutically acceptable salt thereof or unit dosage form to the patient thereby reducing foramen magnum stenosis.
88. The method of item 87, further comprising detecting the reduced foramen magnum stenosis.
89. A method of treating otitis media (e.g. acute otitis media) or ear infection in a subject optionally in a subject diagnosed with otitis media (e.g. acute otitis media) or ear infection said method comprising administering a therapeutically effective amount of a CNP, CNP conjugate or pharmaceutically acceptable salt thereof or unit dosage form to the patient thereby reducing otitis media (e.g. acute otitis media), or ear infection or reducing incidence of ear infection.
90. The method of item 89, further comprising detecting the reduced otitis media (e.g. acute otitis media) or ear infection.
91. A method of treating obstructive sleep apnea syndrome in a subject, optionally in a subject diagnosed with sleep apnea syndrome, said method comprising administering a therapeutically effective amount of a CNP, CNP conjugate or pharmaceutically acceptable salt thereof or unit dosage form to the subject, thereby reducing incidence or severity of sleep apnea.
92. The method of item 91, further comprising detecting the reduced incidence or severity of obstructive sleep apnea syndrome.
93. A method of treating or preventing a disease or condition associated with an impairment in neuromuscular function, such as a neurodegenerative disease in a subject, said method comprising administering a therapeutically effective amount of, an inhibitor of FGFR3 signaling, an NPR-B agonist or an NPR-C agonist, to said subject, optionally wherein the neurodegenerative disease is selected from the group consisting of Parkinson's Disease, Alzheimer's Disease, Multiple Sclerosis, Amyotrophic lateral sclerosis (ALS), ataxias such as Friedreich's ataxia (FRDA), and Huntington's disease (HD).
94. The method of item 93, wherein said disease or condition associated with an impairment in neuromuscular function is a disease or condition in which mitochondrial dysfunction is present.
95. The method of item 93 or 94, wherein the administration of the therapeutically effective amount of, an inhibitor of FGFR3 signaling, an NPR-B agonist or an NPR-C agonist, to said subject results in improved skeletal muscle function or improved neuromuscular function, or a slowing, or delaying or reduction of disease progression, such as a reduced rate of loss or decline of muscle function, or reduced rate of loss or decline of neuromuscular function.
96. The method of any one of items 34-95, wherein the subject has a chondrodysplasia disease, such as a disease selected from the group consisting of achondroplasia, hypochondroplasia, and thanatophoric dysplasia.
97. The method of any one of items 34-95, wherein the subject has not been diagnosed with achondroplasia.
98. The method of any one of items 34-95, wherein the subject has not been diagnosed with a chondrodysplasia disease, such as a disease selected from the group consisting of achondroplasia, hypochondroplasia, and thanatophoric dysplasia.
99. The method of any one of items 34-95, wherein the subject has not been diagnosed with bone dysplasia or a bone disorder, such as a disorder selected from the group consisting of achondroplasia, hypochondroplasia, short stature, Noonan syndrome and SHOX deficiency.
100. The method of any one of items 34-99, wherein the subject has been diagnosed with impaired muscle function.
101. The method of any one of items 34-100, wherein the subject is a human subject of at least 18 years of age, or a human subject whose bone epiphysis has closed.
102. The method of any one of items 34-101, wherein the inhibitor of FGFR3 signaling is an FGFR3 antagonist or an NPR-B agonist.
103. The method of any one of items 34-102, wherein the NPR-B agonist is or comprises a C-type natriuretic peptide (CNP).
104. The method of any one of items of any one of items 34-103, wherein the NPR-C agonist is or comprises a C-type natriuretic peptide (CNP).
105. The method of any one of items 34-100, wherein the NPR-B agonist or NPR-C agonist is administered as a CNP conjugate or a pharmaceutically acceptable salt thereof.
106. The method of any one of items 34-105, wherein the NPR-B agonist or NPR-C agonist is a prodrug of CNP.
107. The method of item 103 or 104, wherein the CNP conjugate or prodrug of CNP is a compound of formula (IIf′), formula (III), compound (1), or a pharmaceutically acceptable salt thereof.
108. The method according to any one of items 103 to 107, wherein said method comprises administering successive doses of a therapeutically effective amount of the CNP conjugate and/or prodrug of CNP to the subject, wherein the sustained exposure of free CNP in the blood plasma of the patient between successive doses of the therapeutically effective amount of the CNP conjugate and/or prodrug of CNP is at least about 1 pmol/L, such as at least about 4 pmol/L, such as between about 4 pmol/L to about 30 pmol/L.
109. The method according to item 108, wherein said method comprises administering successive doses of a therapeutically effective amount of the CNP conjugate and/or prodrug of CNP to the subject, wherein the sustained exposure of free CNP in the blood plasma of the patient between successive doses of the therapeutically effective amount of the CNP conjugate and/or prodrug of CNP is at least about 9 pmol/L
110. The method according to item 103 or 104, wherein the therapeutically effective amount of the CNP conjugate and/or prodrug of are administered daily or weekly.
111. The method according to any one of items 104-107, wherein said method comprises administering an infusion, such as a i.v. or s.c. infusion, of the CNP to the subject, wherein said infusion results in a sustained exposure of free CNP in the blood plasma of the patient over a period of at least 1 hour of at least 1 pmol/L, such as at least about 4 pmol/L, such as at least about 9 pmol/L or about 4 pmol/L to about 30 pmol/L.
112. The method of any one of items 34 to 112, wherein the NPR-B agonist is or comprises vosoritide (SEQ ID NO:30).
113. The method of item 102, wherein the FGFR3 antagonist is an FGFR3 tyrosine kinase inhibitor.
114. The method of item 113, wherein the FGFR3 tyrosine kinase inhibitor is selected from the group consisting of infigratinib, pemigatinib, futibatinib, erdafitinib and TYRA-300.

### Examples

### Materials and Methods

All materials were commercially available except where stated otherwise.

### Example 1: Synthesis of Compound (1)

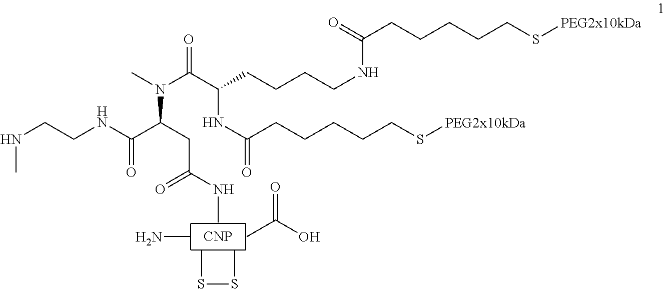

Compound (1) was synthesized as described in WO2017/118693 for conjugate 11i.

Compound (1) can also be represented as follows:

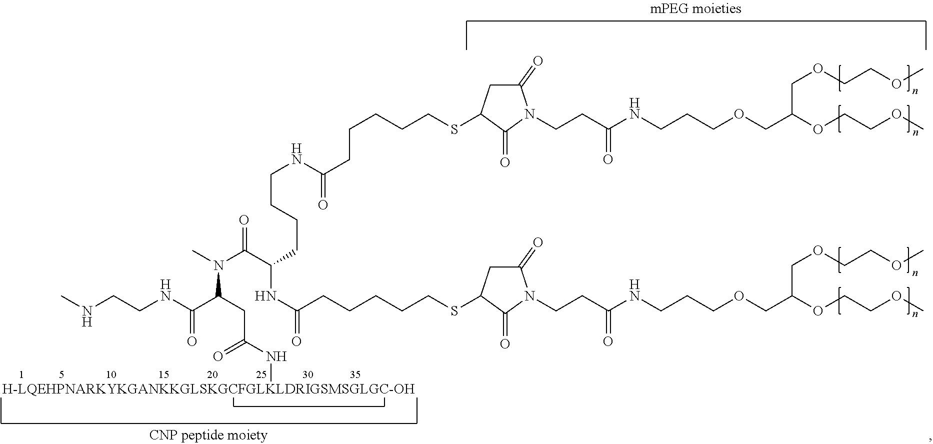

wherein n ranges from about 200 to about 250, such as from 200 to 250 corresponding to about 10 kDa.

Compound (1) is a long-acting essentially inactive prodrug consisting of CNP-38 transiently bound to a carrier molecule, mPEG via a reversible linker. mPEG acts as an inert carrier, extending CNP-38 circulation time in the body through a shielding effect that minimizes CNP-38 clearance and largely inactivates CNP-38 until its release. Compound (1) releases active CNP via auto-cleavage of the reversible linker in a controlled-manner based on physiologic pH and temperature. As such, compound (1) is designed to provide sustained exposure of active CNP over 7 days, allowing an optimal pharmacokinetic profile for use in ACH. The drug product formulation comprising compound (1) is a lyophilized powder in a single-use vial, i.e. compound (1) 3.9 mg CNP-38/vial. Prior to use, the lyophilized powder must be reconstituted with sterile water for injection from a prefilled syringe. After reconstitution the concentration is 3.6 mg CNP-38/ml. This solution will be administered by subcutaneous injection via syringe and needle. With reference to compound (1), the dose level CNP/kg or CNP-38/kg refers to the amount of CNP moiety (μg) present in the dose of compound (1), per kilogram of patient's bodyweight.

### Example 2 In Vivo Evaluation of the Effect of Compound (1) in Fgfr3^{Y367C/+} Mice

Compound (1) was tested in a murine mouse model with a thanatophoric dysplasia FGFR3 gain-of-function genotype (Fgfr3^{Y367C/+}) expressing an achondroplasia-like phenotype.

The Fgfr3^{Y367C/+} mouse model was selected as it displays several relevant characteristics of human dwarfism and because the mutant allele is expressed at sites and levels similar to normal FGFR3 (Lorget 2012 Am J Hum Genet 2012 Dec. 7; 91(6):1108-14).

Fgfr3^{Y367C/+} mice were generated by crossing CMV-Cre mice (C57BL/6J) to mice exhibiting the germline transmission of the Y367C mutation corresponding to the human Y373C (Thanatophoric Dysplasia) mutation. The Y367C mutation was introduced into exon 9 of the mouse Fgfr3 gene along with a NEO cassette flanked by LoxP recombination sites. Due to the CMV promoter, the expression of the Fgfr3 Fgfr3Y^{Y367C/+} is ubiquitous. Off-spring consisted of both Fgfr3Y^{Y367C/+} and Fgfr3+/+ wild-type (WT) littermates.

Throughout the study period, irradiated global diet pellet and deionized water was available to animals ad libitum. No known contaminants that would be expected to interfere with this study were detected in the feed or water.

Environmental controls were set to maintain temperatures of 18° to 26° C. (64° to 79° F.) with a relative humidity of 30% to 70%. These parameters were recorded at least once daily. A 12:12 hour light:dark cycle was maintained.

Newborn mice harboring the Fgfr3^{Y367C/+} mutation were administered via subcutaneous injection, with Compound (1) at two different dose levels (Group 1 and Group 2), or vehicle, for 15 days starting at day 1 after birth, in accordance with Table 1 below. Vehicle consisted of formulation buffer without Compound (1).

TABLE 1
Mouse survival data

| | | | Number | Total |
|---|---|---|---|---|
| | | | of mice | number |
| Dosing | | | surviving | of mice |
| group | Genotype | Dose | to day 15 | included |
| Group 1 | Fgfr3^{Y367C/+} | 5.6 mg/kg/day | 12 | 14 |
| | | Compound (1) | | |
| Group 2 | Fgfr3^{Y367C/+} | 1.2 mg/kg/every | 11 | 13 |
| | | third day | | |
| | | Compound (1) | | |
| Vehicle | Fgfr3^{Y367C/+} | Vehicle | 11 | 17 |

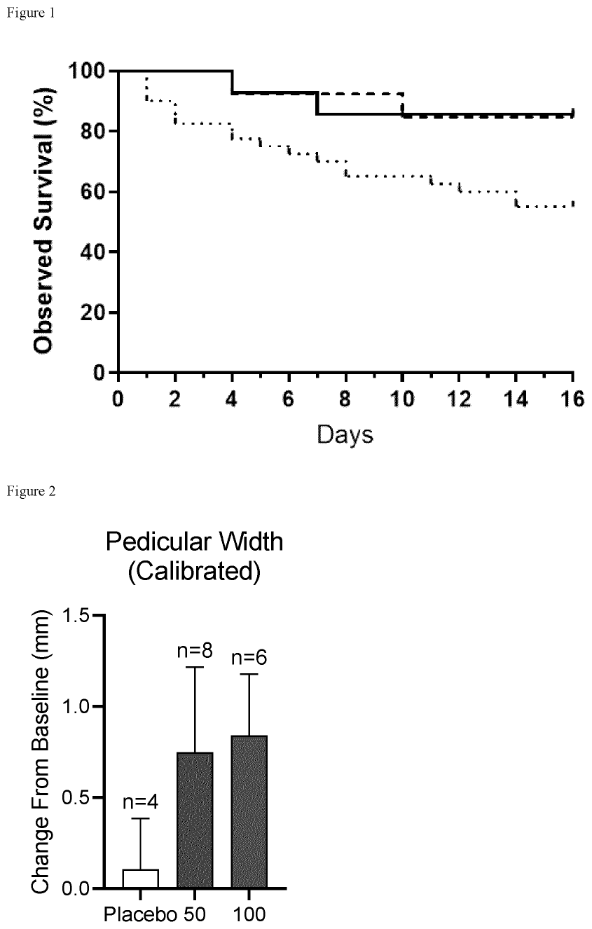

Treatment with Compound (1) was well tolerated, and no adverse clinical signs were observed during the studies. Survival of pups was monitored daily during the 15 days of treatment and registered in treatment diaries. This survival data is presented in FIG. 1. FIG. 1 also includes historic survival data (n=23) of untreated pups from the same breeding population.

FIG. 1 shows that treatment of Fgfr3^{Y367C/+} pups with Compound (1) at either dose level, significantly improved survival (P<0.05, Log-rank (Mantel-Cox) test) when compared with untreated pups (vehicle treated and historic survival combined). The increased survival rates in the treated groups were observed to be due to a marked reduction in the registered incidence of maternal infanticide. Increased incidence of maternal infanticide has been described in mouse models with muscle weakness as a major phenotypical trait (e.g., Sullivan 2014, Hum Mol Genet 23(5): 1250-1259) making it plausible that increased survival is correlated with an improved muscle strength phenotype as a result of treatment with Compound (1).

### Example 3—Phase 1 Study

To date, a Phase 1, randomized, double-blind, placebo-controlled, single-ascending dose trial in healthy adult male subjects has been completed. This trial was designed to evaluate the safety, tolerability, and pharmacokinetics of single subcutaneous unit doses of compound (1) up to 150 μg CNP/kg. Compound (1) was generally well tolerated at all investigated doses. A single dose of compound (1) delivered free CNP-38 in a dose-dependent manner with a long and dose-independent mean apparent t_{1/2} of 120 h. The most commonly reported (≥10% of subjects) treatment-emergent adverse events (TEAEs) among subjects who received compound (1) were headache, contact dermatitis, postural dizziness, (orthostatic) tachycardia, dizziness, and orthostatic hypotension. Most TEAEs were mild or moderate. There were no serious adverse effects (AEs) or deaths reported and none of the AEs were considered to be dose-limiting or led to trial discontinuation. Local tolerability findings were predominantly mild to moderate redness. Two AEs related to injection sites, mild injection site pain (10 μg CNP/kg) and mild injection site discomfort (75 μg CNP/kg) were reported. No clinically relevant dose-dependent trends were observed on blood pressure, heart rate, or safety laboratory test results at all investigated doses. Also, no evidence of immunogenic response was observed in any subject.

### Example 4—Phase 2 Study

A Phase 2, multicenter, double-blind, randomized, placebo-controlled, dose escalation trial evaluating safety, efficacy, and pharmacokinetics of subcutaneous doses of compound (1) administered once weekly for 52 weeks in prepubertal children with achondroplasia followed by an Open-Label Extension Period. The study enrolled a minimum of 60 male and female prepubertal children with ACH aged 2 to 10 years old. The clinical trial is listed on www.clinicaltrials.gov under the ClinicalTrials.gov Identifier: NCT04085523. The objectives of the study were as follow:

### Primary:

In prepubertal children with achondroplasia (ACH) at 52 weeks
To determine the safety of once weekly subcutaneous (SC) doses of compound (1)
To evaluate the effect of once weekly SC doses of compound (1) on annualized height velocity (AHV)

### Secondary:

To evaluate the effect of once weekly SC doses of compound (1) on body proportionality (upper to lower body segment ratio) in prepubertal children with ACH at 52 weeks
To evaluate the pharmacokinetic (PK) properties of once weekly SC doses of compound (1)
To assess the potential immunogenic response to once weekly SC doses of compound (1)

### Exploratory:

To evaluate the effect of once weekly SC doses of compound (1) on functional health and physical well being of treated subjects

### Inclusion Criteria

1. Clinical diagnosis of ACH with genetic confirmation
2. Age between 2 to 10 years old (inclusive) at Screening Visit
3. Prepubertal (Stage 1 breasts for girls or testicular volume<4 ml for boys) at Screening Visit
4. Able to stand without assistance
5. Caregiver willing and able to administer subcutaneous injections of study drug
6. Written, signed informed consent of the parent(s) or legal guardian(s) of the participant and written assent of the participant as required by the institutional review board/human research ethics committee/independent ethics committee (IRB/HREC/IEC)

### Exclusion Criteria

1. Clinically significant findings at Screening that:
   are expected to require surgical intervention during participation in the trial or
   are musculoskeletal in nature, such as Salter-Harris fractures and severe hip pain or
   otherwise are considered by investigator or Medical Monitor to make a participant unfit to receive study drug or undergo trial related procedures
2. Have received treatment (>3 months) of human growth hormone (hGH) or other medications known to affect stature or body proportionality at any time
3. Have received any dose of medications intended to affect stature or body proportionality within the previous 6 months of Screening Visit
4. Have received any study drug or device intended to affect stature or body proportionality at any time
5. History or presence of injury or disease of the growth plate(s), other than ACH, that affects growth potential of long bones
6. History of any bone-related surgery that affects growth potential of long bones, such as orthopedic reconstructive surgery and osteotomy (Limb-lengthening with full recovery is allowed with a minimum of 12 months of bone healing. Foramen magnum decompression and laminectomy with full recovery are allowed with minimum of 6 months of bone healing. History of 8 plate epiphysiodesis is allowed, but the plates must have been removed prior to Screening with minimum 4 weeks of healing.)
7. Have a form of skeletal dysplasia other than ACH or known medical conditions that result in short stature or abnormal growth [such as severe achondroplasia with developmental delay and acanthosis nigricans (SADDAN), hypochondroplasia, growth hormone deficiency, Turner syndrome, pseudoachondroplasia, inflammatory bowel disease, chronic renal insufficiency, active celiac disease¹, Vitamin D deficiency², untreated hypothyroidism³, poorly controlled diabetes mellitus (HbA1c≥8.0%) or diabetic complications⁴]¹Celiac disease responsive to a gluten-free diet is allowed²Vitamin D deficiency or insufficiency treated with supplementation is allowed. Vitamin D deficiency is defined as 25(OH)D level<20 ng/mL (<49.9 nmol/L), insufficiency is defined as 25(OH)D level 20-30 ng/mL (49.92-74.86 nmol/L). Participants with Vitamin D deficiency or insufficiency must be on Vitamin D regimen before randomization.³Participants with hypothyroidism must be clinically euthyroid for 3 months prior to Screening Visit and, in the opinion of the investigator, have achieved any catch-up growth expected from thyroxine replacement⁴Participants with diabetes mellitus must have been on stable medication regimen for 3 months prior to randomization (dose adjustments are allowed but addition or discontinuation of medications in this time period is disallowed)
8. History or presence of malignant disease, other than basal cell epithelioma/carcinoma or completely resected squamous skin cancer with no recurrence for 12 months per medical records
9. History or presence of the following:
   Chronic anemia (resolved iron deficiency anemia is allowed)
   Significant cardiovascular disease per the judgement of the investigator, such as congenital heart disease (uncomplicated patent ductus arteriosus and atrial or ventricular septal defect with repair are allowed), aortic insufficiency, clinically significant arrhythmias, congestive heart failure with NYHA class II and above, or other conditions that impair regulation of blood pressure or heart rate
   Condition that impacts hemodynamic stability (such as autonomic dysfunction, orthostatic intolerance)
   History of chronic renal insufficiency
   Chronic or recurrent illness that can affect hydration or volume status. This may include conditions associated with decreased nutritional intake or increased volume loss
   Bone fracture within 6 months prior to Screening Visit (within 2 months for fracture of digits)
   Any disease or condition that, in the opinion of the investigator, may make the participant unlikely to fully complete the trial, may confound interpretation of trial results, or presents undue risk from receiving study drug
10. Child has significant electrocardiogram abnormalities, including evidence of a previous myocardial infarction, left ventricular hypertrophy, flat T waves (particularly in the inferior leads) or more than minor non-specific ST-T wave changes or:
   QRS>90 milliseconds (msec)
   QT interval corrected using Fridericia's formula (QTcF)>440 msec
   PR interval>170 msec
   Complete right or left bundle branch block
11. Requires, or anticipated to require, chronic (>4 weeks) or repeated (more than twice per year) treatment with oral corticosteroids during participation in the trial (low and mid-dose inhaled corticosteroids are allowed. High-dose inhaled corticosteroids are not allowed).
12. Use of medication known to prolong the QT/QTc interval within 7 days or 5 half-lives (whichever is longer) prior to the Screening Visit.
13. Ongoing treatment with any medication that affects blood pressure or heart rate
14. Known hypersensitivity to the components of the study drug [trehalose, tris(hydroxymethyl)aminomethane, succinate and PEG]
15. Any other reason that in the opinion of the investigator would prevent the child from complying with the trial requirements or completing the trial

This trial is a multicenter trial consisting of two treatment periods. A 52 week double-blind, randomized, placebo controlled, dose escalation trial evaluating up to 5 different dose levels of weekly compound (1) administered subcutaneously in prepubertal children 2 to 10 years old, inclusive, with ACH.

TABLE 2
Unit doses of CNP

| | | Approximate Number |
|---|---|---|
| Cohort | Planned Dose Level | Of Participants |
| 1 | 6 μg CNP/kg/week | 9 on compound (1), |
| | | 3 on placebo |
| 2 | 20 μg CNP/kg/week | ≥9 on compound (1), |
| | | ≥3 on placebo |
| 3 | 50 μg CNP/kg/week | >9 on compound (1), |
| | | ≥3 on placebo |
| 4 | 100 μg CNP/kg/week | ≥9 on compound (1), |
| | | ≥3 on placebo |
| 5 | >100 μg CNP/kg/week* | ≥9 on compound (1), |
| | | ≥3 on placebo |

*At a dose to be determined, if needed, based on emerging data

TABLE 3
Baseline demographics - status end of 2021

| | Cohort 1 | Cohort 2 | Cohort 3 |
|---|---|---|---|
| Demographics | (N = 13) | (N = 15) | (N = 14) |
| Age (years) | | | |
| Mean (SD) | 6.4 (2.82) | 6.2 (3.13) | 5.6 (2.91) |
| Age Group (years), | | | |
| n (%) | | | |
| 2-5 years | 5 (38.5) | 7 (46.7) | 6 (42.9) |
| 5-8 years | 4 (30.8) | 5 (33.3) | 4 (28.6) |
| >8 years | 4 (30.8) | 3 (20.0) | 4 (28.6) |
| Sex, n (%) | | | |
| Female | 7 (53.8) | 5 (33.3) | 5 (35.7) |
| Male | 6 (46.2) | 10 (66.7) | 9 (64.3) |
| Age at ACH Diagnosis, | | | |
| n (%) | | | |
| Pre-birth | 0 | 1 (6.7) | 4 (28.6) |
| At Birth | 4 (30.8) | 3 (20.0) | 2 (14.3) |
| 0-6 months | 9 (69.2) | 9 (60.0) | 7 (50.0) |
| >6-12 months | 0 | 2 (13.3) | 1 (7.1) |
| >12 months | 0 | 0 | 0 |
| Height SDS | | | |
| Mean SD | −5.4 (1.10) | −5.0 (0.66) | −4.7 (0.82) |

Table 3 describes the baseline demographics for cohorts 1, 2 and 3, with each cohort also containing placebo subjects. Following completion of the trial and unblinding of the data, the baseline demographics for cohorts 1, 2, 3 and 4 and the placebo subjects is displayed in Table 3.

TABLE 4
Baseline demographics following unblinding of the trial

| | | | | | Pooled |
|---|---|---|---|---|---|
| | Cohort 1 | Cohort 2 | Cohort 3 | Cohort 4 | Placebo |
| Demographics | (N = 10) | (N = 11) | (N = 10) | (N = 11) | (N = 15) |
| Age (years) | | | | | |
| Mean (SD) | 6.5 (2.59) | 6.3 (2.90) | 5.2 (2.99) | 5.8 (2.61) | 5.9 (3.11) |
| Age Group (years), | | | | | |
| n (%) | | | | | |
| 2-5 years | 3 (30.0) | 5 (45.5) | 5 (50.0) | 3 (27.3) | 8 (53.3) |
| 5-8 years | 4 (40.0) | 4 (36.4) | 3 (30.0) | 6 (54.5) | 2 (13.3) |
| >8 years | 3 (30.0) | 2 (18.2) | 2 (20.0) | 2 (18.2) | 5 (33.3) |
| Sex, n (%) | | | | | |
| Female | 7 (70.0) | 3 (27.3) | 3 (30.0) | 6 (54.5) | 5 (33.3) |
| Male | 3 (30.0) | 8 (72.7) | 7 (70.0) | 5 (45.5) | 10 (66.7) |
| Age at ACH Diagnosis, | | | | | |
| n (%) | | | | | |
| Pre-birth | 0 | 1 (9.1) | 2 (20.0) | 0 | 3 (20.0) |
| At Birth | 2 (20.0) | 4 (36.4) | 2 (20.0) | 1 (9.1) | 5 (33.3) |
| 0-6 months | 8 (80.0) | 4 (36.4) | 6 (50.0) | 8 (72.7) | 7 (46.7) |
| >6-12 months | 0 | 2 (18.2) | 0 | 1 (9.1) | 0 |
| >12 months | 0 | 0 | 0 | 1 (9.1) | 0 |
| Height SDS | | | | | |
| Mean SD | −5.5 (1.05) | −4.9 (0.67) | −4.8 (0.80) | −4.9 (0.83) | −4.9 (0.96) |

### Example 5—Measurements of Vital Signs

Participants should rest for at least 5 minutes before vital sign measurement and vital signs should be taken prior to any blood draws. The following vital signs should be measured:
Heart Rate (measured in beats per minute)
Blood Pressure (seated BP will be taken measured in mm Hg)
Temperature [measured in degrees Celsius (° C.) or Fahrenheit (° F.)]

All vital signs above must be measured at all visits. Orthostatic heart rate (HR) and systolic/diastolic BP will also be measured at Visit 1 prior to administration of study drug when the participant is at rest for 5 minutes (preferably supine) and again after standing at 3 minutes for assessment of orthostatic hypotension. Orthostatic hypotension is defined as decrease in SBP of ≥20 mmHg (Stewart et al., Pediatrics, 141, 1-13, 2018). Accompanying tachycardia is defined as change of heart rate increment of ≥40 bpm and absolute orthostatic HR≥130 bpm (for ages 13 years and younger) (Singer et al., Journal of Pediatrics, 160, 222-226, 2012). Additionally, at Visit 1 and Visit 7 (in the Open-Label Extension Period), all vital signs must be monitored at 1 hour and 2 hours post injection of the study drug. For participants that undergo blood collection for pharmacokinetic analyses at 8, 24, and 48 hours following first dose (at Visit 1 in the Randomized Period and Visit 7 in the Open-Label Extension Period), orthostatic systolic/diastolic BP and HR will be measured prior to each PK blood collection.

### Example 6—Safety

In 57 patients from the trial followed for up to 65 weeks, no withdrawals or discontinuations from the trial for any reason was reported. There were no serious adverse events related to the trial drug. Also, injection site tolerability was generally very good, with a total of 13 AEs related to trial drug or procedures, including 8 injection site reactions occurring among more than 1900 injections. There were no AEs reported on orthostatism. Importantly, no treatment emergent anti-CNP antibodies were detected.

### Example 7—Pharmacokinetics Data

Sparse PK samples are collected in all subjects (if body weight allows) prior to and at 8, 24, 48 h post the initial dose and post-dose at 1 (trough), 2 (any time during the week), 3 (any time during the week), 6 (trough), 9 (any time) and 12 (trough) months+every 3 months for the duration of the study. A population PK model was used to characterize the sparse PK data. A dose related increase in exposure was observed across the four dosing cohorts, 6 μg/kg, 20 μg/kg, 50 μg/kg and 100 μg/kg. Sustained exposure to free CNP was observed, and a half-life for free CNP of ˜110 hours was estimated.

TABLE 5
Predicted median free CNP Cₘₐₓ for the subjects

| | BW 10 kg | BW 35 kg |
|---|---|---|
| Dose | Cₘₐₓ (pg/ml) | Cₘₐₓ (pg/ml) |
| μg/kg/week | Median (9-95^{th} percentile) | Median (9-95^{th} percentile) |
| 6 | 2.9 (1.6-5.0) | 1.8 (1.1-3.3) |
| 20 | 10.4 (5.8-18.9) | 6.6 (3.8-12.6) |
| 50 | 26.5 (15.2-48.1) | 18.3 (10.3-31.5) |
| 100 | 53.1 (30.9-96.4) | 36.6 (20.2-65.9) |

TABLE 6
Estimated half-lives for free CNP in the subjects

| Dose μg/kg/week | Estimated half-life (h) |
|---|---|
| 6 | 101 |
| 20 | 106 |
| 50 | 108 |
| 100 | 110 |

Blood samples for determination of CNP-38 were acidified immediately after collection, using citrate acid buffer, to stabilize the prodrug in the sample thereby avoiding further liberation of CNP-38 from the prodrug. CNP-38 was determined in plasma (heparin) following protein precipitation and solid phase extraction and analyzed using liquid chromatography and tandem mass spectrometry detection. CNP-38 (38 amino acid peptide) was used as reference material and CNP-38 labelled with deuterium (D30-CNP-38) as internal standard (IS). Positive ions were monitored in multiple-reaction monitoring mode. The quantification was performed using the peak area ratios of analyte versus IS. The calibration curve fitting was done by 1/concentration-weighted linear regression. The calibration range in non-acidified plasma was 1.38 to 138 pmol/l.

TABLE 7
Preliminary mean free CNP Levels from NCT04085523

| | | | | TIME | MEAN | SD | |
|---|---|---|---|---|---|---|---|
| COHORT | DOSE | | VISIT | (days) | (pmol/L) | (pmol/L) | NOBS |
| 1 | 6 | μg/kg | WEEK 0 | 0.96 | 1.86 | 1.12 | 17 |
| 1 | 6 | μg/kg | WEEK 4 | 27.98 | 1.38 | 0.00* | 7 |
| 1 | 6 | μg/kg | WEEK 8 | 55.97 | 1.68 | 0.46 | 7 |
| 1 | 6 | μg/kg | WEEK 12 | 79.02 | 1.85 | 0.67 | 8 |
| 1 | 6 | μg/kg | WEEK 26 | 181.99 | 1.48 | 0.19 | 7 |
| 1 | 6 | μg/kg | WEEK 39 | 271.94 | 2.02 | 0.58 | 8 |
| 1 | 6 | μg/kg | WEEK 52 | 363.90 | 1.58 | 0.39 | 7 |
| 2 | 20 | μg/kg | WEEK 0 | 1.01 | 3.49 | 1.51 | 22 |
| 2 | 20 | μg/kg | WEEK 4 | 27.94 | 3.27 | 2.18 | 10 |
| 2 | 20 | μg/kg | WEEK 8 | 56.27 | 3.79 | 1.63 | 9 |
| 2 | 20 | μg/kg | WEEK 12 | 84.00 | 3.84 | 3.15 | 9 |
| 2 | 20 | μg/kg | WEEK 26 | 182.00 | 2.77 | 1.49 | 11 |
| 2 | 20 | μg/kg | WEEK 39 | 273.47 | 5.19 | 3.58 | 8 |
| 3 | 50 | μg/kg | WEEK 0 | 1.00 | 10.58 | 3.59 | 15 |
| 3 | 50 | μg/kg | WEEK 4 | 27.99 | 7.29 | 2.49 | 10 |
| 3 | 50 | μg/kg | WEEK 8 | 56.00 | 9.24 | 5.31 | 9 |
| 3 | 50 | μg/kg | WEEK 12 | 83.91 | 7.62 | 3.88 | 10 |
| 4 | 100 | μg/kg | WEEK 0 | 0.99 | 15.29 | 7.00 | 18 |

Observations below the lower limit of quantification (LLOQ) have been set to the LLOQ (1.38 pM)
*All observations below LLOQ
NOBS: number of observations at the given week
Time (days): the median time post the 1^{st} dose
Placebo subjects not included

### Example 8—Neutralizing Antibodies

All subjects in the trial are being monitored for binding and, if relevant, neutralizing antibodies to CNP. No anti-CNP binding antibodies have been detected upon 1 to 9 months of repeated weekly exposure to compound (1). Samples are collected in all subjects at screening visit, prior to initial dose, and post-dose at 1, 3, 6, 9 months+every 3 months for the duration of the study. Samples are analyzed using a fully validated anti-CNP antibody assay. All post-dose samples have been confirmed negative for anti-CNP antibodies.

### Assay Description

A sensitive assay was developed to detect, confirm and quasi-quantify binding antibodies against CNP-38 (and CNP-22) and was designed as a bridging ECL (electro-chemiluminescence) immunoassay. Prior to analysis of clinical samples, the assay was fully validated following regulatory requirements from both EMAA and FDA. In this approach, biotinylated and ruthenylated CNP-38 were prepared as a master mix. Samples were incubated at minimum required dilution (MRD) in the master mix to form complex and then plated on an MSD streptavidin plate where the biotinylated CNP-38 could also bind. The samples were then detected by the ruthenium labelled CNP-38 within the bound complexes, using an MSD S600 plate reader. The presence of anti-drug antibody present was determined by comparing the signal to a statistically derived threshold, the assay cut point.

Height of subjects enrolled in the study of Example 3 was measured using a wall-mounted stadiometer. The same equipment was used at all visits for the same child. All instruments were calibrated as required by local requirements or per instrument manufacturer guidance prior to use. These data demonstrate that weekly subcutaneous treatment of achondroplasia patients aged 2 to 10 years with compound (1) at 100 ug CNP-38/kg for a period of 52 weeks or greater has a beneficial effect on bone growth and corresponds to an annualized growth velocity of 5.42 cm/year, which is significantly higher than the annual growth velocity in the placebo treated cohort of 4.35 cm/year (p=0.0218).

TABLE 8
Superiority of compound (1) 100 μg CNP-38/kg/week
in AGV compared to placebo

| Treatment Group | AGV (cm/year), n | p-value |
|---|---|---|
| Compound (1) | LS Mean | Compound (1) vs. |
| or Placebo | [95% CI] | Pooled Placebo) |
| 6 μg/kg/week | 4.09, n = 10 | 0.6004 |
| | [3.34, 4.84] | |
| 20 μg/kg/week | 4.52, n = 11 | 0.7022 |
| | [3.82, 5.22] | |
| 50 μg/kg/week | 5.16, n = 10 | 0.0849 |
| | [4.43, 5.90] | |
| 100 μg/kg/week | 5.42, n = 11 | 0.0218 |
| | [4.74, 6.11] | |
| Pooled Placebo | 4.35, n = 15 | N/A |
| | [3.75, 4.94] | |

Also, for the 100 μg CNP-38/kg/week group, a tendency in the acceleration in AGV was observed in the time period 6 to 12 months, compared to the AGV in 0 to 6 months with a AGV of 5.696 and 5.167 cm/year, respectively.

It was also evident that there was a comparable AGV across age groups that was dose dependent (Table 9).

TABLE 9
Comparable AGV across age groups

| | Age <5 years old | Age ≥5 years old |
|---|---|---|
| | AGV (cm/year), n | AGV (cm/year), n |
| Treatment Group | LS Mean, | LS Mean. |
| compound (1) | [95% CI] | [95% CI] |
| 6 μg/kg/week | 4.31, n = 3 | 3.79, n = 7 |
| | [2.52, 6.10] | [2.87, 4.71] |
| 20 μg/kg/week | 4.72, n = 5 | 4.29, n = 6 |
| | [3.30, 6.15] | [3.43, 5.15] |
| 50 μg/kg/week | 5.07, n = 5 | 5.33, n = 5 |
| | [3.62, 6.52] | [4.39, 6.26] |
| 100 μg/kg/week | 5.95, n = 3 | 5.12, n = 8 |
| | [4.03, 7.87] | [4.35, 5.90] |
| Pooled Placebo | 4.53 , n = 8 | 4.29, n = 7 |
| | [3.43, 5.63] | [3.44, 5.14] |

Furthermore, compound (1) administered at 100 μg CNP-38/kg/week demonstrated superiority in achondroplasia specific change in height SDS compared to placebo. A description of achondroplasia specific change in height SDS has previously been disclosed (Orphanet J. Rare Dis. 2021; 16(1): 522).

TABLE 10

| Treatment Group | A Height SDS*, n | p-value |
|---|---|---|
| (compound (1) | LS Mean | (compound (1) vs. |
| Dose Levels or Placebo) | [95% CI] | Pooled Placebo) |
| 6 μg/kg/week | −0.04, n = 10 | 0.8207 |
| | [−0.26, 0.17] | |
| 20 μg/kg/week | 0.03, n = 11 | 0.4107 |
| | [−0.17, 0.23] | |
| 50 μg/kg/week | 0.11, n = 10 | 0.1660 |
| | [−0.10, 0.32] | |
| 100 μg/kg/week | 0.22, n = 11 | 0.0283 |
| | [0.02, 0.41] | |
| Pooled Placebo | −0.08, n = 15 | N/A |
| | [-0.25, 0.10] | |

*ACH-specific height SDS

TABLE 11
Overview of treatment-emergent adverse events

| | 6 | 20 | 50 | 100 | Total |
|---|---|---|---|---|---|
| | μg/kg/week | μg/kg/week | μg/kg/week | μ/kg/week | Placebo |
| | (n = 10) | (n = 11) | (n = 10) | c (n = 11) | (n = 15) |
| Subject with | 9 (90.0) | 11 (100.0) | 10 (100.0) | 10 (90.9) | 14 (93.3) |
| Treatment- | | | | | |
| Emergent Adverse | | | | | |
| Events (TEAE)* | | | | | |
| Grade 1 | 9 (90.0) | 11 (100.0) | 10 (100.0) | 9 (81.8) | 14 (93.3) |
| Grade 2 | 3 (30.0) | 3 (27.3) | 3 (30.0) | 1 (9.1) | 5 (33.3) |
| Serious TEAE | 1 (10.0) | 0 | 1 (10.0) | 0 | 0 |
| Treatment-Related | 3 (30.0) | 2 (18.2) | 3 (30.0) | 2 (18.2) | 5 (33.3) |
| TEAE | | | | | |
| Achondroplasia- | 3 (30.0) | 4 (36.4) | 5 (50.0) | 1 (9.1) | 9 (60.0) |
| Related TEAE** | | | | | |

*No reported Grade 3 (severe) or Grade 4 (life-threatening) TEAEs.
**Adverse events reported by investigator as related to underlying disease

Surprisingly, treatment with compound (1) was associated with a reduction in the reporting of achondroplasia related treatment emergent adverse events from 60% in the placebo group to 9.1% in the 100 μg/kg/week dose group.

TABLE 12
Treatment-Related Adverse Events

| | 6 | 20 | 50 | 100 | Total |
|---|---|---|---|---|---|
| | μg/kg/week | μg/kg/week | μg/kg/week | μg/kg/week | Placebo |
| | (n = 10) | (n = 11) | (n = 10) | (n = 11) | (n = 15) |
| Subjects with at | 3 (30.0) | 2 (18.2) | 3 (30.0) | 2 (18.2) | 5 (33.3) |
| Least | | | | | |
| One Treatment- | | | | | |
| Related TEAE | | | | | |
| Injection site | 2 (20.0) | 1 (9.1) | 3 (30.0) | 2(18.2) | 2 (13.3) |
| reaction* | | | | | |
| Abdominal pain | 0 | 1 (9.1) | 0 | 0 | 0 |
| upper | | | | | |
| Overdose | 0 | 0 | 0 | 0 | 1 (6.7) |
| Dizziness | 0 | 0 | 0 | 0 | 1 (6.7) |
| Sleep terror | 0 | 0 | 0 | 0 | 1 (6.7) |
| Urticaria | 1 (10.0) | 0 | 0 | 0 | 0 |

*Injection site reactions includes preferred terms of injection site reaction, injection site pain, injection site erythema, injectio site discoloration, injection site haemorrage and injection site swelling.

Injections with compound (1) were well tolerated with a low frequency of injection site reactions (ISR). Only 11 ISRs in 8 patients occurred with more than 2,000 injections administered. This was comparable to the event rate in the placebo group with 2 subjects reporting each 1 ISR. All of these were reported as mild in severity.

### Example 9—Pharmacokinetics Data and Determination of Minimum Sustained Free CNP Exposure

During the trial, sparse blood samples were collected at different time points in the dosing interval, and the concentration of free CNP-38 was determined using the methodology described in example 6. Sub-setting the blood samples taken at 6.5 to 7.5 days after last dose, the range of free CNP-38 plasma concentration is shown in Table 11 (PK data cut off: 15 Nov. 2022). For weekly administration of compound (1), this therefore represents the sustained minimum plasma free CNP-38 concentration.

TABLE 12

| Dose CNP | Free CNP-38 | Free CNP-38 |
|---|---|---|
| μg/kg/week | Median (range) pmol/L | Mean (SD) pmol/L |
| 6 | 1.7 (1.4-1.9) | 1.7 (0.2) |
| 20 | 2.9 (1.6-4.2) | 2.9 (9.7) |
| 50 | 6.5 (4-16.9) | 7.2 (2.5) |
| 100 | 13.8 (9.4-23) | 14 (4.2) |
| 150 | 22.3 (19.7-25) | — |

The values are provided as pmol/L CNP-38. Free CNP values may be calculated by a multiplication factor of about 30 to about 50%, to take into account of degradation products of free CNP-38 which remain functional, and native CNP. Values for the 150 μg/kg/week dose are simulated with a population PK model for compound (1). The lower value in the range is the lowest free CNP-38 concentration, also referred to as the trough level or trough concentration. The number of subjects contributing with observations at the specified time interval are: 4, 8, 10 and 6 for the 6, 20, 50 and 100 μg/kg dose groups, respectively.

### Example 10 SF-10 Health Survey

SF-10τH Health Survey for Children is a 10-item parent-completed survey that covers a wide range of domains affecting a child's functional health and well-being (QualityMetric.com), with 5 items primarily related to physical health (PhS), and 5 items primarily related to psyhocosial health (PsS).

Subjects under the Phase 2 study described in Example 4 above, randomised to placebo or treatment with compound (1) at a weekly SC dose of 100 μg/kg, were assessed using the the Short-Form 10 (SF-10) parent or caregiver-completed survey. The survey was conducted at the following time points; baseline and week 52.

SF-10 assesses, inter alia, physical health (PhS). 5 items of the SF-10 were selected for the assessment of physical health parameters as follows:
Limited in things that take some energy
Limited in bending, lifting, stooping
Limited in kind of schoolwork/activities due to physical health
Subjects health in general
How much bodily pain or discomfort

For each response provided by a subject, a value is assigned and used to calculate the overall physical summary score in accordance with established guidelines (See The SF-10 Health Survey for Children User's Guide by QualityMetric). For reference, in accordance with the User's Guide, the SF-10 scale scores were centered so that a score of 50 corresponds to an average score in a 2006 sample (a combination of general population and supplemental disability and chronic condition samples). Table 13 sets out the scores obtained for the different groups.

TABLE 13
SF-10 Physical Summary Scores

| Patient Group | Baseline (S.E.) | Wk 52 (S.E) | Change from Baseline |
|---|---|---|---|
| Compound (1) | 34.80 (6.95) | 40.70 (6.87) | +5.91 (1.65) |
| 100 μg/kg | | | |
| (SC) | | | |
| Placebo | 40.52 (3.88) | 37.48 (4.07) | −3.04 (3.82) |

S.E.—Standard Error

While the subject group scored lower than the population average, both at baseline and at week 52, there was a marked increase in the PhS score in the group treated with compound (1) when compared with the score at baseline. In contrast, the untreated group showed a decreased score at week 52 relative to the score at baseline. Together these data indicates that treatment with compound (1) has a positive impact on the physical health criteria assessed in the SF-10 survey. The SF-10 survey PsS score assesses the childs self esteem, mental health and behaviour. Prior to and after 52 weeks of treatment, for both placebo and treated groups the PsS scores were within or slightly above the normal range, however there was an improvement trend in the PsS score in the group treated with compound (1) after 52 weeks of treatment, whilst the placebo group saw a reduction in the PsS score, indicating that CNP treatment provided an improvement in psychosocial health, and an overall improvement in the quality of life. The improvement in physical health and psycohosocial health was seen across the treatment cohort and was more pronounced in the children 5 years and older.

### Example 11 Clinical Observations of Phase 2 Study Subjects

Subjects under the Phase 2 study described in Example 4 above, were observed over the 52-week, double-blinded study period, for detectable changes in physical health. Although no formal qualitative or quantitative measurement were made, investigators throughout this period reported the following improvements in the physical health of patients participating in the study:
Improvements in muscle strength and endurance
Improvements in posture, core strength and the ability to stand straight and lift
Improvements in ability to sit straight
Improvements in ability to stand for prolonged periods
Reduced complaints of pain with one patient claiming back pain had disappeared
Improvement observed from spinal X-ray

### Example 12. Baseline Demographics Prior to First Dose: Medical History Complications

Fifty-seven children (n=24 female) were enrolled in the ACcomplisH phase 2 trial from North America, Europe, and Oceania. The mean±SD age at enrollment was 5.9±2.8 year. The most prevalent medical history complications reported by age group prior to treatment by age group are shown in Table 14:

TABLE 14

| | All | | | Forman | |
|---|---|---|---|---|---|
| | com- | Sleep | Ear | Magnum | |
| | plications | Apnea | Infection | Stenosis | Kyphosis |
| All patients | 50 | 31 | 24 | 9 | 9 |
| (N = 57) | | | | | |
| ≤5 years | 20 | 17 | 6 | 6 | 5 |
| (N = 24) | | | | | |
| >5-8 years | 16 | 8 | 10 | 2 | 3 |
| (N = 19) | | | | | |
| >8 years | 14 | 5 | 8 | 1 | 1 |
| (N = 14) | | | | | |
| Cohort 1 | 12 | 9 | 5 | 1 | 2 |
| N = 13 | | | | | |
| Cohort 2 | 12 | 5 | 9 | 3 | 2 |
| N = 15 | | | | | |
| Cohort 3 | 14 | 9 | 5 | 3 | 1 |
| N = 14 | | | | | |
| Cohort 4 | 12 | 8 | 5 | 3 | 4 |
| N = 15 | | | | | |

### Example 13

In the phase 2 study reported in Example 4, the incidence of scoliosis and kyphosis in the placebo group (6 patients assessed), cohort 3, 50 μg/kg (9 patients assessed) and cohort 4, 100 ug/kg (9 patients assessed) was assessed at baseline (prior to treatment), and after 52 weeks of treatment. The incidence results for scoliosis are presented in Table 15. The incidence of scoliosis in cohort 3 and cohort 4 reduced after 52 weeks of treatment, and the initial data indicates a trend for a dose dependent reduction in the scoliosis main thoracic Cobb Angle. A similar assessment for kyphosis was also made at baseline and after 52 weeks of treatment, and the initial data is also consistent with a trend for a dose dependent reduction in kyphosis Cobb Angle.

TABLE 15
Overall Scoliosis Incidence

| | | After 52 weeks |
|---|---|---|
| Group | Baseline | of Treatment |
| Placebo | 1 out of 6 | 1 out of 6 |
| Cohort 3 | 3 out of 9 | 0 out of 9 |
| Cohort 4 | 3 out of 9 | 1 out of 9 |

### Example 14

In the phase 2 study reported in Example 4, the interpedicular distance (IPD) and pedicular width (PW) was calculated from AP X-rays (IPD) and lateral X-rays (PW) taken from the placebo group (4 patients assessed), cohort 3, 50 μg/kg (8 patients assessed) and cohort 4, 100 ug/kg (6 patients assessed) at baseline and after 52 weeks of treatment. Whereas there was no clear effect on IPD, there was a trend for patients on treatment to have an increase in pedicular width, as illustrated in FIG. 2.

### Example 15

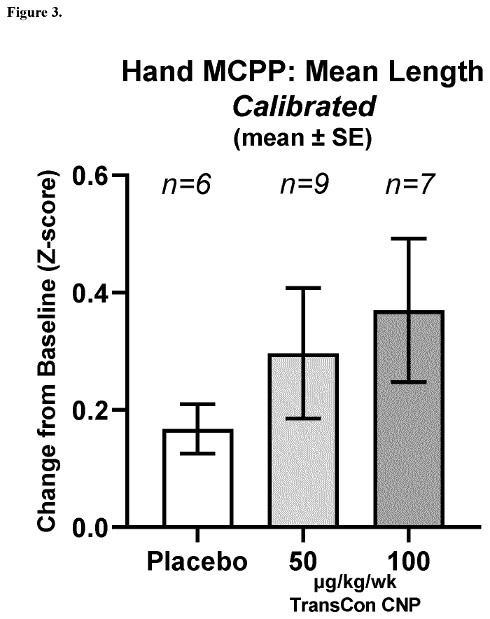

In the phase 2 study reported in Example 4, x-rays of the hand were taken at the placebo group (6 patients assessed), cohort 3, 50 μg/kg (9 patients assessed) and cohort 4, 100 ug/kg (7 patients assessed) and the Hand Metacarpal/Phalangeal Profile (MCPP) was assessed. The results are shown in FIG. 3. The results indicate a clear trend for dose dependent increase in the hand length on treatment.

1. A method of improving muscle function in a subject suffering from a disease or condition in which muscle function is impaired, the method comprising administering a therapeutically effective amount of an inhibitor of FGFR3 signaling, an NPR-B agonist or an NPR-C agonist to the subject.

2. The method according to claim 1, wherein the inhibitor of FGFR3 signaling is an FGFR3 antagonist or an NPR-B agonist.

3. The method according to claim 1, wherein the NPR-B agonist is or comprises a C-type natriuretic peptide (CNP).

4. The method according to claim 1, wherein the NPR-C agonist is or comprises a C-type natriuretic peptide (CNP).

5. The method according to claim 1, wherein the NPR-B agonist or NPR-C agonist is administered as a CNP conjugate or a pharmaceutically acceptable salt thereof.

6. The method according to claim 1, wherein the NPR-B agonist or NPR-C agonist is a prodrug of CNP.

7. The method according to claim 1, wherein the CNP conjugate or prodrug of CNP is a compound of formula (If′), formula (IIf), compound (1), or a pharmaceutically acceptable salt thereof.

8. The method according to claim 1, wherein the method comprises administering successive doses of a therapeutically effective amount of the CNP conjugate and/or prodrug of CNP to the subject, wherein the sustained exposure of free CNP in the blood plasma of the patient between successive doses of the therapeutically effective amount of the CNP conjugate and/or prodrug of CNP is at least about 1 pmol/L, such as at least about 4 pmol/L, such as between about 4 pmol/L to about 30 pmol/L.

9. The method according to claim 8, wherein the method comprises administering successive doses of a therapeutically effective amount of the CNP conjugate and/or prodrug of CNP to the subject, wherein the sustained exposure of free CNP in the blood plasma of the patient between successive doses of the therapeutically effective amount of the CNP conjugate and/or prodrug of CNP is at least about 9 pmol/L.

10. The method according to claim 8, wherein the therapeutically effective amount of the CNP conjugate and/or prodrug are administered daily or weekly.

11. The method according to claim 3, comprising administering an infusion, such as a i.v. or s.c. infusion, of the CNP to the subject, wherein the infusion results in a sustained exposure of free CNP in the blood plasma of the patient over a period of at least 1 hour of at least 1 pmol/L, such as at least about 4 pmol/L, such as at least about 9 pmol/L or about 4 pmol/L to about 30 pmol/L.

12. The method according to claim 1, wherein the NPR-B agonist is or comprises vosoritide (SEQ ID NO:30).

13. The method according to claim 2, wherein the FGFR3 antagonist is an FGFR3 tyrosine kinase inhibitor.

14. The method according to claim 13, wherein the FGFR3 tyrosine kinase inhibitor is selected from the group consisting of infigratinib, pemigatinib, futibatinib, erdafitinib and TYRA-300.

15. The method according to claim 1, wherein the subject is a human subject.

16. The method according to claim 15, wherein the subject is less than 18 years of age.

17. The method according to claim 15, wherein the subject is at least 18 years of age.

18. The method according to claim 1, wherein the subject has closed bone epiphysis.

19. The method according to claim 1, wherein the improvement in muscle function is one or more of:
a) increased skeletal muscle strength,
b) increased skeletal muscle tone,
c) increased skeletal muscle stamina,
d) increased skeletal muscle mass,
e) decreased skeletal muscle fatigue,
f) increased cardiovascular endurance,
g) increased cardiovascular fitness,
h) decreased exercise intolerance,
i) increased exercise capacity,
j) decreased exercise induced fatigue, and
k) decreased hypotonia.

20. The method according to claim 1, wherein the administration gives rise to increased skeletal muscle mass and/or muscle/fat ratio (such as skeletal muscle/fat ratio), in the subject.

21. The method according to claim 1, wherein the subject has hypotonia.

22-91. (canceled)


SEQUENCE LISTING (105 sequences)

SEQ ID NO: 1 - protein, 22 aa, Homo sapiens
  DISULFID 6..22: note = intrachain disulfide bond
GLSKGCFGLKLDRIGSMSGLGC

SEQ ID NO: 2 - protein, 53 aa, Homo sapiens
  DISULFID 37..53: note = intrachain disulfide bond
DLRVDTKSRAAWARLLQEHPNARKYKGANKKGLSKGCFGLKLDRIGSMSGLGC

SEQ ID NO: 3 - protein, 54 aa, synthetic construct
  REGION 1..54: note = G-CNP-53
  DISULFID 38..54: note = intrachain disulfide bond
GDLRVDTKSRAAWARLLQEHPNARKYKGANKKGLSKGCFGLKLDRIGSMSGLGC

SEQ ID NO: 4 - protein, 54 aa, synthetic construct
  REGION 1..54: note = M-CNP-53
  DISULFID 38..54: note = intrachain disulfide bond
MDLRVDTKSRAAWARLLQEHPNARKYKGANKKGLSKGCFGLKLDRIGSMSGLGC

SEQ ID NO: 5 - protein, 54 aa, synthetic construct
  REGION 1..54: note = P-CNP-53
  DISULFID 38..54: note = intrachain disulfide bond
PDLRVDTKSRAAWARLLQEHPNARKYKGANKKGLSKGCFGLKLDRIGSMSGLGC

SEQ ID NO: 6 - protein, 53 aa, synthetic construct
  REGION 1..53: note = CNP-53 M48N
  DISULFID 37..53: note = intrachain disulfide bond
DLRVDTKSRAAWARLLQEHPNARKYKGANKKGLSKGCFGLKLDRIGSNSGLGC

SEQ ID NO: 7 - protein, 36 aa, synthetic construct
  REGION 1..36: note = CNP-53 with deletion of amino acids 15-31
  DISULFID 20..36: note = intrachain disulfide bond
DLRVDTKSRAAWARGLSKGCFGLKLDRIGSMSGLGC

SEQ ID NO: 8 - protein, 52 aa, synthetic construct
  REGION 1..52: note = CNP-52
  DISULFID 36..52: note = intrachain disulfide bond
LRVDTKSRAAWARLLQEHPNARKYKGANKKGLSKGCFGLKLDRIGSMSGLGC

SEQ ID NO: 9 - protein, 51 aa, synthetic construct
  REGION 1..51: note = CNP-51
  DISULFID 35..51: note = intrachain disulfide bond
RVDTKSRAAWARLLQEHPNARKYKGANKKGLSKGCFGLKLDRIGSMSGLGC

SEQ ID NO: 10 - protein, 50 aa, synthetic construct
  REGION 1..50: note = CNP-50
  DISULFID 34..50: note = intrachain disulfide bond
VDTKSRAAWARLLQEHPNARKYKGANKKGLSKGCFGLKLDRIGSMSGLGC

SEQ ID NO: 11 - protein, 49 aa, synthetic construct
  REGION 1..49: note = CNP-49
  DISULFID 33..49: note = intrachain disulfide bond
DTKSRAAWARLLQEHPNARKYKGANKKGLSKGCFGLKLDRIGSMSGLGC

SEQ ID NO: 12 - protein, 48 aa, synthetic construct
  REGION 1..48: note = CNP-48
  DISULFID 32..48: note = intrachain disulfide bond
TKSRAAWARLLQEHPNARKYKGANKKGLSKGCFGLKLDRIGSMSGLGC

SEQ ID NO: 13 - protein, 47 aa, synthetic construct
  REGION 1..47: note = CNP-47
  DISULFID 31..47: note = intrachain disulfide bond
KSRAAWARLLQEHPNARKYKGANKKGLSKGCFGLKLDRIGSMSGLGC

SEQ ID NO: 14 - protein, 46 aa, synthetic construct
  REGION 1..46: note = CNP-46
  DISULFID 30..46: note = intrachain disulfide bond
SRAAWARLLQEHPNARKYKGANKKGLSKGCFGLKLDRIGSMSGLGC

SEQ ID NO: 15 - protein, 45 aa, synthetic construct
  REGION 1..45: note = CNP-45
  DISULFID 29..45: note = intrachain disulfide bond
RAAWARLLQEHPNARKYKGANKKGLSKGCFGLKLDRIGSMSGLGC

SEQ ID NO: 16 - protein, 44 aa, synthetic construct
  REGION 1..44: note = CNP-44
  DISULFID 28..44: note = intrachain disulfide bond
AAWARLLQEHPNARKYKGANKKGLSKGCFGLKLDRIGSMSGLGC

SEQ ID NO: 17 - protein, 35 aa, synthetic construct
  REGION 1..35: note = CNP-44 with a deletion of amino acids 14-22
  DISULFID 19..35: note = intrachain disulfide bond
AAWARLLQEHPNAGLSKGCFGLKLDRIGSMSGLGC

SEQ ID NO: 18 - protein, 36 aa, synthetic construct
  REGION 1..36: note = CNP-44 with a deletion of amino acids 15-22
  DISULFID 20..36: note = intrachain disulfide bond
AAWARLLQEHPNARGLSKGCFGLKLDRIGSMSGLGC

SEQ ID NO: 19 - protein, 43 aa, synthetic construct
  REGION 1..43: note = CNP-43
  DISULFID 27..43: note = intrachain disulfide bond
AWARLLQEHPNARKYKGANKKGLSKGCFGLKLDRIGSMSGLGC

SEQ ID NO: 20 - protein, 42 aa, synthetic construct
  REGION 1..42: note = CNP-42
  DISULFID 26..42: note = intrachain disulfide bond
WARLLQEHPNARKYKGANKKGLSKGCFGLKLDRIGSMSGLGC

SEQ ID NO: 21 - protein, 41 aa, synthetic construct
  REGION 1..41: note = CNP-41
  DISULFID 25..41: note = intrachain disulfide bond
ARLLQEHPNARKYKGANKKGLSKGCFGLKLDRIGSMSGLGC

SEQ ID NO: 22 - protein, 40 aa, synthetic construct
  REGION 1..40: note = CNP-40
  DISULFID 24..40: note = intrachain disulfide bond
RLLQEHPNARKYKGANKKGLSKGCFGLKLDRIGSMSGLGC

SEQ ID NO: 23 - protein, 39 aa, synthetic construct
  REGION 1..39: note = CNP-39
  DISULFID 23..39: note = intrachain disulfide bond
LLQEHPNARKYKGANKKGLSKGCFGLKLDRIGSMSGLGC

SEQ ID NO: 24 - protein, 38 aa, synthetic construct
  REGION 1..38: note = CNP-38
  DISULFID 22..38: note = intrachain disulfide bond
LQEHPNARKYKGANKKGLSKGCFGLKLDRIGSMSGLGC

SEQ ID NO: 25 - protein, 37 aa, synthetic construct
  REGION 1..37: note = CNP-37
  DISULFID 21..37: note = intrachain disulfide bond
QEHPNARKYKGANKKGLSKGCFGLKLDRIGSMSGLGC

SEQ ID NO: 26 - protein, 37 aa, synthetic construct
  REGION 1..37: note = CNP-37 mit Q1pQ (pQ = pyroglutamate)
  SITE 1: note = MISC_FEATURE - X = pyroglutamate
  DISULFID 21..37: note = intrachain disulfide bond
XEHPNARKYKGANKKGLSKGCFGLKLDRIGSMSGLGC

SEQ ID NO: 27 - protein, 38 aa, synthetic construct
  REGION 1..38: note = G-CNP-37
  DISULFID 22..38: note = intrachain disulfide bond
GQEHPNARKYKGANKKGLSKGCFGLKLDRIGSMSGLGC

SEQ ID NO: 28 - protein, 38 aa, synthetic construct
  REGION 1..38: note = P-CNP-37
  DISULFID 22..38: note = intrachain disulfide bond
PQEHPNARKYKGANKKGLSKGCFGLKLDRIGSMSGLGC

SEQ ID NO: 29 - protein, 38 aa, synthetic construct
  REGION 1..38: note = M-CNP-37
  DISULFID 22..38: note = intrachain disulfide bond
MQEHPNARKYKGANKKGLSKGCFGLKLDRIGSMSGLGC

SEQ ID NO: 30 - protein, 39 aa, synthetic construct
  REGION 1..39: note = PG-CNP-37
  DISULFID 23..39: note = intrachain disulfide bond
PGQEHPNARKYKGANKKGLSKGCFGLKLDRIGSMSGLGC

SEQ ID NO: 31 - protein, 39 aa, synthetic construct
  REGION 1..39: note = MG-CNP-37
  DISULFID 23..39: note = intrachain disulfide bond
MGQEHPNARKYKGANKKGLSKGCFGLKLDRIGSMSGLGC

SEQ ID NO: 32 - protein, 37 aa, synthetic construct
  REGION 1..37: note = CNP-37 M32N
  DISULFID 21..37: note = intrachain disulfide bond
QEHPNARKYKGANKKGLSKGCFGLKLDRIGSNSGLGC

SEQ ID NO: 33 - protein, 38 aa, synthetic construct
  REGION 1..38: note = G-CNP-37 M32N
  DISULFID 22..38: note = intrachain disulfide bond
GQEHPNARKYKGANKKGLSKGCFGLKLDRIGSNSGLGC

SEQ ID NO: 34 - protein, 38 aa, synthetic construct
  REGION 1..38: note = G-CNP-37 K14Q
  DISULFID 22..38: note = intrachain disulfide bond
GQEHPNARKYKGANQKGLSKGCFGLKLDRIGSMSGLGC

SEQ ID NO: 35 - protein, 38 aa, synthetic construct
  REGION 1..38: note = G-CNP-37 K14P
  DISULFID 22..38: note = intrachain disulfide bond
GQEHPNARKYKGANPKGLSKGCFGLKLDRIGSMSGLGC

SEQ ID NO: 36 - protein, 37 aa, synthetic construct
  REGION 1..37: note = G-CNP-37 K14Q, deletion of amino acid 15
  DISULFID 21..37: note = intrachain disulfide bond
GQEHPNARKYKGANQGLSKGCFGLKLDRIGSMSGLGC

SEQ ID NO: 37 - protein, 38 aa, synthetic construct
  REGION 1..38: note = G-CNP-37 K14Q, K15Q
  DISULFID 22..38: note = intrachain disulfide bond
GQEHPNARKYKGANQQGLSKGCFGLKLDRIGSMSGLGC

SEQ ID NO: 38 - protein, 36 aa, synthetic construct
  REGION 1..36: note = CNP-36
  DISULFID 20..36: note = intrachain disulfide bond
EHPNARKYKGANKKGLSKGCFGLKLDRIGSMSGLGC

SEQ ID NO: 39 - protein, 35 aa, synthetic construct
  REGION 1..35: note = CNP-35
  DISULFID 19..35: note = intrachain disulfide bond
HPNARKYKGANKKGLSKGCFGLKLDRIGSMSGLGC

SEQ ID NO: 40 - protein, 34 aa, synthetic construct
  REGION 1..34: note = CNP-34
  DISULFID 18..34: note = intrachain disulfide bond
PNARKYKGANKKGLSKGCFGLKLDRIGSMSGLGC

SEQ ID NO: 41 - protein, 33 aa, synthetic construct
  REGION 1..33: note = CNP-33
  DISULFID 17..33: note = intrachain disulfide bond
NARKYKGANKKGLSKGCFGLKLDRIGSMSGLGC

SEQ ID NO: 42 - protein, 32 aa, synthetic construct
  REGION 1..32: note = CNP-32
  DISULFID 16..32: note = intrachain disulfide bond
ARKYKGANKKGLSKGCFGLKLDRIGSMSGLGC

SEQ ID NO: 43 - protein, 31 aa, synthetic construct
  REGION 1..31: note = CNP-31
  DISULFID 15..31: note = intrachain disulfide bond
RKYKGANKKGLSKGCFGLKLDRIGSMSGLGC

SEQ ID NO: 44 - protein, 30 aa, synthetic construct
  REGION 1..30: note = CNP-30
  DISULFID 14..30: note = intrachain disulfide bond
KYKGANKKGLSKGCFGLKLDRIGSMSGLGC

SEQ ID NO: 45 - protein, 29 aa, synthetic construct
  REGION 1..29: note = CNP-29
  DISULFID 13..29: note = intrachain disulfide bond
YKGANKKGLSKGCFGLKLDRIGSMSGLGC

SEQ ID NO: 46 - protein, 28 aa, synthetic construct
  REGION 1..28: note = CNP-28
  DISULFID 12..28: note = intrachain disulfide bond
KGANKKGLSKGCFGLKLDRIGSMSGLGC

SEQ ID NO: 47 - protein, 38 aa, synthetic construct
  REGION 1..38: note = GHKSEVAHRF-CNP-28
  DISULFID 22..38: note = intrachain disulfide bond
GHKSEVAHRFKGANKKGLSKGCFGLKLDRIGSMSGLGC

SEQ ID NO: 48 - protein, 27 aa, synthetic construct
  REGION 1..27: note = CNP-27
  DISULFID 11..27: note = intrachain disulfide bond
GANKKGLSKGCFGLKLDRIGSMSGLGC

SEQ ID NO: 49 - protein, 27 aa, synthetic construct
  REGION 1..27: note = CNP-27 K4Q, K5Q
  DISULFID 11..27: note = intrachain disulfide bond
GANQQGLSKGCFGLKLDRIGSMSGLGC

SEQ ID NO: 50 - protein, 27 aa, synthetic construct
  REGION 1..27: note = CNP-27 K4R, K5R
  DISULFID 11..27: note = intrachain disulfide bond
GANRRGLSKGCFGLKLDRIGSMSGLGC

SEQ ID NO: 51 - protein, 27 aa, synthetic construct
  REGION 1..27: note = CNP-27 K4P, K5R
  DISULFID 11..27: note = intrachain disulfide bond
GANPRGLSKGCFGLKLDRIGSMSGLGC

SEQ ID NO: 52 - protein, 27 aa, synthetic construct
  REGION 1..27: note = CNP-27 K4S, K5S
  DISULFID 11..27: note = intrachain disulfide bond
GANSSGLSKGCFGLKLDRIGSMSGLGC

SEQ ID NO: 53 - protein, 30 aa, synthetic construct
  REGION 1..30: note = GAN-CNP-27 K4P, K5R
  DISULFID 14..30: note = intrachain disulfide bond
GANGANPRGLSRGCFGLKLDRIGSMSGLGC

SEQ ID NO: 54 - protein, 27 aa, synthetic construct
  REGION 1..27: note = CNP-27 K4R, K5R, K9R
  DISULFID 11..27: note = intrachain disulfide bond
GANRRGLSRGCFGLKLDRIGSMSGLGC

SEQ ID NO: 55 - protein, 27 aa, synthetic construct
  REGION 1..27: note = CNP-27 K4R, K5R, K9R, M22N
  DISULFID 11..27: note = intrachain disulfide bond
GANRRGLSRGCFGLKLDRIGSNSGLGC

SEQ ID NO: 56 - protein, 28 aa, synthetic construct
  REGION 1..28: note = P-CNP-27 K4R, K5R, K9R
  DISULFID 12..28: note = intrachain disulfide bond
PGANRRGLSRGCFGLKLDRIGSMSGLGC

SEQ ID NO: 57 - protein, 28 aa, synthetic construct
  REGION 1..28: note = M-CNP-27 K4R, K5R, K9R
  DISULFID 12..28: note = intrachain disulfide bond
MGANRRGLSRGCFGLKLDRIGSMSGLGC

SEQ ID NO: 58 - protein, 38 aa, synthetic construct; identical to SEQ ID NO: 47
  REGION 1..38: note = Human Serum Albumine Fragment - CNP-27
  DISULFID 22..38: note = intrachain disulfide bond

SEQ ID NO: 59 - protein, 38 aa, synthetic construct
  REGION 1..38: note = Human Serum Albumine Fragment - CNP-27 M22N
  DISULFID 22..38: note = intrachain disulfide bond
GHKSEVAHRFKGANKKGLSKGCFGLKLDRIGSNSGLGC

SEQ ID NO: 60 - protein, 39 aa, synthetic construct
  REGION 1..39: note = methionine - Human Serum Albumine Fragment - CNP-27
  DISULFID 23..39: note = intrachain disulfide bond
MGHKSEVAHRFKGANKKGLSKGCFGLKLDRIGSMSGLGC

SEQ ID NO: 61 - protein, 39 aa, synthetic construct
  REGION 1..39: note = proline - Human Serum Albumine Fragment - CNP-27
  DISULFID 23..39: note = intrachain disulfide bond
PGHKSEVAHRFKGANKKGLSKGCFGLKLDRIGSMSGLGC

SEQ ID NO: 62 - protein, 26 aa, synthetic construct
  REGION 1..26: note = CNP-26
  DISULFID 10..26: note = intrachain disulfide bond
ANKKGLSKGCFGLKLDRIGSMSGLGC

SEQ ID NO: 63 - protein, 25 aa, synthetic construct
  REGION 1..25: note = CNP-25
  DISULFID 9..25: note = intrachain disulfide bond
NKKGLSKGCFGLKLDRIGSMSGLGC

SEQ ID NO: 64 - protein, 24 aa, synthetic construct
  REGION 1..24: note = CNP-24
  DISULFID 8..24: note = intrachain disulfide bond
KKGLSKGCFGLKLDRIGSMSGLGC

SEQ ID NO: 65 - protein, 23 aa, synthetic construct
  REGION 1..23: note = CNP-23
  DISULFID 7..23: note = intrachain disulfide bond
KGLSKGCFGLKLDRIGSMSGLGC

SEQ ID NO: 66 - protein, 23 aa, synthetic construct
  REGION 1..23: note = R-CNP-22
  DISULFID 7..23: note = intrachain disulfide bond
RGLSKGCFGLKLDRIGSMSGLGC

SEQ ID NO: 67 - protein, 24 aa, synthetic construct
  REGION 1..24: note = ER-CNP-22
  DISULFID 8..24: note = intrachain disulfide bond
ERGLSKGCFGLKLDRIGSMSGLGC

SEQ ID NO: 68 - protein, 23 aa, synthetic construct
  REGION 1..23: note = R-CNP-22 K4R
  DISULFID 7..23: note = intrachain disulfide bond
RGLSRGCFGLKLDRIGSMSGLGC

SEQ ID NO: 69 - protein, 24 aa, synthetic construct
  REGION 1..24: note = ER-CNP-22 4KR
  DISULFID 8..24: note = intrachain disulfide bond
ERGLSRGCFGLKLDRIGSMSGLGC

SEQ ID NO: 70 - protein, 24 aa, synthetic construct
  REGION 1..24: note = RR-CNP-22
  DISULFID 8..24: note = intrachain disulfide bond
RRGLSRGCFGLKLDRIGSMSGLGC

SEQ ID NO: 71 - protein, 37 aa, synthetic construct
  REGION 1..37: note = HRGP fragment - CNP-22
  DISULFID 21..37: note = intrachain disulfide bond
GHHSHEQHPHGANQQGLSKGCFGLKLDRIGSMSGLGC

SEQ ID NO: 72 - protein, 38 aa, synthetic construct
  REGION 1..38: note = HRGP fragment - CNP-22
  DISULFID 22..38: note = intrachain disulfide bond
GAHHPHEHDTHGANQQGLSKGCFGLKLDRIGSMSGLGC

SEQ ID NO: 73 - protein, 37 aa, synthetic construct
  REGION 1..37: note = HRGP fragment - CNP-22
  DISULFID 21..37: note = intrachain disulfide bond
GHHSHEQHPHGANPRGLSKGCFGLKLDRIGSMSGLGC

SEQ ID NO: 74 - protein, 36 aa, synthetic construct
  REGION 1..36: note = IgG1(FC) fragment - CNP-22
  DISULFID 20..36: note = intrachain disulfide bond
GQPREPQVYTLPPSGLSKGCFGLKLDRIGSMSGLGC

SEQ ID NO: 75 - protein, 39 aa, synthetic construct
  REGION 1..39: note = Human Serum Albumine - CNP-22
  DISULFID 23..39: note = intrachain disulfide bond
GQHKDDNPNLPRGANPRGLSKGCFGLKLDRIGSMSGLGC

SEQ ID NO: 76 - protein, 37 aa, synthetic construct
  REGION 1..37: note = Human Serum Albumine - CNP-22
  DISULFID 21..37: note = intrachain disulfide bond
GERAFKAWAVARLSQGLSKGCFGLKLDRIGSMSGLGC

SEQ ID NO: 77 - protein, 35 aa, synthetic construct
  REGION 1..35: note = osteocrin NPR C inhibitor fragment - CNP-22
  DISULFID 19..35: note = intrachain disulfide bond
FGIPMDRIGRNPRGLSKGCFGLKLDRIGSMSGLGC

SEQ ID NO: 78 - protein, 40 aa, synthetic construct [sequence omitted]

SEQ ID NO: 79 - protein, 37 aa, synthetic construct
  REGION 1..37: note = IgG1(FC) fragment - CNP-22 K4R
  DISULFID 21..37: note = intrachain disulfide bond
GQPREPQVYTGANQQGLSRGCFGLKLDRIGSMSGLGC

SEQ ID NO: 80 - protein, 36 aa, synthetic construct
  REGION 1..36: note = Human Serum Albumine fragment - CNP-22 K4R
  DISULFID 20..36: note = intrachain disulfide bond
GVPQVSTSTGANQQGLSRGCFGLKLDRIGSMSGLGC

SEQ ID NO: 81 - protein, 37 aa, synthetic construct
  REGION 1..37: note = fibronectin fragment - CNP-22 K4R
  DISULFID 21..37: note = intrachain disulfide bond
GQPSSSSQSTGANQQGLSRGCFGLKLDRIGSMSGLGC

SEQ ID NO: 82 - protein, 37 aa, synthetic construct
  REGION 1..37: note = fibronectin fragment - CNP-22 K4R
  DISULFID 21..37: note = intrachain disulfide bond
GQTHSSGTQSGANQQGLSRGCFGLKLDRIGSMSGLGC

SEQ ID NO: 83 - protein, 37 aa, synthetic construct
  REGION 1..37: note = fibronectin fragment - CNP-22 K4R
  DISULFID 21..37: note = intrachain disulfide bond
GSTGQWHSESGANQQGLSRGCFGLKLDRIGSMSGLGC

SEQ ID NO: 84 - protein, 37 aa, synthetic construct
  REGION 1..37: note = zinc finger fragment - CNP-22 K4R
  DISULFID 21..37: note = intrachain disulfide bond
GSSSSSSSSSGANQQGLSRGCFGLKLDRIGSMSGLGC

SEQ ID NO: 85 - protein, 21 aa, synthetic construct
  REGION 1..21: note = CNP-21
  DISULFID 5..21: note = intrachain disulfide bond
LSKGCFGLKLDRIGSMSGLGC

SEQ ID NO: 86 - protein, 20 aa, synthetic construct
  REGION 1..20: note = CNP-20
  DISULFID 4..20: note = intrachain disulfide bond
SKGCFGLKLDRIGSMSGLGC

SEQ ID NO: 87 - protein, 19 aa, synthetic construct
  REGION 1..19: note = CNP-19
  DISULFID 3..19: note = intrachain disulfide bond
KGCFGLKLDRIGSMSGLGC

SEQ ID NO: 88 - protein, 18 aa, synthetic construct
  REGION 1..18: note = CNP-18
  DISULFID 2..18: note = intrachain disulfide bond
GCFGLKLDRIGSMSGLGC

SEQ ID NO: 89 - protein, 17 aa, synthetic construct
  REGION 1..17: note = CNP-17
  DISULFID 1..17: note = intrachain disulfide bond
CFGLKLDRIGSMSGLGC

SEQ ID NO: 90 - protein, 32 aa, synthetic construct
  REGION 1..32: note = BNP fragment - CNP-17 - BNP- fragment
  DISULFID 10..26: note = intrachain disulfide bond
SPKMVQGSGCFGLKLDRIGSMSGLGCKVLRRH

SEQ ID NO: 91 - protein, 38 aa, synthetic construct; identical to SEQ ID NO: 27
  REGION 1..38: note = CNP-38 L1G
  DISULFID 22..38: note = intrachain disulfide bond

SEQ ID NO: 92 - protein, 37 aa, synthetic construct; identical to SEQ ID NO: 25
  REGION 1..37: note = Ac-CNP-37
  MOD_RES 1: note = ACETYLATION
  DISULFID 21..37: note = intrachain disulfide bond

SEQ ID NO: 93 - protein, 37 aa, synthetic construct
  REGION 1..37: note = CNP-37, X = K or R, with the provision that at least one X is R
  VARIANT 8: note = X = Lys, Arg, Pro, Ser or Gln, with the provision that at least one of amino acids 8, 10, 14, 15, 19 or 25 is selected from the group consisting of Arg, Pro, Ser and Gln
  VARIANT 10: note = X = Lys, Arg, Pro, Ser or Gln, with the provision that at least one of amino acids 8, 10, 14, 15, 19 or 25 is selected from the group consisting of Arg, Pro, Ser and Gln
  VARIANT 14: note = X = Lys, Arg, Pro, Ser or Gln, with the provision that at least one of amino acids 8, 10, 14, 15, 19 or 25 is selected from the group consisting of Arg, Pro, Ser and Gln
  VARIANT 15: note = X = Lys, Arg, Pro, Ser or Gln, with the provision that at least one of amino acids 8, 10, 14, 15, 19 or 25 is selected from the group consisting of Arg, Pro, Ser and Gln
  VARIANT 19: note = X = Lys, Arg, Pro, Ser or Gln, with the provision that at least one of amino acids 8, 10, 14, 15, 19 or 25 is selected from the group consisting of Arg, Pro, Ser and Gln
  DISULFID 21..37: note = intrachain disulfide bond
  VARIANT 25: note = X = Lys, Arg, Pro, Ser or Gln, with the provision that at least one of amino acids 8, 10, 14, 15, 19 or 25 is selected from the group consisting of Arg, Pro, Ser and Gln
QEHPNARXYXGANXXGLSXGCFGLXLDRIGSMSGLGC

SEQ ID NO: 94 - protein, 37 aa, synthetic construct
  REGION 1..37: note = mutated CNP-37
  VARIANT 14: note = X is selected from the group consising of Lys, Arg, Pro, Ser and Gln, with the provision that at least one of the amino acids at position 14, 15, 19 and 25 is selected from the group consisting of Arg, Pro, Ser and Gln
  VARIANT 15: note = X is selected from the group consising of Lys, Arg, Pro, Ser and Gln, with the provision that at least one of the amino acids at position 14, 15, 19 and 25 is selected from the group consisting of Arg, Pro, Ser and Gln
  VARIANT 19: note = X is selected from the group consising of Lys, Arg, Pro, Ser and Gln, with the provision that at least one of the amino acids at position 14, 15, 19 and 25 is selected from the group consisting of Arg, Pro, Ser and Gln
  DISULFID 21..37: note = intrachain disulfide bond
  VARIANT 25: note = X is selected from the group consising of Lys, Arg, Pro, Ser and Gln, with the provision that at least one of the amino acids at position 14, 15, 19 and 25 is selected from the group consisting of Arg, Pro, Ser and Gln
QEHPNARKYKGANXXGLSXGCFGLXLDRIGSMSGLGC

SEQ ID NO: 95 - protein, 37 aa, synthetic construct
  REGION 1..37: note = mutated CNP-37
  VARIANT 14..15: note = X X is selected from the group consisting of Lys Arg, Arg Lys, Lys Pro, Pro Lys, Ser Ser, Arg Ser, Ser Arg, Gln Lys, Gln Arg, Lys Gln, Arg Gln, Arg Arg and Gln Gln
  DISULFID 21..37: note = intrachain disulfide bond
QEHPNARKYKGANXXGLSKGCFGLKLDRIGSMSGLGC

SEQ ID NO: 96 - protein, 15 aa, Homo sapiens
FGLKLDRIGSMSGLG

SEQ ID NO: 97 - protein, 38 aa, Synthetic construct
  DISULFID 22..38: note = intrachain disulfide bond
  REGION 1..38: note = CNP-38 N6Q, N14Q
LQEHPQARKYKGAQKKGLSKGCFGLKLDRIGSMSGLGC

SEQ ID NO: 98 - protein, 39 aa, Synthetic construct
  DISULFID 23..39: note = intrachain disulfide bond
PGQEHPQARRYRGAQRRGLSRGCFGLKLDRIGSMSGLGC

SEQ ID NO: 99 - protein, 39 aa, synthetic construct
  DISULFID 23..39: note = intrachain disulfide bond
PGQEHPNARRYRGANRRGLSRGCFGLKLDRIGSMSGLGC

SEQ ID NO: 100 - protein, 39 aa, synthetic construct
  DISULFID 23..39: note = intrachain disulfide bond
PGQEHPQARKYKGAQKKGLSKGCFGLKLDRIGSMSGLGC

SEQ ID NO: 101 - protein, 39 aa, synthetic construct; identical to SEQ ID NO: 98
  DISULFID 23..39: note = intrachain disulfide bond
  MOD_RES 1: note = acetylation

SEQ ID NO: 102 - protein, 39 aa, synthetic construct; identical to SEQ ID NO: 30
  DISULFID 23..39: note = intrachain disulfide bond
  MOD_RES 1: note = acetylation
  MOD_RES 39: note = C-terminal NH2

SEQ ID NO: 103 - protein, 39 aa, synthetic construct; identical to SEQ ID NO: 99
  DISULFID 23..39: note = intrachain disulfide bond
  MOD_RES 1: note = acetylation

SEQ ID NO: 104 - protein, 39 aa, synthetic construct; identical to SEQ ID NO: 99
  DISULFID 23..39: note = intrachain disulfide bond
  MOD_RES 1: note = acetylation
  MOD_RES 39: note = C-terminal NH2

SEQ ID NO: 105 - protein, 39 aa, synthetic construct; identical to SEQ ID NO: 98
  DISULFID 23..39: note = intrachain disulfide bond
  MOD_RES 1: note = acetylation
  MOD_RES 39: note = C-terminal NH2